SEQUENCE LISTING (1401 sequences)

SEQ ID NO: 1 - DNA, 19 bp, Artificial Sequence
  CNAG 03132 5 flanking region primer 1
gcgtggcatttagagagtg

SEQ ID NO: 2 - DNA, 38 bp, Artificial Sequence
  CNAG 03132 5 flanking region primer 2
tcactggccgtcgttttacgagcgtattggatgaggag

SEQ ID NO: 3 - DNA, 39 bp, Artificial Sequence
  CNAG 03132 3 flanking region primer 1
catggtcatagctgtttcctgaaacctcgctttgagacg

SEQ ID NO: 4 - DNA, 18 bp, Artificial Sequence
  CNAG 03132 3 flanking region primer 2
tgacgaagcgacagaatc

SEQ ID NO: 5 - DNA, 20 bp, Artificial Sequence
  CNAG 03132 diagnostic screening primer, pairing with B79
cccaaacactattatcgcag

SEQ ID NO: 6 - DNA, 18 bp, Artificial Sequence
  CNAG 03132 Southern blot probe primer
tactcgtatgaatcggcg

SEQ ID NO: 7 - DNA, 20 bp, Artificial Sequence
  NAT 5 STM primer
tgctagagggcgggagagtt

SEQ ID NO: 8 - DNA, 24 bp, Artificial Sequence
  STM common primer
gcatgccctgcccctaagaattcg

SEQ ID NO: 9 - DNA, 20 bp, Artificial Sequence
  CNAG 05380 5 flanking region primer 1
cctactgcttggcacattac

SEQ ID NO: 10 - DNA, 35 bp, Artificial Sequence
  CNAG 05380 5 flanking region primer 2
ctggccgtcgttttaccgtcgtttctttttgggag

SEQ ID NO: 11 - DNA, 37 bp, Artificial Sequence
  CNAG 05380 3 flanking region primer 1
gtcatagctgtttcctgtccatctccagtctgtgaac

SEQ ID NO: 12 - DNA, 20 bp, Artificial Sequence
  CNAG 05380 3 flanking region primer 2
cgcttcttaggacctcactc

SEQ ID NO: 13 - DNA, 20 bp, Artificial Sequence
  CNAG 05380 diagnostic screening primer, pairing with B79
tctgaaggcggaatctggac

SEQ ID NO: 14 - DNA, 24 bp, Artificial Sequence
  CNAG 05380 Southern blot probe primer
ggagcaaggaactaaagatggtag

SEQ ID NO: 15 - DNA, 20 bp, Artificial Sequence
  NAT 5 STM primer
tgctagagggcgggagagtt

SEQ ID NO: 16 - DNA, 24 bp, Artificial Sequence
  STM common primer
gcatgccctgcccctaagaattcg

SEQ ID NO: 17 - DNA, 20 bp, Artificial Sequence
  CNAG 06719 5 flanking region primer 1
tcgccaactgaacaaacccc

SEQ ID NO: 18 - DNA, 36 bp, Artificial Sequence
  CNAG 06719 5 flanking region primer 2
ctggccgtcgttttactcccgcctaacactttcgtg

SEQ ID NO: 19 - DNA, 36 bp, Artificial Sequence
  CNAG 06719 3 flanking region primer 1
gtcatagctgtttcctgtgtcaagccataaagagcc

SEQ ID NO: 20 - DNA, 20 bp, Artificial Sequence
  CNAG 06719 3 flanking region primer 2
ctgatagttttgccacttgc

SEQ ID NO: 21 - DNA, 22 bp, Artificial Sequence
  CNAG 06719 diagnostic screening primer, pairing with B79
aataccctccttacttccccgc

SEQ ID NO: 22 - DNA, 18 bp, Artificial Sequence
  CNAG 06719 Southern blot probe primer
gtaaaagtttgggcgtgg

SEQ ID NO: 23 - DNA, 20 bp, Artificial Sequence
  NAT 5 STM primer
tgctagagggcgggagagtt

SEQ ID NO: 24 - DNA, 24 bp, Artificial Sequence
  STM common primer
gcatgccctgcccctaagaattcg

SEQ ID NO: 25 - DNA, 20 bp, Artificial Sequence
  CNAG 04093 5 flanking region primer 1
tccaggcgtattcatcccag

SEQ ID NO: 26 - DNA, 38 bp, Artificial Sequence
  CNAG 04093 5 flanking region primer 2
ctggccgtcgttttacaaggattgtgacctcaggctcg

SEQ ID NO: 27 - DNA, 36 bp, Artificial Sequence
  CNAG 04093 3 flanking region primer 1
gtcatagctgtttcctgatgtttctcctccagagcc

SEQ ID NO: 28 - DNA, 19 bp, Artificial Sequence
  CNAG 04093 3 flanking region primer 2
gcaagaaccaaatgacagc

SEQ ID NO: 29 - DNA, 24 bp, Artificial Sequence
  CNAG 04093 diagnostic screening primer, pairing with B79
atcattcctctgacaagtcgggcg

SEQ ID NO: 30 - DNA, 19 bp, Artificial Sequence
  CNAG 04093 Southern blot probe primer
atgtgtcctggatggcttg

SEQ ID NO: 31 - DNA, 20 bp, Artificial Sequence
  NAT 5 STM primer
tgctagagggcgggagagtt

SEQ ID NO: 32 - DNA, 24 bp, Artificial Sequence
  STM common prither
gcatgccctgcccctaagaattcg

SEQ ID NO: 33 - DNA, 18 bp, Artificial Sequence
  CNAG 04518 5 flanking region primer 1
tgtcctttcttgtgaccg

SEQ ID NO: 34 - DNA, 39 bp, Artificial Sequence
  CNAG 04518 5 flanking region primer 2
tcactggccgtcgttttaccagtaacgaagaacgcaatc

SEQ ID NO: 35 - DNA, 40 bp, Artificial Sequence
  CNAG 04518 3 flanking region primer 1
catggtcatagctgtttcctgcacaagccacgattattgc

SEQ ID NO: 36 - DNA, 20 bp, Artificial Sequence
  CNAG 04518 3 flanking region primer 2
tgtatctgtgtgtggactgc

SEQ ID NO: 37 - DNA, 18 bp, Artificial Sequence
  CNAG 04518 diagnostiC screening primer, pairing With B79
acacaaagggaggaatgc

SEQ ID NO: 38 - DNA, 19 bp, Artificial Sequence
  CNAG 04518 Southern blot probe primer
catacactgtgccacgaac

SEQ ID NO: 39 - DNA, 20 bp, Artificial Sequence
  NAT 6 STM primer
atagctaccacacgatagct

SEQ ID NO: 40 - DNA, 24 bp, Artificial Sequence
  STM common primer
gcatgccctgcccctaagaattcg

SEQ ID NO: 41 - DNA, 20 bp, Artificial Sequence
  CNAG 01858 5 flanking region primer 1
tcttcaggaacataggatgg

SEQ ID NO: 42 - DNA, 38 bp, Artificial Sequence
  CNAG 01858 5 flanking region primer 2
tcactggccgtcgttttacgagattgttgggaggattg

SEQ ID NO: 43 - DNA, 39 bp, Artificial Sequence
  CNAG 01858 3 flanking region primer 1
catggtcatagctgtttcctggggcgaataaggtttctg

SEQ ID NO: 44 - DNA, 18 bp, Artificial Sequence
  CNAG 01858 3 flanking region primer 2
tgttttcgctcatccgtc

SEQ ID NO: 45 - DNA, 19 bp, Artificial Sequence
  CNAG 01858 diagnostic screening primer, pairing with B79
aacacagcggaatgacctc

SEQ ID NO: 46 - DNA, 18 bp, Artificial Sequence
  CNAG 01858 Southern blot probe primer
cacttggttcattgctgg

SEQ ID NO: 47 - DNA, 20 bp, Artificial Sequence
  NAT 43 STM primer
ccagctaccaatcacgctac

SEQ ID NO: 48 - DNA, 24 bp, Artificial Sequence
  STM common primer
gcatgccctgcccctaagaattcg

SEQ ID NO: 49 - DNA, 22 bp, Artificial Sequence
  CNAG 02305 5 flanking region primer 1
ccagaaggatgtgatactgagg

SEQ ID NO: 50 - DNA, 36 bp, Artificial Sequence
  CNAG 02305 5 flanking region primer 2
ctggccgtcgttttacgaaatcgttctgaaactgcg

SEQ ID NO: 51 - DNA, 39 bp, Artificial Sequence
  CNAG 02305 3 flanking region primer 1
gtcatagctgtttcctgatcgttcacaggctcaaggctg

SEQ ID NO: 52 - DNA, 22 bp, Artificial Sequence
  CNAG 02305 3 flanking region primer 2
gaggtcattagagggtatttgc

SEQ ID NO: 53 - DNA, 19 bp, Artificial Sequence
  CNAG 02305 diagnostic screening primer, pairing with 879
gcgtggcgaatacatacag

SEQ ID NO: 54 - DNA, 19 bp, Artificial Sequence
  CNAG 02305 Southern blot probe primer
aggtgctacaaagactcgg

SEQ ID NO: 55 - DNA, 24 bp, Artificial Sequence
  NAT 58 STM primer
cgcaaaatcactagccctatagcg

SEQ ID NO: 56 - DNA, 24 bp, Artificial Sequence
  STM common primer
gcatgccctgcccctaagaattcg

SEQ ID NO: 57 - DNA, 19 bp, Artificial Sequence
  CNAG 06156 5 flanking region primer 1
aagatagcacctccgacag

SEQ ID NO: 58 - DNA, 41 bp, Artificial Sequence
  CNAG 06156 5 flanking region primer 2
gctcactggccgtcgttttacgctgttgcgaaagataaagg

SEQ ID NO: 59 - DNA, 41 bp, Artificial Sequence
  CNAG 06156 3 flanking region primer 1
catggtcatagctgtttcctgccaggaatgaactaccaagg

SEQ ID NO: 60 - DNA, 19 bp, Artificial Sequence
  CNAG 06156 3 flanking region primer 2
ttgaagcaggactctccac

SEQ ID NO: 61 - DNA, 19 bp, Artificial Sequence
  CNAG 06156 diagnostic screening primer, pairing with B79
tgcttgtcgtttggttagg

SEQ ID NO: 62 - DNA, 19 bp, Artificial Sequence
  CNAG 06156 Southern blot probe primer
ggtgaaaatgacggtccag

SEQ ID NO: 63 - DNA, 24 bp, Artificial Sequence
  NAT 119 STM primer
ctccccacataaagagagctaaac

SEQ ID NO: 64 - DNA, 24 bp, Artificial Sequence
  STM common primer
gcatgccctgcccctaagaattcg

SEQ ID NO: 65 - DNA, 21 bp, Artificial Sequence
  CNAG 06188 5 flanking region primer 1
ctcttgtcttctgcctattcc

SEQ ID NO: 66 - DNA, 35 bp, Artificial Sequence
  CNAG 06188 5 flanking region primer 2
ctggccgtcgttttacggacatagtgaaactgggg

SEQ ID NO: 67 - DNA, 38 bp, Artificial Sequence
  CNAG 06188 3 flanking region primer 1
gtcatagctgtttcctgtcatcctccgactctaaactg

SEQ ID NO: 68 - DNA, 20 bp, Artificial Sequence
  CNAG 06188 3 flanking region primer 2
gttttcctacttttgtgggc

SEQ ID NO: 69 - DNA, 20 bp, Artificial Sequence
  CNAG 06188 diagnostic screening primer, pairing with B79
agaagacgactgtggtaggc

SEQ ID NO: 70 - DNA, 20 bp, Artificial Sequence
  CNAG 06188 Southern blot probe primer
gagtcagcagcagaagattc

SEQ ID NO: 71 - DNA, 24 bp, Artificial Sequence
  NAT 122 STM primer
acagctccaaacctcgctaaacag

SEQ ID NO: 72 - DNA, 24 bp, Artificial Sequence
  STM common prlMer
gcatgccctgcccctaagaattcg

SEQ ID NO: 73 - DNA, 19 bp, Artificial Sequence
  CNAG 05785 5 flanking region primer 1
catttctacttggttgccg

SEQ ID NO: 74 - DNA, 36 bp, Artificial Sequence
  CNAG 05785 5 flanking region primer 2
ctggccgtcgttttacaggagaaagtgatggtcggc

SEQ ID NO: 75 - DNA, 39 bp, Artificial Sequence
  CNAG 05785 3 flanking region primer 1
gtcatagctgtttcctgttatcgtgagccttcaagcccc

SEQ ID NO: 76 - DNA, 21 bp, Artificial Sequence
  CNAG 05785 3 flanking region primer 2
ccattgtagatgtcggttacg

SEQ ID NO: 77 - DNA, 22 bp, Artificial Sequence
  CNAG 05785 diagnostic screening primer; pairing with B79
ttgcggtctttgtcccttgtgg

SEQ ID NO: 78 - DNA, 24 bp, Artificial Sequence
  CNAG 05785 Southern blot probe primer
ttctccacttgctcgcctatcctc

SEQ ID NO: 79 - DNA, 24 bp, Artificial Sequence
  NAT 125 STM primer
cgctacagccagcgcgcgcaagcg

SEQ ID NO: 80 - DNA, 24 bp, Artificial Sequence
  STM common primer
gcatgccctgcccctaagaattcg

SEQ ID NO: 81 - DNA, 20 bp, Artificial Sequence
  CNAG 05019 5 flanking region primer 1
aaccaccacaatgtcttcag

SEQ ID NO: 82 - DNA, 40 bp, Artificial Sequence
  CNAG 05019 5 flanking region primer 2
gctcactggccgtcgttttacagacccttgtttggttctg

SEQ ID NO: 83 - DNA, 41 bp, Artificial Sequence
  CNAG 05019 3 flanking region primer 1
catggtcatagctgtttcctgagtggagagggagagcaatg

SEQ ID NO: 84 - DNA, 18 bp, Artificial Sequence
  CNAG 05019 3 flanking region primer 2
tttacgaagagcagcgac

SEQ ID NO: 85 - DNA, 18 bp, Artificial Sequence
  CNAG 05019 diagnostic screening prither, pairing with 879
agatagcaacggctttgg

SEQ ID NO: 86 - DNA, 20 bp, Artificial Sequence
  CNAG 05019 Southern blot probe primer
cgcaaaagggaagatggata

SEQ ID NO: 87 - DNA, 20 bp, Artificial Sequence
  NAT 150 STM primer
acatacacccccatcccccc

SEQ ID NO: 88 - DNA, 24 bp, Artificial Sequence
  STM common primer
gcatgccctgcccctaagaattcg

SEQ ID NO: 89 - DNA, 18 bp, Artificial Sequence
  CNAG 02877 5 flanking region primer 1
gtttaccatttcaccgcc

SEQ ID NO: 90 - DNA, 39 bp, Artificial Sequence
  CNAG 02877 5 flanking region primer 2
tcactggccgtcgttttactgatgaggatacattcggag

SEQ ID NO: 91 - DNA, 41 bp, Artificial Sequence
  CNAG 02877 3 flanking region primer 1
catggtcatagctgtttcctgggcttcaacaatcaccattc

SEQ ID NO: 92 - DNA, 18 bp, Artificial Sequence
  CNAG 02877 3 flanking region primer 2
acgaaaagcggatagcag

SEQ ID NO: 93 - DNA, 20 bp, Artificial Sequence
  CNAG 02877 diagnostic screening primer, pairing with B79
agccttattatgaccaaccc

SEQ ID NO: 94 - DNA, 20 bp, Artificial Sequence
  CNAG 02877 Southern blot probe primer
caagttcctttgcctacacc

SEQ ID NO: 95 - DNA, 21 bp, Artificial Sequence
  NAT 159 STM primer
acgcaccagacacacaaccag

SEQ ID NO: 96 - DNA, 24 bp, Artificial Sequence
  STM common primer
gcatgccctgcccctaagaattcg

SEQ ID NO: 97 - DNA, 20 bp, Artificial Sequence
  CNAG 00039 5 flanking region primer 1
tcactctttcggggcatcag

SEQ ID NO: 98 - DNA, 38 bp, Artificial Sequence
  CNAG 00039 5 flanking region primer 2
ctggccgtcgttttacctaactggggatgaaatgtctg

SEQ ID NO: 99 - DNA, 39 bp, Artificial Sequence
  CNAG 00039 3 flanking region primer 1
gtcatagctgtttcctgggggaagagcagaaagagtaac

SEQ ID NO: 100 - DNA, 22 bp, Artificial Sequence
  CNAG 00039 3 flanking region primer 2
gcaatcgtaaacaacacccttg

SEQ ID NO: 101 - DNA, 20 bp, Artificial Sequence
  CNAG 00039 diagnostic screening primer, pairing with B79
cgtctgtcgtttatcggttg

SEQ ID NO: 102 - DNA, 19 bp, Artificial Sequence
  CNAG 00039 Southern blot probe primer
accctgtaacatttgtccg

SEQ ID NO: 103 - DNA, 19 bp, Artificial Sequence
  CNAG 00039 Southern blot probe primer
acttacccttcacgccaag

SEQ ID NO: 104 - DNA, 20 bp, Artificial Sequence
  NAT 177 STM primer
caccaactccccatctccat

SEQ ID NO: 105 - DNA, 24 bp, Artificial Sequence
  STM common primer
gcatgcdctgcccctaagaattcg

SEQ ID NO: 106 - DNA, 20 bp, Artificial Sequence
  CNAG 00039 5 flanking region primer 1
agacattgggcttgacggtg

SEQ ID NO: 107 - DNA, 39 bp, Artificial Sequence
  CNAG 00039 5 flanking region primer 2
tcactggccgtcgttttacacgagttggagaggggttac

SEQ ID NO: 108 - DNA, 39 bp, Artificial Sequence
  CNAG 00039 3 flanking region primer 1
catggtcatagctgtttcctgtagaaaggaatcggggac

SEQ ID NO: 109 - DNA, 20 bp, Artificial Sequence
  CNAG 00039 3 flanking region primer 2
ggagaacgaaaacaaagtgc

SEQ ID NO: 110 - DNA, 19 bp, Artificial Sequence
  CNAG 00039 diagnostic screening primer, pairing with B79
ggttcttgtttatgccctg

SEQ ID NO: 111 - DNA, 20 bp, Artificial Sequence
  CNAG 00039 Southern blot probe primer
aacgagggtagattgcgtcg

SEQ ID NO: 112 - DNA, 20 bp, Artificial Sequence
  NAT 177 STM primer
caccaactccccatctccat

SEQ ID NO: 113 - DNA, 24 bp, Artificial Sequence
  STM common primer
gcatgccctgcccctaagaattcg

SEQ ID NO: 114 - DNA, 19 bp, Artificial Sequence
  CNAG 01014 5 flanking region primer 1
tatcgccgacatctctcag

SEQ ID NO: 115 - DNA, 38 bp, Artificial Sequence
  CNAG 01014 5 flanking region primer 2
tcactggccgtcgttttactttaggacggaggggattg

SEQ ID NO: 116 - DNA, 39 bp, Artificial Sequence
  CNAG 01014 3 flanking region primer 1
catggtcatagctgtttcctggagccgaatcaggaaatc

SEQ ID NO: 117 - DNA, 19 bp, Artificial Sequence
  CNAG 01014 3 flanking region primer 2
cgcaggcttttctactctc

SEQ ID NO: 118 - DNA, 30 bp, Artificial Sequence
  CNAG 01014 diagnostic screening primer, pairing with B79
cgcgcggccgcgaaaacctccacagagcag

SEQ ID NO: 119 - DNA, 19 bp, Artificial Sequence
  CNAG 01014 Southern blot probe primer
ttgcttgctgtctcgtaac

SEQ ID NO: 120 - DNA, 18 bp, Artificial Sequence
  CNAG 01014 Southern blot probe primer
gatttcctgattcggctc

SEQ ID NO: 121 - DNA, 20 bp, Artificial Sequence
  CTAGAGCCCGCCACAACGCT
ctagagcccgccacaacgct

SEQ ID NO: 122 - DNA, 24 bp, Artificial Sequence
  STM common primer
gcatgccctgcccctaagaattcg

SEQ ID NO: 123 - DNA, 21 bp, Artificial Sequence
  CNAG 06762 5 flanking region primer 1
tgggtgagagaaagatgtcag

SEQ ID NO: 124 - DNA, 39 bp, Artificial Sequence
  CNAG 06762 5 flanking region primer 2
tcactggccgtcgttttactgtgagtggttgtgctgtgg

SEQ ID NO: 125 - DNA, 41 bp, Artificial Sequence
  CNAG 06762 3 flanking region primer 1
catggtcatagctgtttcctgggctcataggtgttgtaacg

SEQ ID NO: 126 - DNA, 20 bp, Artificial Sequence
  CNAG 06762 3 flanking region primer 2
agtattcggtggtgacaaag

SEQ ID NO: 127 - DNA, 21 bp, Artificial Sequence
  CNAG 06762 diagnostic screening primer, pairing with B79
tgggtgagagaaagatgtcag

SEQ ID NO: 128 - DNA, 19 bp, Artificial Sequence
  CNAG 06762 Southern blot probe primer
tgttccaccccttgctaac

SEQ ID NO: 129 - DNA, 20 bp, Artificial Sequence
  NAT 218 STM primer
ctccacatccatcgctccaa

SEQ ID NO: 130 - DNA, 24 bp, Artificial Sequence
  STM common primer
gcatgccctgcccctaagaattcg

SEQ ID NO: 131 - DNA, 18 bp, Artificial Sequence
  CNAG 04630 5 flanking region primer 1
atggaaggactttgacgg

SEQ ID NO: 132 - DNA, 40 bp, Artificial Sequence
  CNAG 04630 5 flanking region primer 2
tcactggccgtcgttttactgactcacacttgattctccc

SEQ ID NO: 133 - DNA, 40 bp, Artificial Sequence
  CNAG 04630_3 flanking region primer 1
gatggtcatagctgtttcctgctcattttccctttcgtcg

SEQ ID NO: 134 - DNA, 18 bp, Artificial Sequence
  CNAG 04630 3 flanking region primer 2
taaaggaatgcggctgag

SEQ ID NO: 135 - DNA, 19 bp, Artificial Sequence
  CNAG 04630 diagnostic screening primer, pairing with B79
ccaacagcaacttcgtttc

SEQ ID NO: 136 - DNA, 21 bp, Artificial Sequence
  CNAG 04630 Southern blot probe primer
tctcttctccttggaactgag

SEQ ID NO: 137 - DNA, 20 bp, Artificial Sequence
  NAT 218 STM primer
ctccacatccatcgctccaa

SEQ ID NO: 138 - DNA, 24 bp, Artificial Sequence
  STM common primer
gcatgccctgcccctaagaattcg

SEQ ID NO: 139 - DNA, 19 bp, Artificial Sequence
  CNAG 03401 5 flanking region primer 1
ttcctcgtccaagtaaacc

SEQ ID NO: 140 - DNA, 41 bp, Artificial Sequence
  CNAG 03401 5 flanking region primer 2
gctcactggccgtcgttttacaatctcgcaaaccgcacacg

SEQ ID NO: 141 - DNA, 43 bp, Artificial Sequence
  CNAG 03401 3 flanking region printer 1
catggtcatagctgtttcctgagcgcgaacagggaaatacacc

SEQ ID NO: 142 - DNA, 18 bp, Artificial Sequence
  CNAG 03401 3 flanking region primer 2
tcgtgtaggaatgggtag

SEQ ID NO: 143 - DNA, 20 bp, Artificial Sequence
  CNAG 03401 diagnostic screening primer, pairing with B79
tggtctccccgacgatttac

SEQ ID NO: 144 - DNA, 19 bp, Artificial Sequence
  CNAG 03401 Southern blot probe primer
tatggtcaggaggaagcag

SEQ ID NO: 145 - DNA, 20 bp, Artificial Sequence
  NAT 220 STM primer
cagatctcgaacgataccca

SEQ ID NO: 146 - DNA, 24 bp, Artificial Sequence
  STM common primer
gcatgccctgcccctaagaattcg

SEQ ID NO: 147 - DNA, 22 bp, Artificial Sequence
  CNAG 04594 5 flanking region primer 1
tggcatcgtgctgttacaagtc

SEQ ID NO: 148 - DNA, 45 bp, Artificial Sequence
  CNAG 04594 5 flanking region primer 2
gctcactggccgtcgttttacatttgcggatggacctgccttagg

SEQ ID NO: 149 - DNA, 41 bp, Artificial Sequence
  CNAG 04594 3 flanking region primer 1
catggtcatagctgtttcctgatcaggattcaggacacgac

SEQ ID NO: 150 - DNA, 18 bp, Artificial Sequence
  CNAG 04594 3 flanking region primer 2
tttggttctctctcccac

SEQ ID NO: 151 - DNA, 19 bp, Artificial Sequence
  CNAG 04594 diagndStic screening primer, pairing with B79
attgcttgagaccagcgtc

SEQ ID NO: 152 - DNA, 18 bp, Artificial Sequence
  CNAG 04594 Southern blot probe primer
acggttgctttgctatgc

SEQ ID NO: 153 - DNA, 20 bp, Artificial Sequence
  NAT 220 STM primer
cagatctcgaacgataccca

SEQ ID NO: 154 - DNA, 24 bp, Artificial Sequence
  STM common primer
gcatgccctgcccctaagaattcg

SEQ ID NO: 155 - DNA, 19 bp, Artificial Sequence
  CNAG 07901 5 flanking region primer 1
actcaccttcccgctttag

SEQ ID NO: 156 - DNA, 34 bp, Artificial Sequence
  CNAG 07901 5 flanking region primer 2
ctggccgtcgttttacgtttgagttgaggttgcg

SEQ ID NO: 157 - DNA, 36 bp, Artificial Sequence
  CNAG 07901 3 flanking region primer 1
gtcatagctgtttcctgagaatgttgatggtagcgg

SEQ ID NO: 158 - DNA, 19 bp, Artificial Sequence
  CNAG 07901 3 flanking region primer 2
tccaccgaagaaactgaag

SEQ ID NO: 159 - DNA, 18 bp, Artificial Sequence
  CNAG 07901 diagnostic screening primer, pairing with B79
gggatttggatacgcttg

SEQ ID NO: 160 - DNA, 18 bp, Artificial Sequence
  CNAG 07901 Southern blot probe primer
ttgcggtgggaaagtatc

SEQ ID NO: 161 - DNA, 20 bp, Artificial Sequence
  NAT 225 STM primer
ccatagaactagctaaagca

SEQ ID NO: 162 - DNA, 24 bp, Artificial Sequence
  STM common primer
gcatgccctgcccctaagaattcg

SEQ ID NO: 163 - DNA, 19 bp, Artificial Sequence
  CNAG 04588 5 flanking region primer 1
aaaagaggtccgatgagtg

SEQ ID NO: 164 - DNA, 40 bp, Artificial Sequence
  CNAG 04588 5 flanking region primer 2
gctcactggccgtcgttttacgtgaatgataaaggcgacc

SEQ ID NO: 165 - DNA, 39 bp, Artificial Sequence
  CNAG 04588 3 flanking region primer 1
catggtcatagctgtttcctgtgctgtttcactatccgc

SEQ ID NO: 166 - DNA, 19 bp, Artificial Sequence
  CNAG 04588 3 flanking region primer 2
cgaaagcgactggagtaac

SEQ ID NO: 167 - DNA, 20 bp, Artificial Sequence
  CNAG 04588 diagnostic screening primer, pairing with B79
gaagatgctgaagcgtattc

SEQ ID NO: 168 - DNA, 18 bp, Artificial Sequence
  CNAG 04588 Southern blot probe primer
acacttatccgccattcc

SEQ ID NO: 169 - DNA, 20 bp, Artificial Sequence
  NAT 225 STM primer
ccatagaactagctaaagca

SEQ ID NO: 170 - DNA, 24 bp, Artificial Sequence
  STM common primer
gcatgccctgcccctaagaattcg

SEQ ID NO: 171 - DNA, 18 bp, Artificial Sequence
  CNAG 03998 5 flanking region primer 1
gccattgtgaaagcatag

SEQ ID NO: 172 - DNA, 43 bp, Artificial Sequence
  CNAG 03998 5 flanking region primer 2
gctcactggccgtcgttttacacctgtctatctcactgtccag

SEQ ID NO: 173 - DNA, 39 bp, Artificial Sequence
  CNAG 03998 3 flanking region primer 1
catggtcatagctgtttcctgtgttcagcagcaaaaggc

SEQ ID NO: 174 - DNA, 18 bp, Artificial Sequence
  CNAG 03998 3 flanking region primer 2
gcggagagaaggattatc

SEQ ID NO: 175 - DNA, 20 bp, Artificial Sequence
  CNAG 03998 diagnostic screening primer, pairing with B79
gctgacaacttggaatcatc

SEQ ID NO: 176 - DNA, 20 bp, Artificial Sequence
  CNAG 03998 Southern blot probe primer
gggcttctcttcttcttcac

SEQ ID NO: 177 - DNA, 20 bp, Artificial Sequence
  NAT 234 STM primer
ccagccagcactccgatata

SEQ ID NO: 178 - DNA, 24 bp, Artificial Sequence
  STM common primer
gcatgccctgcccctaagaattcg

SEQ ID NO: 179 - DNA, 20 bp, Artificial Sequence
  CNAG 04804 5 flanking region primer 1
taggtagttcctgctcgtgg

SEQ ID NO: 180 - DNA, 40 bp, Artificial Sequence
  CNAG 04804 5 flanking region primer 2
tcactggccgtcgttttaccatcttgtcctgtaatgaggc

SEQ ID NO: 181 - DNA, 39 bp, Artificial Sequence
  CNAG 04804 3 flanking region primer 1
catggtcatagctgtttcctggattggagggcgatttag

SEQ ID NO: 182 - DNA, 20 bp, Artificial Sequence
  CNAG 04804 3 flanking region primer 2
caacaaggtccaagaaactg

SEQ ID NO: 183 - DNA, 21 bp, Artificial Sequence
  CNAG 04804 diagnostic screening primer, pairing with B79
agaggactgagacttttgtcg

SEQ ID NO: 184 - DNA, 19 bp, Artificial Sequence
  CNAG 04804 Southern blot probe primer
cgaaggggaactggttatc

SEQ ID NO: 185 - DNA, 20 bp, Artificial Sequence
  NAT 240 STM primer
ggtgttggatcggggtggat

SEQ ID NO: 186 - DNA, 24 bp, Artificial Sequence
  STM common primer
gcatgccctgcccctaagaattcg

SEQ ID NO: 187 - DNA, 20 bp, Artificial Sequence
  CNAG 06134 5 flanking region primer 1
gtttgaggctggtaaaaagg

SEQ ID NO: 188 - DNA, 39 bp, Artificial Sequence
  CNAG 06134 5 flanking region primer 2
gctcactggccgtcgttttacatggggaatgaaagcgtg

SEQ ID NO: 189 - DNA, 41 bp, Artificial Sequence
  CNAG 06134 3 flanking region primer 1
catggtcatagctgtttcctgaaggggcgagagtagttcag

SEQ ID NO: 190 - DNA, 21 bp, Artificial Sequence
  CNAG 06134 3 flanking region primer 2
gactgtaaaggagggdataag

SEQ ID NO: 191 - DNA, 19 bp, Artificial Sequence
  CNAG 06134 diagnostic screening primer, pairing with B79
aactcttctcagccttcgg

SEQ ID NO: 192 - DNA, 19 bp, Artificial Sequence
  CNAG 06134 Southern blot probe primer
cgttctccgtcttgatagc

SEQ ID NO: 193 - DNA, 21 bp, Artificial Sequence
  NAT 295 STM primer
acacctacatcaaaccctccc

SEQ ID NO: 194 - DNA, 24 bp, Artificial Sequence
  STM common primer
gcatgccctgcccctaagaattcg

SEQ ID NO: 195 - DNA, 19 bp, Artificial Sequence
  CNAG 04798 5 flanking region primer 1
caatgattaggcttgcgtc

SEQ ID NO: 196 - DNA, 38 bp, Artificial Sequence
  CNAG 04798 5 flanking region primer 2
tcactggccgtcgttttactgttgttgggcagtggtag

SEQ ID NO: 197 - DNA, 39 bp, Artificial Sequence
  CNAG 04798 3 flanking region primer 1
catggtcatagctgtttcctgtttagggaagaggaagcg

SEQ ID NO: 198 - DNA, 18 bp, Artificial Sequence
  CNAG 04798 3 flanking region primer 2
tgtcatccagcagattgg

SEQ ID NO: 199 - DNA, 19 bp, Artificial Sequence
  CNAG 04798 diagnostic screening primer, pairing with B79
tgaatgactgatggacccc

SEQ ID NO: 200 - DNA, 18 bp, Artificial Sequence
  CNAG 04798 Southern blot probe primer
tcggttcttgttttggag

SEQ ID NO: 201 - DNA, 20 bp, Artificial Sequence
  CNAG 04864 5 flanking region primer 1
tcatttcttcgtcctctgag

SEQ ID NO: 202 - DNA, 36 bp, Artificial Sequence
  CNAG 04864 5 flanking region primer 2
ctggccgtcgttttacgctccccataatcagactcc

SEQ ID NO: 203 - DNA, 36 bp, Artificial Sequence
  CNAG 04864 3 flanking region primer 1
gtcatagctgtttcctgacgaatggagtttctggag

SEQ ID NO: 204 - DNA, 20 bp, Artificial Sequence
  CNAG 04864 3 flanking region primer 2
gcgttctccatagttgtagc

SEQ ID NO: 205 - DNA, 20 bp, Artificial Sequence
  CNAG 04864 diagnostic screening primer, pairing with B79
atgtagataaagcacgcacc

SEQ ID NO: 206 - DNA, 20 bp, Artificial Sequence
  CNAG 04864 Southern blot probe primer
cgagttattgtaggttgggc

SEQ ID NO: 207 - DNA, 20 bp, Artificial Sequence
  CNAG 01973 5 flanking region primer 1
gcttgaggttgaaacaggtc

SEQ ID NO: 208 - DNA, 41 bp, Artificial Sequence
  CNAG 01973 5 flanking region primer 2
tcactggccgtcgttttaccagatgtgtgtaagagggtcag

SEQ ID NO: 209 - DNA, 39 bp, Artificial Sequence
  CNAG 01973 3 flanking region primer 1
catggtcatagctgtttcctgtaccgagaacgatttggc

SEQ ID NO: 210 - DNA, 20 bp, Artificial Sequence
  CNAG 01973 3 flanking region primer 2
tcccataagaccacacactg

SEQ ID NO: 211 - DNA, 19 bp, Artificial Sequence
  CNAG 01973 diagnostic screening primer, pairing with B79
tgatgctgaatagacgggg

SEQ ID NO: 212 - DNA, 19 bp, Artificial Sequence
  CNAG 01973 Southern blot probe primer
agccttgacgacatacctg

SEQ ID NO: 213 - DNA, 20 bp, Artificial Sequence
  NAT 6 STM primer
atagctaccacacgatagct

SEQ ID NO: 214 - DNA, 24 bp, Artificial Sequence
  STM common primer
gcatgccctgcccctaagaattcg

SEQ ID NO: 215 - DNA, 19 bp, Artificial Sequence
  CNAG 03914 5 flanking region primer 1
gatgtcgttgttctagagc

SEQ ID NO: 216 - DNA, 35 bp, Artificial Sequence
  CNAG 03914 5 flanking region primer 2
ctggccgtcgttttactttggatgtaagaggtcgg

SEQ ID NO: 217 - DNA, 37 bp, Artificial Sequence
  CNAG 03914 3 flanking region primer 1
gtcatagctgtttcctgcgagtatccttaccgtgtcg

SEQ ID NO: 218 - DNA, 19 bp, Artificial Sequence
  CNAG 03914 3 flanking region primer 2
acactcctgcccaagaaac

SEQ ID NO: 219 - DNA, 19 bp, Artificial Sequence
  CNAG 03914 diagnostic screening primer, pairing with B79
actccactccatbcaaagc

SEQ ID NO: 220 - DNA, 20 bp, Artificial Sequence
  CNAG 03914 Southern blot probe primer
ataagaggtccttttgctcc

SEQ ID NO: 221 - DNA, 20 bp, Artificial Sequence
  NAT 43 STM primer
ccagctaccaatcacgctac

SEQ ID NO: 222 - DNA, 24 bp, Artificial Sequence
  STM common primer
gcatgccctgcccctaagaattcg

SEQ ID NO: 223 - DNA, 19 bp, Artificial Sequence
  CNAG 00828 5 flanking region primer 1
accagtggcaaggttagag

SEQ ID NO: 224 - DNA, 39 bp, Artificial Sequence
  CNAG 00828 5 flanking region primer 2
tcactggccgtcgttttactggcagaagcacaagattag

SEQ ID NO: 225 - DNA, 40 bp, Artificial Sequence
  CNAG 00828 3 flanking region primer 1
catggtcatagctgtttcctggccatttctcgtagcgaac

SEQ ID NO: 226 - DNA, 19 bp, Artificial Sequence
  CNAG 00828 3 flanking region primer 2
actgcttatccccacatcg

SEQ ID NO: 227 - DNA, 20 bp, Artificial Sequence
  CNAG 00828 diagnostic screening primer, pairing with B79
tgtgaagtgtgatgatgtcc

SEQ ID NO: 228 - DNA, 19 bp, Artificial Sequence
  CNAG 00828 Southern blot probe primer
ggttgccactgataaaagc

SEQ ID NO: 229 - DNA, 24 bp, Artificial Sequence
  NAT 58 STM primer
cgcaaaatcactagccctatagcg

SEQ ID NO: 230 - DNA, 24 bp, Artificial Sequence
  STM common primer
gcatgccctgcccctaagaattcg

SEQ ID NO: 231 - DNA, 19 bp, Artificial Sequence
  CNAG 00871 5 flanking region primer 1
atagcgggcaagtgtctac

SEQ ID NO: 232 - DNA, 38 bp, Artificial Sequence
  CNAG 00871 5 flanking region primer 2
tcactggccgtcgttttacccaacctttcattccagtc

SEQ ID NO: 233 - DNA, 42 bp, Artificial Sequence
  CNAG 00871 3 flanking region primer 1
catggtcatagctgtttcctgggatacaaatctcaacccctg

SEQ ID NO: 234 - DNA, 19 bp, Artificial Sequence
  CNAG 00871 3 flanking region primer 2
ggttttcacttcaagagcg

SEQ ID NO: 235 - DNA, 18 bp, Artificial Sequence
  CNAG 00871 diagnostic screening primer, pairing with B79
tccaatagcaccacaacc

SEQ ID NO: 236 - DNA, 19 bp, Artificial Sequence
  CNAG 00871 Southern blot probe primer
aacatccctttactcggcg

SEQ ID NO: 237 - DNA, 24 bp, Artificial Sequence
  NAT 102 STM primer
ccatagcgatatctaccccaatct

SEQ ID NO: 238 - DNA, 24 bp, Artificial Sequence
  STM common primer
gcatgccctgcccctaagaattcg

SEQ ID NO: 239 - DNA, 19 bp, Artificial Sequence
  CNAG 07464 5 flanking region primer 1
ggcatcaggatagaaacgc

SEQ ID NO: 240 - DNA, 41 bp, Artificial Sequence
  CNAG 07464 5 flanking region primer 2
gctcactggccgtcgttttacggggtgagatttgaaggtag

SEQ ID NO: 241 - DNA, 40 bp, Artificial Sequence
  CNAG 07464 3 flanking region primer 1
catggtcatagctgtttcctggatgtgacttggtcttggg

SEQ ID NO: 242 - DNA, 20 bp, Artificial Sequence
  CNAG 07464 3 flanking region prither 2
tggtccagtctcctcattac

SEQ ID NO: 243 - DNA, 18 bp, Artificial Sequence
  CNAG 07464 diagnostic screening primer, pairing with B79
tcttccatctcagcatcg

SEQ ID NO: 244 - DNA, 24 bp, Artificial Sequence
  CNAG 07464 Southern blot probe primer
ccatagcgatatctaccccaatct

SEQ ID NO: 245 - DNA, 20 bp, Artificial Sequence
  NAT 150 STM primer
acatacacccccatcccccc

SEQ ID NO: 246 - DNA, 24 bp, Artificial Sequence
  STM common primer
gcatgccctgcccctaagaattcg

SEQ ID NO: 247 - DNA, 23 bp, Artificial Sequence
  CNAG 00460 5 flanking region primer 1
cgtgggagagtgagtaaagtatg

SEQ ID NO: 248 - DNA, 42 bp, Artificial Sequence
  CNAG 00460 5 flanking region primer 2
gctcactggccgtcgttttaccgggtaagagtggatgtatgg

SEQ ID NO: 249 - DNA, 39 bp, Artificial Sequence
  CNAG 00460 3 flanking region primer 1
catggtcatagctgtttcctgaagggcaccgttcattag

SEQ ID NO: 250 - DNA, 19 bp, Artificial Sequence
  CNAG 00460 3 flanking region primer 2
ttcttaggatgcgggactc

SEQ ID NO: 251 - DNA, 18 bp, Artificial Sequence
  CNAG 00460 diagnostic screening primer, pairing with B79
tgcgttgacaaagtcgtg

SEQ ID NO: 252 - DNA, 22 bp, Artificial Sequence
  CNAG 00460 Southern blot probe primer
tagactccagtaaggtccaatc

SEQ ID NO: 253 - DNA, 24 bp, Artificial Sequence
  NAT 213 STM primer
ctggggattttgatgtgtctatgt

SEQ ID NO: 254 - DNA, 24 bp, Artificial Sequence
  STM common primer
gcatgccctgcccctaagaattcg

SEQ ID NO: 255 - DNA, 18 bp, Artificial Sequence
  CNAG 01431 5 flanking region primer 1
tccatttgggggtttcag

SEQ ID NO: 256 - DNA, 39 bp, Artificial Sequence
  CNAG 01431 5 flanking region primer 2
tcactggccgtcgttttactgtcttcgttcttgccctcc

SEQ ID NO: 257 - DNA, 42 bp, Artificial Sequence
  CNAG 01431 3 flanking region primer 1
catggtcatagctgtttcctgcgttgacagaagaggacaagg

SEQ ID NO: 258 - DNA, 20 bp, Artificial Sequence
  CNAG 01431 3 flanking region primer 2
tgacagatgatgagcagagg

SEQ ID NO: 259 - DNA, 19 bp, Artificial Sequence
  CNAG 01431 diagnostic screening primer, pairing with B79
attccagagcctcacttgc

SEQ ID NO: 260 - DNA, 21 bp, Artificial Sequence
  CNAG 01431 Southern blot probe primer
cctgcttgtcactacactgag

SEQ ID NO: 261 - DNA, 24 bp, Artificial Sequence
  NAT 213 STM primer
ctggggattttgatgtgtctatgt

SEQ ID NO: 262 - DNA, 24 bp, Artificial Sequence
  STM common primer
gcatgccctgcccctaagaattcg

SEQ ID NO: 263 - DNA, 21 bp, Artificial Sequence
  CNAG 03894 5 flanking region primer 1
catctatctcaacggggtatc

SEQ ID NO: 264 - DNA, 39 bp, Artificial Sequence
  CNAG 03894 5 flanking region primer 2
gctcactggccgtcgttttactttttcgggagaacgcag

SEQ ID NO: 265 - DNA, 40 bp, Artificial Sequence
  CNAG 03894 3 flanking region primer 1
catggtcatagctgtttcctgaggaccaagaatgctgagg

SEQ ID NO: 266 - DNA, 22 bp, Artificial Sequence
  CNAG_03894 3 flanking region primer 2
ttctcccttgtcgccgataagc

SEQ ID NO: 267 - DNA, 20 bp, Artificial Sequence
  CNAG 03894 diagnostic screening primer, pairing with B79
tcttatcgctcaccacatcc

SEQ ID NO: 268 - DNA, 19 bp, Artificial Sequence
  CNAG 03894 Southern blot probe primer
ggaacattgggaaaaggtg

SEQ ID NO: 269 - DNA, 20 bp, Artificial Sequence
  NAT 220 STM primer
cagatctcgaacgataccca

SEQ ID NO: 270 - DNA, 24 bp, Artificial Sequence
  STM common primer
gcatgccctgcccctaagaattcg

SEQ ID NO: 271 - DNA, 18 bp, Artificial Sequence
  CNAG 06483 5 flanking region primer 1
tggagcagatggagtttg

SEQ ID NO: 272 - DNA, 35 bp, Artificial Sequence
  CNAG 06483 5 flanking region primer 2
ctggccgtcgttttacgcttctccctatttccagc

SEQ ID NO: 273 - DNA, 36 bp, Artificial Sequence
  CNAG 06483 3 flanking region primer 1
gtcatagctgtttcctgaatggagcccagagaacag

SEQ ID NO: 274 - DNA, 19 bp, Artificial Sequence
  CNAG 06483 3 flanking region primer 2
tctcgctgagggaaaactc

SEQ ID NO: 275 - DNA, 18 bp, Artificial Sequence
  CNAG 06483 diagnostic screening primer, pairing with B79
ccctttctctgcgttttg

SEQ ID NO: 276 - DNA, 18 bp, Artificial Sequence
  CNAG 06483 Southern blot probe primer
caatgaagaactggtgcg

SEQ ID NO: 277 - DNA, 20 bp, Artificial Sequence
  NAT 227 STM primer
tcgtggtttagagggagcgc

SEQ ID NO: 278 - DNA, 24 bp, Artificial Sequence
  STM common primer
gcatgccctgcccctaagaattcg

SEQ ID NO: 279 - DNA, 18 bp, Artificial Sequence
  CNAG 00896 5 flanking region primer 1
aaagggtcacgatggaac

SEQ ID NO: 280 - DNA, 35 bp, Artificial Sequence
  CNAG 00896 5 flanking region primer 2
ctggccgtcgttttacgcgattctttactcctggc

SEQ ID NO: 281 - DNA, 36 bp, Artificial Sequence
  CNAG 00896 3 flanking region primer 1
gtcatagctgtttcctgttctgagacctccgtaacc

SEQ ID NO: 282 - DNA, 19 bp, Artificial Sequence
  CNAG 00896 3 flanking region primer 2
ctgtatcgctttagccacc

SEQ ID NO: 283 - DNA, 19 bp, Artificial Sequence
  CNAG 00896 diagnostic screening primer, pairing with B79
tacaaggacctcacgaacg

SEQ ID NO: 284 - DNA, 20 bp, Artificial Sequence
  CNAG 00896 Southern blot probe primer
acgagcagagggacagatag

SEQ ID NO: 285 - DNA, 20 bp, Artificial Sequence
  NAT 231 STM primer
gagagatcccaacatcacgc

SEQ ID NO: 286 - DNA, 24 bp, Artificial Sequence
  STM common primer
gcatgccctgcccctaagaattcg

SEQ ID NO: 287 - DNA, 18 bp, Artificial Sequence
  CNAG 01977 5 flanking region primer 1
tgggaaaacgaagcactc

SEQ ID NO: 288 - DNA, 35 bp, Artificial Sequence
  CNAG 01977 5 flanking region primer 2
ctggccgtcgttttactaggaggtcactttcgctc

SEQ ID NO: 289 - DNA, 36 bp, Artificial Sequence
  CNAG 01977 3 flanking region primer 1
gtcatagctgtttcctgcggcaaagaacaaggtatg

SEQ ID NO: 290 - DNA, 20 bp, Artificial Sequence
  CNAG 01977 3 flanking region primer 2
tgatgggcttgaacgaacgc

SEQ ID NO: 291 - DNA, 19 bp, Artificial Sequence
  CNAG 01977 diagnostic screening primer, pairing with B79
tgtcggcacacatctactg

SEQ ID NO: 292 - DNA, 22 bp, Artificial Sequence
  CNAG 01977 Southern blot probe primer
gtctcagcctcttgttacagat

SEQ ID NO: 293 - DNA, 20 bp, Artificial Sequence
  NAT 231 STM primer
gagagatcccaacatcacgc

SEQ ID NO: 294 - DNA, 24 bp, Artificial Sequence
  STM common primer
gcatgccctgcccctaagaattcg

SEQ ID NO: 295 - DNA, 19 bp, Artificial Sequence
  CNAG 01069 5 flanking region primer 1
cttttatctatggcgagcg

SEQ ID NO: 296 - DNA, 39 bp, Artificial Sequence
  CNAG 01069 5 flanking region primer 2
gctcactggccgtcgttttactatcgcactgctggaatc

SEQ ID NO: 297 - DNA, 40 bp, Artificial Sequence
  CNAG 01069 3 flanking regiOn primet 1
catggtcatagctgtttcctgctactttccctcacatccg

SEQ ID NO: 298 - DNA, 19 bp, Artificial Sequence
  CNAG 01069 3 flanking region primer 2
tggagggtagatgcgttac

SEQ ID NO: 299 - DNA, 20 bp, Artificial Sequence
  CNAG 01069 diagnostic screening primer, pairing with B79
agagaagcggaagagaaaac

SEQ ID NO: 300 - DNA, 20 bp, Artificial Sequence
  CNAG 01069 Southern blot probe primer
ccttgcgaaaatgtcttctc

SEQ ID NO: 301 - DNA, 21 bp, Artificial Sequence
  NAT 292 STM primer
atttcgatgtatatatgtagc

SEQ ID NO: 302 - DNA, 24 bp, Artificial Sequence
  STM common primer
gcatgccctgcccctaagaattcg

SEQ ID NO: 303 - DNA, 19 bp, Artificial Sequence
  CNAG 06276 5 flanking region primer 1
tcaagtctacgcagtcgtg

SEQ ID NO: 304 - DNA, 40 bp, Artificial Sequence
  CNAG 06276 5 flanking region primer 2
gctcactggccgtcgttttacgcagcaatcaactcactcg

SEQ ID NO: 305 - DNA, 41 bp, Artificial Sequence
  CNAG 06276 3 flanking region primer 1
catggtcatagctgtttcctgaggagagtgggagtatggag

SEQ ID NO: 306 - DNA, 18 bp, Artificial Sequence
  CNAG 06276 3 flanking region primer 2
acccatcaaaaggcgtag

SEQ ID NO: 307 - DNA, 20 bp, Artificial Sequence
  CNAG 06276 diagnoStic screening primer, pairing with 879
accatttgtcaactgtcagg

SEQ ID NO: 308 - DNA, 20 bp, Artificial Sequence
  CNAG 06276 Southern blot probe primer
tcgctcactgacaagaagac

SEQ ID NO: 309 - DNA, 21 bp, Artificial Sequence
  NAT 292 STM primer
atttcgatgtatatatgtagc

SEQ ID NO: 310 - DNA, 24 bp, Artificial Sequence
  STM common primer
gcatgccctgcccctaagaattcg

SEQ ID NO: 311 - DNA, 19 bp, Artificial Sequence
  CNAG 02476 5 flanking region primer 1
tctgctcaatgaaacgggc

SEQ ID NO: 312 - DNA, 45 bp, Artificial Sequence
  CNAG 02476 5 flanking region primer 2
gctcactggccgtcgttttacgcctttgcttcctttgcttcttac

SEQ ID NO: 313 - DNA, 40 bp, Artificial Sequence
  CNAG 02476 3 flanking region primer 1
catggtcatagctgtttcctggtgtcttgaatccaatgcc

SEQ ID NO: 314 - DNA, 20 bp, Artificial Sequence
  CNAG 02476 3 flanking region primer 2
ctgctccatacaaggcttag

SEQ ID NO: 315 - DNA, 20 bp, Artificial Sequence
  CNAG 02476 diagnostic screening primer, pairing with B79
tcgtcagcaattgaaaacct

SEQ ID NO: 316 - DNA, 19 bp, Artificial Sequence
  CNAG 02476 Southern blot probe primer
atcaagtcagaccacgagg

SEQ ID NO: 317 - DNA, 19 bp, Artificial Sequence
  NAT 219 STM primer
ccctaaaacctacagcaat

SEQ ID NO: 318 - DNA, 24 bp, Artificial Sequence
  STM common primer
gcatgccctgcccctaagaattcg

SEQ ID NO: 319 - DNA, 20 bp, Artificial Sequence
  CNAG 01841 5 flanking region primer 1
ccattatcatccgtcaccac

SEQ ID NO: 320 - DNA, 37 bp, Artificial Sequence
  CNAG 01841 5 flanking region primer 2
ctggccgtcgttttacgaagagggaagagagggtaac

SEQ ID NO: 321 - DNA, 36 bp, Artificial Sequence
  CNAG 01841 3 flanking region primer 1
gtcatagctgtttcctgtgaggctgaggaaacagag

SEQ ID NO: 322 - DNA, 18 bp, Artificial Sequence
  CNAG 01841 3 flanking region primer 2
tccttttcccaacctgtc

SEQ ID NO: 323 - DNA, 21 bp, Artificial Sequence
  CNAG 01841 diagnostic screening primer, pairing with B79
ttcagtggttggtctctacag

SEQ ID NO: 324 - DNA, 21 bp, Artificial Sequence
  CNAG 01841 Southern blot probe primer
tggtaaaggtctatcagtgcc

SEQ ID NO: 325 - DNA, 20 bp, Artificial Sequence
  NAT 230 STM primer
atgtaggtagggtgataggt

SEQ ID NO: 326 - DNA, 24 bp, Artificial Sequence
  STM common primer
gcatgccctgcccctaagaattcg

SEQ ID NO: 327 - DNA, 19 bp, Artificial Sequence
  CNAG 04457 5 flanking region primer 1
cttcaaaatccagagcagg

SEQ ID NO: 328 - DNA, 38 bp, Artificial Sequence
  CNAG 04457 5 flanking region primer 2
ctggccgtcgttttacctggtctgatagttcgtattcg

SEQ ID NO: 329 - DNA, 39 bp, Artificial Sequence
  CNAG 04457 3 flanking region primer 1
gtcatagctgtttcctgggacttgaacaatggtatggac

SEQ ID NO: 330 - DNA, 19 bp, Artificial Sequence
  CNAG 04457 3 flanking region primer 2
tttagagcctacggtggtc

SEQ ID NO: 331 - DNA, 22 bp, Artificial Sequence
  CNAG 04457 diagnostic screening primer, peiring with B79
ttgcccgacgagatgctatgac

SEQ ID NO: 332 - DNA, 20 bp, Artificial Sequence
  CNAG 04457 Southern blot probe primer
tccgtctccgatgatttgcg

SEQ ID NO: 333 - DNA, 20 bp, Artificial Sequence
  NAT 230 STM primer
atgtaggtagggtgataggt

SEQ ID NO: 334 - DNA, 24 bp, Artificial Sequence
  STM common primer
gcatgccctgcccctaagaattcg

SEQ ID NO: 335 - DNA, 19 bp, Artificial Sequence
  CNAG 06252 5 flanking region primer 1
tctgadaggttcaatcgcc

SEQ ID NO: 336 - DNA, 41 bp, Artificial Sequence
  CNAG 06252 5 flanking region primer 2
tcactggccgtcgttttacgcatttctacaccgtgattagc

SEQ ID NO: 337 - DNA, 40 bp, Artificial Sequence
  CNAG 06252 3 flanking region primer 1
catggtcatagctgtttcctgggatgtgcggttttgttag

SEQ ID NO: 338 - DNA, 19 bp, Artificial Sequence
  CNAG 06252 3 flanking region primer 2
taacgaagttctccccgtg

SEQ ID NO: 339 - DNA, 20 bp, Artificial Sequence
  CNAG 06252 diagnostic screening primer, pairing with B79
tgagagggaacgaaggatac

SEQ ID NO: 340 - DNA, 20 bp, Artificial Sequence
  CNAG 06252 Southern blot probe primer
cttcgcctgttggaaataag

SEQ ID NO: 341 - DNA, 20 bp, Artificial Sequence
  CNAG 06327 5 flanking region primer 1
ggagaagggaaagatgggtg

SEQ ID NO: 342 - DNA, 41 bp, Artificial Sequence
  CNAG 06327 5 flanking region primer 2
gctcactggccgtcgttttactgtttccactattgggagag

SEQ ID NO: 343 - DNA, 40 bp, Artificial Sequence
  CNAG 06327 3 flanking region primer 1
catggtcatagctgtttcctgaggtgaaggtgtgagattg

SEQ ID NO: 344 - DNA, 18 bp, Artificial Sequence
  CNAG 06327 3 flanking region primer 2
caataatgtcctcgccag

SEQ ID NO: 345 - DNA, 19 bp, Artificial Sequence
  CNAG 06327 diagnostic screening primer, pairing with B79
gtatgaagggaaggtgctg

SEQ ID NO: 346 - DNA, 18 bp, Artificial Sequence
  CNAG 06327 Southern blot probe primer
acgggttacgagaaatgc

SEQ ID NO: 347 - DNA, 19 bp, Artificial Sequence
  CNAG 03790 5 flanking region primer 1
tgagggttgttgagggaag

SEQ ID NO: 348 - DNA, 40 bp, Artificial Sequence
  CNAG 03790 5 flanking region primer 2
gctcactggccgtcgttttacagcactgtatcgccgaaac

SEQ ID NO: 349 - DNA, 42 bp, Artificial Sequence
  CNAG 03790 3 flanking region primer 1
catggtcatagctgtttcctgctcgtagcaatgtagatgtgc

SEQ ID NO: 350 - DNA, 21 bp, Artificial Sequence
  CNAG 03790 3 flanking region primer 2
tcagatgaagacgactacgac

SEQ ID NO: 351 - DNA, 19 bp, Artificial Sequence
  CNAG 03790 diagnostic screening primer, pairing with B79
cttccacaaagccagattc

SEQ ID NO: 352 - DNA, 18 bp, Artificial Sequence
  CNAG 03790 Southern blot probe primer
agattgcgtgaaacccac

SEQ ID NO: 353 - DNA, 19 bp, Artificial Sequence
  CNAG 05112 5 flanking region primer 1
atgtgcgaagtccaagagc

SEQ ID NO: 354 - DNA, 40 bp, Artificial Sequence
  CNAG 05112 5 flanking region primer 2
gctcactggccgtcgttttacgggtgaagacaacagatgg

SEQ ID NO: 355 - DNA, 41 bp, Artificial Sequence
  CNAG 05112 3 flanking region primer 1
catggtcatagctgtttcctgcagaccaactggcgtaatag

SEQ ID NO: 356 - DNA, 19 bp, Artificial Sequence
  CNAG 05112 3 flanking region primer 2
acgaccaaatgctcttcgc

SEQ ID NO: 357 - DNA, 19 bp, Artificial Sequence
  CNAG 05112 diagnostic screening primer, pairing with B79
tcggatacaggcgatagag

SEQ ID NO: 358 - DNA, 19 bp, Artificial Sequence
  CNAG 05112 Southern blot probe primer
tcaacagcagtttcaggtg

SEQ ID NO: 359 - DNA, 24 bp, Artificial Sequence
  NAT 122 STM primer
acagctccaaacctcgctaaacag

SEQ ID NO: 360 - DNA, 24 bp, Artificial Sequence
  STM common primer
gcatgccctgcccctaagaattcg

SEQ ID NO: 361 - DNA, 19 bp, Artificial Sequence
  CNAG 00068 5 flanking region primer 1
tctatcacctctgcgttgc

SEQ ID NO: 362 - DNA, 39 bp, Artificial Sequence
  CNAG 00068 5 flanking region primer 2
tcactggccgtcgttttacattcctaccactatcccctg

SEQ ID NO: 363 - DNA, 40 bp, Artificial Sequence
  CNAG 00068 3 flanking region primer 1
catggtcatagctgtttcctgcggataagatgcctgagac

SEQ ID NO: 364 - DNA, 22 bp, Artificial Sequence
  CNAG 00068 3 flanking region primer 2
ggtatgtttctcggatgtaagg

SEQ ID NO: 365 - DNA, 20 bp, Artificial Sequence
  CNAG 00068 diagnostic screening primer, pairing with B79
catcatcaggtatcagcagc

SEQ ID NO: 366 - DNA, 20 bp, Artificial Sequence
  CNAG 00068 Southern blot probe primer
cttccaacttcaatgttccc

SEQ ID NO: 367 - DNA, 24 bp, Artificial Sequence
  NAT 58 STM primer
cgcaaaatcactagccctatagcg

SEQ ID NO: 368 - DNA, 24 bp, Artificial Sequence
  STM common primer
gcatgccctgcccctaagaattcg

SEQ ID NO: 369 - DNA, 19 bp, Artificial Sequence
  CNAG 04184 5 flanking region primer 1
tcgttcacataactgccac

SEQ ID NO: 370 - DNA, 38 bp, Artificial Sequence
  CNAG 04184 5 flanking region primer 2
tcactggccgtcgttttactcattgtgagcctcgtatg

SEQ ID NO: 371 - DNA, 39 bp, Artificial Sequence
  CNAC 04184 3 flanking region primer 1
catggtcatagctgtttcctgaatggtgacggttcaagg

SEQ ID NO: 372 - DNA, 18 bp, Artificial Sequence
  CNAG 04184 3 flanking region primer 2
cttttccactgaatccgc

SEQ ID NO: 373 - DNA, 19 bp, Artificial Sequence
  CNAG 04184 diagnostic screening primer, pairing with B79
tggtccgatgccgtaaatc

SEQ ID NO: 374 - DNA, 19 bp, Artificial Sequence
  CNAG 04184 Southern blot probe primer
caagggdatgactcactcc

SEQ ID NO: 375 - DNA, 24 bp, Artificial Sequence
  NAT 102 STM primer
ccatagcgatatctaccccaatct

SEQ ID NO: 376 - DNA, 24 bp, Artificial Sequence
  STM common primer
gcatgccctgcccctaagaattcg

SEQ ID NO: 377 - DNA, 18 bp, Artificial Sequence
  CNAG 03018 5 flanking region primer 1
agccgtttgtcattctgc

SEQ ID NO: 378 - DNA, 37 bp, Artificial Sequence
  CNAG 03018 5 flanking region primer 2
tcactggccgtcgttttacgtcgttgctcattttgcg

SEQ ID NO: 379 - DNA, 39 bp, Artificial Sequence
  CNAG 03018 3 flanking region primer 1
catggtcatagctgtttcctggcgaacctccaataaagc

SEQ ID NO: 380 - DNA, 18 bp, Artificial Sequence
  CNAG 03018 3 flanking region primer 2
cccttttttccctgcttc

SEQ ID NO: 381 - DNA, 18 bp, Artificial Sequence
  CNAG 03018 diagnostic screening primer, pairing with B79
aatagggatgccaaccag

SEQ ID NO: 382 - DNA, 18 bp, Artificial Sequence
  CNAG 03018 Southern blot probe primer
caaaaagtgtcagcacgc

SEQ ID NO: 383 - DNA, 20 bp, Artificial Sequence
  NAT 150 STM primer
acatacacccccatcccccc

SEQ ID NO: 384 - DNA, 24 bp, Artificial Sequence
  STM common primer
gcatgccctgcccctaagaattcg

SEQ ID NO: 385 - DNA, 20 bp, Artificial Sequence
  CNAG 01708 5 flanking region primer 1
ccgactcgttcgtctatctg

SEQ ID NO: 386 - DNA, 41 bp, Artificial Sequence
  CNAG 01708 5 flanking region primer 2
tcactggccgtcgttttactggagaggatagatgtgatgac

SEQ ID NO: 387 - DNA, 41 bp, Artificial Sequence
  CNAG 01708 3 flanking region primer 1
catggtcatagctgtttcctgaacttctacccaggtcgtgc

SEQ ID NO: 388 - DNA, 18 bp, Artificial Sequence
  CNAG 01708 3 flanking region primer 2
aactgcccttcttcaagc

SEQ ID NO: 389 - DNA, 20 bp, Artificial Sequence
  CNAG 01708 diagnostic screening primer, pairing with B79
tagcctctcccaatgtccac

SEQ ID NO: 390 - DNA, 20 bp, Artificial Sequence
  CNAG 01708 Southern blot probe primer
cgattcttctttgctgagtc

SEQ ID NO: 391 - DNA, 21 bp, Artificial Sequence
  NAT 159 STM primer
acgcaccagacacacaaccag

SEQ ID NO: 392 - DNA, 24 bp, Artificial Sequence
  STM common primer
gcatgccctgcccctaagaattcg

SEQ ID NO: 393 - DNA, 19 bp, Artificial Sequence
  CNAG 05861 5 flanking region primer 1
ggttttggtcacttcaccg

SEQ ID NO: 394 - DNA, 38 bp, Artificial Sequence
  CNAG 05861 5 flanking region primer 2
tcactggccgtcgttttacagcagaaaggagatgatgc

SEQ ID NO: 395 - DNA, 40 bp, Artificial Sequence
  CNAG 05861 3 flanking region primer 1
catggtcatagctgtttcctggacgacttttacgaccagg

SEQ ID NO: 396 - DNA, 18 bp, Artificial Sequence
  CNAG 05861 3 flanking region primer 2
aaatggaacaagcccgtc

SEQ ID NO: 397 - DNA, 18 bp, Artificial Sequence
  CNAG 05861 diagnostic screening primer, pairing with B79
tctgaagggcgtaaatgc

SEQ ID NO: 398 - DNA, 18 bp, Artificial Sequence
  CNAG 05861 Southern blot probe primer
aaggaatgggttgttggg

SEQ ID NO: 399 - DNA, 24 bp, Artificial Sequence
  NAT 184 STM primer
atatatggctcgagctagatagag

SEQ ID NO: 400 - DNA, 24 bp, Artificial Sequence
  STM common primer
gcatgccctgcccctaagaattcg

SEQ ID NO: 401 - DNA, 20 bp, Artificial Sequence
  CNAG 02066 5 flanking region primer 1
ccccattgtaaacattggtg

SEQ ID NO: 402 - DNA, 36 bp, Artificial Sequence
  CNAG 02066 5 flanking region primer 2
ctggccgtcgttttaccgtgatgtggagttgattcg

SEQ ID NO: 403 - DNA, 36 bp, Artificial Sequence
  CNAG 02066 3 flanking region primer 1
gtcatagctgtttcctgtgctgccaatggaaggttc

SEQ ID NO: 404 - DNA, 20 bp, Artificial Sequence
  CNAG 02066 3 flanking region primer 2
atagtctcgctccttggtgc

SEQ ID NO: 405 - DNA, 19 bp, Artificial Sequence
  CNAG 02066 diagnostic screening primer, pairing with B79
gttcgttatccgctctttg

SEQ ID NO: 406 - DNA, 19 bp, Artificial Sequence
  CNAG 02066 Southern blot probe primer
acataggcgagacagttgc

SEQ ID NO: 407 - DNA, 20 bp, Artificial Sequence
  NAT 191 STM primer
atatggatgtttttagcgag

SEQ ID NO: 408 - DNA, 24 bp, Artificial Sequence
  STM common primer
gcatgccctgcccctaagaattcg

SEQ ID NO: 409 - DNA, 19 bp, Artificial Sequence
  CNAG 03527 5 flanking region primer 1
ccatctcacggttgatacg

SEQ ID NO: 410 - DNA, 35 bp, Artificial Sequence
  CNAG 03527 5 flanking regiOn primer 2
ctggccgtcgttttactcaaacccttatcccaccg

SEQ ID NO: 411 - DNA, 37 bp, Artificial Sequence
  CNAG 03527 3 flanking region primer 1
gtcatagctgtttcctgtctttccctgctttgcctgc

SEQ ID NO: 412 - DNA, 20 bp, Artificial Sequence
  CNAG 03527 3 flanking region primer 2
cgtcatccgtcgtgtaactg

SEQ ID NO: 413 - DNA, 19 bp, Artificial Sequence
  CNAG 03527 diagnostic screening primer, pairing with B79
tctcgcaaggtcagagttc

SEQ ID NO: 414 - DNA, 21 bp, Artificial Sequence
  CNAG 03527 Southern blot probe primer
ctatgagcgacattcacagac

SEQ ID NO: 415 - DNA, 20 bp, Artificial Sequence
  NAT 204 STM primer
gatctdtcgcgcttggggga

SEQ ID NO: 416 - DNA, 24 bp, Artificial Sequence
  STM common primer
gcatgccctgcccctaagaattcg

SEQ ID NO: 417 - DNA, 18 bp, Artificial Sequence
  CNAG 04263 5 flanking region primer 1
tgtttccacacaccgatg

SEQ ID NO: 418 - DNA, 36 bp, Artificial Sequence
  CNAG 04263 5 flanking region primer 2
ctggccgtcgttttacggtggtggtcatctgtatgc

SEQ ID NO: 419 - DNA, 37 bp, Artificial Sequence
  CNAG 04263 3 flanking region primer 1
gtcatagctgtttcctgagaagatggagacgcacacg

SEQ ID NO: 420 - DNA, 19 bp, Artificial Sequence
  CNAG 04263 3 flanking region primer 2
gctattgcgtgaagadtcc

SEQ ID NO: 421 - DNA, 20 bp, Artificial Sequence
  CNAG 04263 diagnostic screening primer, pairing with B79
cgtgattgagagtagcaacc

SEQ ID NO: 422 - DNA, 19 bp, Artificial Sequence
  CNAG 04263 Southern blot probe primer
gtaatgtcagtgagtgcgg

SEQ ID NO: 423 - DNA, 24 bp, Artificial Sequence
  NAT 205 STM primer
tatccccctctccgctctctagca

SEQ ID NO: 424 - DNA, 24 bp, Artificial Sequence
  STM common primer
gcatgccctgcccctaagaattcg

SEQ ID NO: 425 - DNA, 20 bp, Artificial Sequence
  CNAG 00018 5 flanking region primer 1
tttgcctcaatctctctcag

SEQ ID NO: 426 - DNA, 41 bp, Artificial Sequence
  CNAG 00018 5 flanking region primer 2
gctcactggccgtcgttttacggaatacaacatccacctcc

SEQ ID NO: 427 - DNA, 42 bp, Artificial Sequence
  CNAG 00018 3 flanking region primer 1
catggtcatagctgtttcctggcacgggttacattctacatc

SEQ ID NO: 428 - DNA, 20 bp, Artificial Sequence
  CNAG 00018 3 flanking region primer 2
ggggaggacttgaacttgag

SEQ ID NO: 429 - DNA, 18 bp, Artificial Sequence
  CNAG 00018 diagnostic screening primer, pairing with B79
aacaaactgccttcctcc

SEQ ID NO: 430 - DNA, 19 bp, Artificial Sequence
  CNAG 00018 Southern blot probe primer
gcttggttggataaagatg

SEQ ID NO: 431 - DNA, 20 bp, Artificial Sequence
  NAT 211 STM primer
gcggtcgctttatagcgatt

SEQ ID NO: 432 - DNA, 24 bp, Artificial Sequence
  STM common priMer
gcatgccctgcccctaagaattcg

SEQ ID NO: 433 - DNA, 19 bp, Artificial Sequence
  CNAG 05176 5 flanking region primer 1
tgagaaggaggatggtcac

SEQ ID NO: 434 - DNA, 37 bp, Artificial Sequence
  CNAG 05176 5 flanking region primer 2
tcactggccgtcgttttacatgtggatagtgttgcgg

SEQ ID NO: 435 - DNA, 40 bp, Artificial Sequence
  CNAG 05176 3 flanking region primer 1
catggtcatagctgtttcctggcaaaaagggagaatgtcg

SEQ ID NO: 436 - DNA, 19 bp, Artificial Sequence
  CNAG 05176 3 flanking region primer 2
tcatacagccgagttgacc

SEQ ID NO: 437 - DNA, 19 bp, Artificial Sequence
  CNAG 05176 diagnostic screening primer, pairing with B79
tgtgagtatgcctatcccg

SEQ ID NO: 438 - DNA, 19 bp, Artificial Sequence
  CNAG 05176 Southern blot probe primer
tcaccgagactaatggcac

SEQ ID NO: 439 - DNA, 20 bp, Artificial Sequence
  NAT 211 STM primer
gcggtcgctttatagcgatt

SEQ ID NO: 440 - DNA, 24 bp, Artificial Sequence
  STM common primer
gcatgccctgcccctaagaattcg

SEQ ID NO: 441 - DNA, 19 bp, Artificial Sequence
  CNAG 06339 5 flanking region primer 1
tagtgaaagactcggcacc

SEQ ID NO: 442 - DNA, 37 bp, Artificial Sequence
  CNAG 06339 5 flanking region primer 2
tcactggccgtcgttttaccagtcaccgcaaagattc

SEQ ID NO: 443 - DNA, 41 bp, Artificial Sequence
  CNAG 06339 3 flanking region primer 1
catggtcatagctgtttcctgcgcttcgttattacctgttc

SEQ ID NO: 444 - DNA, 22 bp, Artificial Sequence
  CNAG 06339 3 flanking region primer 2
ctccaggataagtagtgtgagg

SEQ ID NO: 445 - DNA, 20 bp, Artificial Sequence
  CNAG 06339 diagnostic screening primer, pairing with B79
gggaaagtagggtaaccacc

SEQ ID NO: 446 - DNA, 20 bp, Artificial Sequence
  CNAG 06339 Southern blot probe primer
gaggagaggtgaataatgcc

SEQ ID NO: 447 - DNA, 24 bp, Artificial Sequence
  NAT 213 STM primer
ctggggattttgatgtgtctatgt

SEQ ID NO: 448 - DNA, 24 bp, Artificial Sequence
  STM common primer
gcatgccctgcccctaagaattcg

SEQ ID NO: 449 - DNA, 19 bp, Artificial Sequence
  CNAG 07593 5 flanking region primer 1
gattgcttggatggacttc

SEQ ID NO: 450 - DNA, 39 bp, Artificial Sequence
  CNAG 07593 5 flanking region primer 2
tcactggccgtcgttttactgcctttcttgccgaactcg

SEQ ID NO: 451 - DNA, 40 bp, Artificial Sequence
  CNAG 07593 3 flanking region primer 1
catggtcatagctgtttcctgtttgaaggaccaggagcag

SEQ ID NO: 452 - DNA, 19 bp, Artificial Sequence
  CNAG 07593 3 flanking region primer 2
tcaagtgacacagtgccac

SEQ ID NO: 453 - DNA, 19 bp, Artificial Sequence
  CNAG 07593 diagnostic screening primer, pairing with B79
ccataatcagacagcgttg

SEQ ID NO: 454 - DNA, 19 bp, Artificial Sequence
  CNAG 07593 Southern blot probe primer
ggtttgggggaatagtgag

SEQ ID NO: 455 - DNA, 20 bp, Artificial Sequence
  NAT 218 STM primer
ctccacatccatcgctccaa

SEQ ID NO: 456 - DNA, 24 bp, Artificial Sequence
  STM common primer
gcatgccctgcccctaagaattcg

SEQ ID NO: 457 - DNA, 20 bp, Artificial Sequence
  CNAG 00670 5 flanking region primer 1
tgatgaagatgctgatgagg

SEQ ID NO: 458 - DNA, 41 bp, Artificial Sequence
  CNAG 00670 5 flanking region primer 2
gctcactggccgtcgttttactcatttggcgtctcacttac

SEQ ID NO: 459 - DNA, 39 bp, Artificial Sequence
  CNAG 00670 3 flanking region primer 1
catggtcatagctgtttcctgtgtgaaaggggagtgatg

SEQ ID NO: 460 - DNA, 19 bp, Artificial Sequence
  CNAG 00670 3 flanking region primer 2
agaatacatcgtggttggc

SEQ ID NO: 461 - DNA, 22 bp, Artificial Sequence
  CNAG 00670 diagnostic screening primer, pairing with B79
ggaagtgtagagtcaggagatg

SEQ ID NO: 462 - DNA, 21 bp, Artificial Sequence
  CNAG 00670 Southern blot probe primer
ggatgaaaggtgaaaggacac

SEQ ID NO: 463 - DNA, 19 bp, Artificial Sequence
  NAT 4 STM primer
aacctttaaatgggtagag

SEQ ID NO: 464 - DNA, 24 bp, Artificial Sequence
  STM common primer
gcatgccctgcccctaagaattcg

SEQ ID NO: 465 - DNA, 18 bp, Artificial Sequence
  CNAG 05010 5 flanking region primer 1
tccttcctttctaagccg

SEQ ID NO: 466 - DNA, 41 bp, Artificial Sequence
  CNAG 05010 5 flanking region primer 2
gctcactggccgtcgttttactgtcccctacttttactccg

SEQ ID NO: 467 - DNA, 41 bp, Artificial Sequence
  CNAG 05010 3 flanking region primer 1
catggtcatagctgtttcctgtgagggtttgataccttgtg

SEQ ID NO: 468 - DNA, 18 bp, Artificial Sequence
  CNAG 05010 3 flanking region primer 2
aagagcaacatccgcaag

SEQ ID NO: 469 - DNA, 19 bp, Artificial Sequence
  CNAG 05010 diagnostic screening primer, pairing with B79
aacagccttgccttacagc

SEQ ID NO: 470 - DNA, 19 bp, Artificial Sequence
  CNAG 05010 Southern blot probe primer
agtggtgaagggctaaatg

SEQ ID NO: 471 - DNA, 19 bp, Artificial Sequence
  NAT 224 STM primer
aacctttaaatgggtagag

SEQ ID NO: 472 - DNA, 24 bp, Artificial Sequence
  STM common ptimer
gcatgccctgcccctaagaattcg

SEQ ID NO: 473 - DNA, 18 bp, Artificial Sequence
  CNAG 04345 5 flanking region primer 1
aaaaggcagtcgtcttcg

SEQ ID NO: 474 - DNA, 40 bp, Artificial Sequence
  CNAG 04345 5 flanking region primer 2
ctggccgtcgttttacccttttagatactcacacaagccc

SEQ ID NO: 475 - DNA, 36 bp, Artificial Sequence
  CNAG 04345 3 flanking region primer 1
gtcatagctgtttcctgttctgggatgacttcacgc

SEQ ID NO: 476 - DNA, 19 bp, Artificial Sequence
  CNAG 04345 3 flanking region primer 2
ttactgctcctggtttggg

SEQ ID NO: 477 - DNA, 18 bp, Artificial Sequence
  CNAG 04345 diagnostic screening primer pairing with B79
cctgcttcacaaaatggc

SEQ ID NO: 478 - DNA, 22 bp, Artificial Sequence
  CNAG 04345 Southern blot probe primer
gagtagtggtgtcagtgtgatg

SEQ ID NO: 479 - DNA, 20 bp, Artificial Sequence
  NAT 225 STM primer
ccatagaactagctaaagca

SEQ ID NO: 480 - DNA, 24 bp, Artificial Sequence
  STM common primer
gcatgccctgcccctaagaattcg

SEQ ID NO: 481 - DNA, 19 bp, Artificial Sequence
  CNAG 02774 5 flanking region primer 1
ttgagccagcctaagcaag

SEQ ID NO: 482 - DNA, 35 bp, Artificial Sequence
  CNAG 02774 5 flanking region primer 2
ctggccgtcgttttactcttcttttctgcggtcac

SEQ ID NO: 483 - DNA, 36 bp, Artificial Sequence
  CNAG 02774 3 flanking region primer 1
gtcatagctgtttcctggcttgatttcttggaccac

SEQ ID NO: 484 - DNA, 19 bp, Artificial Sequence
  CNAG 02774 3 flanking region primer 2
cgagataagaagcaacgdc

SEQ ID NO: 485 - DNA, 19 bp, Artificial Sequence
  CNAG 02774 diagnostic screening primer, pairing with B79
tgccagccaacctaatctg

SEQ ID NO: 486 - DNA, 20 bp, Artificial Sequence
  CNAG 02774 Southern blot probe primer
gagaggcagggagataactc

SEQ ID NO: 487 - DNA, 20 bp, Artificial Sequence
  NAT 230 STM primer
atgtaggtagggtgataggt

SEQ ID NO: 488 - DNA, 24 bp, Artificial Sequence
  STM common primer
gcatgccctgcccctaagaattcg

SEQ ID NO: 489 - DNA, 20 bp, Artificial Sequence
  CNAG 06425 5 flanking region primer 1
ctttcacgaacgcttgattc

SEQ ID NO: 490 - DNA, 35 bp, Artificial Sequence
  CNAG 06425 5 flanking region primer 2
ctggcbgtcgttttacttggacagtttttcggcgg

SEQ ID NO: 491 - DNA, 36 bp, Artificial Sequence
  CNAG 06425 3 flanking region primer 1
gtcatagctgtttcctgatgtgcctgcggatttctc

SEQ ID NO: 492 - DNA, 19 bp, Artificial Sequence
  CNAG 06425 3 flanking region primer 2
tgagaccgctgaaagttcc

SEQ ID NO: 493 - DNA, 19 bp, Artificial Sequence
  CNAG 06425 diagnostic screening primer, pairing with B79
taatcgccgacaaagggtg

SEQ ID NO: 494 - DNA, 18 bp, Artificial Sequence
  CNAG 06425 Southern blot probe primer
ccctttttttgtgcgacc

SEQ ID NO: 495 - DNA, 24 bp, Artificial Sequence
  NAT 288 STM primer
ctatccaactagacctctagctac

SEQ ID NO: 496 - DNA, 24 bp, Artificial Sequence
  STM common primer
gcatgccctgcccctaagaattcg

SEQ ID NO: 497 - DNA, 22 bp, Artificial Sequence
  CNAG 05255 5 flanking region primer 1
atggtcaccgtcacaaggaagc

SEQ ID NO: 498 - DNA, 40 bp, Artificial Sequence
  CNAG 05255 5 flanking region primer 2
ctggccgtcgttttactacacattcggcttggttgcggac

SEQ ID NO: 499 - DNA, 38 bp, Artificial Sequence
  CNAG 05255 3 flanking region primer 1
gtcatagctgtttcctgcgaggtttgtatcccagtcag

SEQ ID NO: 500 - DNA, 20 bp, Artificial Sequence
  CNAG 05255 3 flanking region primer 2
aggggctatctctgcttcac

SEQ ID NO: 501 - DNA, 24 bp, Artificial Sequence
  CNAG 05255 diagnostic screening primer, pairing with B79
ttctgtgcccacctctactctcag

SEQ ID NO: 502 - DNA, 20 bp, Artificial Sequence
  CNAG 05255 Southern blot probe primer
accaaaactcgtcgtaggcg

SEQ ID NO: 503 - DNA, 24 bp, Artificial Sequence
  NAT 288 STM primer
ctatccaactagacctctagctac

SEQ ID NO: 504 - DNA, 24 bp, Artificial Sequence
  STM common primer
gcatgccctgcccctaagaattcg

SEQ ID NO: 505 - DNA, 19 bp, Artificial Sequence
  CNAG 05049 5 flanking region primer 1
atcaggacgagtggcattg

SEQ ID NO: 506 - DNA, 39 bp, Artificial Sequence
  CNAG 05049 5 flanking region primer 2
tcactggccgtcgttttacgagtcttctttgggcatagc

SEQ ID NO: 507 - DNA, 40 bp, Artificial Sequence
  CNAG 05049 3 flanking regicin primer 1
catggtcatagctgtttcctgagtggatgatgaggactcg

SEQ ID NO: 508 - DNA, 21 bp, Artificial Sequence
  CNAG 05049 3 flanking region primer 2
gtttcctatcttcagtcggag

SEQ ID NO: 509 - DNA, 20 bp, Artificial Sequence
  CNAG 05049 diagnostic screening primer, pairing with B79
gatagcagaggagaaaaggc

SEQ ID NO: 510 - DNA, 18 bp, Artificial Sequence
  CNAG 05049 Southern blot probe primer
tatttggggttgaggctg

SEQ ID NO: 511 - DNA, 20 bp, Artificial Sequence
  NAT 123 STM primer
ctatcgaccaaccaacacag

SEQ ID NO: 512 - DNA, 24 bp, Artificial Sequence
  STM common primer
gcatgccctgcccctaagaattcg

SEQ ID NO: 513 - DNA, 18 bp, Artificial Sequence
  CNAG 05222 5 flanking region primer 1
gagggtcctttggttttg

SEQ ID NO: 514 - DNA, 37 bp, Artificial Sequence
  CNAG 05222 5 flanking region primer 2
tcactggccgtcgttttactttcacactcacggttgg

SEQ ID NO: 515 - DNA, 39 bp, Artificial Sequence
  CNAG 05222 3 flanking region primer 1
catggtcatagctgtttcdtggttgccccttttgacaag

SEQ ID NO: 516 - DNA, 19 bp, Artificial Sequence
  CNAG 05222 3 flanking region primer 2
aacttacggcgacaacaac

SEQ ID NO: 517 - DNA, 18 bp, Artificial Sequence
  CNAG 05222 diagnostic screening primer, pairing with B79
tccacataatagccaccg

SEQ ID NO: 518 - DNA, 20 bp, Artificial Sequence
  CNAG 05222 Southern blot probe primer
gctatgttcaggagcgatac

SEQ ID NO: 519 - DNA, 20 bp, Artificial Sequence
  NAT 123 STM primer
ctatcgaccaaccaacacag

SEQ ID NO: 520 - DNA, 24 bp, Artificial Sequence
  STM common primer
gcatgccctgcccctaagaattcg

SEQ ID NO: 521 - DNA, 18 bp, Artificial Sequence
  CNAG 04130 5 flanking region primer 1
cattgacgccatagcaag

SEQ ID NO: 522 - DNA, 38 bp, Artificial Sequence
  CNAG 04130 5 flanking region primer 2
tcactggccgtcgttttacgtgtttggaatgcggtttc

SEQ ID NO: 523 - DNA, 41 bp, Artificial Sequence
  CNAG 04130 3 flanking region primer
catggtcatagctgtttcctgtggatgaggttgaggggaac

SEQ ID NO: 524 - DNA, 21 bp, Artificial Sequence
  CNAG 04130 3 flanking region primer 2
gtagtgtaagggcaggaagag

SEQ ID NO: 525 - DNA, 21 bp, Artificial Sequence
  CNAG 04130 diagnostic screening primer, pairing with B79
ttctactcataagggctacgc

SEQ ID NO: 526 - DNA, 21 bp, Artificial Sequence
  CNAG 04130 Southern blot probe primer
ttctactcataagggctacgc

SEQ ID NO: 527 - DNA, 19 bp, Artificial Sequence
  CNAG 00193 5 flanking region primer 1
gggcagaaatagttggcag

SEQ ID NO: 528 - DNA, 34 bp, Artificial Sequence
  CNAG 00193 5 flanking region primer 2
ctggccgtcgttttactttttccccccgatgtag

SEQ ID NO: 529 - DNA, 36 bp, Artificial Sequence
  CNAG 00193 3 flanking region primer 1
gtcatagctgtttcctgttcccgcaagaacagtgtc

SEQ ID NO: 530 - DNA, 20 bp, Artificial Sequence
  CNAG 00193 3 flanking region primer 2
ttcaggatgtccaaactcag

SEQ ID NO: 531 - DNA, 19 bp, Artificial Sequence
  CNAG 00193 diagnostic screening primer, pairing with B79
aaggggtaagaaatcgggc

SEQ ID NO: 532 - DNA, 19 bp, Artificial Sequence
  CNAG 00193 Southern blot probe primer
tgctgagagacagggaatc

SEQ ID NO: 533 - DNA, 20 bp, Artificial Sequence
  NAT 227 STM primer
tcgtggtttagagggagcgc

SEQ ID NO: 534 - DNA, 24 bp, Artificial Sequence
  STM common primer
gcatgccctgcccctaagaattcg

SEQ ID NO: 535 - DNA, 20 bp, Artificial Sequence
  CNAG 027000 5 flanking region primer 1
tgaaatcagactccagcctc

SEQ ID NO: 536 - DNA, 38 bp, Artificial Sequence
  CNAG 02700 5 flanking region primer 2
tcactggccgtcgttttacgcgaccacatttacactcg

SEQ ID NO: 537 - DNA, 41 bp, Artificial Sequence
  CNAG 02700 3 flanking region primer 1
catggtcatagctgtttcctggccacagaacaataactggg

SEQ ID NO: 538 - DNA, 19 bp, Artificial Sequence
  CNAG 02700 3 flanking region primer 2
gcgtatgaataagcgttgg

SEQ ID NO: 539 - DNA, 18 bp, Artificial Sequence
  CNAG 02700 diagnostic screening primer, pairing with B79
agcaaaacagaagcagcg

SEQ ID NO: 540 - DNA, 18 bp, Artificial Sequence
  CNAG 02700 Southern blot probe primer
agaaagcagcatttgggg

SEQ ID NO: 541 - DNA, 20 bp, Artificial Sequence
  NAT 230 STM primer
atgtaggtagggtgataggt

SEQ ID NO: 542 - DNA, 24 bp, Artificial Sequence
  STM common primer
gcatgccctgcccctaagaattcg

SEQ ID NO: 543 - DNA, 19 bp, Artificial Sequence
  CNAG 04586 5 flanking region primer 1
tccatctcccaatccacag

SEQ ID NO: 544 - DNA, 38 bp, Artificial Sequence
  CNAG 04586 5 flanking region primer 2
tcactggccgtcgttttacgcttgattaggcggataag

SEQ ID NO: 545 - DNA, 41 bp, Artificial Sequence
  CNAG 04586 3 flanking region primer 1
catggtgatagctgtttcctgtggacgaaaagacccctacc

SEQ ID NO: 546 - DNA, 18 bp, Artificial Sequence
  CNAG 04586 3 flanking region primer 2
aacactggaatgtgcgtg

SEQ ID NO: 547 - DNA, 20 bp, Artificial Sequence
  cNAG 04586 diagnostic screening primer, pairing with 879
ttggtggtgcggttttgtgc

SEQ ID NO: 548 - DNA, 18 bp, Artificial Sequence
  CNAG 04586 Southern blot probe primer
tgatgttggttcgtgacc

SEQ ID NO: 549 - DNA, 21 bp, Artificial Sequence
  NAT 159 STM primer
acgcaccagacacacaaccag

SEQ ID NO: 550 - DNA, 24 bp, Artificial Sequence
  STM common primer
gcatgccctgcccctaagaattcg

SEQ ID NO: 551 - DNA, 18 bp, Artificial Sequence
  CNAG 05153 5 flanking region primer 1
aaaaccaagtggaacccg

SEQ ID NO: 552 - DNA, 35 bp, Artificial Sequence
  CNAG 05153 5 flanking region primer 2
ctggccgtcgttttactggttgacgctgttagcac

SEQ ID NO: 553 - DNA, 37 bp, Artificial Sequence
  CNAG 05153 3 flanking region primer 1
gtcatagctgtttcctgataggcaggctggtggaaac

SEQ ID NO: 554 - DNA, 19 bp, Artificial Sequence
  CNAG 05153 3 flanking region primer 2
accacatttgcttccgagc

SEQ ID NO: 555 - DNA, 21 bp, Artificial Sequence
  CNAG 05153 diagnostic screening primer, pairing with JOHE12579
tggctgaactgtaggttagtg

SEQ ID NO: 556 - DNA, 18 bp, Artificial Sequence
  CNAG 05153 Southern blot probe primer
cggcgataaggtttttgc

SEQ ID NO: 557 - DNA, 24 bp, Artificial Sequence
  NAT 290 STM primer
accgacagctcgaacaagcaagag

SEQ ID NO: 558 - DNA, 24 bp, Artificial Sequence
  STM common primer
gcatgccctgcccctaagaattcg

SEQ ID NO: 559 - DNA, 20 bp, Artificial Sequence
  CNAG 02723 5 flanking region primer 1
gcttccttatcggtagggac

SEQ ID NO: 560 - DNA, 39 bp, Artificial Sequence
  CNAG 02723 5 flanking region primer 2
tcactggccgtcgttttacgactggacattgacgaagac

SEQ ID NO: 561 - DNA, 40 bp, Artificial Sequence
  CNAG 02723 3 flanking region primer 1
catggtcatagctgtttcctgtggtgacagtgacgcttac

SEQ ID NO: 562 - DNA, 19 bp, Artificial Sequence
  CNAG 02723 3 flanking region primer 2
attgtcaggatgtgggttc

SEQ ID NO: 563 - DNA, 21 bp, Artificial Sequence
  CNAG 02723 diagnostic screening primer, pairing with B79
gcttactcccaaabtcacacc

SEQ ID NO: 564 - DNA, 18 bp, Artificial Sequence
  CNAG 02723 Southern blot probe primer
atggatttggcagaacgg

SEQ ID NO: 565 - DNA, 24 bp, Artificial Sequence
  NAT 201 STM primer
caccctctatctcgagaaagctcc

SEQ ID NO: 566 - DNA, 24 bp, Artificial Sequence
  STM common primer
gcatgccctgcccctaagaattcg

SEQ ID NO: 567 - DNA, 19 bp, Artificial Sequence
  CNAG 03741 5 flanking region primer 1
gccgtctcctgatgtaatc

SEQ ID NO: 568 - DNA, 36 bp, Artificial Sequence
  CNAG 03741 5 flanking region primer 2
ctggccgtcgttttaccgactgtcatcaacgactgg

SEQ ID NO: 569 - DNA, 36 bp, Artificial Sequence
  CNAG 03741 3 flanking region primer 1
ctggccgtcgttttacgcgtcaacaggaataaaacc

SEQ ID NO: 570 - DNA, 22 bp, Artificial Sequence
  CNAG 03741 3 flanking region primer 2
ctgtgcctttctccaagataag

SEQ ID NO: 571 - DNA, 22 bp, Artificial Sequence
  CNAG 03741 diagnostic screening primer, pairing with B79
ggataagcagaacatagttgcg

SEQ ID NO: 572 - DNA, 21 bp, Artificial Sequence
  CNAG 03741 Southern blot probe primer
tctcttcttcttctgacctgc

SEQ ID NO: 573 - DNA, 24 bp, Artificial Sequence
  NAT 273 STM primer
gagatctttcgggaggtctgbatt

SEQ ID NO: 574 - DNA, 24 bp, Artificial Sequence
  STM common primer
gcatgccctgcccctaagaattcg

SEQ ID NO: 575 - DNA, 18 bp, Artificial Sequence
  CNAG 07411 5 flanking region primer 1
agacccgccaaaacaatc

SEQ ID NO: 576 - DNA, 35 bp, Artificial Sequence
  CNAG 07411 5 flanking region primer 2
ctggccgtcgttttacttggacctacgctgacttc

SEQ ID NO: 577 - DNA, 37 bp, Artificial Sequence
  CNAG 07411 3 flanking region primer 1
gtcatagctgtttcctggacttgcgttctgattttgg

SEQ ID NO: 578 - DNA, 20 bp, Artificial Sequence
  CNAG 07411 3 flanking region primer 2
gctgttatcaagagaatgcc

SEQ ID NO: 579 - DNA, 19 bp, Artificial Sequence
  CNAG 07411 diagnostic screening primer, pairing with B79
acgactggctatcaagcag

SEQ ID NO: 580 - DNA, 19 bp, Artificial Sequence
  CNAG 07411 Southern blot probe primer
tacgaggattgagtctggc

SEQ ID NO: 581 - DNA, 24 bp, Artificial Sequence
  NAT 288 STM primer
ctatccaactagacctctagctac

SEQ ID NO: 582 - DNA, 24 bp, Artificial Sequence
  STM common primer
gcatgccctgcccctaagaattcg

SEQ ID NO: 583 - DNA, 20 bp, Artificial Sequence
  CNAG 03849 5 flanking region primer 1
tagtggttcccgtttgtgcg

SEQ ID NO: 584 - DNA, 40 bp, Artificial Sequence
  CNAG 03849 5 flanking region primer 2
gctcactggccgtcgttttaccttacgcctttttccatcg

SEQ ID NO: 585 - DNA, 41 bp, Artificial Sequence
  CNAG 03849 3 flanking region primer 1
catggtcatagctgtttdctgtcaaacggcgattggtggtc

SEQ ID NO: 586 - DNA, 20 bp, Artificial Sequence
  CNAG 03849 3 flanking region primer 2
tggtgaaggggaagattggg

SEQ ID NO: 587 - DNA, 19 bp, Artificial Sequence
  CNAG 03849 diagnostic screening primer, pairing with B79
aacaagccgaggaatgagg

SEQ ID NO: 588 - DNA, 19 bp, Artificial Sequence
  CNAG 03849 Southern blot probe primer
tgagtgaacgaagagtgcc

SEQ ID NO: 589 - DNA, 20 bp, Artificial Sequence
  NAT 6 STM primer
atagctaccacacgatagct

SEQ ID NO: 590 - DNA, 24 bp, Artificial Sequence
  STM common primer
gcatgccctgcccctaagaattcg

SEQ ID NO: 591 - DNA, 19 bp, Artificial Sequence
  CNAG 04916 5 flanking region primer 1
ttgcgttgagacatcactg

SEQ ID NO: 592 - DNA, 43 bp, Artificial Sequence
  CNAG 04916 5 flanking region primer 2
gctcactggccgtcgttttacgctcctatgttaccaatgttgc

SEQ ID NO: 593 - DNA, 40 bp, Artificial Sequence
  CNAG 04916 3 flanking region primer 1
catggtcatagctgtttcctgacccatttgctttcctggc

SEQ ID NO: 594 - DNA, 20 bp, Artificial Sequence
  CNAG 04916 3 flanking region primer 2
tctcaacgagtcgctttcgc

SEQ ID NO: 595 - DNA, 20 bp, Artificial Sequence
  CNAG 04916 diagnostic screening primer, pairing with B79
aaaagcatcctgcccagcac

SEQ ID NO: 596 - DNA, 20 bp, Artificial Sequence
  CNAG 04916 Southern blot probe primer
gttgaaagtagttgcgttgg

SEQ ID NO: 597 - DNA, 20 bp, Artificial Sequence
  NAT 212 STM primer
agagcgatcgcgttatagat

SEQ ID NO: 598 - DNA, 24 bp, Artificial Sequence
  STM common primer
gcatgccctgcccctaagaattcg

SEQ ID NO: 599 - DNA, 19 bp, Artificial Sequence
  CNAG 04012 5 flanking region primer 1
ttggtggagtgattgaccc

SEQ ID NO: 600 - DNA, 36 bp, Artificial Sequence
  CNAG 04012 5 flanking region primer 2
ctggccgtcgttttacaatcaaaccagaaggagagc

SEQ ID NO: 601 - DNA, 39 bp, Artificial Sequence
  CNAG 04012 3 flanking region primer 1
gtcatagctgtttcctgaggaggataaaggaagagagtg

SEQ ID NO: 602 - DNA, 19 bp, Artificial Sequence
  CNAG 04012 3 flanking region primer 2
ttatgctggtggtgcctac

SEQ ID NO: 603 - DNA, 19 bp, Artificial Sequence
  CNAG 04012 diagnostic screening primer, pairing with B79
accgaagaggatgaggttg

SEQ ID NO: 604 - DNA, 20 bp, Artificial Sequence
  CNAG 04012 Southern blot probe primer
cagtggtggttgagattgac

SEQ ID NO: 605 - DNA, 20 bp, Artificial Sequence
  NAT 212 STM primer
agagcgatcgcgttatagat

SEQ ID NO: 606 - DNA, 24 bp, Artificial Sequence
  STM common primer
gcatgccctgcccctaagaattcg

SEQ ID NO: 607 - DNA, 20 bp, Artificial Sequence
  CNAG 04090 5 flanking region primer 1
gagtagaggagtggattggg

SEQ ID NO: 608 - DNA, 39 bp, Artificial Sequence
  CNAG 04090 5 flanking region primer 2
gctcactggccgtcgttttacccattgttcaagcagagc

SEQ ID NO: 609 - DNA, 42 bp, Artificial Sequence
  CNAG 04090 3 flanking region primer 1
catggtcatagctgtttcctgggcttactgctatcttgatgc

SEQ ID NO: 610 - DNA, 24 bp, Artificial Sequence
  CNAG 04090 3 flanking region primer 2
gaaagaggaactatatactgggtc

SEQ ID NO: 611 - DNA, 20 bp, Artificial Sequence
  CNAG 04090 diagnostic screening primer, pairing with B79
gtcttttgctccttgaaacc

SEQ ID NO: 612 - DNA, 20 bp, Artificial Sequence
  CNAG 04090 Southern blot probe primer
accaggtgtaaacccagtcc

SEQ ID NO: 613 - DNA, 20 bp, Artificial Sequence
  NAT 220 STM primer
cagatctcgaacgataccca

SEQ ID NO: 614 - DNA, 24 bp, Artificial Sequence
  STM common primer
gcatgccctgcccctaagaattcg

SEQ ID NO: 615 - DNA, 21 bp, Artificial Sequence
  CNAG 05375 5 flanking region primer 1
ggaagtattactgtcccatag

SEQ ID NO: 616 - DNA, 43 bp, Artificial Sequence
  CNAG 05375 5 flanking region primer 2
gctcactggccgtcgttttacagaaacaggagagagagtagcc

SEQ ID NO: 617 - DNA, 39 bp, Artificial Sequence
  CNAG 05375 3 flanking region primer 1
catggtcatagctgtttcctgtcttggatttcaacgggg

SEQ ID NO: 618 - DNA, 20 bp, Artificial Sequence
  CNAG 05375 3 flanking region primer 2
gtagttccgttatttgctgg

SEQ ID NO: 619 - DNA, 22 bp, Artificial Sequence
  CNAG 05375 diagnostic screening primer, pairing with B79
ggttgaagatggatgagtagac

SEQ ID NO: 620 - DNA, 20 bp, Artificial Sequence
  CNAG 05375 Southern blot probe primer
cattcacctcccttagaagc

SEQ ID NO: 621 - DNA, 19 bp, Artificial Sequence
  NAT 224 STM priMer
aacctttaaatgggtagag

SEQ ID NO: 622 - DNA, 24 bp, Artificial Sequence
  STM common primer
gcatgccctgcccctaagaattcg

SEQ ID NO: 623 - DNA, 20 bp, Artificial Sequence
  CNAG 07797 5 flanking region primer 1
agggagaacaccacaggatg

SEQ ID NO: 624 - DNA, 38 bp, Artificial Sequence
  CNAG 07797 5 flanking region primer 2
tcactggccgtcgttttacaggaatggaggtggtgaac

SEQ ID NO: 625 - DNA, 40 bp, Artificial Sequence
  CNAG 07797 3 flanking region primer 1
catggtcatagctgtttcctgggaaagtcagtgatgctcc

SEQ ID NO: 626 - DNA, 18 bp, Artificial Sequence
  CNAG 07797 3 flanking region primer 2
gctttcttccttttcccg

SEQ ID NO: 627 - DNA, 18 bp, Artificial Sequence
  CNAG 07797 diagnostic screening primer, pairing with B79
atcaatggtttcgtcggc

SEQ ID NO: 628 - DNA, 18 bp, Artificial Sequence
  CNAG 07797 Southern blot probe primer
ggggcgttgaaaatgatg

SEQ ID NO: 629 - DNA, 20 bp, Artificial Sequence
  NAT 231 STM primer
gagagatcccaacatcacgc

SEQ ID NO: 630 - DNA, 24 bp, Artificial Sequence
  STM common primer
gcatgccctgcccctaagaattcg

SEQ ID NO: 631 - DNA, 22 bp, Artificial Sequence
  CNAG 05538 5 flanking region primer 1
ggtctcaacaaagcacttactg

SEQ ID NO: 632 - DNA, 40 bp, Artificial Sequence
  CNAG 05538 5 flanking region priMer 2
tcactggccgtcgttttacgtaatagtcaggcggtttctc

SEQ ID NO: 633 - DNA, 41 bp, Artificial Sequence
  CNAG 05538 3 flanking region primer 1
catggtcatagctgtttcctgagttaccagaggaggagtgg

SEQ ID NO: 634 - DNA, 20 bp, Artificial Sequence
  CNAG 05538 3 flanking region primer 2
gattctdtttaccctcagcc

SEQ ID NO: 635 - DNA, 20 bp, Artificial Sequence
  CNAG 05538 diagnostic screening primer, pairing with B79
tccgattctattgattttgc

SEQ ID NO: 636 - DNA, 20 bp, Artificial Sequence
  CNAG 05538 Southern blot probe primer
gtatcatccattttcctggc

SEQ ID NO: 637 - DNA, 20 bp, Artificial Sequence
  NAT 240 STM primer
ggtgttggatcggggtggat

SEQ ID NO: 638 - DNA, 24 bp, Artificial Sequence
  STM common primer
gcatgccctgcccctaagaattcg

SEQ ID NO: 639 - DNA, 18 bp, Artificial Sequence
  CNAG 00332 5 flanking region primer 1
tgattgtcgtccttgtgg

SEQ ID NO: 640 - DNA, 39 bp, Artificial Sequence
  CNAG 00332 5 flanking region primer 2
gctcactggccgtcgttttactttgcgtgactgttctgc

SEQ ID NO: 641 - DNA, 42 bp, Artificial Sequence
  CNAG 00332 3 flanking region primer 1
catggtcatagctgtttcctgcggtttgcgaagatacagaac

SEQ ID NO: 642 - DNA, 18 bp, Artificial Sequence
  CNAG 00332 3 flanking region priMer 2
tatggatgcgtgggaatg

SEQ ID NO: 643 - DNA, 18 bp, Artificial Sequence
  CNAG 00332 diagnostic screening primer, pairing with B79
gccagaccaatcaaatgc

SEQ ID NO: 644 - DNA, 19 bp, Artificial Sequence
  CNAG 00332 Southern blot probe primer
gaagaagggttgaatgtcg

SEQ ID NO: 645 - DNA, 24 bp, Artificial Sequence
  NAT 290 STM primer
accgacagctcgaacaagcaagag

SEQ ID NO: 646 - DNA, 24 bp, Artificial Sequence
  STM common primer
gcatgccctgcccctaagaattcg

SEQ ID NO: 647 - DNA, 19 bp, Artificial Sequence
  CNAG 04023 5 flanking region primer 1
agcaggatagacagcgaag

SEQ ID NO: 648 - DNA, 35 bp, Artificial Sequence
  CNAG 04023 5 flanking region primer 2
ctggccgtcgttttactgtccttgataggtcggag

SEQ ID NO: 649 - DNA, 35 bp, Artificial Sequence
  CNAG 04023 3 flanking region primer 1
gtcatagctgtttcctgtggaacacattggaggag

SEQ ID NO: 650 - DNA, 18 bp, Artificial Sequence
  CNAG 04023 3 flanking region primer 2
aatgggtgtatgatggcg

SEQ ID NO: 651 - DNA, 19 bp, Artificial Sequence
  CNAG 04023 diagnostic screening primer, pairing with B79
cagaacgcattgtttgtgc

SEQ ID NO: 652 - DNA, 19 bp, Artificial Sequence
  CNAG 04023 Southern blot probe primer
gtagtaaacgccttgagcc

SEQ ID NO: 653 - DNA, 20 bp, Artificial Sequence
  CNAG 05311 5 flanking region primer 1
caacaagttccatcacatcg

SEQ ID NO: 654 - DNA, 40 bp, Artificial Sequence
  CNAG 05311 5 flanking region primer 2
gctcactggccgtcgttttacgggtaaaacctggcttagg

SEQ ID NO: 655 - DNA, 39 bp, Artificial Sequence
  CNAG 05311 3 flanking region primer 1
catggtcatagctgtttcctgggatttggatgaacctgg

SEQ ID NO: 656 - DNA, 21 bp, Artificial Sequence
  CNAG 05311 3 flanking region primer 2
gagaaagaggtttgtcgtgag

SEQ ID NO: 657 - DNA, 18 bp, Artificial Sequence
  CNAG 05311 diagnostic screening primer, pairing with B79
ttctactttccgccaagg

SEQ ID NO: 658 - DNA, 19 bp, Artificial Sequence
  CNAG 05311 Southern blot probe primer
gagatgggtcactaatcgc

SEQ ID NO: 659 - DNA, 19 bp, Artificial Sequence
  CNAG 03086 5 flanking region primer 1
aatctcctttgcccgactc

SEQ ID NO: 660 - DNA, 38 bp, Artificial Sequence
  CNAG 03086 5 flanking region primer 2
tcactggccgtcgttttacataccttgtctttgcgggc

SEQ ID NO: 661 - DNA, 41 bp, Artificial Sequence
  CNAG 03086 3 flanking region primer 1
catggtcatagctgtttcctgattgaaggtggcagtagatg

SEQ ID NO: 662 - DNA, 18 bp, Artificial Sequence
  CNAG 03086 3 flanking region primer 2
aaaatgtgggcggatgag

SEQ ID NO: 663 - DNA, 19 bp, Artificial Sequence
  CNAG 03086 diagnostic screening primer, pairing with B79
tcagtcgggaagagtttgc

SEQ ID NO: 664 - DNA, 19 bp, Artificial Sequence
  CNAG 03086 Southern blot probe primer
aggcgagagtaagaatgcg

SEQ ID NO: 665 - DNA, 20 bp, Artificial Sequence
  NAT 191 STM primer
atatggatgtttttagcgag

SEQ ID NO: 666 - DNA, 24 bp, Artificial Sequence
  STM common primer
gcatgccctgcccctaagaattcg

SEQ ID NO: 667 - DNA, 18 bp, Artificial Sequence
  CNAG 03336 5 flanking region primer 1
gtgccttattccaatggg

SEQ ID NO: 668 - DNA, 34 bp, Artificial Sequence
  CNAG 03336 5 flanking region primer 2
ctggccgtcgttttacttcttgctgcctgtttgg

SEQ ID NO: 669 - DNA, 35 bp, Artificial Sequence
  CNAG 03336 3 flanking region primer 1
ctggccgtcgttttacccaacaataatcacgcagg

SEQ ID NO: 670 - DNA, 20 bp, Artificial Sequence
  CNAG 03336 3 flanking region primer 2
ccatcctttttttagcggtc

SEQ ID NO: 671 - DNA, 19 bp, Artificial Sequence
  CNAG 03336 diagnostic screening primer, pairing with B79
cgaggtgattgatgtgagc

SEQ ID NO: 672 - DNA, 20 bp, Artificial Sequence
  CNAG 03336 Southern blot probe primer
ttcaagagcggactggacag

SEQ ID NO: 673 - DNA, 20 bp, Artificial Sequence
  NAT 204 STM primer
gatctctcgcgcttggggga

SEQ ID NO: 674 - DNA, 24 bp, Artificial Sequence
  STM common primer
gcatgccctgcccctaagaattcg

SEQ ID NO: 675 - DNA, 19 bp, Artificial Sequence
  CNAG 03229 5 flanking region primer 1
gtgggttactttgggtgag

SEQ ID NO: 676 - DNA, 37 bp, Artificial Sequence
  CNAG 03229 5 flanking region primer 2
tcactggccgtcgttttacaaaagggacttgctgtcg

SEQ ID NO: 677 - DNA, 40 bp, Artificial Sequence
  CNAG 03229 3 flanking region primer 1
catggtcatagctgtttcctggaaagaaaggaaatcgccg

SEQ ID NO: 678 - DNA, 18 bp, Artificial Sequence
  CNAG 03229 3 flanking region primer 2
tggaagatggtgacgaag

SEQ ID NO: 679 - DNA, 18 bp, Artificial Sequence
  CNAG 03229 diagnostic screening primer, pairing with B79
ttgcgatttccgactacc

SEQ ID NO: 680 - DNA, 18 bp, Artificial Sequence
  CNAG 03229 Southern blot probe primer
tggaggcgaataatggtc

SEQ ID NO: 681 - DNA, 20 bp, Artificial Sequence
  NAT 227 STM primer
tcgtggtttagagggagcgc

SEQ ID NO: 682 - DNA, 24 bp, Artificial Sequence
  STM common primer
gcatgccctgcccctaagaattcg

SEQ ID NO: 683 - DNA, 19 bp, Artificial Sequence
  CNAG 01883 5 flanking region primer 1
tactcgctgcttagggttc

SEQ ID NO: 684 - DNA, 35 bp, Artificial Sequence
  CNAG 01883 5 flanking region primer 2
ctggccgtcgttttacatggcatactccaggtctg

SEQ ID NO: 685 - DNA, 36 bp, Artificial Sequence
  CNAG 01883 3 flanking region primer 1
gtcatagctgtttcctgccctccatttacttctccg

SEQ ID NO: 686 - DNA, 22 bp, Artificial Sequence
  CNAG 01883 3 flanking region primer 2
tagcagtagggctcctgattcg

SEQ ID NO: 687 - DNA, 19 bp, Artificial Sequence
  CNAG 01883 diagnostic screening primer, pairing with B79
ttggaggtctttatcagcg

SEQ ID NO: 688 - DNA, 19 bp, Artificial Sequence
  CNAG 01883 Southern blot probe primer
ggagaagtaaatggagggg

SEQ ID NO: 689 - DNA, 24 bp, Artificial Sequence
  NAT 125 STM primer
cgctacagdcagcgcgcgcaagcg

SEQ ID NO: 690 - DNA, 24 bp, Artificial Sequence
  STM common primer
gcatgccctgcccctaagaattcg

SEQ ID NO: 691 - DNA, 20 bp, Artificial Sequence
  CNAG 04837 5 flanking region primer 1
tgaaacagtgaatcaggcac

SEQ ID NO: 692 - DNA, 40 bp, Artificial Sequence
  CNAG 04837 5 flanking region primer 2
gctcactggccgtcgttttactcagtgtcaacggtcattg

SEQ ID NO: 693 - DNA, 42 bp, Artificial Sequence
  CNAG 04837 3 flanking region primer 1
catggtcatagctgtttcctggaaaggggtagggataagttg

SEQ ID NO: 694 - DNA, 20 bp, Artificial Sequence
  CNAG 04837 3 flanking region primer 2
actgaacgacaagagacgac

SEQ ID NO: 695 - DNA, 18 bp, Artificial Sequence
  CNAG 04837 diagnostic screening primer, pairing with B79
taagagaaaaggggctgc

SEQ ID NO: 696 - DNA, 21 bp, Artificial Sequence
  CNAG 04837 Southern blot probe primer 1
tgtccccatcaactctacaac

SEQ ID NO: 697 - DNA, 20 bp, Artificial Sequence
  CNAG 04837 Southern blot probe primer 2
cttctgctttcaccattagc

SEQ ID NO: 698 - DNA, 21 bp, Artificial Sequence
  NAT 146 STM primer
actagccccccctcaccacct

SEQ ID NO: 699 - DNA, 24 bp, Artificial Sequence
  STM common primer
gcatgccctgbccctaagaattcg

SEQ ID NO: 700 - DNA, 19 bp, Artificial Sequence
  CNAG 00514 5 flanking region primer 1
ggtgtttccatacgaaccg

SEQ ID NO: 701 - DNA, 38 bp, Artificial Sequence
  CNAG 00514 5 flanking region primer 2
ctggccgtcgttttacggagccatttcttacttatgcg

SEQ ID NO: 702 - DNA, 35 bp, Artificial Sequence
  CNAG 00514 3 flankihg region primer 1
gtcatagctgtttcctgaatcccaaggaccatctg

SEQ ID NO: 703 - DNA, 20 bp, Artificial Sequence
  CNAG 00514 3 flanking region primer 2
gctgagaaggagaggatagc

SEQ ID NO: 704 - DNA, 20 bp, Artificial Sequence
  CNAG 00514 diagnostic screening primer, pairing with B79
gtggttgaaagtattaggcg

SEQ ID NO: 705 - DNA, 19 bp, Artificial Sequence
  CNAG 00514 Southern blot probe primer
gaggattggtgatggactg

SEQ ID NO: 706 - DNA, 24 bp, Artificial Sequence
  NAT 201 STM primer
caccctctatctcgagaaagctcc

SEQ ID NO: 707 - DNA, 24 bp, Artificial Sequence
  STM common primer
gcatgccctgcccctaagaattcg

SEQ ID NO: 708 - DNA, 20 bp, Artificial Sequence
  CNAG 03409 5 flanking region primer 1
cctcaccgattcattcactg

SEQ ID NO: 709 - DNA, 38 bp, Artificial Sequence
  CNAG 03409 5 flanking region primer 2
ctggccgtcgttttacttttctcccttcttcgcccgac

SEQ ID NO: 710 - DNA, 37 bp, Artificial Sequence
  CNAG 03409 3 flanking region primer 1
gtcatagctgtttcctgaggttcaagcccagattcag

SEQ ID NO: 711 - DNA, 20 bp, Artificial Sequence
  CNAG 03409 3 flanking region primer 2
agtatctacggtgctggtgc

SEQ ID NO: 712 - DNA, 20 bp, Artificial Sequence
  CNAG 03409 diagnostic screening primer, pairing with B79
tgaggatgatgactggggac

SEQ ID NO: 713 - DNA, 20 bp, Artificial Sequence
  CNAG 03409 Southern blot probe primer 1
acttccaatgctcaaccccg

SEQ ID NO: 714 - DNA, 20 bp, Artificial Sequence
  CNAG 03409 Southern blot probe primer 2
gcaggacaagaaggagagtg

SEQ ID NO: 715 - DNA, 24 bp, Artificial Sequence
  NAT 201 STM primer
caccctctatctcgagaaagctcc

SEQ ID NO: 716 - DNA, 24 bp, Artificial Sequence
  STM common primer
gcatgccctgcccctaagaattcg

SEQ ID NO: 717 - DNA, 19 bp, Artificial Sequence
  CNAG 05392 5 flanking region primer 1
aagttgtgaaaccaccagg

SEQ ID NO: 718 - DNA, 38 bp, Artificial Sequence
  CNAG 05392 5 flanking region primer 2
tcactggccgtcgttttactcaccctgtagatgtgtgg

SEQ ID NO: 719 - DNA, 42 bp, Artificial Sequence
  CNAG 05392 3 flanking region primer 1
catggtcatagctgtttcctgggagtccctgtgaagtaaaag

SEQ ID NO: 720 - DNA, 20 bp, Artificial Sequence
  CNAG 05392 3 flanking region primer 2
tgagccaaagtagcagtcag

SEQ ID NO: 721 - DNA, 19 bp, Artificial Sequence
  CNAG 05392 diagnostic screening primer, pairing with B79
gaatggaccttggaaaacg

SEQ ID NO: 722 - DNA, 20 bp, Artificial Sequence
  CNAG 05392 Southern blot probe primer
cggtctgtgtgcttcttcac

SEQ ID NO: 723 - DNA, 20 bp, Artificial Sequence
  NAT 204 STM primer
gatctctcgcgcttggggga

SEQ ID NO: 724 - DNA, 24 bp, Artificial Sequence
  STM common primer
gcatgccctgcccctaagaattcg

SEQ ID NO: 725 - DNA, 20 bp, Artificial Sequence
  CNAG 04895 5 flanking region primer 1
ttcgtagttagccagcagag

SEQ ID NO: 726 - DNA, 38 bp, Artificial Sequence
  CNAG 04895 5 flanking region primer 2
tcactggccgtcgttttacgttcttgatgtggaggagc

SEQ ID NO: 727 - DNA, 40 bp, Artificial Sequence
  CNAG 04895 3 flanking region primer 1
catggtcatagctgtttcctgcccaacccttatgcgtatg

SEQ ID NO: 728 - DNA, 18 bp, Artificial Sequence
  CNAG 04895 3 flanking region primer 2
agcgtctccctcaaaaac

SEQ ID NO: 729 - DNA, 18 bp, Artificial Sequence
  CNAG 04895 diagnostic screening primer, pairing with B79
ttagcgaccgaaaaaggg

SEQ ID NO: 730 - DNA, 19 bp, Artificial Sequence
  CNAG 04895 Southern blot probe primer
aagatgacgcagaactggg

SEQ ID NO: 731 - DNA, 20 bp, Artificial Sequence
  NAT 204 STM primer
gatctctcgcgcttggggga

SEQ ID NO: 732 - DNA, 24 bp, Artificial Sequence
  STM common primer
gcatgccctgcccctaagaattcg

SEQ ID NO: 733 - DNA, 19 bp, Artificial Sequence
  CNAG 04176 5 flanking region primer 1
ttgacacggggactaatcg

SEQ ID NO: 734 - DNA, 40 bp, Artificial Sequence
  CNAG 04176 5 flanking region primer 2
gctcactggccgtcgttttacgattagcccagtcttagcg

SEQ ID NO: 735 - DNA, 41 bp, Artificial Sequence
  CNAG 04176 3 flanking region primer 1
catggtcatagctgtttcctgagaagcagggagcaaaacgg

SEQ ID NO: 736 - DNA, 19 bp, Artificial Sequence
  CNAG 04176 3 flanking region primer 2
ccgaaaagttgagcaagac

SEQ ID NO: 737 - DNA, 23 bp, Artificial Sequence
  CNAG 04176 diagnostic screening primer, pairing with B79
cgatagcctttgtagtaggtctc

SEQ ID NO: 738 - DNA, 20 bp, Artificial Sequence
  CNAG 04176 Southern blot probe primer
gggatttgattatctggagg

SEQ ID NO: 739 - DNA, 24 bp, Artificial Sequence
  NAT 205 STM primer
tatccccctctccgctctctagca

SEQ ID NO: 740 - DNA, 24 bp, Artificial Sequence
  STM common primer
gcatgccctgcccctaagaattcg

SEQ ID NO: 741 - DNA, 19 bp, Artificial Sequence
  CNAG 07724 5 flanking region primer 1
caaagaaaacgctcactcc

SEQ ID NO: 742 - DNA, 40 bp, Artificial Sequence
  CNAG 07724 5 flanking region primer 2
tcactggccgtcgttttacggatagaacaagacaagagcg

SEQ ID NO: 743 - DNA, 41 bp, Artificial Sequence
  CNAG 07724 3 flanking region primer 1
catggtcatagctgtttcctgacttcagtcgttaccagcac

SEQ ID NO: 744 - DNA, 19 bp, Artificial Sequence
  CNAG 07724 3 flanking region primer 2
tctttgagcacatcaggtg

SEQ ID NO: 745 - DNA, 19 bp, Artificial Sequence
  CNAG 07724 diagnostic screening primer, pairing with B79
ccgtcattatcgtgtctcc

SEQ ID NO: 746 - DNA, 19 bp, Artificial Sequence
  CNAG 07724 Southern blbt probe primer
gcttcggaggagttgatag

SEQ ID NO: 747 - DNA, 24 bp, Artificial Sequence
  NAT 205 STM primer
tatccccctctccgctctctagca

SEQ ID NO: 748 - DNA, 24 bp, Artificial Sequence
  STM common primer
gcatgccctgcccctaagaattcg

SEQ ID NO: 749 - DNA, 19 bp, Artificial Sequence
  CNAG 06751 5 flanking region primer 1
aaaaacggctgaaggctcg

SEQ ID NO: 750 - DNA, 40 bp, Artificial Sequence
  CNAG 06751 5 flanking region primer 2
gctcactggccgtcgttttactggcgagttggtatctgag

SEQ ID NO: 751 - DNA, 40 bp, Artificial Sequence
  CNAG 06751 3 flanking region primer 1
catggtcatagctgtttcctgaagatgagagagaaggggg

SEQ ID NO: 752 - DNA, 20 bp, Artificial Sequence
  CNAG 06751 3 flanking region pfimer 2
agaagtggtatgaccgtttg

SEQ ID NO: 753 - DNA, 19 bp, Artificial Sequence
  CNAG 06751 diagnostic screening primer, pairing with B79
tccatacattgttgagggc

SEQ ID NO: 754 - DNA, 18 bp, Artificial Sequence
  CNAG 06751 Southern blot probe primer
attgcccaagcggataag

SEQ ID NO: 755 - DNA, 22 bp, Artificial Sequence
  NAT 208 STM primer
tggtcgcgggagatcgtggttt

SEQ ID NO: 756 - DNA, 24 bp, Artificial Sequence
  STM common primer
gcatgccctgcccctaagaattcg

SEQ ID NO: 757 - DNA, 18 bp, Artificial Sequence
  CNAG 01438 5 flanking region primer 1
atcacacgcaatcccatc

SEQ ID NO: 758 - DNA, 39 bp, Artificial Sequence
  CNAG 01438 5 flanking region primer 2
gctcactggccgtcgttttacagcaagatggaaggaagc

SEQ ID NO: 759 - DNA, 39 bp, Artificial Sequence
  CNAG 01438 3 flanking region primer 1
catggtcatagctgtttcctggcgacgaaatcataaggc

SEQ ID NO: 760 - DNA, 19 bp, Artificial Sequence
  CNAG 01438 3 flanking region primer 2
tgagaactggacgaactgc

SEQ ID NO: 761 - DNA, 18 bp, Artificial Sequence
  CNAG 01438 diagnostic screening primer, pairing with B79
aatgatgcgtcggacttc

SEQ ID NO: 762 - DNA, 20 bp, Artificial Sequence
  CNAG 01438 Southern blot probe primer
ggaagagaaagacttgcgag

SEQ ID NO: 763 - DNA, 24 bp, Artificial Sequence
  NAT 122 STM primer
acagctccaaacctcgctaaacag

SEQ ID NO: 764 - DNA, 24 bp, Artificial Sequence
  STM common primer
gcatgccctgcccctaagaattcg

SEQ ID NO: 765 - DNA, 20 bp, Artificial Sequence
  CNAG 00791 5 flanking region primer 1
gcatccattttatcggttcg

SEQ ID NO: 766 - DNA, 41 bp, Artificial Sequence
  CNAG 00791 5 flanking region primer 2
gctcactggccgtcgttttacttctgcttgaaggagtcggg

SEQ ID NO: 767 - DNA, 40 bp, Artificial Sequence
  CNAG 00791 3 flanking region primer 1
catggtcatagctgtttcctgcggttatcttgcgtaaagc

SEQ ID NO: 768 - DNA, 19 bp, Artificial Sequence
  CNAG 00791 3 flanking region primer 2
tgtctcacccatcacgaac

SEQ ID NO: 769 - DNA, 18 bp, Artificial Sequence
  CNAG 00791 diagnostic screening primer, pairing with B79
attcgctcaggcagtttc

SEQ ID NO: 770 - DNA, 19 bp, Artificial Sequence
  CNAG 00791 Southern blot probe primer
gcgatgatgaggactcttg

SEQ ID NO: 771 - DNA, 21 bp, Artificial Sequence
  NAT 146 STM primer
actagccccccctcaccacct

SEQ ID NO: 772 - DNA, 24 bp, Artificial Sequence
  STM common primer
gcatgccctgcccctaagaattcg

SEQ ID NO: 773 - DNA, 19 bp, Artificial Sequence
  CNAG 02241 5 flanking region primer 1
atagcaaacccatacctcg

SEQ ID NO: 774 - DNA, 37 bp, Artificial Sequence
  CNAG 02241 5 flanking region primer 2
tcactggccgtcgttttactttgtttggtagtggggg

SEQ ID NO: 775 - DNA, 40 bp, Artificial Sequence
  CNAG 02241 3 flanking region primer 1
catggtcatagctgtttcctgcgagaactctggagcattc

SEQ ID NO: 776 - DNA, 21 bp, Artificial Sequence
  CNAG 02241 3 flanking region primer 2
gatgtgttgtggactgtatgc

SEQ ID NO: 777 - DNA, 20 bp, Artificial Sequence
  CNAG 02241 diagnostic screening primer, pairing with B79
tcccatcataggagtggacg

SEQ ID NO: 778 - DNA, 20 bp, Artificial Sequence
  CNAG 02241 Southern blot probe primer
cttgggagcaaataagtgtg

SEQ ID NO: 779 - DNA, 20 bp, Artificial Sequence
  NAT 219 STM primer
ccctaaaaccctacagcaat

SEQ ID NO: 780 - DNA, 24 bp, Artificial Sequence
  STM common primer
gcatgccctgcccctaagaattcg

SEQ ID NO: 781 - DNA, 19 bp, Artificial Sequence
  CNAG 02566 5 flanking region primer 1
tcgcacggcataggattag

SEQ ID NO: 782 - DNA, 38 bp, Artificial Sequence
  CNAG 02566 5 flanking region primer 2
tcactggccgtcgttttacatgccgccagaatgttgag

SEQ ID NO: 783 - DNA, 40 bp, Artificial Sequence
  CNAG 02566 3 flanking region primer 1
catggtcatagctgtttcctgtgatgttgatggagactgg

SEQ ID NO: 784 - DNA, 20 bp, Artificial Sequence
  CNAG 02566 3 flanking region primer 2
actgcttcttcaccctccac

SEQ ID NO: 785 - DNA, 18 bp, Artificial Sequence
  CNAG 02566 diagnostic screening primer, pairing with B79
atctgccgcaacattcac

SEQ ID NO: 786 - DNA, 21 bp, Artificial Sequence
  CNAG 02566 SoUthern blot probe, primer
gttggtgcttcatagtgactg

SEQ ID NO: 787 - DNA, 20 bp, Artificial Sequence
  NAT 219 STM primer
ccctaaaaccctacagcaat

SEQ ID NO: 788 - DNA, 24 bp, Artificial Sequence
  STM common primer
gcatgccctgcccctaagaattcg

SEQ ID NO: 789 - DNA, 19 bp, Artificial Sequence
  CNAG 01626 5 flanking region primer 1
ggatgatggaatcgtatgc

SEQ ID NO: 790 - DNA, 38 bp, Artificial Sequence
  CNAG 01626 5 flanking region primer 2
tcactggccgtcgttttacattatccaccctcggcttc

SEQ ID NO: 791 - DNA, 42 bp, Artificial Sequence
  CNAG 01626 3 flanking region primer 1
catggtcatagctgtttcctggcgaacggtatgacaactatg

SEQ ID NO: 792 - DNA, 22 bp, Artificial Sequence
  CNAG 01626 3 flanking region primer 2
ggacgaaaacattgctctcaac

SEQ ID NO: 793 - DNA, 19 bp, Artificial Sequence
  CNAG 01626 diagnostic screening primer, pairing with B79
tcagcagcagcaggtaaac

SEQ ID NO: 794 - DNA, 18 bp, Artificial Sequence
  CNAG 01626 Southern blot probe primer
cccagaccgttttgaatg

SEQ ID NO: 795 - DNA, 18 bp, Artificial Sequence
  NAT 232 STM primer
ctttaaaggtggtttgtg

SEQ ID NO: 796 - DNA, 24 bp, Artificial Sequence
  STM common primer
gcatgccctgcccctaagaattcg

SEQ ID NO: 797 - DNA, 20 bp, Artificial Sequence
  CNAG 05940 5 flanking region primer 1
tcttgaccgtcgccagttac

SEQ ID NO: 798 - DNA, 39 bp, Artificial Sequence
  CNAG 05940 5 flanking region primer 2
tcactggccgtcgttttactggctcaaaggcaaagtcgg

SEQ ID NO: 799 - DNA, 40 bp, Artificial Sequence
  CNAG 05940 3 flanking region primer 1
catggtcatagctgtttcctgaggtgattttgggagtcag

SEQ ID NO: 800 - DNA, 19 bp, Artificial Sequence
  CNAG 05940 3 flanking region primer 2
ggctgagaatgatgtgacg

SEQ ID NO: 801 - DNA, 19 bp, Artificial Sequence
  CNAG 05940 diagnostic screening primer, pairing with B79
ggaaagaagacggatagcg

SEQ ID NO: 802 - DNA, 19 bp, Artificial Sequence
  CNAG 05940 Southern blot probe primer
gtctccaggaaagttcgtg

SEQ ID NO: 803 - DNA, 18 bp, Artificial Sequence
  NAT 232 STM primer
ctttaaaggtggtttgtg

SEQ ID NO: 804 - DNA, 24 bp, Artificial Sequence
  STM common primer
gcatgccctgcccctaagaattcg

SEQ ID NO: 805 - DNA, 22 bp, Artificial Sequence
  CNAG 04352 5 flanking region primer 1
gtgattatgtgaacccgttacc

SEQ ID NO: 806 - DNA, 39 bp, Artificial Sequence
  CNAG 04352 5 flanking region primer 2
tcactggccgtcgttttacttttagaggtgatgactgcg

SEQ ID NO: 807 - DNA, 41 bp, Artificial Sequence
  CNAG 04352 3 flanking region primer 1
catggtcatagctgtttcctgtgctgctggaggaagagaac

SEQ ID NO: 808 - DNA, 19 bp, Artificial Sequence
  CNAG 04352 3 flanking region primer 2
agtgagtcgttgatgtggc

SEQ ID NO: 809 - DNA, 19 bp, Artificial Sequence
  CNAG 04352 diagnostic screening primer, pairing with B79
atagtgaatacgggtcggg

SEQ ID NO: 810 - DNA, 19 bp, Artificial Sequence
  CNAG 04352 Southern blot probe primer
gctgaaacaggaggcatag

SEQ ID NO: 811 - DNA, 20 bp, Artificial Sequence
  NAT 234 STM primer
ccagccagcactccgatata

SEQ ID NO: 812 - DNA, 24 bp, Artificial Sequence
  STM common primer
gcatgccctgcccctaagaattcg

SEQ ID NO: 813 - DNA, 18 bp, Artificial Sequence
  CNAG 03768 5 flanking region primer 1
aatgtccttgcggttgac

SEQ ID NO: 814 - DNA, 40 bp, Artificial Sequence
  CNAG 03768 5 flanking region primer 2
gctcactggccgtcgttttacaatagtccccgtcgttgtc

SEQ ID NO: 815 - DNA, 41 bp, Artificial Sequence
  CNAG 03768 3 flanking region primer 1
catggtcatagctgtttcctgcgatggatgtttggtggtag

SEQ ID NO: 816 - DNA, 19 bp, Artificial Sequence
  CNAG 03768 3 flanking region primer 2
aaaggggttttggtggctc

SEQ ID NO: 817 - DNA, 20 bp, Artificial Sequence
  CNAG 03768 diagnostic screening primer, pairing with B79
tggatttggcagagaagaag

SEQ ID NO: 818 - DNA, 19 bp, Artificial Sequence
  CNAG 03768 Southern blot probe primer
aagacacttggctctccag

SEQ ID NO: 819 - DNA, 20 bp, Artificial Sequence
  NAT 234 STM primer
ccagccagcactccgatata

SEQ ID NO: 820 - DNA, 24 bp, Artificial Sequence
  STM common primer
gcatgccctgcccctaagaattcg

SEQ ID NO: 821 - DNA, 20 bp, Artificial Sequence
  CNAG 02322 5 flanking region primer 1
cgctcactcacctttcaaac

SEQ ID NO: 822 - DNA, 35 bp, Artificial Sequence
  CNAG 02322 5 flanking region primer 2
ctggccgtcgttttacacatcggcttttacggagc

SEQ ID NO: 823 - DNA, 37 bp, Artificial Sequence
  CNAG 02322 3 flanking region primer 1
gtcatagctgtttcctgctcggttaccagtcactacg

SEQ ID NO: 824 - DNA, 20 bp, Artificial Sequence
  CNAG 02322 3 flanking region primer 2
ccaccaagattcagagttcg

SEQ ID NO: 825 - DNA, 19 bp, Artificial Sequence
  CNAG 02322 diagnostic screening primer, pairing with B79
gagatttcggtggatttcc

SEQ ID NO: 826 - DNA, 18 bp, Artificial Sequence
  CNAG 02322 Southern blot probe primer
aagcaaccctcagcattg

SEQ ID NO: 827 - DNA, 20 bp, Artificial Sequence
  NAT 240 STM primer
ggtgttggatcggggtggat

SEQ ID NO: 828 - DNA, 24 bp, Artificial Sequence
  STM common primer
gcatgccctgcccctaagaattcg

SEQ ID NO: 829 - DNA, 20 bp, Artificial Sequence
  CNAG 06818 5 flanking region primer 1
tgtattctgcccacctccac

SEQ ID NO: 830 - DNA, 39 bp, Artificial Sequence
  CNAG 06818 5 flanking region primer
tcactggccgtcgttttaccctctttgctggtctctgag

SEQ ID NO: 831 - DNA, 41 bp, Artificial Sequence
  CNAG 06818 3 flanking region primer 1
catggtcatagctgtttcctgtctgctgctacatcaggttc

SEQ ID NO: 832 - DNA, 19 bp, Artificial Sequence
  CNAG 06818 3 flanking region primer 2
aacctctgtatcgctgacg

SEQ ID NO: 833 - DNA, 22 bp, Artificial Sequence
  CNAG 06818 diagnostic screening primer, pairing with B79
gggtgaaagacaataggtaagg

SEQ ID NO: 834 - DNA, 21 bp, Artificial Sequence
  CNAG 06818 Southern blot probe primer
aaagtgaagagaggaggtcag

SEQ ID NO: 835 - DNA, 20 bp, Artificial Sequence
  NAT 240 STM primer
ggtgttggatcggggtggat

SEQ ID NO: 836 - DNA, 24 bp, Artificial Sequence
  STM common primer
gcatgccctgcccctaagaattcg

SEQ ID NO: 837 - DNA, 19 bp, Artificial Sequence
  CNAG 07011 5 flanking region primer 1
ccaacaaatagcaaagccg

SEQ ID NO: 838 - DNA, 38 bp, Artificial Sequence
  CNAG 07011 5 flanking region primer 2
tcactggccgtcgttttacacaccaaagccaagaacac

SEQ ID NO: 839 - DNA, 41 bp, Artificial Sequence
  CNAG 07011 3 flanking region primer 1
catggtcatagctgtttcctggctaatgcctgaaacagacc

SEQ ID NO: 840 - DNA, 19 bp, Artificial Sequence
  CNAG 07011 3 flanking region primer 2
tcggacacccaatcaatac

SEQ ID NO: 841 - DNA, 18 bp, Artificial Sequence
  CNAG 07011 diagnostic screening primer, pairing with B79
ttgtgttgttcggtgagg

SEQ ID NO: 842 - DNA, 20 bp, Artificial Sequence
  CNAG 07011 Southern blot probe primer
tgggtgatacagaggaaaac

SEQ ID NO: 843 - DNA, 24 bp, Artificial Sequence
  NAT 273 STM primer
gagatctttcgggaggtctggatt

SEQ ID NO: 844 - DNA, 24 bp, Artificial Sequence
  STM common primer
gcatgccctgcccctaagaattcg

SEQ ID NO: 845 - DNA, 20 bp, Artificial Sequence
  CNAG 04036 5 flanking region primer 1
ccagttgaacgcttctaaac

SEQ ID NO: 846 - DNA, 39 bp, Artificial Sequence
  CNAG 04036 5 flanking region primer 2
tcactggccgtcgttttactgatggtgtgggaacaatac

SEQ ID NO: 847 - DNA, 40 bp, Artificial Sequence
  CNAG 04036 3 flanking region primer 1
catggtcatagctgtttcctgttctccttgcctatgtgcc

SEQ ID NO: 848 - DNA, 20 bp, Artificial Sequence
  CNAG 04036 3 flanking region primer 2
cagcaacttactttggaggc

SEQ ID NO: 849 - DNA, 19 bp, Artificial Sequence
  CNAG 04036 diagnostic screening primer, pairing with B79
atagacagcagcgggatac

SEQ ID NO: 850 - DNA, 18 bp, Artificial Sequence
  CNAG 04036 Southern blot probe primer
tcacagatgcctttctcg

SEQ ID NO: 851 - DNA, 24 bp, Artificial Sequence
  NAT 273 STM primer
gagatctttcgggaggtctggatt

SEQ ID NO: 852 - DNA, 24 bp, Artificial Sequence
  STM common primer
gcatgccctgcccctaagaattcg

SEQ ID NO: 853 - DNA, 19 bp, Artificial Sequence
  CNAG 07506 5 flanking region primer 1
cgtaatgacactggagagc

SEQ ID NO: 854 - DNA, 42 bp, Artificial Sequence
  CNAG 07506 5 flanking region primer 2
gctcactggccgtcgttttactgttgccgtaggaaccgcttc

SEQ ID NO: 855 - DNA, 40 bp, Artificial Sequence
  CNAG 07506 3 flanking region primer 1
catggtcatagctgtttcctgtcccaaagcctcttctcag

SEQ ID NO: 856 - DNA, 18 bp, Artificial Sequence
  CNAG 07506 3 flanking region primer 2
gtgagaccctgatagacc

SEQ ID NO: 857 - DNA, 19 bp, Artificial Sequence
  CNAG 07506 diagnostic screening primer, pairing with B79
gactcaaagggagatgacg

SEQ ID NO: 858 - DNA, 19 bp, Artificial Sequence
  CNAG 07506 Southern blot probe primer
gcttttgcctgctattgac

SEQ ID NO: 859 - DNA, 20 bp, Artificial Sequence
  NAT 296 STM primer
cgcccgccctcactatccac

SEQ ID NO: 860 - DNA, 24 bp, Artificial Sequence
  STM common primer
gcatgccctgcccctaagaattcg

SEQ ID NO: 861 - DNA, 19 bp, Artificial Sequence
  CNAG 06223 5 flanking region primer 1
ggatgaaggggaagacttg

SEQ ID NO: 862 - DNA, 38 bp, Artificial Sequence
  CNAG 06223 5 flanking region primer 2
tcactggccgtcgttttaccttccgttcaaaatcgtcg

SEQ ID NO: 863 - DNA, 41 bp, Artificial Sequence
  CNAG 06223 3 flanking region primer 1
catggtcatagctgtttcctggtctaccaccaccatcacac

SEQ ID NO: 864 - DNA, 20 bp, Artificial Sequence
  CNAG 06223 3 flanking region primer 2
gccgtccattcatacatctg

SEQ ID NO: 865 - DNA, 19 bp, Artificial Sequence
  CNAG 06223 diagnostic screening primer, pairing with B79
ggtgtcaatagttgggtgg

SEQ ID NO: 866 - DNA, 19 bp, Artificial Sequence
  CNAG 06223 Southern blot probe primer
agagcagaagcaaatgacc

SEQ ID NO: 867 - DNA, 20 bp, Artificial Sequence
  NAT 210 STM primer
ctagagcccgccacaacgct

SEQ ID NO: 868 - DNA, 24 bp, Artificial Sequence
  STM common primer
gcatgccctgcccctaagaattcg

SEQ ID NO: 869 - DNA, 24 bp, Artificial Sequence
  CNAG 06283 5 flanking region primer 1
ccctttgtccttacccacattttc

SEQ ID NO: 870 - DNA, 39 bp, Artificial Sequence
  CNAG 06283 5 flanking region primer 2
tcactggccgtcgttttactcgttcgggcacttcttgac

SEQ ID NO: 871 - DNA, 41 bp, Artificial Sequence
  CNAG 06283 3 flanking region primer 1
catggtcatagctgtttcctgcgagtaaggagacggttgag

SEQ ID NO: 872 - DNA, 22 bp, Artificial Sequence
  CNAG 06283 3 flanking region primer 2
ggttagggggataaaataggag

SEQ ID NO: 873 - DNA, 21 bp, Artificial Sequence
  CNAG 06283 diagnostic screening primer, pairing with B79
gagacattctttctgacgagc

SEQ ID NO: 874 - DNA, 20 bp, Artificial Sequence
  CNAG 06283 Southern blot probe primer
tggcttgaagggtcttggtc

SEQ ID NO: 875 - DNA, 20 bp, Artificial Sequence
  NAT 234 STM primer
ccagccagcactccgatata

SEQ ID NO: 876 - DNA, 24 bp, Artificial Sequence
  STM common primer
gcatgccctgcccctaagaattcg

SEQ ID NO: 877 - DNA, 22 bp, Artificial Sequence
  CNAG 04583 5 flanking region primer 1
gctcgctacattttcaggtatc

SEQ ID NO: 878 - DNA, 39 bp, Artificial Sequence
  CNAG 04583 5 flanking region primer 2
tcactggccgtcgttttacgagagggtcggaaatagaag

SEQ ID NO: 879 - DNA, 41 bp, Artificial Sequence
  CNAG 04583 3 flanking region primer 1
catggtcatagctgtttcctggggaaggagtaatgcctgac

SEQ ID NO: 880 - DNA, 18 bp, Artificial Sequence
  CNAG 04583 3 flanking region primer 2
aaccgtgatggagattgg

SEQ ID NO: 881 - DNA, 20 bp, Artificial Sequence
  CNAG 04583 diagnostic screening primer, pairing with B79
cggtgtgcgattctaacttc

SEQ ID NO: 882 - DNA, 19 bp, Artificial Sequence
  CNAG 04583 Southern blot probe primer
ggggactgttttgatgacc

SEQ ID NO: 883 - DNA, 24 bp, Artificial Sequence
  NAT 102 STM primer
ccatagcgatatctaccccaatct

SEQ ID NO: 884 - DNA, 24 bp, Artificial Sequence
  STM common primer
gcatgccctgcccctaagaattcg

SEQ ID NO: 885 - DNA, 19 bp, Artificial Sequence
  CNAG 03261 5 flanking region primer 1
ggcgataaagacgacgaac

SEQ ID NO: 886 - DNA, 35 bp, Artificial Sequence
  CNAG 03261 5 flanking region primer 2
ctggccgtcgttttacggactattcgtggcttagc

SEQ ID NO: 887 - DNA, 35 bp, Artificial Sequence
  CNAG 03261 3 flanking region primer 1
gtcatagctgtttcctgtgagaccaatgtgttgcc

SEQ ID NO: 888 - DNA, 24 bp, Artificial Sequence
  CNAG 03261 3 flanking region primer 2
cctcaacccctttctgatttatcc

SEQ ID NO: 889 - DNA, 22 bp, Artificial Sequence
  CNAG 03261 diagnostic screening primer, pairing with B79
tagtcggtgatgagagttgcgg

SEQ ID NO: 890 - DNA, 20 bp, Artificial Sequence
  CNAG 03261 Southern blot probe primer
gcaaatctgcttctgggatg

SEQ ID NO: 891 - DNA, 19 bp, Artificial Sequence
  CNAG 04908 5 flanking region primer 1
gatgacgaatagcacggtc

SEQ ID NO: 892 - DNA, 37 bp, Artificial Sequence
  CNAG 04908 5 flanking region primer 2
tcactggccgtcgttttacggatgggaagattgttgg

SEQ ID NO: 893 - DNA, 39 bp, Artificial Sequence
  CNAG 04908 3 flanking region primer 1
gatggtcatagctgtttcctgttgcttacgcctttgtcc

SEQ ID NO: 894 - DNA, 18 bp, Artificial Sequence
  CNAG 04908 3 flanking region primer 2
atggttgttcacgcacag

SEQ ID NO: 895 - DNA, 18 bp, Artificial Sequence
  CNAG 04908 diagnostic screening primer, pairing with B79
aaacaaaagccgagggag

SEQ ID NO: 896 - DNA, 19 bp, Artificial Sequence
  CNAG 04908 Southern blot probe primer
aacttcccaccccttcatc

SEQ ID NO: 897 - DNA, 24 bp, Artificial Sequence
  NAT 242 STM primer
gtagcgataggggtgtcgctttag

SEQ ID NO: 898 - DNA, 24 bp, Artificial Sequence
  STM common primer
gcatgccctgcccctaagaattcg

SEQ ID NO: 899 - DNA, 19 bp, Artificial Sequence
  CNAG 01317 5 flanking region primer 1
aagcaccaagaagcaagtg

SEQ ID NO: 900 - DNA, 37 bp, Artificial Sequence
  CNAG 01317 5 flanking region primer 2
tcactggccgtcgttttacattgacacctgccgattc

SEQ ID NO: 901 - DNA, 40 bp, Artificial Sequence
  CNAG 01317 3 flanking region primer 1
catggtcatagctgtttcctgtactatcagcagaagcggc

SEQ ID NO: 902 - DNA, 20 bp, Artificial Sequence
  CNAG 01317 3 flanking region primer 2
aatgataggcggaagtaagg

SEQ ID NO: 903 - DNA, 19 bp, Artificial Sequence
  CNAG 01317 diagnostic screening primer, pairing with 879
ccacctcgcagtatcaaac

SEQ ID NO: 904 - DNA, 20 bp, Artificial Sequence
  CNAG 01317 Southern blot probe primer
agggtcagggagatgatgtg

SEQ ID NO: 905 - DNA, 19 bp, Artificial Sequence
  CNAG 00559 5 flanking region primer 1
ggtgtttccatacgaaccg

SEQ ID NO: 906 - DNA, 38 bp, Artificial Sequence; identical to SEQ ID NO: 701
  CNAG 00559 5 flanking region primer 2

SEQ ID NO: 907 - DNA, 35 bp, Artificial Sequence; identical to SEQ ID NO: 702
  CNAG 00559 3 flanking region primer 1

SEQ ID NO: 908 - DNA, 20 bp, Artificial Sequence
  CNAG 00559 3 flanking region primer 2
gctgagaaggagaggatagc

SEQ ID NO: 909 - DNA, 20 bp, Artificial Sequence
  CNAG 00559 diagnostic screening primer, pairing with B79
gtggttgaaagtattaggcg

SEQ ID NO: 910 - DNA, 19 bp, Artificial Sequence
  CNAG 00559 Southern blot probe primer
tctcttcgtttgctacacg

SEQ ID NO: 911 - DNA, 24 bp, Artificial Sequence
  NAT 242 STM primer
gtagcgataggggtgtcgctttag

SEQ ID NO: 912 - DNA, 24 bp, Artificial Sequence
  STM common primer
gcatgccctgcccctaagaattcg

SEQ ID NO: 913 - DNA, 18 bp, Artificial Sequence
  CNAG 07940 5 flanking region primer 1
caaggtaatgattggggc

SEQ ID NO: 914 - DNA, 38 bp, Artificial Sequence
  CNAG 07940 5 flanking region primer 2
tcactggccgtcgttttacgcaataatggaaggtctcg

SEQ ID NO: 915 - DNA, 39 bp, Artificial Sequence
  CNAG 07940 3 flanking region primer 1
catggtcatagctgtttcctgatcaagaatgacacgccg

SEQ ID NO: 916 - DNA, 19 bp, Artificial Sequence
  CNAG 07940 3 flanking region primer 2
aacttgtatggtggtgccg

SEQ ID NO: 917 - DNA, 19 bp, Artificial Sequence
  CNAG 07940 diagnostic screening primer, pairing with B79
tcacttgctcttctccctg

SEQ ID NO: 918 - DNA, 18 bp, Artificial Sequence
  CNAG 07940 Southern blot probe primer
gtgacgaatgcgaaactc

SEQ ID NO: 919 - DNA, 20 bp, Artificial Sequence
  NAT 191 STM primer
atatggatgtttttagcgag

SEQ ID NO: 920 - DNA, 24 bp, Artificial Sequence
  STM common primer
gcatgccctgcccctaagaattcg

SEQ ID NO: 921 - DNA, 18 bp, Artificial Sequence
  CNAG 05535 5 flanking region primer 1
agcaacaacggcatagtg

SEQ ID NO: 922 - DNA, 39 bp, Artificial Sequence
  CNAG 05535 5 flanking region primer 2
tcactggccgtcgttttacacgacagtgagagtcctgac

SEQ ID NO: 923 - DNA, 43 bp, Artificial Sequence
  CNAG 05535 3 flanking region primer 1
catggtcatagctgtttcctgctggagcgaggaaatagtaatg

SEQ ID NO: 924 - DNA, 18 bp, Artificial Sequence
  CNAG 05535 3 flanking region primer 2
acacaaagcgagtggttg

SEQ ID NO: 925 - DNA, 19 bp, Artificial Sequence
  CNAG 05535 diagnostic screening primer, pairing with B79
ggcagtgtcagatgaaagc

SEQ ID NO: 926 - DNA, 19 bp, Artificial Sequence
  CNAG 05535 Southern blot probe primer
catcggctacatcttcctc

SEQ ID NO: 927 - DNA, 21 bp, Artificial Sequence
  CNAG 02516 5 flanking region primer 1
gcatcacacgaacgattctac

SEQ ID NO: 928 - DNA, 38 bp, Artificial Sequence
  CNAG 02516 5 flanking region primer 2
tcactggccgtcgttttacattgttggcggtggaaacg

SEQ ID NO: 929 - DNA, 41 bp, Artificial Sequence
  CNAG 02516 3 flanking region primer 1
catggtcatagctgtttcctgaggtttgggagcagtaaggc

SEQ ID NO: 930 - DNA, 19 bp, Artificial Sequence
  CNAG 02516 3 flanking region primer 2
gatgtttgtgaccgattcg

SEQ ID NO: 931 - DNA, 19 bp, Artificial Sequence
  CNAG 02516 diagnostic screening primer, pairing with B79
gcgaaggtttaccgataag

SEQ ID NO: 932 - DNA, 20 bp, Artificial Sequence
  CNAG 02516 Southern blot probe primer
tatggggttaggagcgttgg

SEQ ID NO: 933 - DNA, 20 bp, Artificial Sequence
  NAT 210 STM primer
ctagagcccgccacaacgct

SEQ ID NO: 934 - DNA, 24 bp, Artificial Sequence
  STM common primer
gcatgccctgcccctaagaattcg

SEQ ID NO: 935 - DNA, 19 bp, Artificial Sequence
  CNAG 04226 5 flanking region primer 1
gccctttttggtatgacag

SEQ ID NO: 936 - DNA, 38 bp, Artificial Sequence
  CNAG 04226 5 flanking region primer 2
tcactggccgtcgttttactgtgagggattccttgttc

SEQ ID NO: 937 - DNA, 39 bp, Artificial Sequence
  CNAG 04226 3 flanking region primer 1
catggtcatagctgtttcctggttggaaacaagcagtcg

SEQ ID NO: 938 - DNA, 20 bp, Artificial Sequence
  CNAG 04226 3 flanking region primer 2
tcagaagaatcaagaggtcg

SEQ ID NO: 939 - DNA, 19 bp, Artificial Sequence
  CNAG 04226 diagnostic screening primer, pairing with B79
acttacattgccttcctcg

SEQ ID NO: 940 - DNA, 19 bp, Artificial Sequence
  CNAG 04226 Southern blot probe primer
atacatcagcctgtcctgc

SEQ ID NO: 941 - DNA, 19 bp, Artificial Sequence
  CNAG 05170 5 flanking region primer 1
tgccttcgctctgtaactc

SEQ ID NO: 942 - DNA, 34 bp, Artificial Sequence
  CNAG 05170 5 flanking region primer 2
ctggccgtcgttttaccctttttgaggctcgctg

SEQ ID NO: 943 - DNA, 36 bp, Artificial Sequence
  CNAG 05170 3 flanking region primer 1
gtcatagctgtttcctgaaatgaaggagtgtgcggg

SEQ ID NO: 944 - DNA, 20 bp, Artificial Sequence
  CNAG 05170 3 flanking region primer 2
aataggacctcaacaccagg

SEQ ID NO: 945 - DNA, 18 bp, Artificial Sequence
  CNAG 05170 diagnostic screening primer, pairing with B79
aacaagaagcccctttcg

SEQ ID NO: 946 - DNA, 18 bp, Artificial Sequence
  CNAG 05170 Southern blot probe primer
agtttttggacccgagtg

SEQ ID NO: 947 - DNA, 18 bp, Artificial Sequence
  NAT 232 STM primer
ctttaaaggtggtttgtg

SEQ ID NO: 948 - DNA, 24 bp, Artificial Sequence
  STM common primer
gcatgccctgcccctaagaattcg

SEQ ID NO: 949 - DNA, 20 bp, Artificial Sequence
  CNAG 03902 5 flanking region primer 1
gcaaagtcaagaaggtcagg

SEQ ID NO: 950 - DNA, 35 bp, Artificial Sequence
  CNAG 03902 5 flanking region primer 2
ctggccgtcgttttacttcttctttggctcgtccg

SEQ ID NO: 951 - DNA, 39 bp, Artificial Sequence
  CNAG 03902 3 flanking region primer 1
gtcatagctgtttcctggggagatgaaatgtgcgtttag

SEQ ID NO: 952 - DNA, 22 bp, Artificial Sequence
  CNAG 03902 3 flanking region primer 2
atgctttcgccgtcatctcgtc

SEQ ID NO: 953 - DNA, 20 bp, Artificial Sequence
  CNAG 03902 diagnostic screening primer, pairing with B79
tgattacccctgtcaaactc

SEQ ID NO: 954 - DNA, 19 bp, Artificial Sequence
  CNAG 03902 Southern blot probe primer
cagaatcgggaagtaatgg

SEQ ID NO: 955 - DNA, 18 bp, Artificial Sequence
  NAT 232 STM primer
ctttaaaggtggtttgtg

SEQ ID NO: 956 - DNA, 24 bp, Artificial Sequence
  STM common primer
gcatgccctgcccctaagaattcg

SEQ ID NO: 957 - DNA, 20 bp, Artificial Sequence
  CNAG 06097 5 flanking region primer 1
cagtccgaagtttacaatcg

SEQ ID NO: 958 - DNA, 36 bp, Artificial Sequence
  CNAG 06097 5 flanking region primer 2
ctggccgtcgttttactttcgtcgtgtggagttggc

SEQ ID NO: 959 - DNA, 37 bp, Artificial Sequence
  CNAG 06097 3 flanking region primer 1
gtcatagctgtttcctgagattggagggtttgagggc

SEQ ID NO: 960 - DNA, 19 bp, Artificial Sequence
  CNAG 06097 3 flanking region primer 2
tccaccgaagaaactgaag

SEQ ID NO: 961 - DNA, 20 bp, Artificial Sequence
  CNAG 06097 diagnostic screening primer, pairing with B79
aggtttgagaggtttggaag

SEQ ID NO: 962 - DNA, 18 bp, Artificial Sequence
  CNAG 06097 Southern blot probe primer
attatcggagcgttgtgg

SEQ ID NO: 963 - DNA, 20 bp, Artificial Sequence
  CNAG 03431 5 flanking region primer
aagcactctgtagcctgttg

SEQ ID NO: 964 - DNA, 39 bp, Artificial Sequence
  CNAG 03431 5 flanking region primer 2
gctcactggccgtcgttttaccttgaagatgatgcgtgc

SEQ ID NO: 965 - DNA, 40 bp, Artificial Sequence
  CNAG 03431 3 flanking region primer 1
catggtcatagctgtttcctgcgctcatagagaatgctgg

SEQ ID NO: 966 - DNA, 19 bp, Artificial Sequence
  CNAG 03431 3 flanking region primer 2
taggagtgtctgccaaagc

SEQ ID NO: 967 - DNA, 19 bp, Artificial Sequence
  CNAG 03431 diagnostic screening primer, pairing with B79
tcatcctcatcccgaatac

SEQ ID NO: 968 - DNA, 19 bp, Artificial Sequence
  CNAG 03431 Southern blot probe primer
actcttcgtgagacgcatc

SEQ ID NO: 969 - DNA, 24 bp, Artificial Sequence
  NAT 290 STM primer
accgacagctcgaacaagcaagag

SEQ ID NO: 970 - DNA, 24 bp, Artificial Sequence
  STM common primer
gcatgccctgcccctaagaattcg

SEQ ID NO: 971 - DNA, 21 bp, Artificial Sequence
  CNAG 00830 5 flanking region primer 1
ccgactaataggtgtgctgtc

SEQ ID NO: 972 - DNA, 41 bp, Artificial Sequence
  CNAG 00830 5 flanking region primer 2
gctcactggccgtcgttttacgagatgagatgagttgcgtg

SEQ ID NO: 973 - DNA, 41 bp, Artificial Sequence
  CNAG 00830 3 flanking region primer 1
catggtcatagctgtttcctgttgtgtccccatcactaccg

SEQ ID NO: 974 - DNA, 19 bp, Artificial Sequence
  CNAG 00830 3 flanking region primer 2
catccaaagaagcgaaacg

SEQ ID NO: 975 - DNA, 24 bp, Artificial Sequence
  CNAG 00830 diagnostic screening primer, pairing with B79
gaagagaagtagtggatagactcg

SEQ ID NO: 976 - DNA, 19 bp, Artificial Sequence
  CNAG 00830 Southern blot probe primer
tgatgccgtaaatcgtagg

SEQ ID NO: 977 - DNA, 21 bp, Artificial Sequence
  NAT 292 STM primer
atttcgatgtatatatgtagc

SEQ ID NO: 978 - DNA, 24 bp, Artificial Sequence
  STM common primer
gcatgccctgcccctaagaattcg

SEQ ID NO: 979 - DNA, 20 bp, Artificial Sequence
  CNAG 07443 5 flanking region primer 1
atctcttctctcggcgtgtc

SEQ ID NO: 980 - DNA, 40 bp, Artificial Sequence
  CNAG 07443 5 flanking region primer 2
gctcactggccgtcgttttacacaggaaaaggaggagtcg

SEQ ID NO: 981 - DNA, 40 bp, Artificial Sequence
  CNAG 07443 3 flanking region primer 1
catggtcatagctgtttcctgaagtctttccgtctgaccc

SEQ ID NO: 982 - DNA, 18 bp, Artificial Sequence
  CNAG 07443 3 flanking region primer 2
tcgcatcccttgtattcg

SEQ ID NO: 983 - DNA, 19 bp, Artificial Sequence
  CNAG 07443 diagnostic screening primer, pairing with B79
tcaataatcagccgcctac

SEQ ID NO: 984 - DNA, 19 bp, Artificial Sequence
  CNAG 07443 Southern blot probe primer
ttagattccgcataggtcc

SEQ ID NO: 985 - DNA, 21 bp, Artificial Sequence
  NAT 295 STM primer
acacctacatcaaaccctccc

SEQ ID NO: 986 - DNA, 24 bp, Artificial Sequence
  STM common primer
gcatgccctgcccctaagaattcg

SEQ ID NO: 987 - DNA, 19 bp, Artificial Sequence
  CNAG 05186 5 flanking region primer 1
acaacggtcaagaagggtc

SEQ ID NO: 988 - DNA, 39 bp, Artificial Sequence
  CNAG 05186 5 flanking region primer 2
gctcactggccgtcgttttacaagtaaaggaacgcacgg

SEQ ID NO: 989 - DNA, 41 bp, Artificial Sequence
  CNAG 05186 3 flanking region primer 1
catggtcatagctgtttcctgcgcttgagacgggagtaatc

SEQ ID NO: 990 - DNA, 20 bp, Artificial Sequence
  CNAG 05186 3 flanking region primer 2
gaggtaaggatgattggacc

SEQ ID NO: 991 - DNA, 23 bp, Artificial Sequence
  CNAG 05186 diagnostic screening primer, pairing with B79
gtgtgaagtgagtagtaacgagc

SEQ ID NO: 992 - DNA, 22 bp, Artificial Sequence
  CNAG 05186 Southern blot probe primer
ttccctatctctcttcataccc

SEQ ID NO: 993 - DNA, 20 bp, Artificial Sequence
  NAT 296 STM primer
cgcccgccctcactatccac

SEQ ID NO: 994 - DNA, 24 bp, Artificial Sequence
  STM common primer
gcatgccctgcccctaagaattcg

SEQ ID NO: 995 - DNA, 20 bp, Artificial Sequence
  CNAG 03561 5 flanking region primer 1
tgggcgagacgagatagaac

SEQ ID NO: 996 - DNA, 36 bp, Artificial Sequence
  CNAG 03561 5 flanking region primer 2
ctggccgtcgttttacggataaagatgattcgggtc

SEQ ID NO: 997 - DNA, 36 bp, Artificial Sequence
  CNAG 03561 3 flanking region primer 1
gtcatagctgtttcctgtgatggctgatttcccgtg

SEQ ID NO: 998 - DNA, 36 bp, Artificial Sequence; identical to SEQ ID NO: 997
  CNAG 03561 3 flanking region primer 2

SEQ ID NO: 999 - DNA, 20 bp, Artificial Sequence
  CNAG 03561 diagnostic screening primer, pairing with B79
aagaaagacggtagcggtgg

SEQ ID NO: 1000 - DNA, 20 bp, Artificial Sequence
  CNAG 03561 Southern blot probe primer
ccacctcttcatccaatagc

SEQ ID NO: 1001 - DNA, 20 bp, Artificial Sequence
  NAT 43 STM primer
ccagctaccaatcacgctac

SEQ ID NO: 1002 - DNA, 24 bp, Artificial Sequence
  STM common primer
gcatgccctgcccctaagaattcg

SEQ ID NO: 1003 - DNA, 19 bp, Artificial Sequence
  CNAG 04878 5 flanking region primer 1
aagtccgtgctggctaatc

SEQ ID NO: 1004 - DNA, 35 bp, Artificial Sequence
  CNAG 04878 5 flanking region primer 2
ctggccgtcgttttaccttttcgctgccttattcg

SEQ ID NO: 1005 - DNA, 41 bp, Artificial Sequence
  CNAG 04878 3 flanking region primer 1
gtcatagctgtttcctgaaaacagggcatacggtgtcgtgg

SEQ ID NO: 1006 - DNA, 20 bp, Artificial Sequence
  CNAG 04878 3 flanking region primer 2
gctgaccatctcttccctac

SEQ ID NO: 1007 - DNA, 22 bp, Artificial Sequence
  CNAG 04878 diagnostic screening primer, pairing with B79
ccttgtttgtgtctaccgtatc

SEQ ID NO: 1008 - DNA, 19 bp, Artificial Sequence
  CNAG 04878 Southern blot probe primer
agtgggaatcgtcgttgac

SEQ ID NO: 1009 - DNA, 20 bp, Artificial Sequence
  NAT 116 STM primer
gcacccaagagctccatctc

SEQ ID NO: 1010 - DNA, 24 bp, Artificial Sequence
  STM common primer
gcatgccctgcccctaagaattcg

SEQ ID NO: 1011 - DNA, 19 bp, Artificial Sequence
  CNAG 03279 5 flanking region primer 1
cgtttccagttatcgcttc

SEQ ID NO: 1012 - DNA, 41 bp, Artificial Sequence
  CNAG 03279 5 flanking region primer 2
gctcactggccgtcgttttacgcaatgacccactttttgac

SEQ ID NO: 1013 - DNA, 41 bp, Artificial Sequence
  CNAG 03279 3 flanking region primer 1
catggtcatagctgtttcctggctttgcctgatgagttttg

SEQ ID NO: 1014 - DNA, 19 bp, Artificial Sequence
  CNAG 03279 3 flanking region primer 2
gtaacccactcggaagttg

SEQ ID NO: 1015 - DNA, 19 bp, Artificial Sequence
  CNAG 03279 diagnostic screening primer, pairing with B79
ttgtctgtgaggtggaagg

SEQ ID NO: 1016 - DNA, 20 bp, Artificial Sequence
  CNAG 03279 Southern blot probe primer
caggctcgtttgtagccttc

SEQ ID NO: 1017 - DNA, 24 bp, Artificial Sequence
  NAT 122 STM primer
acagctccaaacctcgctaaacag

SEQ ID NO: 1018 - DNA, 24 bp, Artificial Sequence
  STM common primer
gcatgccctgcccctaagaattcg

SEQ ID NO: 1019 - DNA, 18 bp, Artificial Sequence
  CNAG 06921 5 flanking region primer 1
gacaaagcaacaacagcg

SEQ ID NO: 1020 - DNA, 37 bp, Artificial Sequence
  CNAG 06921 5 flanking region primer 2
tcactggccgtcgttttactcggatgacagatggttg

SEQ ID NO: 1021 - DNA, 39 bp, Artificial Sequence
  CNAG 06921 3 flanking region primer 1
catggtcatagctgtttcctgaaagattgggcgtctctg

SEQ ID NO: 1022 - DNA, 18 bp, Artificial Sequence
  CNAG 06921 3 flanking region primer 2
atcatcagcgacatccag

SEQ ID NO: 1023 - DNA, 18 bp, Artificial Sequence
  CNAG 06921 diagnostic screening primer, pairing with B79
aaagaacatacacgccgc

SEQ ID NO: 1024 - DNA, 18 bp, Artificial Sequence
  CNAG 06921 Southern blot probe primer
accatctggatttctgcc

SEQ ID NO: 1025 - DNA, 21 bp, Artificial Sequence
  NAT 159 STM primer
acgcaccagacacacaaccag

SEQ ID NO: 1026 - DNA, 24 bp, Artificial Sequence
  STM common primer
gcatgccctgcccctaagaattcg

SEQ ID NO: 1027 - DNA, 20 bp, Artificial Sequence
  CNAG 02555 5 flanking region primer 1
agaggaacacggtatggtcg

SEQ ID NO: 1028 - DNA, 40 bp, Artificial Sequence
  CNAG 02555 5 flanking region primer 2
gctcactggccgtcgttttaccaacaacggtgaaccaaag

SEQ ID NO: 1029 - DNA, 41 bp, Artificial Sequence
  CNAG 02555 3 flanking region primer 1
catggtcatagctgtttcctgcagagtggaggtagaaacgg

SEQ ID NO: 1030 - DNA, 18 bp, Artificial Sequence
  CNAG 02555 3 flanking region primer 2
tctgaaatgctggctgtc

SEQ ID NO: 1031 - DNA, 20 bp, Artificial Sequence
  CNAG 02555 diagnostic screening primer, pairing with B79
cccctcacaacttgctaatg

SEQ ID NO: 1032 - DNA, 20 bp, Artificial Sequence
  CNAG 02555 Southern blot probe primer
ccgatgtcgacgtatgattg

SEQ ID NO: 1033 - DNA, 18 bp, Artificial Sequence
  NAT 270 STM primer
ggtggtttgtgtgggtag

SEQ ID NO: 1034 - DNA, 24 bp, Artificial Sequence
  STM common primer
gcatgccctgcccctaagaattcg

SEQ ID NO: 1035 - DNA, 19 bp, Artificial Sequence
  CNAG 03710 5 flanking region primer 1
aagacaagaagcagcagcg

SEQ ID NO: 1036 - DNA, 40 bp, Artificial Sequence
  CNAG 03710 5 flanking region primer 2
gctcactggccgtcgttttactatcattcccatccgtcgc

SEQ ID NO: 1037 - DNA, 42 bp, Artificial Sequence
  CNAG 03710 3 flanking region primer 1
catggtcatagctgtttcctgcgctcaataacatcgtcagac

SEQ ID NO: 1038 - DNA, 20 bp, Artificial Sequence
  CNAG 03710 3 flanking region primer 2
ccagccttcatctctcttcc

SEQ ID NO: 1039 - DNA, 20 bp, Artificial Sequence
  CNAG 03710 diagnostic screening primer, pairing with B79
cagaactgataactggctgc

SEQ ID NO: 1040 - DNA, 20 bp, Artificial Sequence
  CNAG 03710 Southern blot probe primer
tcctctgcaccaagactgtg

SEQ ID NO: 1041 - DNA, 24 bp, Artificial Sequence
  NAT 290 STM primer
accgacagctcgaacaagcaagag

SEQ ID NO: 1042 - DNA, 24 bp, Artificial Sequence
  STM common primer
gcatgccctgcccctaagaattcg

SEQ ID NO: 1043 - DNA, 20 bp, Artificial Sequence
  CNAG 03183 5 flanking region primer 1
ctctatgagggcaaagtcag

SEQ ID NO: 1044 - DNA, 40 bp, Artificial Sequence
  CNAG 03183 5 flanking region primer 2
gctcactggccgtcgttttactgttgttgctgttggctcg

SEQ ID NO: 1045 - DNA, 40 bp, Artificial Sequence
  CNAG 03183 3 flanking region primer 1
catggtcatagctgtttcctgttcttccaatcccagccac

SEQ ID NO: 1046 - DNA, 19 bp, Artificial Sequence
  CNAG 03183 3 flanking region primer 2
tacgcttcacagattacgc

SEQ ID NO: 1047 - DNA, 18 bp, Artificial Sequence
  CNAG 03183 diagnostic screening primer, pairing with B/9
tatacgtccaagacatcc

SEQ ID NO: 1048 - DNA, 20 bp, Artificial Sequence
  CNAG 03183 Southern blot probe primer
ctgattccgaggcttactcg

SEQ ID NO: 1049 - DNA, 21 bp, Artificial Sequence
  NAT 292 STM primer
atttcgatgtatatatgtagc

SEQ ID NO: 1050 - DNA, 24 bp, Artificial Sequence
  STM common primer
gcatgccctgcccctaagaattcg

SEQ ID NO: 1051 - DNA, 20 bp, Artificial Sequence
  CNAG 04637 5 flanking region primer 1
ccttcaagccaaaatgagac

SEQ ID NO: 1052 - DNA, 40 bp, Artificial Sequence
  CNAG 04637 5 flanking region primer 2
gctcactggccgtcgttttacgagatggagagaagcaacg

SEQ ID NO: 1053 - DNA, 43 bp, Artificial Sequence
  CNAG 04637 3 flanking region primer 1
catggtcatagctgtttcctgggttgacggatttactgaagtc

SEQ ID NO: 1054 - DNA, 20 bp, Artificial Sequence
  CNAG 04637 3 flanking region primer 2
tggaagatgactcactcggg

SEQ ID NO: 1055 - DNA, 19 bp, Artificial Sequence
  CNAG 04637 diagnostic screening primer, pairing with B79
tccatcaaagatacacccg

SEQ ID NO: 1056 - DNA, 20 bp, Artificial Sequence
  CNAG 04637 Southern blot probe primer
ttacatcttgaggcttagcg

SEQ ID NO: 1057 - DNA, 20 bp, Artificial Sequence
  NAT 296 STM primer
cgcccgccctcactatccac

SEQ ID NO: 1058 - DNA, 24 bp, Artificial Sequence
  STM common primer
gcatgccctgcccctaagaattcg

SEQ ID NO: 1059 - DNA, 19 bp, Artificial Sequence
  CNAG 04398 5 flanking region primer 1
agtctggagaaaaggggtc

SEQ ID NO: 1060 - DNA, 34 bp, Artificial Sequence
  CNAG 04398 5 flanking region primer 2
ctggccgtcgttttacgcaaacacacattctcgg

SEQ ID NO: 1061 - DNA, 36 bp, Artificial Sequence
  CNAG 04398 3 flanking region primer 1
gtcatagctgtttcctgttggctaatgtagggttgg

SEQ ID NO: 1062 - DNA, 18 bp, Artificial Sequence
  CNAG 04398 3 flanking region primer 2
ccccttcccatttttcac

SEQ ID NO: 1063 - DNA, 22 bp, Artificial Sequence
  CNAG 04398 diagnostic screening primer, pairing with B79
gccactttctgatttccgtatc

SEQ ID NO: 1064 - DNA, 18 bp, Artificial Sequence
  CNAG 04398 Southern blot probe primer
tatttgctgcctgaccac

SEQ ID NO: 1065 - DNA, 20 bp, Artificial Sequence
  NAT 225 STM primer
ccatagaactagctaaagca

SEQ ID NO: 1066 - DNA, 24 bp, Artificial Sequence
  STM common primer
gcatgccctgcccctaagaattcg

SEQ ID NO: 1067 - DNA, 20 bp, Artificial Sequence
  CNAG 01454 5 flanking region primer 1
ggaagtttggaggaggatag

SEQ ID NO: 1068 - DNA, 37 bp, Artificial Sequence
  CNAG 01454 5 flanking region primer 2
tcactggccgtcgttttaccagattcgtggtcaaagc

SEQ ID NO: 1069 - DNA, 40 bp, Artificial Sequence
  CNAG 01454 3 flanking region primer 1
catggtcatagctgtttcctggccttgaatgatgaccagc

SEQ ID NO: 1070 - DNA, 18 bp, Artificial Sequence
  CNAG 01454 3 flanking region primer 2
cggatttgagatgatgcc

SEQ ID NO: 1071 - DNA, 22 bp, Artificial Sequence
  CNAG 01454 diagnostic screening primer, pairing with B79
gttagaggagtcattctttggc

SEQ ID NO: 1072 - DNA, 20 bp, Artificial Sequence
  CNAG 01454 Southern blot probe primer
caaaaggtcaaggaaaggtg

SEQ ID NO: 1073 - DNA, 24 bp, Artificial Sequence
  NAT 58 STM primer
cgcaaaatcactagccctatagcg

SEQ ID NO: 1074 - DNA, 24 bp, Artificial Sequence
  STM common primer
gcatgccctgcccctaagaattcg

SEQ ID NO: 1075 - DNA, 18 bp, Artificial Sequence
  CNAG 04268 5 flanking region primer 1
tcgtctttgtgtggttgc

SEQ ID NO: 1076 - DNA, 38 bp, Artificial Sequence
  CNAG 04268 5 flanking region primer 2
tcactggccgtcgttttacacttatggcgacttacggg

SEQ ID NO: 1077 - DNA, 40 bp, Artificial Sequence
  CNAG 04268 3 flanking region primer 1
catggtcatagctgtttcctggcaatgtctgtatcaccgc

SEQ ID NO: 1078 - DNA, 19 bp, Artificial Sequence
  CNAG 04268 3 flanking region primer 2
cgtccgcctgtattttatc

SEQ ID NO: 1079 - DNA, 18 bp, Artificial Sequence
  CNAG 04268 diagnostic screening primer, pairing with B79
atgatgctgttttgcccc

SEQ ID NO: 1080 - DNA, 18 bp, Artificial Sequence
  CNAG 04268 Southern blot probe primer
ttctttccacatcggtcc

SEQ ID NO: 1081 - DNA, 24 bp, Artificial Sequence
  NAT 102 STM primer
ccatagcgatatctaccccaatct

SEQ ID NO: 1082 - DNA, 24 bp, Artificial Sequence
  STM common primer
gcatgccctgcccctaagaattcg

SEQ ID NO: 1083 - DNA, 22 bp, Artificial Sequence
  CNAG 00031 5 flanking region primer 1
atccaatcatcggcttcccagc

SEQ ID NO: 1084 - DNA, 35 bp, Artificial Sequence
  CNAG 00031 5 flanking region primer 2
ctggccgtcgttttacacttgaactcccatctgcg

SEQ ID NO: 1085 - DNA, 37 bp, Artificial Sequence
  CNAG 00031 3 flanking region primer 1
gtcatagctgtttcctggaaatgcgatgaatgggttg

SEQ ID NO: 1086 - DNA, 21 bp, Artificial Sequence
  CNAG 00031 3 flanking region primer 2
cctcttgccctaatgagatag

SEQ ID NO: 1087 - DNA, 18 bp, Artificial Sequence
  CNAG 00031 diagnostic screening primer, pairing with B79
ccgtgttcgcaatctatg

SEQ ID NO: 1088 - DNA, 19 bp, Artificial Sequence
  CNAG 00031 Southern blot probe primer
gaatcagcatttgggttcg

SEQ ID NO: 1089 - DNA, 20 bp, Artificial Sequence
  NAT 116 STM primer
gcacccaagagctccatctc

SEQ ID NO: 1090 - DNA, 24 bp, Artificial Sequence
  STM common primer
gcatgccctgcccctaagaattcg

SEQ ID NO: 1091 - DNA, 19 bp, Artificial Sequence
  CNAG 07922 5 flanking region primer 1
cctcttccgctattttgtg

SEQ ID NO: 1092 - DNA, 39 bp, Artificial Sequence
  CNAG 07922 5 flanking region primer 2
gctcactggccgtcgttttaccattgctgttgtgggaac

SEQ ID NO: 1093 - DNA, 41 bp, Artificial Sequence
  CNAG 07922 3 flanking region primer 1
catggtcatagctgtttcctgaagtggagttggaagaggcg

SEQ ID NO: 1094 - DNA, 22 bp, Artificial Sequence
  CNAG 07922 3 flanking region primer 2
caactatccaatacctgttccg

SEQ ID NO: 1095 - DNA, 19 bp, Artificial Sequence
  CNAG 07922 diagnostic screening primer, pairing with B79
cacggcttgtcttgaagtc

SEQ ID NO: 1096 - DNA, 18 bp, Artificial Sequence
  CNAG 07922 Southern blot probe primer
ttgacggttttggcacag

SEQ ID NO: 1097 - DNA, 20 bp, Artificial Sequence
  NAT 116 STM primer
gcacccaagagctccatctc

SEQ ID NO: 1098 - DNA, 24 bp, Artificial Sequence
  STM common primer
gcatgccctgcccctaagaattcg

SEQ ID NO: 1099 - DNA, 19 bp, Artificial Sequence
  CNAG 04841 5 flanking region primer 1
tgaggtgttccgtgtattg

SEQ ID NO: 1100 - DNA, 35 bp, Artificial Sequence
  CNAG 04841 5 flanking region primer 2
ctggccgtcgttttacctcttgactggtaatgggg

SEQ ID NO: 1101 - DNA, 35 bp, Artificial Sequence
  CNAG 04841 3 flanking region primer 1
gtcatagctgtttcctgattcccaaggtgacgaag

SEQ ID NO: 1102 - DNA, 20 bp, Artificial Sequence
  CNAG 04841 3 flanking region primer 2
tctccaataagttcaggcac

SEQ ID NO: 1103 - DNA, 22 bp, Artificial Sequence
  CNAG 04841 diagnostic screening primer, pairing with B79
taaagacggaggtgggagtagg

SEQ ID NO: 1104 - DNA, 18 bp, Artificial Sequence
  CNAG 04841 Southern blot probe primer
tgagattgggaaagagcg

SEQ ID NO: 1105 - DNA, 24 bp, Artificial Sequence
  NAT 119 STM primer
ctccccacataaagagagctaaac

SEQ ID NO: 1106 - DNA, 24 bp, Artificial Sequence
  STM common primer
gcatgccctgcccctaagaattcg

SEQ ID NO: 1107 - DNA, 20 bp, Artificial Sequence
  CNAG 01948 5 flanking region primer 1
cttggtaaatccgtattccg

SEQ ID NO: 1108 - DNA, 35 bp, Artificial Sequence
  CNAG 01948 5 flanking region primer 2
ctggccgtcgttttaccctttcgcttcaacctacg

SEQ ID NO: 1109 - DNA, 35 bp, Artificial Sequence
  CNAG 01948 3 flanking region primer 1
gtcatagctgtttcctgtgatttggcgactctgag

SEQ ID NO: 1110 - DNA, 20 bp, Artificial Sequence
  CNAG 01948 3 flanking region primer 2
tgtgtttctcatcgtaagcg

SEQ ID NO: 1111 - DNA, 22 bp, Artificial Sequence
  CNAG 01948 diagnostic screening primer, pairing with B79
ttgacttcccaggttacactac

SEQ ID NO: 1112 - DNA, 19 bp, Artificial Sequence
  CNAG 01948 Southern blot probe primer
cattgggactcttgtaggg

SEQ ID NO: 1113 - DNA, 24 bp, Artificial Sequence
  NAT 119 STM primer
ctccccacataaagagagctaaac

SEQ ID NO: 1114 - DNA, 24 bp, Artificial Sequence
  STM common primer
gcatgccctgcccctaagaattcg

SEQ ID NO: 1115 - DNA, 19 bp, Artificial Sequence
  CNAG 07435 5 flanking region primer 1
caaccttcaaagtcttgcc

SEQ ID NO: 1116 - DNA, 39 bp, Artificial Sequence
  CNAG 07435 5 flanking region primer 2
tcactggccgtcgttttacaacgaggagtcatactgtgc

SEQ ID NO: 1117 - DNA, 41 bp, Artificial Sequence
  CNAG 07435 3 flanking region primer 1
catggtcatagctgtttcctgaaaggcgtcaaagggcgaag

SEQ ID NO: 1118 - DNA, 20 bp, Artificial Sequence
  CNAG 07435 3 flanking region primer 2
ggtttggagcgtattgattg

SEQ ID NO: 1119 - DNA, 19 bp, Artificial Sequence
  CNAG 07435 diagnostic screening primer, pairing with B79
aagagcggcattgaatcgg

SEQ ID NO: 1120 - DNA, 20 bp, Artificial Sequence
  CNAG 07435 Southern blot probe primer
gcgtcactttggacatactc

SEQ ID NO: 1121 - DNA, 20 bp, Artificial Sequence
  NAT 123 STM primer
ctatcgaccaaccaacacag

SEQ ID NO: 1122 - DNA, 24 bp, Artificial Sequence
  STM common primer
gcatgccctgcccctaagaattcg

SEQ ID NO: 1123 - DNA, 22 bp, Artificial Sequence
  CNAG 00505 5 flanking region primer 1
tttcaggcaccaacaggcacag

SEQ ID NO: 1124 - DNA, 38 bp, Artificial Sequence
  CNAG 00505 5 flanking region primer 2
ctggccgtcgttttacttccgatggtgcccttgagtag

SEQ ID NO: 1125 - DNA, 38 bp, Artificial Sequence
  CNAG 00505 3 flanking region primer 1
gtcatagctgtttcctgggtcttccaaatgtcacagtc

SEQ ID NO: 1126 - DNA, 19 bp, Artificial Sequence
  CNAG 00505 3 flanking region primer 2
aaagaagagacgcctcacg

SEQ ID NO: 1127 - DNA, 20 bp, Artificial Sequence
  CNAG 00505 diagnostic screening primer, pairing with B79
tgatagacaccacggcagtc

SEQ ID NO: 1128 - DNA, 20 bp, Artificial Sequence
  CNAG 00505 Southern blot probe primer
cgaggattgtcaaatgaagg

SEQ ID NO: 1129 - DNA, 24 bp, Artificial Sequence
  NAT 125 STM primer
cgctacagccagcgcgcgcaagcg

SEQ ID NO: 1130 - DNA, 24 bp, Artificial Sequence
  STM common primer
gcatgccctgcccctaagaattcg

SEQ ID NO: 1131 - DNA, 18 bp, Artificial Sequence
  CNAG 03115 5 flanking region primer 1
cgaaggaagtaaaacggg

SEQ ID NO: 1132 - DNA, 37 bp, Artificial Sequence
  CNAG 03115 5 flanking region primer 2
ctggccgtcgttttacaagcactcaccactctcctac

SEQ ID NO: 1133 - DNA, 36 bp, Artificial Sequence
  CNAG 03115 3 flanking region primer 1
gtcatagctgtttcctgtgcttggatagacgacgag

SEQ ID NO: 1134 - DNA, 20 bp, Artificial Sequence
  CNAG 03115 3 flanking region primer 2
tcaatcaaagcgttcggggg

SEQ ID NO: 1135 - DNA, 20 bp, Artificial Sequence
  CNAG 03115 diagnostic screening primer, pairing with B79
aatccgacttctcagccctg

SEQ ID NO: 1136 - DNA, 20 bp, Artificial Sequence
  CNAG 03115 Southern blot probe primer
atctgagggaagggctacag

SEQ ID NO: 1137 - DNA, 20 bp, Artificial Sequence
  NAT 177 STM primer
caccaactccccatctccat

SEQ ID NO: 1138 - DNA, 24 bp, Artificial Sequence
  STM common primer
gcatgccctgcccctaagaattcg

SEQ ID NO: 1139 - DNA, 19 bp, Artificial Sequence
  CNAG 04807 5 flanking region primer 1
gcgtttcctcgtgaacttc

SEQ ID NO: 1140 - DNA, 37 bp, Artificial Sequence
  CNAG 04807 5 flanking region primer 2
ctggccgtcgttttacggaagaagggttgagacactc

SEQ ID NO: 1141 - DNA, 36 bp, Artificial Sequence
  CNAG 04807 3 flanking region primer 1
gtcatagctgtttcctgggcaagcataatggcaaag

SEQ ID NO: 1142 - DNA, 18 bp, Artificial Sequence
  CNAG 04807 3 flanking region primer 2
ggatttccgaagccaaac

SEQ ID NO: 1143 - DNA, 19 bp, Artificial Sequence
  CNAG 04807 diagnostic screening primer, pairing with B79
tggcacagttcaatgttcc

SEQ ID NO: 1144 - DNA, 18 bp, Artificial Sequence
  CNAG 04807 Southern blot probe primer
cgacaatacaaacacgcc

SEQ ID NO: 1145 - DNA, 20 bp, Artificial Sequence
  NAT 177 STM primer
caccaactccccatctccat

SEQ ID NO: 1146 - DNA, 24 bp, Artificial Sequence
  STM common primer
gcatgccctgcccctaagaattcg

SEQ ID NO: 1147 - DNA, 19 bp, Artificial Sequence
  CNAG 02435 5 flanking region primer 1
atcgttagcagggtgagtc

SEQ ID NO: 1148 - DNA, 42 bp, Artificial Sequence
  CNAG 02435 5 flanking region primer 2
gctcactggccgtcgttttactcctattcagggactatcacc

SEQ ID NO: 1149 - DNA, 39 bp, Artificial Sequence
  CNAG 02435 3 flanking region primer 1
catggtcatagctgtttcctgacctattttccacgcagc

SEQ ID NO: 1150 - DNA, 18 bp, Artificial Sequence
  CNAG 02435 3 flanking region primer 2
tcagcctcgcacataatc

SEQ ID NO: 1151 - DNA, 18 bp, Artificial Sequence
  CNAG 02435 diagnostic screening primer, pairing with B79
cgagccgaaaagaacaag

SEQ ID NO: 1152 - DNA, 20 bp, Artificial Sequence
  CNAG 02435 Southern blot probe primer
gattatctgtttggttcggc

SEQ ID NO: 1153 - DNA, 24 bp, Artificial Sequence
  NAT 184 STM primer
atatatggctcgagctagatagag

SEQ ID NO: 1154 - DNA, 24 bp, Artificial Sequence
  STM common primer
gcatgccctgcccctaagaattcg

SEQ ID NO: 1155 - DNA, 20 bp, Artificial Sequence
  CNAG 02364 5 flanking region primer 1
gaagtcaacaggaagaagcc

SEQ ID NO: 1156 - DNA, 34 bp, Artificial Sequence
  CNAG 02364 5 flanking region primer 2
ctggccgtcgttttactttgcgtgaaggtgtagg

SEQ ID NO: 1157 - DNA, 36 bp, Artificial Sequence
  CNAG 02364 3 flanking region primer 1
gtcatagctgtttcctgtgctcatcttcacggctac

SEQ ID NO: 1158 - DNA, 19 bp, Artificial Sequence
  CNAG 02364 3 flanking region primer 2
cattatccgcctgatgttg

SEQ ID NO: 1159 - DNA, 19 bp, Artificial Sequence
  CNAG 02364 diagnostic screening primer, pairing with B79
gcacttccaacaaagccac

SEQ ID NO: 1160 - DNA, 19 bp, Artificial Sequence
  CNAG 02364 Southern blot probe primer
gactttttgcgtgaaggtg

SEQ ID NO: 1161 - DNA, 24 bp, Artificial Sequence
  NAT 184 STM primer
atatatggctcgagctagatagag

SEQ ID NO: 1162 - DNA, 24 bp, Artificial Sequence
  STM common primer
gcatgccctgcccctaagaattcg

SEQ ID NO: 1163 - DNA, 20 bp, Artificial Sequence
  CNAG 05420 5 flanking region primer 1
cgcctttgaggtattcactc

SEQ ID NO: 1164 - DNA, 39 bp, Artificial Sequence
  CNAG 05420 5 flanking region primer 2
tcactggccgtcgttttacggtgttggtcacgaatctac

SEQ ID NO: 1165 - DNA, 40 bp, Artificial Sequence
  CNAG 05420 3 flanking region primer 1
catggtcatagctgtttcctgcggctttacgaaatgagtg

SEQ ID NO: 1166 - DNA, 21 bp, Artificial Sequence
  CNAG 05420 3 flanking region primer 2
cctgaaagacatagcgacaag

SEQ ID NO: 1167 - DNA, 19 bp, Artificial Sequence
  CNAG 05420 diagnostic screening primer, pairing with B79
atcctgtcttgctgtggtc

SEQ ID NO: 1168 - DNA, 18 bp, Artificial Sequence
  CNAG 05420 Southern blot probe primer
tccagcatcgtcattgag

SEQ ID NO: 1169 - DNA, 20 bp, Artificial Sequence
  NAT 191 STM primer
atatggatgtttttagcgag

SEQ ID NO: 1170 - DNA, 24 bp, Artificial Sequence
  STM common primer
gcatgccctgcccctaagaattcg

SEQ ID NO: 1171 - DNA, 19 bp, Artificial Sequence
  CNAG 05093 5 flanking region primer 1
aatgtattgccaccgtgcc

SEQ ID NO: 1172 - DNA, 41 bp, Artificial Sequence
  CNAG 05093 5 flanking region primer 2
tcactggccgtcgttttacttggggtgatgtgttctcgtcc

SEQ ID NO: 1173 - DNA, 41 bp, Artificial Sequence
  CNAG 05093 3 flanking region primer 1
catggtcatagctgtttcctgacaggcagattgggaagtgc

SEQ ID NO: 1174 - DNA, 19 bp, Artificial Sequence
  CNAG 05093 3 flanking region primer 2
tgtatgccgtgggaacaag

SEQ ID NO: 1175 - DNA, 24 bp, Artificial Sequence
  CNAG 05093 diagnostic screening primer, pairing with B79
cgaagtctcaaacctatctactcg

SEQ ID NO: 1176 - DNA, 22 bp, Artificial Sequence
  CNAG 05093 Southern blot probe primer
ttgctgaggcgaacttgttggc

SEQ ID NO: 1177 - DNA, 24 bp, Artificial Sequence
  NAT 201 STM primer
caccctctatctcgagaaagctcc

SEQ ID NO: 1178 - DNA, 24 bp, Artificial Sequence
  STM common primer
gcatgccctgcccctaagaattcg

SEQ ID NO: 1179 - DNA, 19 bp, Artificial Sequence
  CNAG 06814 5 flanking region primer 1
tgggaatggcaaaaggcac

SEQ ID NO: 1180 - DNA, 35 bp, Artificial Sequence
  CNAG 06814 5 flanking region primer 2
ctggccgtcgttttacggtattttcgtccagtaaa

SEQ ID NO: 1181 - DNA, 35 bp, Artificial Sequence
  CNAG 06814 3 flanking region primer 1
gtcatagctgtttcctgtgcagcctatgcctcctt

SEQ ID NO: 1182 - DNA, 19 bp, Artificial Sequence
  CNAG 06814 3 flanking region primer 2
taagtgtgttcacggtgcg

SEQ ID NO: 1183 - DNA, 19 bp, Artificial Sequence
  CNAG 06814 diagnostic screening primer, pairing with B79
tggttaccgaagtagggag

SEQ ID NO: 1184 - DNA, 19 bp, Artificial Sequence
  CNAG 06814 Southern blot probe primer
atttggaggtggagttctg

SEQ ID NO: 1185 - DNA, 22 bp, Artificial Sequence
  NAT 208 STM primer
tggtcgcgggagatcgtggttt

SEQ ID NO: 1186 - DNA, 24 bp, Artificial Sequence
  STM common primer
gcatgccctgcccctaagaattcg

SEQ ID NO: 1187 - DNA, 21 bp, Artificial Sequence
  CNAG 05431 5 flanking region primer 1
catcagtcttgcttcttctgc

SEQ ID NO: 1188 - DNA, 40 bp, Artificial Sequence
  CNAG 05431 5 flanking region primer 2
tcactggccgtcgttttacagagtcagaataggtgttggg

SEQ ID NO: 1189 - DNA, 40 bp, Artificial Sequence
  CNAG 05431 3 flanking region primer 1
catggtcatagctgtttcctgagggagaggattgaagtgg

SEQ ID NO: 1190 - DNA, 20 bp, Artificial Sequence
  CNAG 05431 3 flanking region primer 2
cattactttgactgggaggg

SEQ ID NO: 1191 - DNA, 19 bp, Artificial Sequence
  CNAG 05431 diagnostic screening primer, pairing with B79
cacggcaacttatgctctc

SEQ ID NO: 1192 - DNA, 20 bp, Artificial Sequence
  CNAG 05431 Southern blot probe primer
actcggtgttggtgaaacgg

SEQ ID NO: 1193 - DNA, 22 bp, Artificial Sequence
  NAT 208 STM primer
tggtcgcgggagatcgtggttt

SEQ ID NO: 1194 - DNA, 24 bp, Artificial Sequence
  STM common primer
gcatgccctgcccctaagaattcg

SEQ ID NO: 1195 - DNA, 20 bp, Artificial Sequence
  CNAG 05642 5 flanking region primer 1
agagggataagagcctgacg

SEQ ID NO: 1196 - DNA, 38 bp, Artificial Sequence
  CNAG 05642 5 flanking region primer 2
tcactggccgtcgttttacagaaggaagttgggaagcg

SEQ ID NO: 1197 - DNA, 41 bp, Artificial Sequence
  CNAG 05642 3 flanking region primer 1
catggtcatagctgtttcctgatgattacagaggggaggac

SEQ ID NO: 1198 - DNA, 19 bp, Artificial Sequence
  CNAG 05642 3 flanking region primer 2
tgtatcccaagcagcaaag

SEQ ID NO: 1199 - DNA, 20 bp, Artificial Sequence
  CNAG 05642 diagnostic screening primer, pairing with B79
ctcggaaataaatgtggagc

SEQ ID NO: 1200 - DNA, 20 bp, Artificial Sequence
  CNAG 05642 Southern blot probe primer
tacctgccagtgcttcaacc

SEQ ID NO: 1201 - DNA, 20 bp, Artificial Sequence
  NAT 210 STM primer
ctagagcccgccacaacgct

SEQ ID NO: 1202 - DNA, 24 bp, Artificial Sequence
  STM common primer
gcatgccctgcccctaagaattcg

SEQ ID NO: 1203 - DNA, 19 bp, Artificial Sequence
  CNAG 03212 5 flanking region primer 1
gacacccaaacaagcaaac

SEQ ID NO: 1204 - DNA, 39 bp, Artificial Sequence
  CNAG 03212 5 flanking region primer 2
gctcactggccgtcgttttacctggggtgtttgttttcg

SEQ ID NO: 1205 - DNA, 41 bp, Artificial Sequence
  CNAG 03212 3 flanking region primer 1
catggtcatagctgtttcctggggttcagattttggatacc

SEQ ID NO: 1206 - DNA, 19 bp, Artificial Sequence
  CNAG 03212 3 flanking region primer 2
ggcgaggtgttgtaaatcc

SEQ ID NO: 1207 - DNA, 22 bp, Artificial Sequence
  CNAG 03212 diagnostic screening primer, pairing with B79
gcgtatctggcatcttgttatc

SEQ ID NO: 1208 - DNA, 18 bp, Artificial Sequence
  CNAG 03212 Southern blot probe primer
tgaggaatgccaatggtg

SEQ ID NO: 1209 - DNA, 20 bp, Artificial Sequence
  NAT 211 STM primer
gcggtcgctttatagcgatt

SEQ ID NO: 1210 - DNA, 24 bp, Artificial Sequence
  STM common primer
gcatgccctgcccctaagaattcg

SEQ ID NO: 1211 - DNA, 18 bp, Artificial Sequence
  CNAG 03212 5 flanking region primer 1
gcgaaactttgtgagtgc

SEQ ID NO: 1212 - DNA, 40 bp, Artificial Sequence
  CNAG 03212 5 flanking region primer 2
gctcactggccgtcgttttaccaagcagcaagatggagtc

SEQ ID NO: 1213 - DNA, 40 bp, Artificial Sequence
  CNAG 03212 3 flanking region primer 1
catggtcatagctgtttcctgtcgtaagttcggtgtcagc

SEQ ID NO: 1214 - DNA, 20 bp, Artificial Sequence
  CNAG 03212 3 flanking region primer 2
aagtcttattatgggcgagg

SEQ ID NO: 1215 - DNA, 19 bp, Artificial Sequence
  CNAG 03212 diagnostic screening primer, pairing with B79
tgtcccgttgataggtgtc

SEQ ID NO: 1216 - DNA, 20 bp, Artificial Sequence
  CNAG 03212 Southern blot probe primer
tcctctccctcctattcagg

SEQ ID NO: 1217 - DNA, 20 bp, Artificial Sequence
  NAT 211 STM primer
gcggtcgctttatagcgatt

SEQ ID NO: 1218 - DNA, 24 bp, Artificial Sequence
  STM common primer
gcatgccctgcccctaagaattcg

SEQ ID NO: 1219 - DNA, 19 bp, Artificial Sequence
  CNAG 07924 5 flanking region primer 1
acctaatcaggaaagcccc

SEQ ID NO: 1220 - DNA, 39 bp, Artificial Sequence
  CNAG 07924 5 flanking region primer 2
tcactggccgtcgttttaccattccttctccgaaatctc

SEQ ID NO: 1221 - DNA, 40 bp, Artificial Sequence
  CNAG 07924 3 flanking region primer 1
catggtcatagctgtttcctgttatcctcccgaataccgc

SEQ ID NO: 1222 - DNA, 18 bp, Artificial Sequence
  CNAG 07924 3 flanking region primer 2
ccgcaccaaactatttcg

SEQ ID NO: 1223 - DNA, 18 bp, Artificial Sequence
  CNAG 07924 diagnostic screening primer, pairing with B79
atcgcacgaaaggcaaag

SEQ ID NO: 1224 - DNA, 20 bp, Artificial Sequence
  CNAG 07924 Southern blot probe primer
tctaaacccgtctctgactg

SEQ ID NO: 1225 - DNA, 20 bp, Artificial Sequence
  NAT 218 STM primer
ctccacatccatcgctccaa

SEQ ID NO: 1226 - DNA, 24 bp, Artificial Sequence
  STM common primer
gcatgccctgcccctaagaattcg

SEQ ID NO: 1227 - DNA, 22 bp, Artificial Sequence
  CNAG 00156 5 flanking region primer 1
cccgattttgctttttagtgcg

SEQ ID NO: 1228 - DNA, 36 bp, Artificial Sequence
  CNAG 00156 5 flanking region primer 2
ctggccgtcgttttacttgccgtctttgcttgggtc

SEQ ID NO: 1229 - DNA, 36 bp, Artificial Sequence
  CNAG 00156 3 flanking region primer 1
gtcatagctgtttcctgatcaagtccactcccgaag

SEQ ID NO: 1230 - DNA, 19 bp, Artificial Sequence
  CNAG 00156 3 flanking region primer
tagatgatgagggcgaacg

SEQ ID NO: 1231 - DNA, 20 bp, Artificial Sequence
  CNAG 00156 diagnostic screening primer, pairing with B79
gcttcgttggttcgttagtc

SEQ ID NO: 1232 - DNA, 20 bp, Artificial Sequence
  CNAG 00156 Southern blot probe primer
caatccgtttctctcacctc

SEQ ID NO: 1233 - DNA, 20 bp, Artificial Sequence
  NAT 231 STM primer
gagagatcccaacatcacgc

SEQ ID NO: 1234 - DNA, 24 bp, Artificial Sequence
  STM common primer
gcatgccctgcccctaagaattcg

SEQ ID NO: 1235 - DNA, 19 bp, Artificial Sequence
  CNAG 01173 5 flanking region primer 1
agtatcccaagcatccctc

SEQ ID NO: 1236 - DNA, 39 bp, Artificial Sequence
  CNAG 01173 5 flanking region primer 2
tcactggccgtcgttttacggcaggagcaaagtgtattc

SEQ ID NO: 1237 - DNA, 39 bp, Artificial Sequence
  CNAG 01173 3 flanking region primer 1
catggtcatagctgtttcctgtatggtggcatctgaacc

SEQ ID NO: 1238 - DNA, 20 bp, Artificial Sequence
  CNAG 01173 3 flanking region primer 2
ctgacaagtttaccaggacg

SEQ ID NO: 1239 - DNA, 20 bp, Artificial Sequence
  CNAG 01173 diagnostic screening primer, pairing with B79
gcttctgtgtgtctcctctg

SEQ ID NO: 1240 - DNA, 20 bp, Artificial Sequence
  CNAG 01173 Southern blot probe primer
gagaaagaaggattccgaag

SEQ ID NO: 1241 - DNA, 24 bp, Artificial Sequence
  NAT 242 STM primer
gtagcgataggggtgtcgctttag

SEQ ID NO: 1242 - DNA, 24 bp, Artificial Sequence
  STM common primer
gcatgccctgcccctaagaattcg

SEQ ID NO: 1243 - DNA, 19 bp, Artificial Sequence
  CNAG 06871 5 flanking region primer 1
tcaggatgggagtgtttgc

SEQ ID NO: 1244 - DNA, 37 bp, Artificial Sequence
  CNAG 06871 5 flanking region primer 2
tcactggccgtcgttttacccttctccttttgcttgg

SEQ ID NO: 1245 - DNA, 41 bp, Artificial Sequence
  CNAG 06871 3 flanking region primer 1
catggtcatagctgtttcctgcgctcttattaccgcttcag

SEQ ID NO: 1246 - DNA, 20 bp, Artificial Sequence
  CNAG 06871 3 flanking region primer 2
tcggtatttcagtagggatg

SEQ ID NO: 1247 - DNA, 19 bp, Artificial Sequence
  CNAG 06871 diagnostic screening primer, pairing with B79
cgactgctggatgtatgag

SEQ ID NO: 1248 - DNA, 18 bp, Artificial Sequence
  CNAG 06871 Southern blot probe primer
cttgactgcctcatctgc

SEQ ID NO: 1249 - DNA, 21 bp, Artificial Sequence
  NAT 295 STM primer
acacctacatcaaaccctccc

SEQ ID NO: 1250 - DNA, 24 bp, Artificial Sequence
  STM common primer
gcatgccctgcccctaagaattcg

SEQ ID NO: 1251 - DNA, 18 bp, Artificial Sequence
  CNAG 04774 5 flanking region primer 1
atttgaatagccgcaccc

SEQ ID NO: 1252 - DNA, 35 bp, Artificial Sequence
  CNAG 04774 5 flanking region primer 2
ctggccgtcgttttacattgggggatgctggaaag

SEQ ID NO: 1253 - DNA, 36 bp, Artificial Sequence
  CNAG 04774 3 flanking region primer 1
gtcatagctgtttcctgggtggactgccaaactatg

SEQ ID NO: 1254 - DNA, 19 bp, Artificial Sequence
  CNAG 04774 3 flanking region primer 2
aggcatttctgttcctccg

SEQ ID NO: 1255 - DNA, 19 bp, Artificial Sequence
  CNAG 04774 diagnostic screening primer, pairing with B79
cgatagacggaggatgaag

SEQ ID NO: 1256 - DNA, 18 bp, Artificial Sequence
  CNAG 04774 Southern blot probe primer
atcgttgcttccgcttag

SEQ ID NO: 1257 - DNA, 21 bp, Artificial Sequence
  NAT 146 STM primer
actagccccccctcaccacct

SEQ ID NO: 1258 - DNA, 24 bp, Artificial Sequence
  STM common primer
gcatgccctgcccctaagaattcg

SEQ ID NO: 1259 - DNA, 18 bp, Artificial Sequence
  CNAG 00841 5 flanking region primer 1
tcggcaagtttagcaagc

SEQ ID NO: 1260 - DNA, 39 bp, Artificial Sequence
  CNAG 00841 5 flanking region primer 2
gctcactggccgtcgttttacttatttctgggctgtcgc

SEQ ID NO: 1261 - DNA, 40 bp, Artificial Sequence
  CNAG 00841 3 flanking region primer 1
catggtcatagctgtttcctgtctgtggagtcagccaaac

SEQ ID NO: 1262 - DNA, 23 bp, Artificial Sequence
  CNAG 00841 3 flanking region primer 2
cggtatcctcgtgagtctatttc

SEQ ID NO: 1263 - DNA, 21 bp, Artificial Sequence
  CNAG 00841 diagnostic screening primer, pairing with B79
gcagttgctgaagtgaaagac

SEQ ID NO: 1264 - DNA, 20 bp, Artificial Sequence
  CNAG 00841 Southern blot probe primer
tgggatagaagaaggctgtg

SEQ ID NO: 1265 - DNA, 24 bp, Artificial Sequence
  NAT 205 STM primer
tatccccctctccgctctctagca

SEQ ID NO: 1266 - DNA, 24 bp, Artificial Sequence
  STM common primer
gcatgccctgcccctaagaattcg

SEQ ID NO: 1267 - DNA, 18 bp, Artificial Sequence
  CNAG 04836 5 flanking region primer 1
tagaaaagccacaacccc

SEQ ID NO: 1268 - DNA, 37 bp, Artificial Sequence
  CNAG 04836 5 flanking region primer 2
tcactggccgtcgttttacgagtttcttttggggtgg

SEQ ID NO: 1269 - DNA, 40 bp, Artificial Sequence
  CNAG 04836 3 flanking region primer 1
catggtcatagctgtttcctgacgagaaggggaatgatgg

SEQ ID NO: 1270 - DNA, 19 bp, Artificial Sequence
  CNAG 04836 3 flanking region primer 2
tgggcatcttgctcattac

SEQ ID NO: 1271 - DNA, 19 bp, Artificial Sequence
  CNAG 04836 diagnostic screening primer, pairing with B79
cggagagcaaatagcacag

SEQ ID NO: 1272 - DNA, 20 bp, Artificial Sequence
  CNAG 04836 Southern blot probe primer
aggtgtcgtatcgtatttgc

SEQ ID NO: 1273 - DNA, 20 bp, Artificial Sequence
  NAT 123 STM primer
ctatcgaccaaccaacacag

SEQ ID NO: 1274 - DNA, 24 bp, Artificial Sequence
  STM common primer
gcatgccctgcccctaagaattcg

SEQ ID NO: 1275 - DNA, 19 bp, Artificial Sequence
  CNAG 02603 5 flanking region primer 1
tcaaaacgcctttcggaac

SEQ ID NO: 1276 - DNA, 40 bp, Artificial Sequence
  CNAG 02603 5 flanking region primer 2
gctcactggccgtcgttttacacaaatggcagttccctcg

SEQ ID NO: 1277 - DNA, 43 bp, Artificial Sequence
  CNAG 02603 3 flanking region primer 1
catggtcatagctgtttcctgatctctccctcacttcagtcgg

SEQ ID NO: 1278 - DNA, 19 bp, Artificial Sequence
  CNAG 02603 3 flanking region primer 2
taggaccaacttcagaccg

SEQ ID NO: 1279 - DNA, 20 bp, Artificial Sequence
  CNAG 02603 diagnostic screening primer, pairing with B79
gcctatggaagaagacaacg

SEQ ID NO: 1280 - DNA, 20 bp, Artificial Sequence
  CNAG 02603 Southern blot probe primer
gtcgtgtagttttgaatggc

SEQ ID NO: 1281 - DNA, 19 bp, Artificial Sequence
  NAT 224 STM primer
aacctttaaatgggtagag

SEQ ID NO: 1282 - DNA, 24 bp, Artificial Sequence
  STM common primer
gcatgccctgcccctaagaattcg

SEQ ID NO: 1283 - DNA, 20 bp, Artificial Sequence
  CNAG 03059 5 flanking region primer 1
aatggcgttaccgacgagag

SEQ ID NO: 1284 - DNA, 36 bp, Artificial Sequence
  CNAG 03059 5 flanking region primer 2
ctggccgtcgttttacggtattgagagtgccgagtg

SEQ ID NO: 1285 - DNA, 38 bp, Artificial Sequence
  CNAG 03059 3 flanking region primer 1
gtcatagctgtttcctgcagactccctcttcttacctg

SEQ ID NO: 1286 - DNA, 24 bp, Artificial Sequence
  CNAG 03059 3 flanking region primer 2
cgttactgcgaggttattactgtg

SEQ ID NO: 1287 - DNA, 19 bp, Artificial Sequence
  CNAG 03059 diagnostic screening primer, pairing with B79
aaagactcacccttcaacg

SEQ ID NO: 1288 - DNA, 20 bp, Artificial Sequence
  CNAG 03059 Southern blot probe primer
cttttttccgctggtttctc

SEQ ID NO: 1289 - DNA, 18 bp, Artificial Sequence
  NAT 232 STM primer
ctttaaaggtggtttgtg

SEQ ID NO: 1290 - DNA, 24 bp, Artificial Sequence
  STM common primer
gcatgccctgcccctaagaattcg

SEQ ID NO: 1291 - DNA, 21 bp, Artificial Sequence
  CNAG 00883 5 flanking region primer 1
cgacagaaacaagtccaagtg

SEQ ID NO: 1292 - DNA, 36 bp, Artificial Sequence
  CNAG 00883 5 flanking region primer 2
ctggccgtcgttttacgtagtgaaatgaggattgcg

SEQ ID NO: 1293 - DNA, 35 bp, Artificial Sequence
  CNAG 00883 3 flanking region primer 1
gtcatagctgtttcctgtacctgcttttcttgcgg

SEQ ID NO: 1294 - DNA, 18 bp, Artificial Sequence
  CNAG 00883 3 flanking region primer 2
acaagcccgaatctcaag

SEQ ID NO: 1295 - DNA, 19 bp, Artificial Sequence
  CNAG 00883 diagnostic screening primer, pairing with B79
cttttcactcaatgtcccg

SEQ ID NO: 1296 - DNA, 19 bp, Artificial Sequence
  CNAG 00883 Southern blot probe primer
taagaactgttgccctgcc

SEQ ID NO: 1297 - DNA, 18 bp, Artificial Sequence
  CNAG 01551 5 flanking region primer 1
tccgttgagatagcgttg

SEQ ID NO: 1298 - DNA, 39 bp, Artificial Sequence
  CNAG 01551 5 flanking region primer 2
tcactggccgtcgttttacatggtggaggtgtaggactg

SEQ ID NO: 1299 - DNA, 39 bp, Artificial Sequence
  CNAG 01551 3 flanking region primer 1
catggtcatagctgtttcctgttattccccctcaagagc

SEQ ID NO: 1300 - DNA, 20 bp, Artificial Sequence
  CNAG 01551 3 flanking region primer 2
gcggcccattcttcttgtcg

SEQ ID NO: 1301 - DNA, 18 bp, Artificial Sequence
  CNAG 01551 diagnostic screening primer, pairing with B79
aaccttgcctaacaaccc

SEQ ID NO: 1302 - DNA, 20 bp, Artificial Sequence
  CNAG 01551 Southern blot probe primer
ggagtatggctgaaatctgg

SEQ ID NO: 1303 - DNA, 24 bp, Artificial Sequence
  NAT 273 STM primer
gagatctttcgggaggtctggatt

SEQ ID NO: 1304 - DNA, 24 bp, Artificial Sequence
  STM common primer
gcatgccctgcccctaagaattcg

SEQ ID NO: 1305 - DNA, 20 bp, Artificial Sequence
  CNAG 04353 5 flanking region primer 1
tgcgagagagaatgggagac

SEQ ID NO: 1306 - DNA, 38 bp, Artificial Sequence
  CNAG 04353 5 flanking region primer 2
tcactggccgtcgttttacggttccatagcgacatcag

SEQ ID NO: 1307 - DNA, 41 bp, Artificial Sequence
  CNAG 04353 3 flanking region primer 1
catggtcatagctgtttcctgcgaaggttgtttgtgatacg

SEQ ID NO: 1308 - DNA, 20 bp, Artificial Sequence
  CNAG 04353 3 flanking region primer 2
cccaacaactcatcaatctc

SEQ ID NO: 1309 - DNA, 18 bp, Artificial Sequence
  CNAG 04353 diagnostic screening primer, pairing with B79
gcattatcgtttcccgac

SEQ ID NO: 1310 - DNA, 22 bp, Artificial Sequence
  CNAG 04353 Southern blot probe primer
ttgaggtaggtggagtgttgcc

SEQ ID NO: 1311 - DNA, 20 bp, Artificial Sequence
  CNAG 03346 5 flanking region primer 1
cgccctttctattgttacac

SEQ ID NO: 1312 - DNA, 39 bp, Artificial Sequence
  CNAG 03346 5 flanking region primer 2
tcactggccgtcgttttacgatgacaggagggatgaatc

SEQ ID NO: 1313 - DNA, 43 bp, Artificial Sequence
  CNAG 03346 3 flanking region primer 1
catggtcatagctgtttcctggggagaataacgactcaatgtc

SEQ ID NO: 1314 - DNA, 18 bp, Artificial Sequence
  CNAG 03346 3 flanking region primer 2
tcattgctgactgggaag

SEQ ID NO: 1315 - DNA, 18 bp, Artificial Sequence
  CNAG 03346 diagnostic screening primer, pairing with B79
aaagaggcggtgttgaag

SEQ ID NO: 1316 - DNA, 19 bp, Artificial Sequence
  CNAG 03346 Southern blot probe primer
agccaggtaatcttggagg

SEQ ID NO: 1317 - DNA, 21 bp, Artificial Sequence
  NAT 295 STM primer
acacctacatcaaaccctccc

SEQ ID NO: 1318 - DNA, 24 bp, Artificial Sequence
  STM common primer
gcatgccctgcccctaagaattcg

SEQ ID NO: 1319 - DNA, 18 bp, Artificial Sequence
  CNAG 00239 5 flanking region primer 1
aggattatcgcctgaacc

SEQ ID NO: 1320 - DNA, 37 bp, Artificial Sequence
  CNAG 00239 5 flanking region primer 2
tcactggccgtcgttttacatgttgttgaagagggcg

SEQ ID NO: 1321 - DNA, 41 bp, Artificial Sequence
  CNAG 00239 3 flanking region primer 1
catggtcatagctgtttcctgaacgacgaccatcgcagtag

SEQ ID NO: 1322 - DNA, 18 bp, Artificial Sequence
  CNAG 00239 3 flanking region primer 2
catcccgagttggaaaac

SEQ ID NO: 1323 - DNA, 20 bp, Artificial Sequence
  CNAG 00239 diagnostic screening primer, pairing with B79
cactggcgaagtcaagtatg

SEQ ID NO: 1324 - DNA, 20 bp, Artificial Sequence
  CNAG 00239 Southern blot probe primer
ctactctgtggtggcgtaag

SEQ ID NO: 1325 - DNA, 20 bp, Artificial Sequence
  NAT 296 STM primer
cgcccgccctcactatccac

SEQ ID NO: 1326 - DNA, 24 bp, Artificial Sequence
  STM common primer
gcatgccctgcccctaagaattcg

SEQ ID NO: 1327 - DNA, 19 bp, Artificial Sequence
  CNAG 03129 5 flanking region primer 1
gcaggcgaaaactattagg

SEQ ID NO: 1328 - DNA, 41 bp, Artificial Sequence
  CNAG 03129 5 flanking region primer 2
gctcactggccgtcgttttacgtggggaagtcctcaaatac

SEQ ID NO: 1329 - DNA, 42 bp, Artificial Sequence
  CNAG 03129 3 flanking region primer 1
catggtcatagctgtttcctgcgacgacatctcaagtcagag

SEQ ID NO: 1330 - DNA, 20 bp, Artificial Sequence
  CNAG 03129 3 flanking region primer 2
tgatgggacgataaaggttc

SEQ ID NO: 1331 - DNA, 19 bp, Artificial Sequence
  CNAG 03129 diagnostic screening primer, pairing with B79
tgctttctctaatagcggc

SEQ ID NO: 1332 - DNA, 19 bp, Artificial Sequence
  CNAG 03129 Southern blot probe primer
gtgcttttgaagttccgtc

SEQ ID NO: 1333 - DNA, 18 bp, Artificial Sequence
  CNAG 03826 5 flanking region primer 1
aatgctgatttggcaggg

SEQ ID NO: 1334 - DNA, 39 bp, Artificial Sequence
  CNAG 03826 5 flanking region primer 2
tcactggccgtcgttttaccgatggaagatgttgtcagc

SEQ ID NO: 1335 - DNA, 39 bp, Artificial Sequence
  CNAG 03826 3 flanking region primer 1
catggtcatagctgtttcctgacaaagcgaaaggcagac

SEQ ID NO: 1336 - DNA, 18 bp, Artificial Sequence
  CNAG 03826 3 flanking region primer 2
cgaaagtgatgcttcagc

SEQ ID NO: 1337 - DNA, 20 bp, Artificial Sequence
  CNAG 03826 diagnostic screening primer, pairing with B79
ggaaggtcaggtcctatttg

SEQ ID NO: 1338 - DNA, 20 bp, Artificial Sequence
  CNAG 02671 5 flanking region primer 1
cgccgagttatctgacaaag

SEQ ID NO: 1339 - DNA, 41 bp, Artificial Sequence
  CNAG 02671 5 flanking region primer 2
tcactggccgtcgttttacttctccaaacaccgcccttgac

SEQ ID NO: 1340 - DNA, 40 bp, Artificial Sequence
  CNAG 02671 3 flanking region primer 1
catggtcatagctgtttcctgtaacggggcttagaagtgc

SEQ ID NO: 1341 - DNA, 18 bp, Artificial Sequence
  CNAG 02671 3 flanking region primer 2
ttcatcaaaggtctggcg

SEQ ID NO: 1342 - DNA, 20 bp, Artificial Sequence
  CNAG 02671 diagnostic screening primer, pairing with B79
cctgactcatcggcttactc

SEQ ID NO: 1343 - DNA, 18 bp, Artificial Sequence
  CNAG 02671 Southern blot probe primer
actcctgcttgcgttttc

SEQ ID NO: 1344 - DNA, 20 bp, Artificial Sequence
  CNAG 02134 5 flanking region primer 1
ggggtaactatcatcatccg

SEQ ID NO: 1345 - DNA, 38 bp, Artificial Sequence
  CNAG 02134 5 flanking region primer 2
tcactggccgtcgttttactctcgctccttttccttcc

SEQ ID NO: 1346 - DNA, 42 bp, Artificial Sequence
  CNAG 02134 3 flanking region primer 1
catggtcatagctgtttcctggcagtagaaggtgatacaacg

SEQ ID NO: 1347 - DNA, 19 bp, Artificial Sequence
  CNAG 02134 3 flanking region primer 2
cgttacggttggacaatgg

SEQ ID NO: 1348 - DNA, 19 bp, Artificial Sequence
  CNAG 02134 diagnostic screening primer, pairing with B79
cgtagaaacaacgcacgag

SEQ ID NO: 1349 - DNA, 21 bp, Artificial Sequence
  CNAG 02134 Southern blot probe primer
caagtattctccagggttcag

SEQ ID NO: 1350 - DNA, 20 bp, Artificial Sequence
  CNAG 02936 5 flanking region primer 1
gcgagtgatgacgagagttc

SEQ ID NO: 1351 - DNA, 39 bp, Artificial Sequence
  CNAG 02936 5 flanking region primer 2
tcactggccgtcgttttacgccttgaccgtctcttctac

SEQ ID NO: 1352 - DNA, 42 bp, Artificial Sequence
  CNAG 02936 3 flanking region primer 1
catggtcatagctgtttcctggtctactcgtcactttcatcg

SEQ ID NO: 1353 - DNA, 21 bp, Artificial Sequence
  CNAG 02936 3 flanking region primer 2
cgtcacaacaagcactctcac

SEQ ID NO: 1354 - DNA, 19 bp, Artificial Sequence
  CNAG 02936 diagnostic screening primer, pairing with B79
ggatgttcaggtgccaatc

SEQ ID NO: 1355 - DNA, 18 bp, Artificial Sequence
  CNAG 07460 5 flanking region primer 1
tcggcattggcatacttg

SEQ ID NO: 1356 - DNA, 39 bp, Artificial Sequence
  CNAG_07460 5 flanking region primer 2
gctcactggccgtcgttttacgtgatttgagcggtgaag

SEQ ID NO: 1357 - DNA, 42 bp, Artificial Sequence
  CNAG_07460 3 flanking region primer 1
catggtcatagctgtttcctgccaaagtggactcaaataggg

SEQ ID NO: 1358 - DNA, 22 bp, Artificial Sequence
  CNAG_07460 3 flanking region primer 2
ccgttactttccaaatcaggag

SEQ ID NO: 1359 - DNA, 20 bp, Artificial Sequence
  CNAG 07460 diagnostic screening primer, pairing with B79
cgtctgaacctgattctgag

SEQ ID NO: 1360 - DNA, 19 bp, Artificial Sequence
  CNAG 07460 Southern blot probe primer
ccgatgaccaaaagtaggc

SEQ ID NO: 1361 - DNA, 19 bp, Artificial Sequence
  CNAG 07460 Southern blot probe primer
cagagttgattgccgaaag

SEQ ID NO: 1362 - DNA, 19 bp, Artificial Sequence
  CNAG 05436 5 flanking region primer 1
gcccttttgctttaccttc

SEQ ID NO: 1363 - DNA, 41 bp, Artificial Sequence
  CNAG 05436 5 flanking region primer 2
tcactggccgtcgttttaccgtagagggatgattttgagac

SEQ ID NO: 1364 - DNA, 40 bp, Artificial Sequence
  CNAG 05436 3 flanking region primer 1
catggtcatagctgtttcctggcatccttcgtgctaacag

SEQ ID NO: 1365 - DNA, 18 bp, Artificial Sequence
  CNAG 05436 3 flanking region primer 2
gccttgaagaacaaacgg

SEQ ID NO: 1366 - DNA, 19 bp, Artificial Sequence
  CNAG 05436 diagnostic screening primer, pairing with B79
gggataggaggcattgatg

SEQ ID NO: 1367 - DNA, 20 bp, Artificial Sequence
  CNAG 05436 Southern blot probe primer
caccaaagtggatttccttg

SEQ ID NO: 1368 - DNA, 21 bp, Artificial Sequence
  CNAG 05436 Southern blot probe primer
ctcggacaaatcactctactg

SEQ ID NO: 1369 - DNA, 21 bp, Artificial Sequence
  CNAG 02698 5 flanking region primer 1
ctacagataatgtgctcagcc

SEQ ID NO: 1370 - DNA, 37 bp, Artificial Sequence
  CNAG 02698 5 flanking region primer 2
tcactggccgtcgttttactttgttccatagccagcg

SEQ ID NO: 1371 - DNA, 40 bp, Artificial Sequence
  CNAG 02698 3 flanking region primer 1
catggtcatagctgtttcctggttctgtcttgaagggtgc

SEQ ID NO: 1372 - DNA, 20 bp, Artificial Sequence
  CNAG 02698 3 flanking region primer 2
cggtgaagttggctaagaac

SEQ ID NO: 1373 - DNA, 19 bp, Artificial Sequence
  CNAG 02698 diagnostic screening primer, pairing with B79
gaaagggctaagaagcgac

SEQ ID NO: 1374 - DNA, 20 bp, Artificial Sequence
  CNAG 02698 Southern blot probe primer
aaactctccatctgtgtccc

SEQ ID NO: 1375 - DNA, 20 bp, Artificial Sequence
  CNAG 04600 5 flanking region primer 1
gctgttggctgattgtttac

SEQ ID NO: 1376 - DNA, 39 bp, Artificial Sequence
  CNAG 04600 5 flanking region primer 2
tcactggccgtcgttttacttacagtatcccaccgtccc

SEQ ID NO: 1377 - DNA, 40 bp, Artificial Sequence
  CNAG 04600 3 flanking region primer 1
catggtcatagctgtttcctgtgggagtgatgtctttggc

SEQ ID NO: 1378 - DNA, 18 bp, Artificial Sequence
  CNAG 04600 3 flanking region primer 2
ccctcaaaagttgcgaag

SEQ ID NO: 1379 - DNA, 18 bp, Artificial Sequence
  CNAG 04600 diagnostic screening primer, pairing with B79
ctggattttggaatgggc

SEQ ID NO: 1380 - DNA, 18 bp, Artificial Sequence
  CNAG 04600 Southern blot probe primer
ttacatttgccagcggac

SEQ ID NO: 1381 - DNA, 28 bp, Artificial Sequence
  B1894
ttttacgctttttgcagattccgccaaa

SEQ ID NO: 1382 - DNA, 21 bp, Artificial Sequence
  B195
gaccactgtttctttcgttct

SEQ ID NO: 1383 - DNA, 18 bp, Artificial Sequence
  B3662
ctttcaatcgtccaagcg

SEQ ID NO: 1384 - DNA, 20 bp, Artificial Sequence
  B3663
ccccagttatccaaaaagtc

SEQ ID NO: 1385 - DNA, 18 bp, Artificial Sequence
  B5789
caagaaatggagcgtgag

SEQ ID NO: 1386 - DNA, 20 bp, Artificial Sequence
  B5790
cagtgttgtaaagcgtgatg

SEQ ID NO: 1387 - DNA, 20 bp, Artificial Sequence
  B1720
atcccttttcaccgtcgctc

SEQ ID NO: 1388 - DNA, 21 bp, Artificial Sequence
  B1721
cgtcgtggatgagaatagtcc

SEQ ID NO: 1389 - DNA, 21 bp, Artificial Sequence
  B671
gtttgttgcctgagaactggg

SEQ ID NO: 1390 - DNA, 24 bp, Artificial Sequence
  B674
atcactcaactcggtcctctcgtg

SEQ ID NO: 1391 - DNA, 21 bp, Artificial Sequence
  B678
ttcagggaacttgggaacagc

SEQ ID NO: 1392 - DNA, 18 bp, Artificial Sequence
  B1598
caggagcagaaacaaagc

SEQ ID NO: 1393 - DNA, 19 bp, Artificial Sequence
  B1718
tgaccgcctgtagattgtc

SEQ ID NO: 1394 - DNA, 19 bp, Artificial Sequence
  B1719
tagtcccaccacctgaaac

SEQ ID NO: 1395 - DNA, 20 bp, Artificial Sequence
  B6838
gggagggtcgaggatgtaga

SEQ ID NO: 1396 - DNA, 25 bp, Artificial Sequence
  B672
gtagatactgagagcctgcttggtg

SEQ ID NO: 1397 - DNA, 22 bp, Artificial Sequence
  B677
aatctccttaccagccattcgg

SEQ ID NO: 1398 - DNA, 19 bp, Artificial Sequence
  B2695
tcgtctttggcaagcagtc

SEQ ID NO: 1399 - DNA, 19 bp, Artificial Sequence
  B6840
gaagtcgtggtggtcagca

SEQ ID NO: 1400 - DNA, 22 bp, Artificial Sequence
  B679
cgcccttgctccttcttctatg

SEQ ID NO: 1401 - DNA, 22 bp, Artificial Sequence
  B680
tactcgtcgtattcgctcttcg

(19) United States
(12) Patent Application Publication
(10) Pub. No.: US 2018/0305728 A1
(43) Pub. Date: Oct. 25, 2018

(54) NOVEL GENE REGULATING VIRULENCE OF CRYPTOCOCCUS NEOFORMANS, AND USE THEREOF

(71) Applicant: AMTIXBIO CO., LTD., Seoul (KR)

(72) Inventors: Yong-Sun BAHN, Seoul (KR); Kwang-Woo JUNG, Seoul (KR); Dong-Hoon YANG, Namyangju, Gyeonggi-do (KR)

(21) Appl. No.: 15/563,345

(22) PCT Filed: Mar. 30, 2016
(86) PCT No.: PCT/KR2016/003255

§ 371 Date: Jun. 20, 2018

(30) Foreign Application Priority Data
Mar. 30, 2015 (KR) 10-2015-0044617
Mar. 30, 2015 (KR) 10-2015-0044621

(51) Int. Cl. C12Q 1/02; C12Q 1/18; C07K 14/375; C12Q 1/6895; A61P 31/04

(52) U.S. Cl. CPC C12Q 1/025; C12Q 1/18; A61K 39/00; C12Q 1/6895; A61P 31/04; C07K 14/375

(57) ABSTRACT

The present invention relates to a novel gene regulating the virulence of Cryptococcus neoformans, and a use thereof. According to the present invention, anti-Cryptococcal or anti-fungal drug candidate materials can be effectively screened for. In addition, the present invention relates to a method for screening for drug candidate materials, which can bring a synergistic effect by being co-administered with a commercially available anti-Cryptococcal drug or anti-fungal drug. Furthermore, provided is a pharmaceutical composition having an anti-Cryptococcal or anti-fungal effect by increasing or decreasing the expression of transcription factors. The present inventors have performed a large-scale virulence test by using insect and animal models so as to identity transcription factors, and have analyzed a complex correlation between the transcription factors and in vivo and in vitro phenotypes of pathogenicity.

### TECHNICAL FIELD

The present invention relates to novel genes that regulate the virulence of a Cryptococcus neoformans strain, and to the use thereof. Moreover, the present invention relates to a method for screening an antifungal agent and a method for screening an agent for treating meningitis, in which the methods comprise measuring the expression of genes that are involved in regulation of the virulence of a Cryptococcus neoformans strain.

### BACKGROUND ART

In the past, fungal infections were mainly topical infections such as athlete's foot, jock itch, or oral thrush, and rarely systemic infections. However, recently, systemic infections have frequently occurred such that they accounted for a high frequency of total infections in hospitals.

Antifungal agents developed so far can be largely classified according to chemical structure into two groups: those having an azole structure, and those having no azole structure. The azole-based antifungal agents include ketoconazole, fluconazole, itraconazole, voriconazole and the like, and the non-azole-based antifungal agents include terbinafine, flucytosine, amphotericin B, caspofungin and the like.

Ketoconazole, fluconazole, itraconazole and voriconazole, which have an azole structure, and allylamine-based antifungal agents, such as naftifine and terbinafine, have similar mechanisms of action. These two classes of antifungal agents act to inhibit enzymes required in the process in which lanosterol is converted into ergosterol that is the main component of the fungal cell membrane. The azole-based antifungal agents inhibit microsomal enzymes, and the allylamine-based antifungal agents inhibit squalene epoxidase, thereby exhibiting the above-described effect. Flucytocin (5-FC) is a metabolic antagonist that inhibits nucleic acid synthesis, exhibits antifungal activity by non-competitively antagonizing the overlapping coding of fungal RNA and DNA synthesis. Amphotericin B having a polyene structure exhibits antifungal activity by binding to ergosterol in the fungal cell membrane to induce depolarization of the cell membrane and forming a hole to induce loss of intracellular inclusions. Caspofungin, an echinocandin-based antifungal agent, has an activity of reversibly inhibiting fungal cell wall formation, and differs from the above-mentioned antifungal agents, which act on the cell membrane, in that it acts on the cell wall. The azole-based drug, when administered to patients with reduced liver function, may cause death by hepatitis, and for this reason, a liver function test should precede administration of the azole-based drug. It was reported that flutocytosin has dose-dependent bone marrow suppression and liver toxicity, and can cause enterocolitis. Such side effects further increase when renal function is reduced, and for this reason, monitoring of renal function in patients is very important. In addition, flutocytosin is contraindicated for pregnant women. The major toxicity of amphotericin B is glomerular nephrotoxicity resulting from renal artery vasoconstriction, which is dose-dependent. Thus, when the total cumulative dose of amphotericin B is 4 to 5 g, the possibility of permanent renal function impairment will increase. Furthermore, nephrotoxicity, including the excessive loss of potassium, magnesium and bicarbonate caused by renal tubular toxicity, and a decrease in erythropoietin production, may occur. In addition, as acute responses, symptoms, including thrombophlebitis, rigor, tremor and hyperventilation, may appear.

Meanwhile, Cryptococcus neoformans is a basidiomycete fungal pathogen that causes meningoencephalitis in immunocompromised populations, and is responsible for more than 600,000 deaths annually worldwide (Non-Patent Document 1). However, limited therapeutic options are available for treating cryptococcosis (Non-Patent Document 2). Thus, a complete understanding of diverse biological features of Cryptococcus is urgently required for developing novel therapeutic targets and methods. To this end, the signaling cascades governing the general biological features and pathogenicity of Cryptococcus neoformans have been extensively studied over the past decades. This study has made it possible to understand several key metabolic and signaling pathways in Cryptococcus neoformans, including those involving Ras, cAMP/protein kinase A, Rim101, calmodulin/calcineurin, three MAPKs (Cpk1, Mpk1 and Hog1), the unfolded protein response (UPR), and iron/copper uptake (Non-Patent Document 3, Non-Patent Document 4, and Non-Patent Document 5). The present inventors have found that impairment of function of Irel and Hxl1 (HAC1 and XBP1-like gene) proteins, newly identified in Cryptococcus neoformans, and genes encoding the proteins, shows an antifungal effect or a meningitis-treating effect (Patent Document 1 and Patent Document 2). Most of known signaling cascades are composed of sensor/receptor-like proteins and kinases/phosphatases, and are often equipped with unique adaptor or scaffolding proteins to enhance the specificity of each signaling pathway to prevent aberrant crosstalk between the signaling pathways. Nevertheless, each signaling cascade ultimately activates or represses a single transcription factor (TF) or multiple transcription factors, thereby up-regulating or down-regulating effector proteins of transcription factor through transcription factor binding to a specific region of promoter in a target gene that regulates the transcription level of transcription factor. Thus, transcription factor is regarded as a major regulator of gene expression in a given signaling pathway. Particularly, repertoires of transcription factors are often more divergent among species than are those of other signaling components. This appears particularly true in the case of C. neoformans, as described in the results of recent genome analyses (Non-Patent Document 6). For example, the UPR signaling pathway which is important in endoplasmic reticulum (ER) stress responses and Cryptococcus neoformans virulence is composed of evolutionarily highly conserved Irel kinase and Hxl1 transcription factor downstream of the Irel kinase. Therefore, C. neoformans appears to possess numerous evolutionarily conserved signaling cascades featuring divergent sets of TFs, which might govern the characteristics of C. neoformans that are unique compared with those of other fungi.

To understand C. neoformans transcription factor networks on a global scale, the present inventors constructed a high-quality gene-deletion mutant through homologous recombination methods for 155 putative C. neoformans transcription factors previously predicted, using a DNA-binding domain (DBD) transcription factor database to identify sequence-specific DNA-binding transcription factors in organisms whose genome sequences were analyzed (Non-Patent Document 7 and Non-Patent Document 8). The constructed transcription factor knockout (TFKO) strains were analyzed for 30 distinct in vitro phenotypic traits, which cover growth, differentiation, stress responses, antifungal resistance and virulence-factor production. Moreover, the present inventors performed a large-scale virulence test using an insect host model and signature-tagged mutagenesis (STM) scoring in a murine host model, and thus analyzed phenotypes resulting from deletion of various transcription factors in Cryptococcus neoformans strains, thereby constructing a comprehensive phenotypic data set (phenome) of the transcription factors, thereby completing the present invention.

Furthermore, the phenome of Cryptococcus neoformans transcription factors according to the present invention can be easily accessed online (http://tf.cryptococcus.org), and provides a unique opportunity to understand general biological features of C. neoformans, and also provides novel targets required for the treatment of cryptococcosis.

### PRIOR ART DOCUMENTS

### Patent Documents

(Patent Document 1) Korean Patent No. 10-1311196.
(Patent Document 2) Korean Patent No. 10-1403862.

### Non-Patent Documents

(Non-Patent Document 1) Park, B. J., and et al., 2009, AIDS, 23: 525-530.
(Non-Patent Document 2) Perfect, J. R., et al., 2010, Clinical infectious diseases, 2010 update by the infectious diseases society of America, 50: 291-322.
(Non-Patent Document 3) Bahn, Y. S., and Jung, K. W., 2013, Eukaryot. Cell. 12: 1564-1577.
(Non-Patent Document 4) Jung. W. H. and Kronstad. J. W., 2008, Cell. Microbiol., 10: 277-284.
(Non-Patent Document 5) Ding, C. et al., 2013, Cell Host Microbe, 13: 265-276.
(Non-Patent Document 6) Janbon, G., and et al., 2014, PLoS Genet. 10: e1004261.
(Non-Patent Document 7) Kummerfeld, S. K., and Teichmann, S. A., 2006, Nucleic Acids Res., 34: D74-81.
(Non-Patent Document 8) Wilson, D., and et al., 2008, Nucleic Acids Res. 36: D88-D92.
(Non-Patent Document 9) Cheon. S. A., and et al., 2011, PLoS Pathog. 7: e1002177.
(Non-Patent Document 10) Kim. M. S., and et al., 2010, Genetics. 185: 1207-1219.
(Non-Patent Document 11) Song, M. H., and et al., 2012, Eukaryot. Cell. 11: 53-67.
(Non-Patent Document 12) Liu. O. W. and et al., 2008, Cell. 135: 174-188.
(Non-Patent Document 13) Homann, O. R., and et al., 2009, PLoS Genet. 5: e 1000783.
(Non-Patent Document 14) Inglis. D. O., and et al., 2014, Eukaryot. Cell. 13: 878-883.
(Non-Patent Document 15) Davidson, R. C., and et al., 2002, Microbiology, 148: 2607-2615.
(Non-Patent Document 16) Kim. M. S., and et al., 2009, Biochem. Biophy. Res. Commun. 390: 983-988.
(Non-Patent Document 17) Lin, X., and Heitman, J., 2006, Annu. Rev. Microbiol. 60: 69-105.
(Non-Patent Document 18) Garcia-Rodas, R., and Zaragoza. O., 2012, Med Microbiol. 64: 147-161.
(Non-Patent Document 19) McFadden, D. C., and Casadevall, A. 2001, Med. Mycol. 39 Suppl 1:19-30.
(Non-Patent Document 20) Bahn, Y. S. and et al., 2004, Eukaryotic. Cell. 3: 1476-1491.
(Non-Patent Document 21) Mylonakis, E. et. al., 2005, Infect. Imraun. 73: 3842-3850.
(Non-Patent Document 22) Haynes, B. C., and et al., 2011, PLoS Pathog. 7: e1002411.
(Non-Patent Document 23) Wickes, B. L., and et al., 1997, Mol. Microbiol. 26: 951-960.
(Non-Patent Document 24) Bahn, Y. S., and et al., 2006, Mol. Biol. Cell, 17: 3122-3135.
(Non-Patent Document 25) Jiang, N., and et al., 2009, FEMS Microbiol. Lett. 296: 84-90.
(Non-Patent Document 26) Pukkila-Worley, R., and et al., 2005, Eukaryot. Cell 4: 190-201.
(Non-Patent Document 27) Chang, Y. C., and et al., 2007, Mol. Microbiol., 64: 614-629.
(Non-Patent Document 28) Chun, C. D., and et al., 2007, PLoS Pathog., 3: e22.
(Non-Patent Document 29) Cramer, K. L., and et al., 2006, Eukaryot. Cell, 5: 1147-1156.
(Non-Patent Document 30) Idnurm, A., and Heitman, J. 2005, PLoS Biol. 3: e95.
(Non-Patent Document 31) Alder, A., and et al., 2011, J. Biol. Chem., 286: 20977-20990.
(Non-Patent Document 32) Chun. C. D., and et al., 2011, Cell host & microbe, 9: 243-251.
(Non-Patent Document 33) Jung, W., and et al., 2011, Fungal Genet. Biol. 48: 154-165.
(Non-Patent Document 34) Livak. K. J. and Schmittgen, T. D., 2001, Methods. 25: 402-408.
(Non-Patent Document 35) Cowen, L. E., 2008, Nat. Rev. Microbiol. 6: 187-198.
(Non-Patent Document 36) Vermes, A., and et al., 2000, J Antimicrob. Chemother. 46: 171-179.
(Non-Patent Document 37) Choi, J. and et al., 2013, Nucleic Acids Res. 41: D714-719.
(Non-Patent Document 38) Hunter, S. et al., 2012, Nucleic Acids Res. 40: D306-312.
(Non-Patent Document 39) Bendtsen, J. D. and et al., 2004, J. Mol. Biol. 340: 783-795.
(Non-Patent Document 40) Nakai, K. and Horton, P., 1999, Trends Biochem. Sci. 24: 34-36.
(Non-Patent Document 41) Emanuelsson, O., and et al., 2000, J. Mol. Biol., 300: 1005-1016.
(Non-Patent Document 42) Emanuelsson, O., and et al., 1999, Protein Sci., 8: 978-984.
(Non-Patent Document 43) Bendtsen, J. D., and et al., 2004, Protein Eng. Des. Sel. 17: 349-356.
(Non-Patent Document 44) Cokol, M., and et al., 2000, EMBO reports 1: 411-415.
(Non-Patent Document 45) Sonnhammer, E. L., and et al., 1998, Proc. Int. Conf. Intelli. Syst. Mol. Biol 6: 175-812.
(Non-Patent Document 46) Jung, K. et al., 2008, BMC Genomics, 9: 586.

### DISCLOSURE

### Technical Problem

Therefore, it is an object of the present invention to construct transcription factor networks in a Cryptococcus neoformans strain and to provide various uses of the transcription factors.

It is an object of the present invention to provide a method for screening an agent for preventing or treating fungal infection caused by a Cryptococcus neoformans strain, or a method for screening an agent for treating meningitis.

Another object of the present invention is to provide a method for screening a candidate that may have a synergistic effect when administered in combination with a conventional antifungal agent or meningitis-treating agent.

Still another object of the present invention is to provide a pharmaceutical composition that exhibits an antifungal effect or a meningitis-treating effect by increasing or inhibiting the expression of a transcription factor that regulates the virulence of a Cryptococcus neoformans strain and a gene that encodes the transcription factor.

Yet another object of the present invention is to provide a pharmaceutical composition that exhibits an antifungal effect or a meningitis-treating effect by increasing or inhibiting the expression of a transcription factor that regulates the antifungal agent susceptibility of a Cryptococcus neoformans strain, a transcription factor that regulates the growth of the strain, a transcription factor that regulates the mating of the strain, a transcription factor that regulates the responses to various external stresses, and genes that encode the transcription factors.

### Technical Solution

To achieve the above objects, the present invention provides a Cryptococcus neoformans strain deposited under accession number KCCM51291.

The present invention also provides a method for screening an antifungal agent, an antifungal agent for co-administration, or a meningitis-treating agent, the method comprising measuring an increase or decrease in the expression of a virulence regulatory gene in Cryptococcus neoformans.

In one embodiment, the present invention provides a method for screening an antifungal agent, comprising the steps of: (a) bringing a sample to be analyzed into contact with a cell line (deposited under accession number KCCM51291) containing an antifungal agent-targeting gene; (b) measuring the expression of the antifungal agent-targeting gene in the cell line; and (c) determining that the sample is the antifungal agent, when the expression of the antifungal agent-targeting gene is measured to be down-regulated or up-regulated, wherein the antifungal agent-targeting gene is any one gene selected from the group consisting of an antifungal agent resistance regulatory gene, a growth regulatory gene, a mating regulatory gene, and a gene that regulates responses to external stress.

In the embodiment of the method for screening an antifungal agent, the antifungal agent resistance regulatory gene is a gene that regulates resistance to any one antifungal agent selected from the group consisting of azole-, polyene-, 5-flucytocin- and phenylpyrazole-based antifungal agents.

In the embodiment of the method for screening an antifungal agent, as the antifungal agent-targeting gene, a gene, which increases sensitivity to the azole-based antifungal agent when its expression is down-regulated, is any one gene selected from the group consisting of bzp3, hlh3, bzp/hxl1, sre1, riw101, bap2, hlh1, yap4, pip2, miz1, mln1, hob6, mbf1, met32, fzc46, bap1, fzc14, fzc2, liv4, hsf2, zfc6, fzc45, fzc30, asg1, ste12, liv1, fzc22, fzc31, pan1, bzp2, sp1/crz1, bzp5, hlh2, sxi1 alpha, fzc34, fzc40, fzc38 and fzcl7; a gene that increases sensitivity to the polyene-based antifungal agent is any one selected from the group consisting of hob1, mbs1, jjj1, ert1, ecm22, gat201, zap104, spl/crz1, fzc6, bzp5, hlh1, pip2, hcm1, bzp2, usv101, hob4, ste12, hob5, grf1, hel2, fzc45, asg1, fzc22, hob6, pan1, liv4, cuf1, fzc49, fzc1, bwc2, fap1, fzc44, fzc8, fzc23, gat204, nrg1, pip201, hlh2, rim101, fzc38, hlh3, bzp3, mln1, met32, zfc2, fzc40, fzc31 and rum1; a gene that increases sensitivity to the 5-flucytocin-based antifungal agent is any one gene selected from the group consisting of nrg1, zfc2, bap1, mbs1, fzc6, bap2, bzp3, jjj1, hlh1, pip2, apn2, fzc46, hap2, fzc51, bzp5, hcm1 and fzc19; and a gene that increases sensitivity to the phenylpyrazole-based antifungal agent is any one gene selected from the group consisting of usv101, ada2, bap1, fzc6, hlh1, pip2, fzc46, hap2, bzp1/hxl1, fkh2, liv1, bap2, bzp2, fzc21, hlh3, yrm101, bzp5, gln3, zfc8, ddt1, fzc22, hob6, rlm1, mln1, liv4, pan1, fzc35, yrm103, fzc3, asg1, fzc41, fzc43, fzc51, hap1, fzc38, met32 and fzc32.

In the embodiment of method for screening an antifungal agent, as the antifungal agent-targeting gene, a gene, which increases sensitivity to the azole-based antifungal agent when its expression is up-regulated, is any one gene selected from the group consisting of hob1, hap2, skn7, nrg1, mbs1, ppr1, jjj1, hcm1, ada2, fzc9, gat7, ert1, fkh2, ecm22, ddt1, gat1, yrm103, cuf1 and fzc51; a gene that increases sensitivity to the polyene-based antifungal agent is any one selected from the group consisting of sre1, bap1, fzc51, skn7, clr1, bzp5, atf1 and fzc4; a gene that increases sensitivity to the 5-flucytocin-based antifungal agent is any one gene selected from the group consisting of hlh3, rim101, gat204, hob3, fzc50, znf2 and rds2; and a gene that increases sensitivity to the phenylpyrazole-based antifungal agent is any one gene selected from the group consisting of nrg1, jjj1, sp1/crz1, skn7, gat7, fap1, zfc2, gat204, znf2, hel2, fzc50 and sre1.

In the embodiment of the method for screening an antifungal agent, as the antifungal agent-targeting gene whose expression is down-regulated, a growth regulatory gene is temperature-independent or temperature-dependent. The temperature-independent growth regulatory gene is any one gene selected from the group consisting of bzp2, cuf1, hob1, gat5, fzc6 and nrg1; and the temperature-dependent growth regulatory gene that regulates growth at a temperature of 37° C. to 39° C. is any one gene selected from the group consisting of hxl1, crz1, atf1, ada2, liv4, aro80, usv101, fzc31, fzc30, mln1, fzc30, fzc1, miz1, apn2, gat6, mbs2, sre1 and ert1. Furthermore, as the antifungal agent-targeting gene whose expression is up-regulated, an antifungal agent-targeting gene that regulates growth at 39° C. is any one gene of mini and fzc46.

In the embodiment of the method for screening an antifungal agent, as the antifungal agent-targeting gene whose expression is down-regulated, a mating regulatory gene is any one gene selected from the group consisting of bzp2, usv101, fzc1, zap104 and skn7; and as the antifungal agent-targeting gene whose expression is up-regulated, a mating regulatory gene is any one gene selected from the group consisting of hlh1, hap2, skn7 and gat1.

In the embodiment of the method for screening an antifungal agent, the antifungal agent-targeting gene, which regulates responses to external stress when its expression is down-regulated, may be an antifungal agent-targeting gene that regulates responses to an osmotic stress induced by any one selected from the group consisting of 1M to 1.5M sodium chloride (NaCl), 1M to 1.5M potassium chloride (KCl) and 2M sorbitol. Specifically, the antifungal agent-targeting gene that regulates responses to the osmotic stress induced by the sodium chloride is any one gene selected from the group consisting of rim101, skn7, ada2, fzc42, hcm1, gat7, bzp2, hob1, hap2, hob6, aro8001, pan1, fzc34, bap1, fzc19, fzc51, fzc43, fzc13, gat5 and met32; the antifungal agent-targeting gene that regulates responses to the osmotic stress induced by the potassium chloride is any one gene selected from the group consisting of bzp2, hob2, nrg1, hap2, ada2, fzc6, yrm103, fzc44, fzc32, hob1, rim101, bzp4 and fzc35; and the antifungal agent-targeting gene that regulates responses to the osmotic stress induced by the sorbitol is any one gene selected from the group consisting of bzp2, hob1 and fzc6. Moreover, among antifungal agent-targeting genes that regulates responses to an oxidative stress induced by any one selected from the group consisting of 2.5 mM to 3.5 mM hydrogen peroxide (H₂O₂), 0.7 mM to 0.8 mM tert-butyl hydroperoxide (TH), 0.02 mM to 0.03 mM menadione, and 2 mM to 3 mM diamide (DA), the antifungal agent-targeting gene that regulates responses to the oxidative stress induced by the hydrogen peroxide is any one gene selected from the group consisting of bap1, sre1, usv101, fzc50, fzc31, hob1, ada2, cuf1, gat204, ste12, fzc9, gat1, fzc21, nrg1, bzp2, gat5, pan1, met32, fzc4, rim101, hob6, fzc13, hlh1, fzc46, sip402, fzc27, hob5, hob4, sp1(crz1), fzc22, bzp3, liv1, miz1 and gat201; the antifungal agent-targeting gene that regulates responses to the oxidative stress induced by the tert-butyl hydroperoxide is any one gene selected from the group consisting of sre1, ada2, rim101, bap2, bap1, usv101, fzc31, hob1, fzc34, ecm22, fzc15, fzc44, zfc4, fzc49, yrm103, fzc21, zfc2, gat5, pan1, met32, hob4, liv1, mwc2, skn7, hcm1, fzc51, fzc1, ppr1, atf1, grf1, bzp5, gat8, clr1, hlh2, rlm1, fzc6, asg1, hob2, and zap103; the antifungal agent-targeting gene that regulates responses to the oxidative stress induced by the menadione is any one gene selected from the group consisting of bap1, fzc37, usv101, nrg1, bzp2, fzc4, fzc34, fzc35, hel2, ecm22, fzc6, gat6, jjj1, fzc44, fzc3 and fzc26; and the antifungal agent-targeting gene that regulates responses to the oxidative stress induced by the diamide is any one gene selected from the group consisting of bap1, hob1, bap2, bap2, pip2, bzp5, hsf2, sre1, fzc21, zfc2, fzc31, bzp2, gat5, pan1, met32, fzc4, fzc34, hob6, hlh1, fzc46, sip402, fzc27, miz1, fzc19, hlh3, fkh2, mln1, gat6, fap1, fzc8, fzc49, fzc3, fzc30, rum1 and fzc38. In addition, among antifungal agent-targeting genes that regulates responses to endoplasmic reticulum (ER) stress induced by 0.3 μg/ml tunicamycin (TM) or 20 mM dithiothreitol (DTT), the antifungal agent-targeting gene that regulates responses to the endoplasmic reticulum stress induced by the tunicamycin is any one gene selected from the group consisting of bzp1 (hxl1), sre1, hlh1, bzp3, pip2, rlm1, met32, ste12, rim101, sp1 (crz1), fzc21, gat7, mln1, fzc2, fzc44, liv4, fzc40 and fzc38; and the antifungal agent-targeting gene that regulates responses to the endoplasmic reticulum stress induced by the dithiothreitol is any one gene selected from the group consisting of bzp1 (hxl1), sre1, bzp2, cuf1, hob1 clr1, ada2, rlm1, gat5, hap2, nrg1, usv101, fzc31, gat201, hlh2, apn2, fzc25 and ddt1. In addition, among antifungal agent-targeting genes that regulates responses to a genotoxic stress induced by 0.03% to 0.06% methyl methanesulfonate (MM) or 50 mM to 100 mM hydroxyurea (HU), the antifungal agent-targeting gene that regulates responses to the genotoxic stress induced by the methyl methanesulfonate is any one gene selected from the group consisting of bzp1 (hxl1), fzc6, hob1, sre1, gat5, gat6, miz1, bzp2, jjj1, fzc40, fzc38, fzc4, hcm101, fzc1 and apn2; and the antifungal agent-targeting gene that regulates responses to the genotoxic stress induced by the hydroxyurea is any one gene selected from the group consisting of hob1, sre1, gat5, gat6, mbs1, skn7, ada2, bzp1 (hxl1), fzc6, bzp2, jjj1, hcm1, nrg1 and hlh2. In addition, among antifungal agent-targeting genes that regulates responses to a cell wall or cell membrane stress induced by any one selected from the group consisting of 3 mg/ml to 5 mg/ml calcofluor white (CFW), 0.8% to 1% Congo red (CR), and 0.03% sodium dodecyl sulfate (SDS), the antifungal agent-targeting gene that regulates responses to the cell wall or cell membrane stress induced by the CFW is any one gene selected from the group consisting of bzp1 (hxl1), sp1 (crz1), hob1, hap2, bzp2, nrg1, bap2, rim101 and pip2; the antifungal agent-targeting gene that regulates responses to the cell wall or cell membrane stress induced by the CR is any one gene selected from the group consisting of sp1 (crz1), hob1, bzp1 (hxl1), cuf1, hlh3, hap2, bzp2, nrg1, bap2 and rim101; and the antifungal agent-targeting gene that regulates responses to the cell wall or cell membrane stress induced by the SDS is any one gene selected from the group consisting of sp1 (crz1), hob1, sre1, pip2, fzc21, gat7, hob3, usv101, gat201, fzc7, asg1, rum1, hap2, bzp2, nrg1, cuf1, gat5, gat6, jjj1, pan1, bzp3, rlm1, bap1, clr1, zfc4, clr4, gat1, fzc31, hob5, asg101, ert1, ecm22, zfc6, bzp5, sxi1 alpha, fap1, sip4, rds2, fzc26 and fzc30. In addition, an antifungal agent-targeting gene that regulates responses to a heavy-metal stress induced by 20 M to 30 M cadmium sulfate (CdSO₄) is any one gene selected from the group consisting of cuf1, hap2, fzc6, skn7, fzc37, bzp2, gat5, yox101, mln1, pip2, hcm1, hob6, fzc46, hob5, mbs2, fzc35, aro8001, fzc19, fzc51, aro80, ccd4, fzc47, bzp4, fap1, fzc8, pip201, gln3, yrm101, zfc8, hob7, rum1 and fzc10.

In the embodiment of the method for screening an antifungal agent, the antifungal agent-targeting gene, which regulates responses to external stress when their expression is up-regulated, may be an antifungal agent-targeting genes that regulates an osmotic stress induced by any one selected from the group consisting of 1 M to 1.5 M sodium chloride (NaCl), 1 M to 1.5 M potassium chloride (KCl) and 2 M sorbitol. Specifically, the antifungal agent-targeting gene that regulates responses to the osmotic stress induced by the sodium chloride is any one gene selected from the group consisting of hlh3, hel2 and cuf1; the antifungal agent-targeting gene that regulates responses to the osmotic stress induced by the potassium chloride is any one gene of fzc36 and yrm103; and the antifungal agent-targeting gene that regulates responses to the osmotic stress induced by the sorbitol is fzc36. In addition, among antifungal agent-targeting genes that regulate responses to an oxidative stress induced by any one selected from the group consisting of 2.5 mM to 3.5 mM hydrogen peroxide (H₂O₂), 0.7 mM to 0.8 mM tert-butyl hydroperoxide (TH), 0.02 mM to 0.03 Mm menadione, and 2 mM to 3 mM diamide (DA), the antifungal agent-targeting gene that regulates responses to the oxidative stress induced by the hydrogen peroxide is any one gene selected from the group consisting of fzc45, asg101, mbs2, fzc35, bwc2, fzc7 and znf2; the antifungal agent-targeting gene that regulates responses to the oxidative stress induced by the tert-butyl hydroperoxide is any one gene selected from the group consisting of fzc33, fap1, clr3 and ddt1; the antifungal agent-targeting gene that regulates responses to the oxidative stress induced by the menadione is any one gene selected from the group consisting of zfc2, fzc50, cuf1, hap2 and sip4; and the antifungal agent-targeting gene that regulates responses to the oxidative stress induced by the diamide is any one gene selected from the group consisting of fzc50, sip4, pip201, nrg1, gat1, znf2, asg101, skn7, gat7, jjj1, hlh5, fzc26 and fzc20. In addition, among antifungal agent-targeting genes that regulate responses to an endoplasmic reticulum stress induced by 0.3 μg/ml tunicamycin (TM) or 20 mM dithiothreitol (DTT), the antifungal agent-targeting gene that regulates responses to the endoplasmic reticulum stress induced by the tunicamycin is any one gene selected from the group consisting of bzp2, nrg1, hap2, cuf1, mbs1, ppr1, fzc6, skn7, zfc2, hob1, gat5, clr1, bap1, bwc2, hcm1, hel2, gat6, jjj1, hob3, zfc4, zfc3 and clr4; and the antifungal agent-targeting gene that regulates responses to the endoplasmic reticulum stress induced by the dithiothreitol is any one gene selected from the group consisting of yap4, hlh1, bzp3, pip2, pan1, mbs1, met32, gat1, fkh2, fzc11, gat203, sip401, stb4 and fzc20. In addition, among antifungal agent-targeting genes that regulate responses to a genotoxic stress induced by 0.03% to 0.06% methyl methanesulfonate (MM) or 50 mM to 100 mM hydroxyurea (HU), the antifungal agent-targeting gene that regulates responses to the genotoxic stress induced by the methyl methanesulfonate is yox1; and the antifungal agent-targeting gene that regulates responses to the genotoxic stress induced by the hydroxyurea is fzc20. In addition, among antifungal agent-targeting genes that regulate responses to a cell wall or cell membrane stress induced by any one selected from the group consisting of 3 mg/ml to 5 mg/ml calcofluor white (CFW), 0.8% to 1% Congo red (CR), and 0.03% sodium dodecyl sulfate (SDS), the antifungal agent-targeting gene that regulates responses to the cell wall or cell membrane stress induced by the CFW is any one gene of fzc9 and grf1; and the antifungal agent-targeting gene that regulates responses to the cell wall or cell membrane stress induced by the SDS is any one gene selected from the group consisting of fzc6, fzc1, zfc1, hsf3, bwc2, skn7, fzc50, fzc22, fzc51 and fzc8. In addition, an antifungal agent-targeting gene that regulates responses to a heavy-metal stress induced by 20 μM to 30 λM cadmium sulfate (CdSO₄) is any one gene selected from the group consisting of bzp1 (hxl1), gat201, znf2, sip4, rds2, sre1, gat7, rlm1, clr1, zfc3, ada2, gat204, fzc7, asg101, atf1, hlh2, fzc39 and hsf3.

In another embodiment, the present invention provides a method for screening an antifungal agent, an antifungal agent for co-administration or an agent for treating meningitis, the method comprising the steps of: (a) bringing a sample to be analyzed into contact with a cell comprising a virulence regulatory gene; (b) measuring the expression of the virulence regulatory gene in the cell; and (c) determining that the sample is an antifungal agent, when the expression of the virulence regulatory gene is measured to be down-regulated or up-regulated.

In the embodiment of the method for screening an antifungal agent, an antifungal agent for co-administration or an agent for treating meningitis, the virulence regulatory gene may be a gene that regulates Cryptococcus neoformans pathogenicity. Specifically, the gene whose expression is down-regulated is any one gene selected from the group consisting of usv101, fzc1, bap1, hob1, zfc2, fzc50, fzc31, bzp2, fzc9, ddt1, mal13, fzc2, fzc43, fzc22, hih1, mbs2, rum1, fzc5, aro80, clr1, pip2, fzc37, gat5, fzc49, cef3, fzc33, fzcl2 and zfc5; and the gene whose expression is up-regulated is any one selected from the group consisting of fzcl7, fzc40, aro8001, fzc38, fzc24 and ert1.

In the embodiment of the method for screening an antifungal agent, an antifungal agent for co-administration or an agent for treating meningitis, the virulence regulatory gene regulates the production of any one selected from the group consisting of capsule, melanin and urease.

In the embodiment of the method for screening an antifungal agent, an antifungal agent for co-administration or an agent for treating meningitis, the gene that reduces capsule production is any one gene selected from the group consisting of bap1, rds2, zap104, fzc47, gat204, fzc33, fzc45, hsf2, bzp4, hob5, fzc16, hob3, zfc4, mcm1, liv4, hob4 and liv1; and the gene that increases capsule production is any one gene selected from the group consisting of hob7, clr3, fzc51, fzc1, fkh2, nrg1, usv101, fzc29, bzp3, zfc3, fzc14, sre1, fzc30, hlh4, fzc36, crl6, mln1, fzc46, clr1, fzcl7, jjj1, fzc49, fzc18, hcm1, fzc24, hlh3 and hpa1.

In the embodiment of the method for screening an antifungal agent, an antifungal agent for co-administration or an agent for treating meningitis, the gene that reduces melanin production is any one gene selected from the group consisting of bzp4, fzc8, hob1, usv101, liv1, mbs2, fzc5, fzc25 and ert1; and the gene that increases melanin production is any one gene selected from the group consisting of bzp2, fkh2, bap1, bzp3, hlh1, sip4, rds2, sip401, fzc1, gat1, ada2, nrg1, fzc31 and hlh2.

In the embodiment of the method for screening an antifungal agent, an antifungal agent for co-administration or an agent for treating meningitis, the gene that reduces urease production is any one gene selected from the group consisting of zap104, sre1, gat201, fzc46, hlh1 and fzc21; and the gene that increases urease production is any one gene selected from the group consisting of rim1, atf1, fkh2, fzc1, usv101, bap1, sxi1 alpha, mln1, fzc26, skn7, zfc7, hob7, fzc14 and hob4.

In the embodiment of the method for screening an antifungal agent, an antifungal agent for co-administration or an agent for treating meningitis, the cell is Cryptococcus neoformans.

The term “sample” as used herein with reference to the screening method means an unknown candidate that is used in screening to examine whether it influences the expression of a gene or the amount or activity of a protein. Examples of the sample include, but are not limited to, chemical substances, nucleotides, antisense-RNA, siRNA (small interference RNA) and natural extracts.

The term “antifungal agent” as used herein is meant to include inorganic antifungal agents, organic natural extract-based antifungal agents, organic aliphatic compound-based antifungal agents, and organic aromatic compound-based antifungal agents, which serve to inhibit the propagation of bacteria and/or fungi. Examples of the inorganic antifungal agents include, but are not limited to, chlorine compounds (especially sodium hypochlorite), peroxides (especially hydrogen peroxide), boric acid compounds (especially boric acid and sodium borate), copper compounds (especially copper sulfate), zinc compounds (especially zinc sulfate and zinc chloride), sulfur-based compounds (especially sulfur, calcium sulfate, and hydrated sulfur), calcium compounds (especially calcium oxide), silver compounds (especially thiosulfite silver complexes, and silver nitrate), iodine, sodium silicon fluoride, and the like. Examples of the organic natural extract-based antifungal agents include hinokithiol, Phyllostachys pubescens extracts, creosote oil, and the like.

The term “meningitis” as used herein is meant to include various inflammatory diseases occurring in the subarachnoid space between the arachnoid and the pia mater, for example, those caused by invasion of viruses or bacteria into the subarachnoid space, inflammation caused by a certain chemical substance, and those caused by the spread of cancer cells into the cerebrospinal fluid space.

### Advantageous Effects

The present invention may effectively screen a composition having an antifungal effect or a meningitis-treating effect by measuring the expression level of a transcription factor that regulates virulence in a Cryptococcus neoformans strain. In addition, the present invention may provide a pharmaceutical composition, which exhibits an antifungal effect or a meningitis-treating effect, by up-regulating or down-regulating the expression of a transcription factor that regulates Cryptococcus neoformans virulence.

### DESCRIPTION OF DRAWINGS

FIGS. 1A and 1B show transcription factors required for the temperature-dependent growth of Cryptococcus neoformans.

FIGS. 2A to 2D show transcription factors involved in sexual differentiation of Cryptococcus neoformans. Specifically, FIG. 2A shows the results of a mating assay in which the WT strain H99 and each TF mutant were cocultured with the opposite mating type KN99a strain on V8 media and incubated at room temperature in the dark for 7 days; FIG. 2B shows the cell-fusion efficiency of each TF mutant calculated relative to that of control strains (NAT-marked wild-type strain (YSB119)×NEO-marked wild-type a strain (YSB121)); FIG. 2C shows transcription factors involved in pheromone gene expression. In FIG. 2C, indicated transcription factor mutants were cocultured with the KN99a strain on V8 medium at room temperature for 18 to 24 hours, and then RNA expression was analyzed. FIG. 2D is a schematic diagram showing the role of transcription factors in various mating stages of Cryptococcus neoformans.

FIGS. 3A and 3B show transcription factors involved in mating efficiency (sexual differentiation) in Cryptococcus neoformans (3A—positive regulators; 3B—negative regulators).

FIGS. 4A to 4F show transcription factors involved in virulence-factor production in Cryptococcus neoformans. Specifically, FIG. 4A shows the sizes of capsules produced in the wild-type strain H99 and transcription factor mutants; FIGS. 4B and 4C show transcription factors involved in capsule production in Cryptococcus neoformans (4B—negative regulators; 4C—positive regulators); and FIGS. 4D and 4E show the correlation between the expression of LAC1, which is the major laccase involved in melanin synthesis, and transcription factors. Specifically, FIG. 4D shows the results obtained by spotting transcription factor mutants on Niger seed agar medium (containing 0.1 and 0.3% glucose) and photographing the plates while culturing the mutants at 37° C.; and FIG. 4E shows the results of Northern blot analyses performed using a LAC1-specific probe for total RNA isolated from cells under glucose-rich (0 hr) and glucose-depleted conditions (1 and 2 hr).

FIGS. 5A to 5C show transcription factors involved in melanin production in Cryptococcus neoformans (5A—positive regulators; 5B and 5C—negative regulators).

FIGS. 6A and 6B shows transcription factors required for urease production in Cryptococcus neoformans (6A—negative regulators; 6B—positive regulators).

FIGS. 7A to 7G show transcription factors involved in Cryptococcus neoformans virulence in Galleria mellonella killing assay. Specifically, FIGS. 7A to 7F show the results obtained for various transcription factor deletions, and FIG. 7G shows the results of identifying virulence-related transcription factors in Cryptococcus neoformans by signature-tagged mutagenesis (STM)-based murine infectivity assay.

FIGS. 8A to 8D show that transcription factors regulating sterol biosynthesis genes govern general environmental stress responses and adaptation in Cryptococcus neoformans. FIG. 8A shows the results of observing the susceptibility of eight transcription factor mutants to antifungal drugs; FIG. 8B shows the results of Northern blot analysis performed using an ERG11-specific probe for the susceptibility of various transcription factor mutants to fluconazole (FCZ); FIG. 8C shows the results of Northern blot analysis performed using an ERG gene-specific probe for the susceptibility of a sre1 mutant and hob1 mutant to fluconazole (FCZ); FIG. 8D shows the results of observing the responses of a sre1 mutant and hob1 mutant to stress-inducing agents; and FIG. 8E shows a proposed model for the role of Sre1 and Hob1 in the sterol homeostasis and general stress responses of Cryptococcus neoformans.

FIGS. 9A and 9B show transcription factors involved in the virulence of Cryptococcus neoformans.

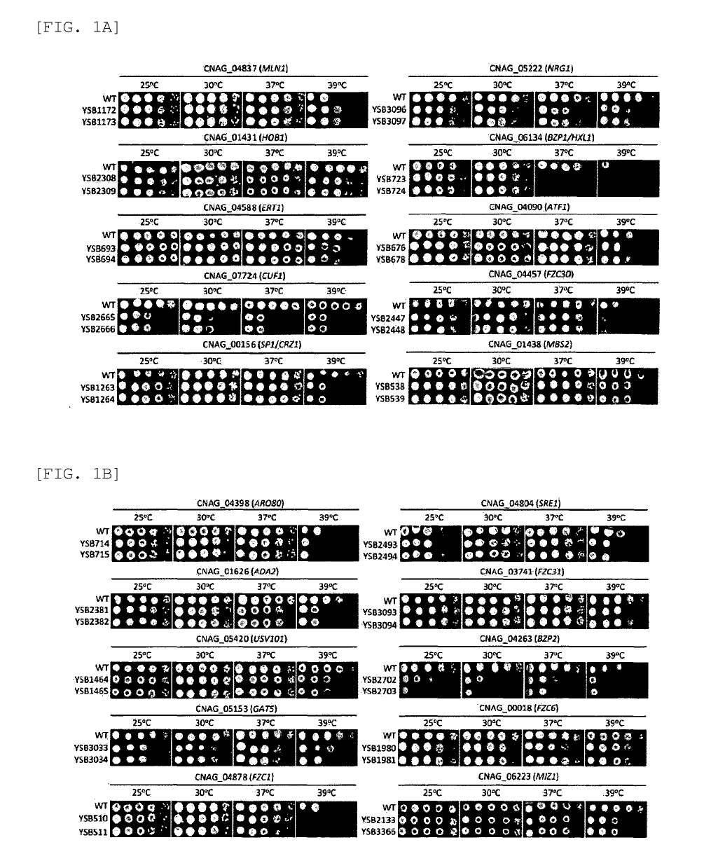

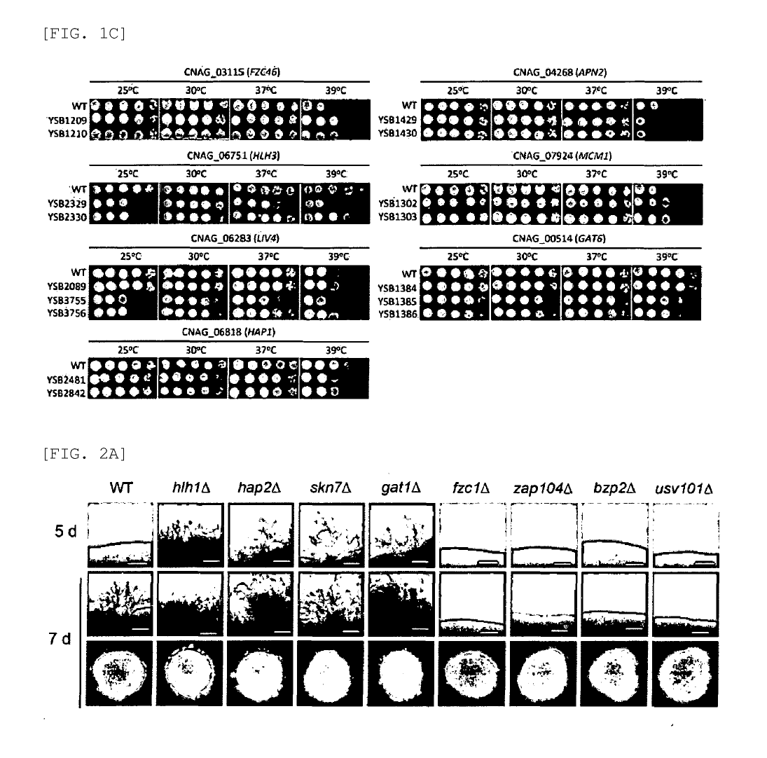

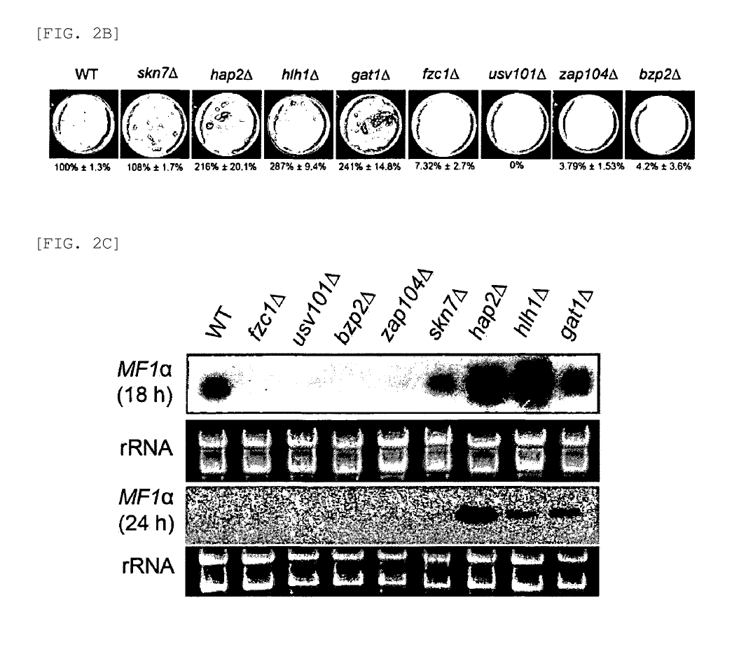

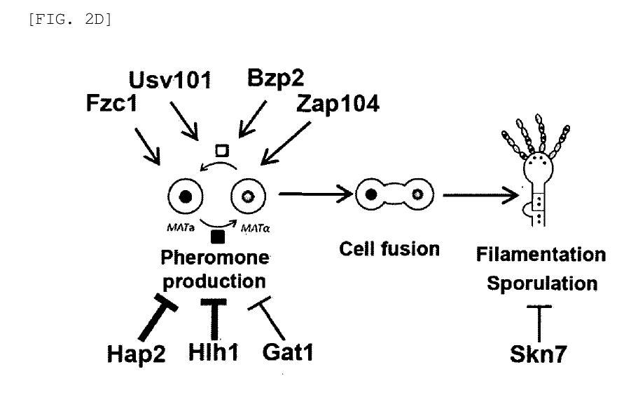

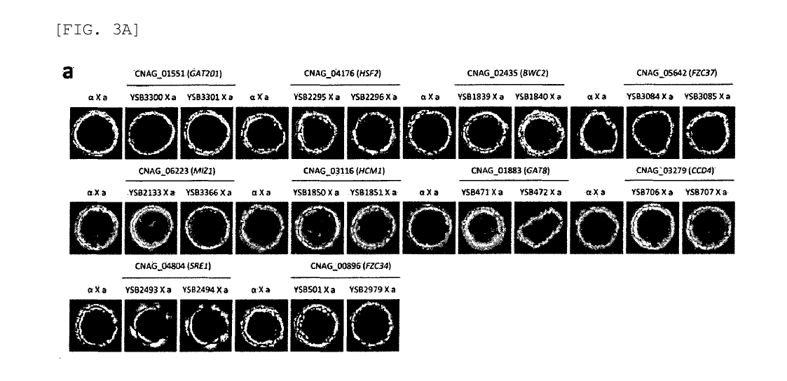

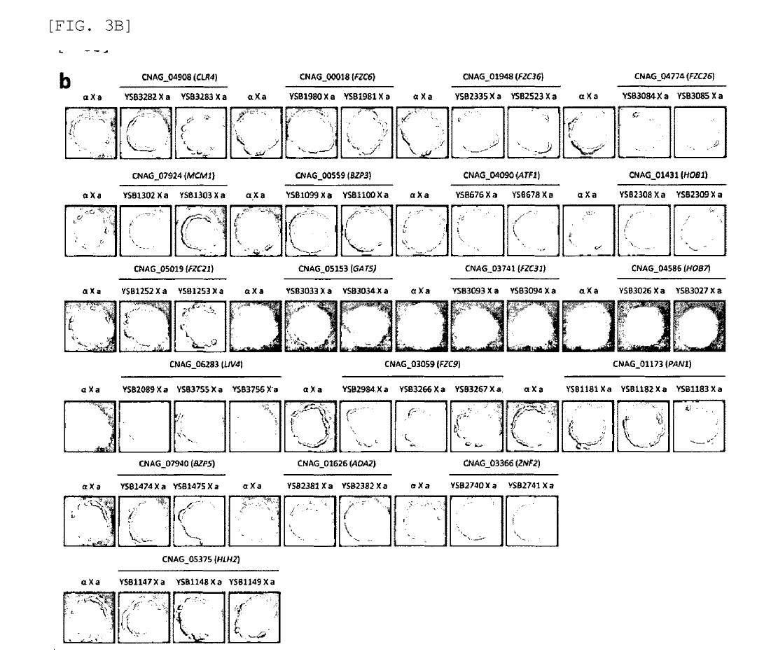

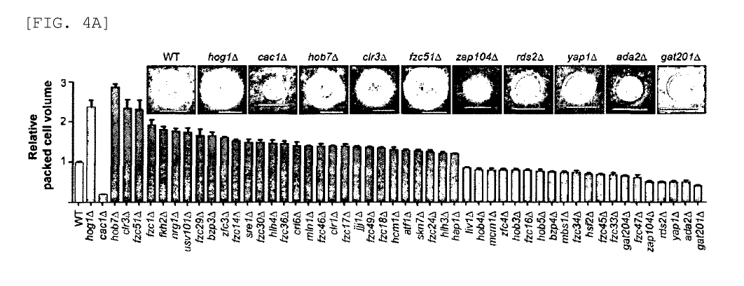

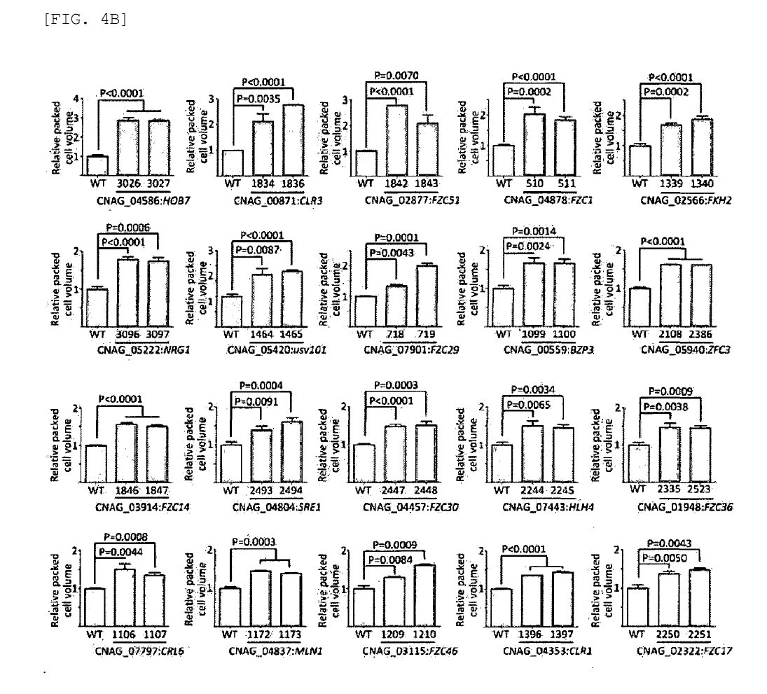

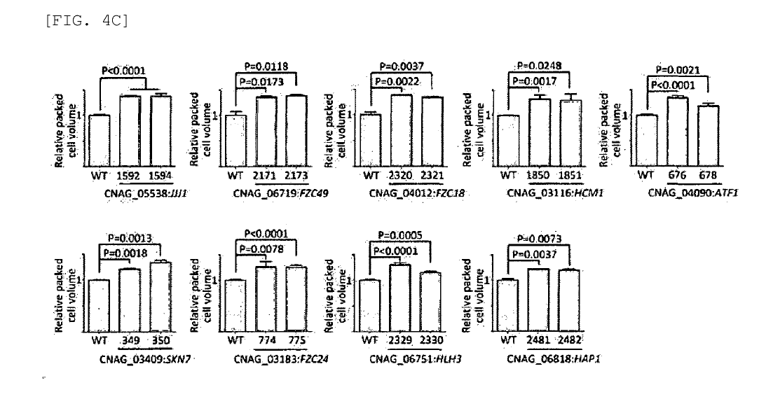

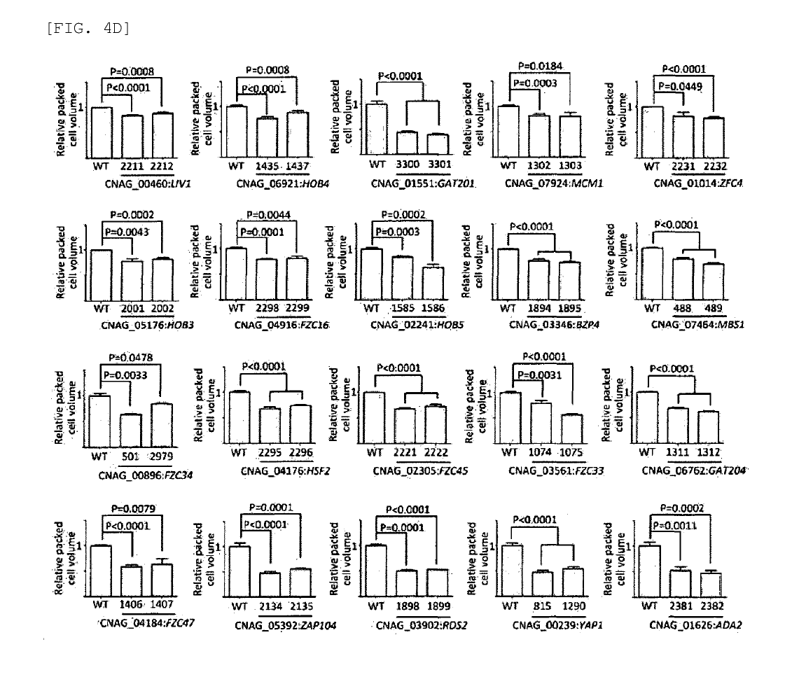

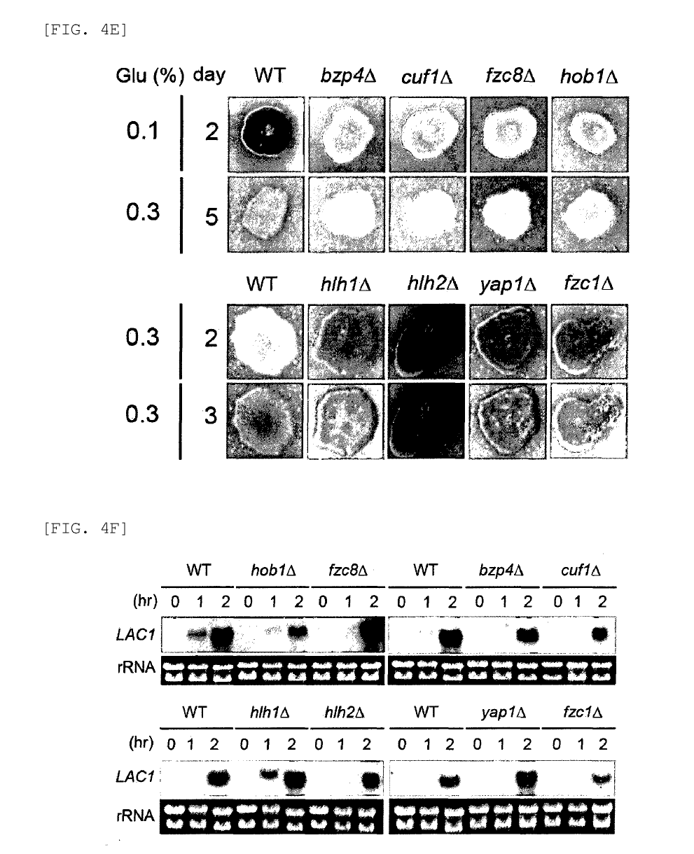

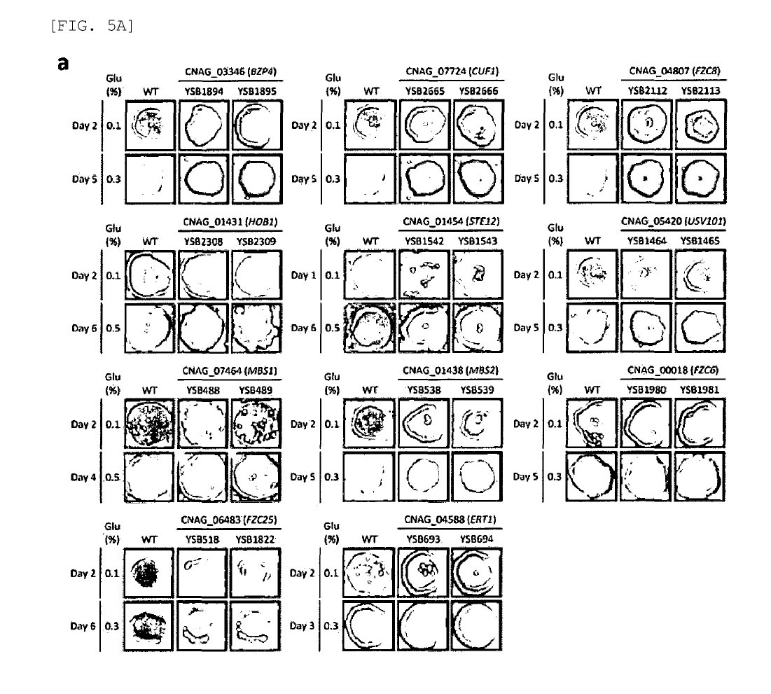

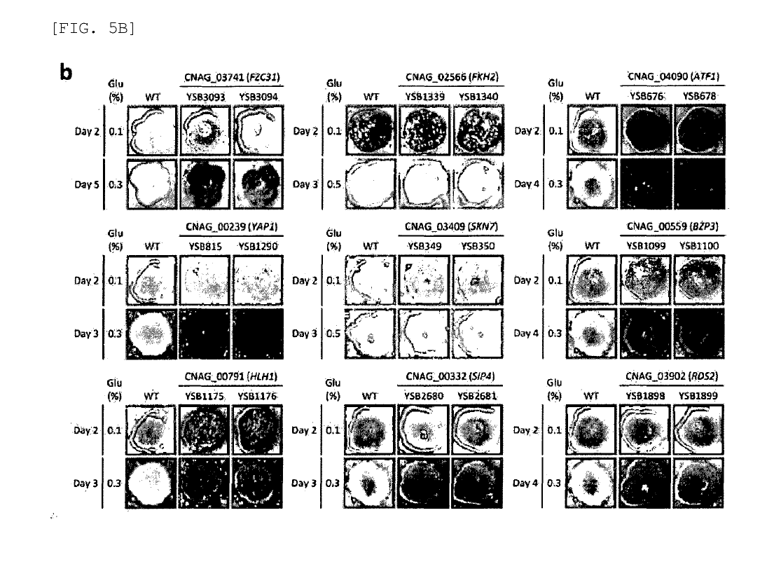

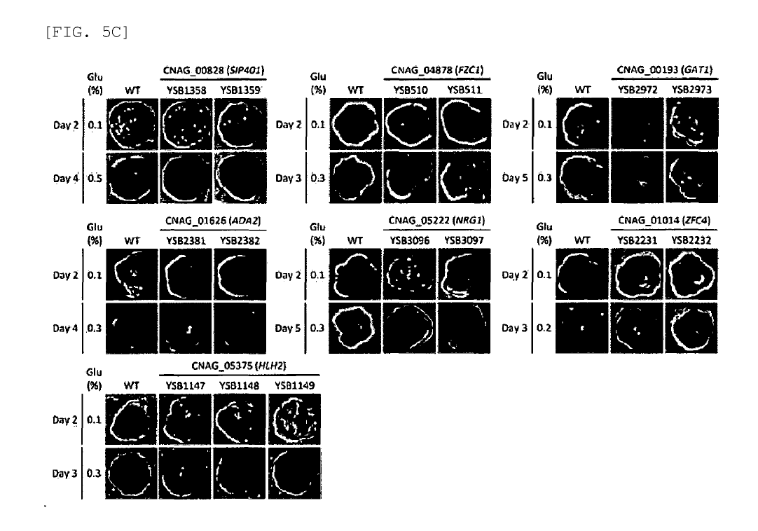

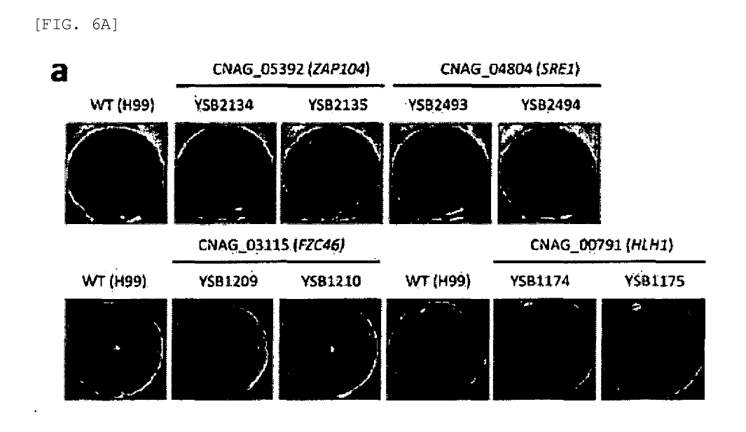

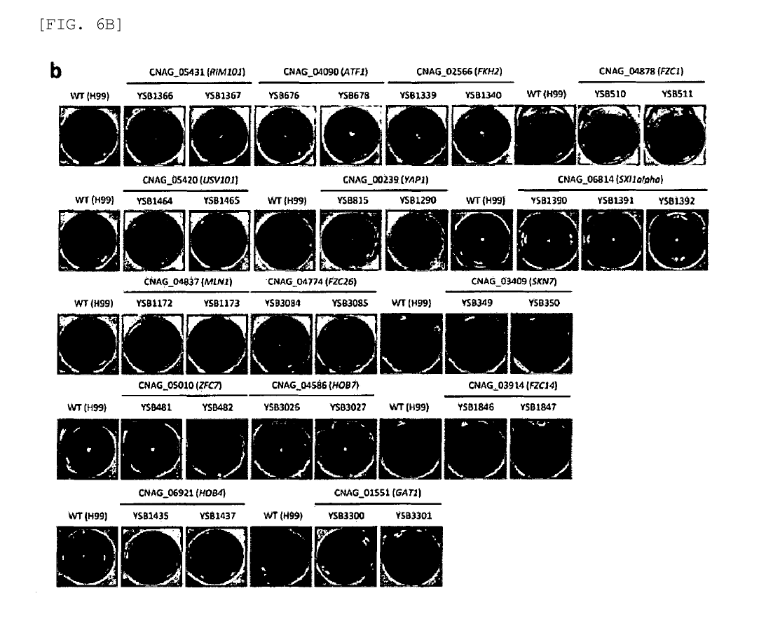

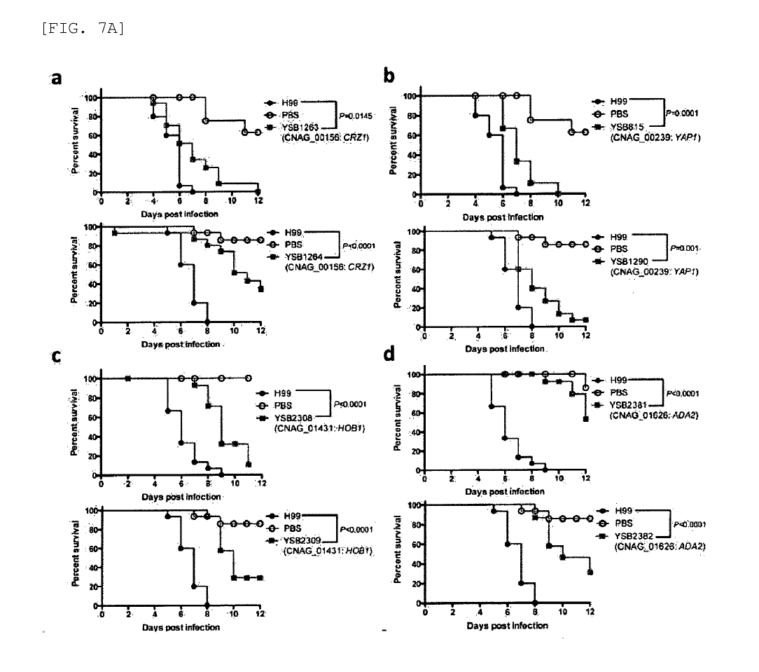

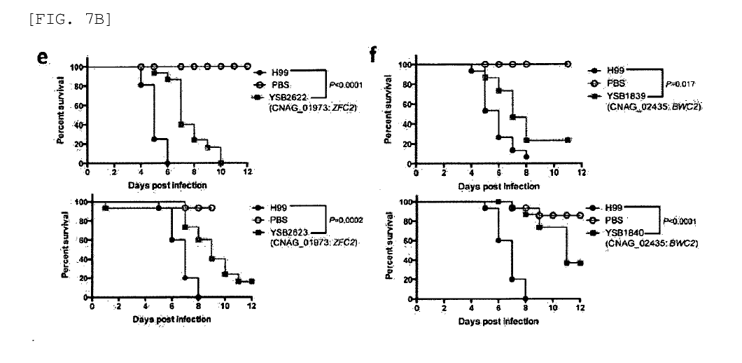

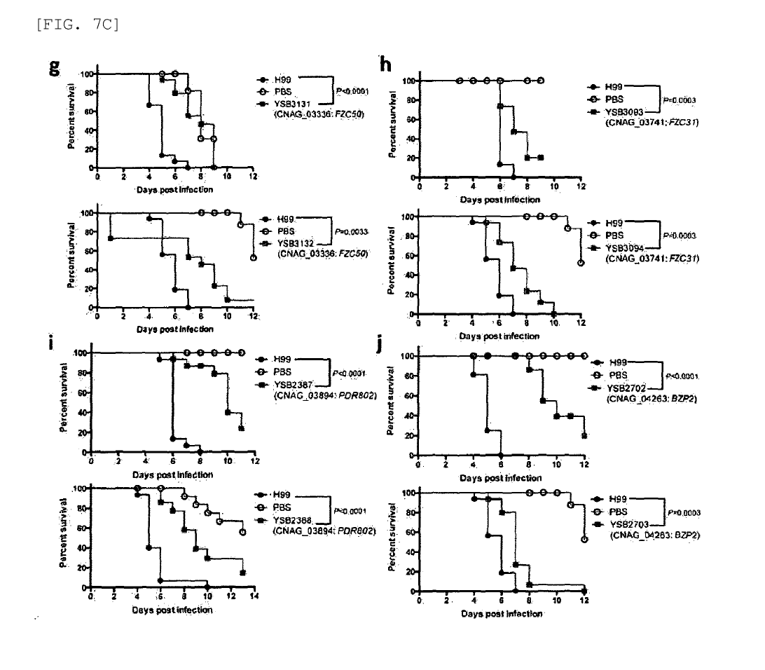

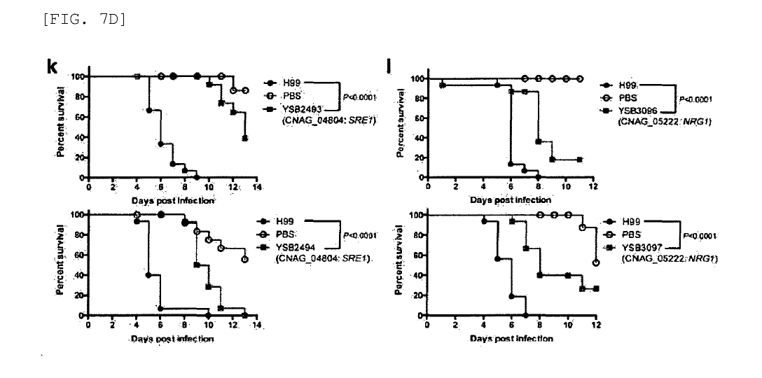

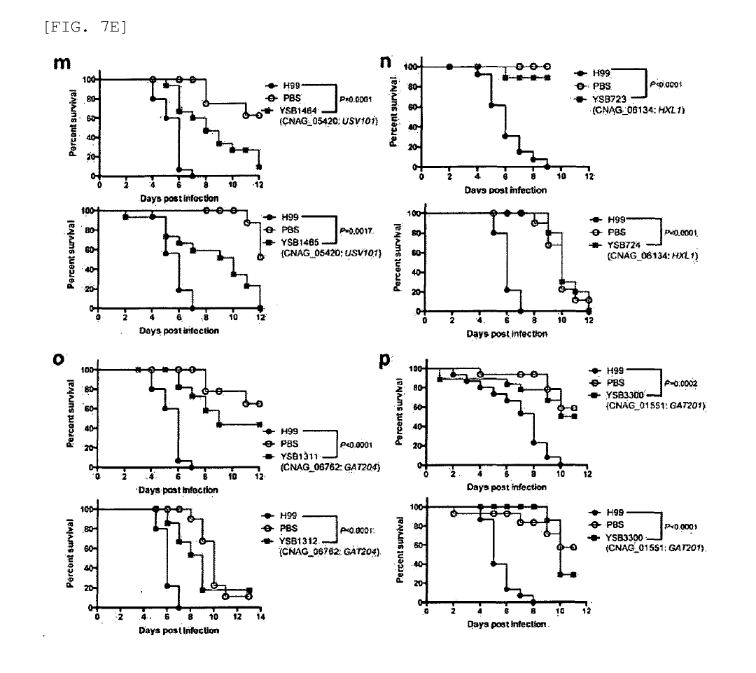

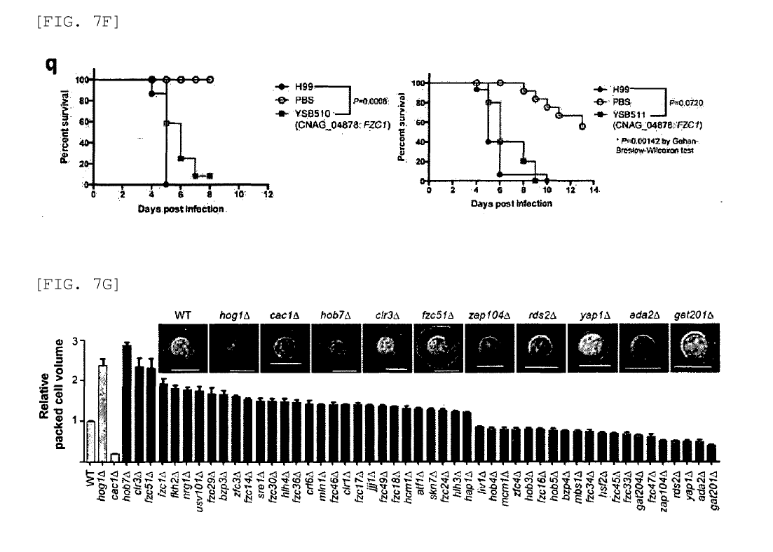

### MODE FOR INVENTION

Hereinafter, the present invention will be described in further detail with reference to examples. It will be obvious to those skilled in the art that these examples are for illustrative purposes and are not intended to limit the scope of the present invention as defined in the appended claims.

### Example 1: Construction of Transcription Factor Mutants

1.1: Selection of Transcription Factors and Transcription Factor Mutants

Putative transcription factors were screened using the published DBD transcription factor (TF) prediction database (http://www.transcriptionfactor.org/) (Non-Patent Document 8). The Cryptococcus neoformans H99 strain (a serotype A genome-sequence platform strain) contains 188 transcription factors (148 predicted from Pfam and 96 from SUPERFAMILY). Because these transcription factors were predicted based on the first version of the annotated H99 genome database, the present inventors updated this database with reference to the most recent version (version 7) of the annotated H99 genome database (Non-Patent Document 6), which resulted in a final prediction of 155 transcription factors (Table 1 below). The result of Orthologue mapping based on the BLAST e-value matrix demonstrated that Cryptococcus neoformans contains several evolutionarily distinct groups of transcription factors. The Cryptococcus DNA binding domain (DBD) transcription factors were classified based on their DNA binding domains (DBDs). 44% of these transcription factors (78) contain a fungal Zn2-Cys6 DBD, and among these, 40 also harbor a fungal-specific transcription factor domain. Several transcription factors contain more than two transcription factor domains (Table 1).

TABLE 1
List of Cryptococcus neoformans transcription factors predicted
based on DNA-binding domain database

| | H99 | | |
|---|---|---|---|
| No. | ID | TF domains | Gene Name |
| 1 | 02566 | “Winged helix” DNA-binding domain/Fork head | FKH2 |
| | | domain | |
| 2 | 00791 | Helix-loop-helix DNA-binding domain | HLH1 |
| 3 | 01069 | Fungal Zn(2)-Cys(6) binuclear cluster domain/ | FZC11 |
| | | Fungal specific transcription factor domain | |
| 4 | 07464 | APSES domain/Ankyrin repeat #2 | MBS1 |
| 5 | 03401 | Glucocorticoid receptor-like (DNA-binding domain)/ | GAT203 |
| | | GATA zinc finger | |
| 6 | 04588 | Fungal Zn(2)-Cys(6) binuclear cluster domain | ERT1 |
| 7 | 00828 | Fungal Zn(2)-Cys(6) binuclear cluster domain/ | SIP401 |
| | | Fungal specific transcription factor domain | |
| 8 | 03561 | Fungal Zn(2)-Cys(6) binuclear cluster domain/ | FZC33 |
| | | Fungal specific transcription factor domain | |
| 9 | 06762 | Glucocorticoid receptor-like (DNA-binding domain)/ | GAT204 |
| | | GATA zinc finger | |
| 10 | 06276 | Fungal Zn(2)-Cys(6) binuclear cluster domain/ | CEP3 |
| | | Fungal specific transcription factor domain | |
| 11 | 05785 | Fungal Zn(2)-Cys(6) binuclear cluster domain | STB4 |
| 12 | 03132 | Fungal Zn(2)-Cys(6) binuclear cluster domain | FZC5 |
| 13 | 01438 | KilA-N domain/Ankyrin repeats (many copies) | MBS2 |
| 14 | 07593 | bZIP transcription factor/Domain of unknown | YAP4 |
| | | function (DUF3425) | |
| 15 | 04837 | Helix-loop-helix DNA-binding domain | MLN1 |
| 16 | 05093 | Homeobox domain | HOB6 |
| 17 | 05642 | Fungal Zn(2)-Cys(6) binuclear cluster domain/ | FZC37 |
| | | Fungal specific transcription factor domain | |
| 18 | 05431 | Zinc-finger double domain/C2H2-type zinc finger | RIM101 |
| 19 | 04398 | Fungal specific transcription factor domain/ | AR080 |
| | | Fungal Zn(2)-Cys(6) binuclear cluster domain | |
| 20 | 04878 | Fungal Zn(2)-Cys(6) binuclear cluster domain/ | FZC1 |
| | | Fungal specific transcription factor domain | |
| 21 | 03183 | Fungal Zn(2)-Cys(6) binuclear cluster domain/ | FZC24 |
| | | Fungal specific transcription factor domain | |
| 22 | 03710 | Fungal Zn(2)-Cys(6) binuclear cluster domain/ | ECM22 |
| | | Fungal specific transcription factor domain | |
| 23 | 03279 | Fungal Zn(2)-Cys(6) binuclear cluster domain/ | CCD4 |
| | | Fungal specific transcription factor domain | |
| 24 | 04637 | Helix-turn-helix/lambda repressor-like DNA-binding | MBF1 |
| | | domains | |
| 25 | 06425 | Fungal Zn(2)-Cys(6) binuclear cluster domain/ | PPR1 |
| | | Fungal specific transcription factor domain | |
| 26 | 04345 | Fungal Zn(2)-Cys(6) binuclear cluster domain | ARO8001 |
| 27 | 04184 | Fungal Zn(2)-Cys(6) binuclear cluster domain/ | FZC47 |
| | | Fungal specific transcription factor domain | |
| 28 | 02774 | Fungal Zn(2)-Cys(6) binuclear cluster domain/ | MAL13 |
| | | Fungal specific transcription factor domain | |
| 29 | 00670 | Fungal Zn(2)-Cys(6) binuclear cluster domain | FZC12 |
| 30 | 00068 | Zinc-finger double domain/C2H2-type zinc finger | MET32 |
| 31 | 05010 | C2H2-type zinc finger | ZFC7 |
| 32 | 04090 | bZIP transcription factor/Basic region leucine | ATF1 |
| | | zipper | |
| 33 | 06134 | bZIP transcription factor/Basic region leucine | BZP1 (HXL1) |
| | | zipper | |
| 34 | 04630 | bZIP transcription factor/Basic region leucine | BAP2 |
| | | zipper | |
| 35 | 07901 | Fungal Zn(2)-Cys(6) binuclear cluster domain/ | FZC29 |
| | | Fungal specific transcription factor domain | |
| 36 | 01173 | Beta-trefoil DNA-binding domain/p53-like | PAN1 |
| | | transcription factors/DNA-binding protein LAG-1 | |
| | | (CSL) | |
| 37 | 03115 | Fungal Zn(2)-Cys(6) binuclear cluster domain/ | FZC46 |
| | | Fungal specific transcription factor domain | |
| 38 | 07924 | SRF-type transcription factor (DNA-binding and | MCM1 |
| | | dimerisation | |
| 39 | 07435 | CCAAT-binding transcription factor (CBF-B/NF-YA) | HAP2 |
| | | subunit B | |
| 40 | 02555 | Fungal Zn(2)-Cys(6) binuclear cluster domain/ | SIP402 |
| | | Fungal specific transcription factor domain | |
| 41 | 04594 | Fungal Zn(2)-Cys(6) binuclear cluster domain/ | FZC27 |
| | | Fungal specific transcription factor domain | |
| 42 | 06188 | Fungal Zn(2)-Cys(6) binuclear cluster domain | FZC15 |
| 43 | 05170 | Fungal Zn(2)-Cys(6) binuclear cluster domain | PIP2 |
| 44 | 02241 | Homeodomain-like/Helix-turn-helix domain | HOB5 |
| 45 | 06921 | Homeobox domain | HOB4 |
| 46 | 05186 | GRF zinc finger | GRF1 |
| 47 | 00896 | Fungal Zn(2)-Cys(6) binuclear cluster domain/ | FZC34 |
| | | Fungal specific transcription factor domain | |
| 48 | 00039 | C2H2-type zinc finger | ZFC6 |
| 49 | 07940 | Basic region leucine zipper/bZIP transcription | BZP5 |
| | | factor | |
| 50 | 06814 | Homeobox KN domain | SX11alpha |
| 51 | 01454 | STE like transcription factor/Zinc-finger double | STE12 |
| | | domain/ | |
| | | C2H2-type zinc finger | |
| 52 | 03527 | C2H2-type zinc finger/C3HC4-type zinc finger | HEL2 |
| 53 | 05255 | Fungal Zn(2)-Cys(6) binuclear cluster domain/ | FZC2 |
| | | Fungal specific transcription factor domain | |
| 54 | 05112 | Fungal Zn(2)-Cys(6) binuclear cluster domain/ | FZC42 |
| | | Fungal specific transcription factor domain | |
| 55 | 01883 | Glucocorticoid receptor-like (DNA-binding domain)/ | GAT8 |
| | | GATA zinc finger | |
| 56 | 04353 | C2H2-type zinc finger | CLR1 |
| 57 | 05375 | Helix-loop-helix DNA-binding domain | HLH2 |
| 58 | 03998 | SRF-type transcription factor (DNA-binding and | RLM1 |
| | | dimerisation | |
| 59 | 00239 | bZIP transcription factor/Basic region leucine | BAP1 |
| | | zipper | |
| 60 | 06871 | Fungal Zn(2)-Cys(6) binuclear cluster domain/ | FZC41 |
| | | Fungal specific transcription factor domain | |
| 61 | 00156 | C2H2-type zinc finger | SP1 (CRZ1) |
| 62 | 04268 | GRF zinc finger | APN2 |
| 63 | 05420 | Zinc-finger double domain/C2H2-type zinc finger | USV101 |
| 64 | 00018 | Fungal Zn(2)-Cys(6) binuclear cluster domain | FZC6 |
| 65 | 03346 | bZIP transcription factor | BZP4 |
| 66 | 00514 | Glucocorticoid receptor-like (DNA-binding domain)/ | GAT6 |
| | | GATA zinc finger | |
| 67 | 05538 | SRR1 domain/C2H2-type zinc finger/DnaJ domain | JJJ1 |
| 68 | 03409 | “Winged helix” DNA-binding domain/CheY-like/ | SKN7 |
| | | HSF-type DNA-binding | |
| 69 | 06339 | Fungal Zn(2)-Cys(6) binuclear cluster domain/ | FZC35 |
| | | Fungal specific transcription factor domain | |
| 70 | 07011 | Fungal Zn(2)-Cys(6) binuclear cluster domain/ | FZC22 |
| | | Fungal specific transcription factor domain | |
| 71 | 07506 | NF-X1 type zinc finger #3/R3H domain | FAP1 |
| 72 | 04807 | Fungal Zn(2)-Cys(6) binuclear cluster domain | FZC8 |
| 73 | 02435 | PYP-like sensor domain (PAS domain)/ | BWC2 |
| | | Glucocorticoid receptor-like (DNA-binding domain)/ | (CWC2) |
| | | GATA zinc finger/AT hook motif | |
| 74 | 02364 | Fungal Zn(2)-Cys(6) binuclear cluster domain | FZC19 |
| 75 | 03116 | “Winged helix” DNA-binding domain/Fork head | HCM1 |
| | | domain | |
| 76 | 02877 | Zinc-finger double domain/Fungal Zn(2)-Cys(6) | FZC51 |
| | | binuclear cluster domain | |
| 77 | 00559 | bZIP transcription factor/Basic region leucine | BZP3 |
| | | zipper | |
| 78 | 03914 | Fungal Zn(2)-Cys(6) binuclear cluster domain/ | FZC14 |
| | | Fungal specific transcription factor domain | |
| 79 | 00871 | bZIP transcription factor/Basic region leucine | CLR3 |
| | | zipper | |
| 80 | 06483 | Fungal Zn(2)-Cys(6) binuclear cluster domain | FZC25 |
| 81 | 07797 | Putative FMN-binding domain | CRL6 |
| 82 | 05019 | Fungal specific transcription factor domain | FZC21 |
| 83 | 05380 | Fungal Zn(2)-Cys(6) binuclear cluster domain/ | FZC44 |
| | | Fungal specific transcription factor domain | |
| 84 | 04518 | Fungal specific transcription factor domain/ | ZFC5 |
| | | C2H2-type zinc finger | |
| 85 | 05176 | Homeobox domain | HOB3 |
| 86 | 05861 | Fork head domain | FKH101 |
| 87 | 00460 | Helix-loop-helix DNA-binding domain | LIV1 |
| 88 | 02305 | Fungal Zn(2)-Cys(6) binuclear cluster domain/ | FZC45 |
| | | Fungal specific transcription factor domain | |
| 89 | 03849 | Fungal specific transcription factor domain/ | ASG1 |
| | | Fungal Zn(2)-Cys(6) binuclear cluster domain | |
| 90 | 01014 | C2H2-type zinc finger | ZFC4 |
| 91 | 01858 | Homeobox domain | HOB2 |
| 92 | 06719 | Fungal Zn(2)-Cys(6) binuclear cluster domain/ | FZC49 |
| | | Fungal specific transcription factor domain | |
| 93 | 04093 | Fungal Zn(2)-Cys(6) binuclear cluster domain/ | YRM103 |
| | | Fungal specific transcription factor domain | |
| 94 | 04176 | “Winged helix” DNA-binding domain/HSF-type | HSF2 |
| | | DNA-binding | |
| 95 | 01431 | Homeobox domain | HOB1 |
| 96 | 07443 | Helix-loop-helix DNA-binding domain | HLH4 |
| 97 | 04916 | Fungal Zn(2)-Cys(6) binuclear cluster domain | FZC16 |
| 98 | 05392 | Zinc-finger double domain/C2H2-type zinc finger | ZAP104 |
| 99 | 02322 | Fungal Zn(2)-Cys(6) binuclear cluster domain | FZC17 |
| 100 | 04012 | Fungal Zn(2)-Cys(6) binuclear cluster domain | FZC18 |
| 101 | 00505 | Fungal Zn(2)-Cys(6) binuclear cluster domain | FZC28 |
| 102 | 06751 | Helix-loop-helix DNA-binding domain | HLH3 |
| 103 | 04841 | Fungal Zn(2)-Cys(6) binuclear cluster domain/ | FZC43 |
| | | Fungal specific transcription factor domain | |
| 104 | 03212 | “Winged helix” DNA-binding domain | HCM101 |
| 105 | 01626 | Zinc finger, ZZ type/Myb-like DNA-binding domain/ | ADA2 |
| | | SWIRM domain | |
| 106 | 3894 | Fungal Zn(2)-Cys(6) binuclear cluster domain | PDR802 |
| 107 | 2066 | Fungal Zn(2)-Cys(6) binuclear cluster domain | FZC13 |
| 108 | 6818 | Fungal Zn(2)-Cys(6) binuclear cluster domain/ | HAP1 |
| | | Fungal specific transcription factor domain | |
| 109 | 5940 | C2H2-type zinc finger | ZFC3 |
| 110 | 1948 | Fungal Zn(2)-Cys(6) binuclear cluster domain/ | FZC36 |
| | | Fungal specific transcription factor domain | |
| 111 | 4804 | Helix-loop-helix DNA-binding domain | SRE1 |
| 112 | 4352 | Zinc-finger double domain/C2H2-type zinc finger | ZAP103 |
| 113 | 3768 | Fungal Zn(2)-Cys(6) binuclear cluster domain/ | FZC32 |
| | | Fungal specific transcription factor domain | |
| 114 | 1977 | Fungal Zn(2)-Cys(6) binuclear cluster domain | FZC39 |
| 115 | 4895 | Fungal Zn(2)-Cys(6) binuclear cluster domain/ | FZC3 |
| | | Fungal specific transcription factor domain | |
| 116 | 4583 | WSTF, HB1, Itc1p, MBD9 motif 2/3/DDT domain | DDT1 |
| 117 | 1973 | Zinc-finger double domain/C2H2-type zinc finger/ | ZFC2 |
| | | Fungal specific transcription factor domain | |
| 118 | 2603 | Fungal specific transcription factor domain/ | ZFC1 |
| | | C2H2-type zinc finger | |
| 119 | 4036 | “Winged helix” DNA-binding domain/HSF-type | HSF3 |
| | | DNA-binding | |
| 120 | 6156 | Fungal Zn(2)-Cys(6) binuclear cluster domain/ | FZC7 |
| | | Fungal specific transcription factor domain | |
| 121 | 03431 | Fungal Zn(2)-Cys(6) binuclear cluster domain/ | FZC48 |
| | | Fungal specific transcription factor domain | |
| 122 | 07922 | Fungal Zn(2)-Cys(6) binuclear cluster domain/ | FZC4 |
| | | Fungal specific transcription factor domain | |
| 123 | 00031 | Fungal Zn(2)-Cys(6) binuclear cluster domain/ | MLR1 |
| | | Fungal specific transcription factor domain | |
| 124 | 03018 | Fungal Zn(2)-Cys(6) binuclear cluster domain/ | ASG101 |
| | | Fungal specific transcription factor domain | |
| 125 | 04263 | Glucocorticoid receptor-like (DNA-binding domain)/ | BZP2 |
| | | Basic region leucine zipper/bZIP transcription | |
| | | factor/GATA zinc finger | |
| 126 | 01708 | GATA zinc finger | GAT7 |
| 127 | 00332 | Fungal Zn(2)-Cys(6) binuclear cluster domain | SIP4 |
| 128 | 07724 | Copper fist DNA binding domain | CUF1 |
| 129 | 03902 | Fungal Zn(2)-Cys(6) binuclear cluster domain/PAS | RDS2 |
| | | fold | |
| 130 | 03366 | Zinc-finger double domain/C2H2-type zinc finger | ZNF2 |
| 131 | 05222 | C2H2-type zinc finger/Zinc-finger double domain | NRG1 |
| 132 | 05049 | Fungal Zn(2)-Cys(6) binuclear cluster domain/ | P1P201 |
| | | Fungal specific transcription factor domain | |
| 133 | 02516 | Helix-loop-helix DNA-binding domain | HLH5 |
| 134 | 04774 | Fungal Zn(2)-Cys(6) binuclear cluster domain/ | FZC26 |
| | | Fungal specific transcription factor domain | |
| 135 | 03059 | Fungal Zn(2)-Cys(6) binuclear cluster domain | FZC9 |
| 136 | 00193 | Glucocorticoid receptor-like (DNA-binding domain)/ | GAT1 |
| | | GATA zinc finger | |
| 137 | 03336 | Fungal Zn(2)-Cys(6) binuclear cluster domain/ | FZC50 |
| | | Fungal specific transcription factor domain | |
| 138 | 03086 | Fungal Zn(2)-Cys(6) binuclear cluster domain/ | FZC20 |
| | | Fungal specific transcription factor domain | |
| 139 | 03229 | Homeobox domain/Homeobox KN domain | YOX101 |
| 140 | 01841 | Glucocorticoid receptor-like (DNA-binding domain)/ | GLN3 |
| | | GATA zinc finger | |
| 141 | 02476 | Fungal Zn(2)-Cys(6) binuclear cluster domain/ | YRM101 |
| | | Fungal specific transcription factor domain | |
| 142 | 05153 | Glucocorticoid receptor-like (DNA-binding domain)/ | GAT5 |
| | | GATA zinc finger/AT hook motif | |
| 143 | 02700 | C2H2-type zinc finger | ZFC8 |
| 144 | 04586 | Homeobox domain | HOB7 |
| 145 | 2723 | Fungal Zn(2)-Cys(6) binuclear cluster domain/ | FZC23 |
| | | Fungal specific transcription factor domain | |
| 146 | 3741 | Fungal Zn(2)-Cys(6) binuclear cluster domain/AT | FZC31 |
| | | hook motif | |
| 147 | 4457 | Fungal Zn(2)-Cys(6) binuclear cluster domain/AT | FZC30 |
| | | hook motif | |
| 148 | 6283 | Homeodomain-like | LIV4 |
| 149 | 7411 | C5HC2 zinc finger/PHD-finger/ARID/BRIGHT DNA | RUM1 |
| | | binding | |
| 150 | 4836 | Fungal Zn(2)-Cys(6) binuclear cluster domain/ | FZC10 |
| | | Fungal specific transcription factor domain | |
| 151 | 841 | Fungal Zn(2)-Cys(6) binuclear cluster domain/ | FZC40 |
| | | Fungal specific transcription factor domain | |
| 152 | 6223 | MIZ/SP-RING zinc finger | MIZ1 |
| 153 | 830 | Fungal Zn(2)-Cys(6) binuclear cluster domain/ | FZC38 |
| | | Fungal specific transcription factor domain | |
| 154 | 1551 | Glucocorticoid receptor-like (DNA-binding domain)/ | GAT201 |
| | | GATA zinc finger | |
| 155 | 4908 | bZIP transcription factor/Basic region leucine | CLR4 |
| | | zipper | |

To analyze the functions of the transcription factors, the present inventors deleted 155 putative transcription factor genes out of 178 using homologous recombination. To perform a large-scale in vivo virulence test, dominant nourseothricin-resistance markers (NATs) containing a series of signature tags were employed.

The genotypes of all transcription factor mutant strains were confirmed by performing Southern blot analysis to verify both the gene deletion and the absence of any ectopic integration of each gene-disruption cassette in transcription factor mutant strains. To accurately validate the phenotype and exclude unlinked mutational effects, the present inventors generated more than two independent transcription factor mutants for all 155 transcription factors, including four known transcription factors (HXL1, ATF1, MBS1 and SKN7) (Non-Patent Document 9, Non-Patent Document 10 and Non-Patent Document 11), and thus obtained a total of 322 strains (see Table 2 below).

TABLE 2
Transcription factor mutants

| | H99 1D | Designated name | TF class | Strain informatin |
|---|---|---|---|---|
| 1 | 02566 | FKH2 | FKH | YSB1339 |
| 2 | | | | YSB1340 |
| 3 | 00791 | HLH1 | HLH | YSB1175 |
| 4 | | | | YSB1176 |
| 5 | 07464 | MBS1 | APS | YSB488 |
| 6 | | | | YSB489 |
| 7 | 03401 | GAT203 | GAT | YSB569 |
| 8 | | | | YSB570 |
| 9 | 04588 | ERT1 | FZC | YSB693 |
| 10 | | | | YSB694 |
| 11 | 00828 | SIP401 | FZC | YSB1358 |
| 12 | | | | YSB1359 |
| 13 | 03561 | FZC33 | FZC | YSB1074 |
| 14 | | | | YSB1075 |
| 15 | 06762 | GAT204 | GAT | YSB1311 |
| 16 | | | | YSB1312 |
| 17 | 06276 | CEP3 | FZC | YSB847 |
| 18 | | | | YSB848 |
| 19 | 05785 | STB4 | FZC | YSB1013 |
| 20 | | | | YSB1014 |
| 21 | 01438 | MBS2 | APS | YSB538 |
| 22 | | | | YSB539 |
| 23 | 07593 | YAP4 | BZP | YSB1587 |
| 24 | | | | YSB1661 |
| 25 | 04837 | MLN1 | HLH | YSB1172 |
| 26 | | | | YSB1173 |
| 27 | 05093 | HOB6 | HOM | YSB1255 |
| 28 | | | | YSB1256 |
| 29 | 05642 | FZC37 | FZC | YSB1329 |
| 30 | | | | YSB1330 |
| 31 | 05431 | RIM101 | C2Z | YSB1366 |
| 32 | | | | YSB1367 |
| 33 | 04398 | ARO80 | FZC | YSB714 |
| 34 | | | | YSB715 |
| 35 | 04878 | FZC1 | FZC | YSB510 |
| 36 | | | | YSB511 |
| 37 | 03183 | FZC24 | FZC | YSB774 |
| 38 | | | | YSB775 |
| 39 | 03710 | ECM22 | FZC | YSB476 |
| 40 | | | | YSB478 |
| 41 | 03279 | CCD4 | HOM | YSB706 |
| 42 | | | | YSB707 |
| 43 | 04637 | MBF1 | HTH | YSB768 |
| 44 | | | | YSB769 |
| 45 | 06425 | PPR1 | FZC | YSB1046 |
| 46 | | | | YSB1047 |
| 47 | 04345 | ARO8001 | FZC | YSB661 |
| 48 | | | | YSB662 |
| 49 | 04184 | FZC47 | FZC | YSB1406 |
| 50 | | | | YSB1407 |
| 51 | 02774 | MAL13 | FZC | YSB506 |
| 52 | | | | YSB507 |
| 53 | 00670 | FZC12 | FZC | YSB467 |
| 54 | | | | YSB468 |
| 55 | 05010 | ZFC7 | C2Z | YSB481 |
| 56 | | | | YSB482 |
| 57 | 04090 | ATF1 | BZP | YSB676 |
| 58 | | | | YSB678 |
| 59 | 06134 | BZP1(HXL1) | BZP | YSB723 |
| 60 | | | | YSB724 |
| 61 | 04630 | YAP2 | BZP | YSB1416 |
| 62 | | | | YSB1417 |
| 63 | 07901 | FZC29 | FZC | YSB718 |
| 64 | | | | YSB719 |
| 65 | 03115 | FZC46 | FZC | YSB1209 |
| 66 | | | | YSB1210 |
| 67 | 07924 | MCM1 | SRF | YSB1302 |
| 68 | | | | YSB1303 |
| 69 | 07435 | HAP2 | CCA | YSB1104 |
| 70 | | | | YSB1105 |
| 71 | 02555 | SIP402 | FZC | YSB529 |
| 72 | | | | YSB530 |
| 73 | 04594 | FZC27 | FZC | YSB582 |
| 74 | | | | YSB583 |
| 75 | 06188 | FZC15 | FZC | YSB646 |
| 76 | | | | YSB647 |
| 77 | 05170 | PIP2 | FZC | YSB1249 |
| 78 | | | | YSB1250 |
| 79 | 02241 | HOB5 | HOM | YSB1585 |
| 80 | | | | YSB1586 |
| 81 | 06921 | HOB4 | HOM | YSB1435 |
| 82 | | | | YSB1437 |
| 83 | 05186 | GRF1 | GRF | YSB796 |
| 84 | | | | YSB797 |
| 85 | 00039 | ZFC6 | C2Z | YSB1953 |
| 86 | | | | YSB1954 |
| 87 | 01454 | STE12 | C2Z | YSB1542 |
| 88 | | | | YSB1543 |
| 89 | 03527 | HEL2 | C2Z | YSB1382 |
| 90 | | | | YSB1383 |
| 91 | 05255 | FZC2 | FZC | YSB1050 |
| 92 | | | | YSB1051 |
| 93 | 01883 | GAT8 | GAT | YSB471 |
| 94 | | | | YSB472 |
| 95 | 04353 | CLR1 | C2Z | YSB1396 |
| 96 | | | | YSB1397 |
| 97 | 03998 | RLM1 | SRF | YSB1300 |
| 98 | | | | YSB1301 |
| 99 | 00239 | YAP1 | BZP | YSB815 |
| 100 | | | | YSB1290 |
| 101 | 06871 | FZC41 | FZC | YSB1334 |
| 102 | | | | YSB1335 |
| 103 | 00156 | SP1(CRZ1) | C2Z | YSB1263 |
| 104 | | | | YSB1264 |
| 105 | 04268 | APN2 | GRF | YSB1429 |
| 106 | | | | YSB1430 |
| 107 | 05420 | USV101 | C2Z | YSB1464 |
| 108 | | | | YSB1465 |
| 109 | 00018 | FZC6 | FZC | YSB1980 |
| 110 | | | | YSB1981 |
| 111 | 03346 | BZP4 | BZP | YSB1894 |
| 112 | | | | YSB1895 |
| 113 | 05538 | JJJ1 | C2Z | YSB1532 |
| 114 | | | | YSB1594 |
| 115 | 03409 | SKN7 | HSF | YSB349 |
| 116 | | | | YSB350 |
| 117 | 06339 | FZC35 | FZC | YSB1341 |
| 118 | | | | YSB1342 |
| 119 | 07506 | FAP1 | NFX | YSB813 |
| 120 | | | | YSB817 |
| 121 | 04807 | FZC8 | FZC | YSB2112 |
| 122 | | | | YSB2113 |
| 123 | 02435 | BWC2 | GAT | YSB1839 |
| 124 | | | | YSB1840 |
| 125 | 02364 | FZC19 | FZC | YSB2115 |
| 126 | | | | YSB2116 |
| 127 | 03116 | HCM1 | FKH | YSB1850 |
| 128 | | | | YSB1851 |
| 129 | 02877 | FZC51 | FZC | YSB1842 |
| 130 | | | | YSB1843 |
| 131 | 00559 | BZP3 | BZP | YSB1099 |
| 132 | | | | YSB1100 |
| 133 | 03914 | FZC14 | FZC | YSB1846 |
| 134 | | | | YSB1847 |
| 135 | 00871 | CLR3 | BZP | YSB1834 |
| 136 | | | | YSB1836 |
| 137 | 06483 | FZC25 | FZC | YSB518 |
| 138 | | | | YSB1822 |
| 139 | 07797 | CRL6 | FKH | YSB1106 |
| 140 | | | | YSB1107 |
| 141 | 05019 | FZC21 | FZC | YSB1252 |
| 142 | | | | YSB1253 |
| 143 | 05380 | FZC44 | FZC | YSB2182 |
| 144 | | | | YSB2183 |
| 145 | 04518 | ZFC5 | C2Z | YSB2177 |
| 146 | | | | YSB2178 |
| 147 | 05176 | HOB3 | HOM | YSB2001 |
| 148 | | | | YSB2002 |
| 149 | 05861 | FKH101 | FKH | YSB1855 |
| 150 | | | | YSB1856 |
| 151 | 00460 | LIV1 | HLH | YSB2211 |
| 152 | | | | YSB2212 |
| 153 | 02305 | FZC45 | FZC | YSB2221 |
| 154 | | | | YSB2222 |
| 155 | 03849 | ASG1 | FZC | YSB3013 |
| 156 | | | | YSB3014 |
| 157 | 01014 | ZFC4 | C2Z | YSB2231 |
| 158 | | | | YSB2232 |
| 159 | 01858 | HOB2 | HOM | YSB2282 |
| 160 | | | | YSB2283 |
| 161 | 06719 | FZC49 | FZC | YSB2171 |
| 162 | | | | YSB2173 |
| 163 | 04093 | YRM103 | FZC | YSB2298 |
| 164 | | | | YSB2299 |
| 165 | 04176 | HSF2 | HSF | YSB2295 |
| 166 | | | | YSB2296 |
| 167 | 01431 | HOB1 | HOM | YSB2308 |
| 168 | | | | YSB2309 |
| 169 | 07443 | HLH4 | HOM | YSB2244 |
| 170 | | | | YSB2245 |
| 171 | 04916 | FZC16 | FZC | YSB2326 |
| 172 | | | | YSB2327 |
| 173 | 05392 | ZAP104 | C2Z | YSB2134 |
| 174 | | | | YSB2135 |
| 175 | 02322 | FZC17 | FZC | YSB2250 |
| 176 | | | | YSB2251 |
| 177 | 04012 | FZC18 | FZC | YSB2320 |
| 178 | | | | YSB2321 |
| 179 | 00505 | FZC28 | FZC | YSB2337 |
| 180 | | | | YSB2338 |
| 181 | 06751 | HLH3 | HLH | YSB2329 |
| 182 | | | | YSB2330 |
| 183 | 04841 | FZC43 | FZC | YSB517 |
| 184 | | | | YSB2334 |
| 185 | 03212 | HCM101 | FKH | YSB2390 |
| 186 | | | | YSB2391 |
| 187 | 01626 | ADA2 | MYB | YSB2381 |
| 188 | | | | YSB2382 |
| 189 | 03894 | PDR802 | FZC | YSB2387 |
| 190 | | | | YSB2388 |
| 191 | 02066 | FZC13 | FZC | YSB2517 |
| 192 | | | | YSB2518 |
| 193 | 06818 | HAP1 | FZC | YSB2481 |
| 194 | | | | YSB2482 |
| 195 | 05940 | ZFC3 | C2Z | YSB2108 |
| 196 | | | | YSB2386 |
| 197 | 01948 | FZC36 | FZC | YSB2335 |
| 198 | | | | YSB2523 |
| 199 | 04804 | SRE1 | HLH | YSB2493 |
| 200 | | | | YSB2494 |
| 201 | 04352 | ZAP103 | C2Z | YSB2540 |
| 202 | | | | YSB2541 |
| 203 | 03768 | FZC32 | FZC | YSB2385 |
| 204 | | | | YSB2526 |
| 205 | 04895 | FZC3 | FZC | YSB2611 |
| 206 | | | | YSB2664 |
| 207 | 04583 | DDT1 | DDT | YSB1583 |
| 208 | | | | YSB2633 |
| 209 | 01973 | ZFC2 | C2Z | YSB2622 |
| 210 | | | | YSB2623 |
| 211 | 06156 | FZC7 | FZC | YSB2704 |
| 212 | | | | YSB2705 |
| 213 | 03431 | FZC48 | FZC | YSB2646 |
| 214 | | | | YSB2647 |
| 215 | 07922 | FZC4 | FZC | YSB2724 |
| 216 | | | | YSB2725 |
| 217 | 00031 | MLR1 | FZC | YSB2727 |
| 218 | | | | YSB2728 |
| 219 | 03018 | ASG101 | FZC | YSB2697 |
| 220 | | | | YSB2698 |
| 221 | 04263 | BZP2 | BZP | YSB2702 |
| 222 | | | | YSB2703 |
| 223 | 01708 | GAT7 | GAT | YSB2699 |
| 224 | | | | YSB2700 |
| 225 | 00332 | SIP4 | FZC | YSB2680 |
| 226 | | | | YSB2681 |
| 227 | 07724 | CUF1 | CDB | YSB2665 |
| 228 | | | | YSB2666 |
| 229 | 03902 | RDS2 | FZC | YSB1898 |
| 230 | | | | YSB1899 |
| 231 | 03366 | ZNF2 | C2Z | YSB2740 |
| 232 | | | | YSB2741 |
| 233 | 05222 | NRG1 | C2Z | YSB3096 |
| 234 | | | | YSB3097 |
| 235 | 05049 | PIP201 | FZC | YSB3099 |
| 236 | | | | YSB3100 |
| 237 | 02516 | HLH5 | HLH | YSB2609 |
| 238 | | | | YSB3059 |
| 239 | 04774 | FZC26 | FZC | YSB3084 |
| 240 | | | | YSB3085 |
| 241 | 03336 | FZC50 | FZC | YSB3131 |
| 242 | | | | YSB3132 |
| 243 | 03086 | FZC20 | FZC | YSB3128 |
| 244 | | | | YSB3129 |
| 245 | 03229 | YOX101 | HOM | YSB3134 |
| 246 | | | | YSB3136 |
| 247 | 01841 | GLN3 | GAT | YSB3154 |
| 248 | | | | YSB3155 |
| 249 | 02476 | YRM101 | FZC | YSB2997 |
| 250 | | | | YSB2998 |
| 251 | 05153 | GAT5 | GAT | YSB3033 |
| 252 | | | | YSB3034 |
| 253 | 02700 | ZFC8 | C2Z | YSB3031 |
| 254 | | | | YSB3032 |
| 255 | 04586 | HOB7 | HOM | YSB3026 |
| 256 | | | | YSB3027 |
| 257 | 02723 | FZC23 | FZC | YSB3105 |
| 258 | | | | YSB3106 |
| 259 | 03741 | FZC31 | FZC | YSB3093 |
| 260 | | | | YSB3094 |
| 261 | 04457 | FZC30 | FZC | YSB2447 |
| 262 | | | | YSB2448 |
| 263 | 04836 | FZC10 | FZC | YSB3083 |
| 264 | | | | YSB3368 |
| 265 | 06223 | MIZ1 | MIZ | YSB2133 |
| 266 | | | | YSB3366 |
| 267 | 01551 | GAT201 | GAT | YSB3300 |
| 268 | | | | YSB3301 |
| 269 | 04908 | CLR4 | BZP | YSB3282 |
| 270 | | | | YSB3283 |
| 271 | 07940 | BZP5 | BZP | YSB1474 |
| 272 | | | | YSB1475 |
| 273 | 05112 | FZC42 | FZC | YSB687 |
| 274 | | | | YSB690 |
| 275 | 00193 | GAT1 | GAT | YSB2972 |
| 276 | | | | YSB2973 |
| 277 | 06814 | SXI1alpha | HOM | YSB1390 |
| 278 | | | | YSB1391 |
| 279 | | | | YSB1392 |
| 280 | 00896 | FZC34 | FZC | YSB501 |
| 281 | | | | YSB2979 |
| 282 | 07411 | RUM1 | PHZ | YSB3164 |
| 283 | | | | YSB3747 |
| 284 | 00830 | ZC38 | FZC | YSB777 |
| 285 | | | | YSB3791 |
| 286 | 01059 | FZC11 | FZC | YSB845 |
| 287 | | | | YSB846 |
| 288 | | | | YSB2983 |
| 289 | 03132 | FZC5 | FZC | YSB1400 |
| 290 | | | | YSB1401 |
| 291 | | | | YSB1404 |
| 292 | 00068 | MET32 | C2Z | YSB1178 |
| 293 | | | | YSB1179 |
| 294 | | | | YSB1180 |
| 295 | 01173 | PAN1 | P53 | YSB1181 |
| 296 | | | | YSB1182 |
| 297 | | | | YSB1183 |
| 298 | 00514 | GAT6 | GAT | YSB1384 |
| 299 | | | | YSB1385 |
| 300 | | | | YSB1386 |
| 301 | 07011 | FZC22 | FZC | YSB1688 |
| 302 | | | | YSB1689 |
| 303 | | | | YSB2974 |
| 304 | 02603 | ZFC1 | C2Z | YSB2573 |
| 305 | | | | YSB2574 |
| 306 | | | | YSB2575 |
| 307 | 04036 | HSF3 | HSF | YSB2527 |
| 308 | | | | YSB2528 |
| 309 | | | | YSB2529 |
| 310 | 03059 | FZC9 | FZC | YSB2984 |
| 311 | | | | YSB3266 |
| 312 | | | | YSB3267 |
| 313 | 06283 | LIV4 | MYB | YSB2089 |
| 314 | | | | YSB3755 |
| 315 | | | | YSB3756 |
| 316 | 00841 | FZC40 | FZC | YSB3088 |
| 317 | | | | YSB3758 |
| 318 | 01977 | FZC39 | FZC | YSB1820 |
| 319 | | | | YSB2621 |
| 320 | 05375 | HLH2 | HLH | YSB1147 |
| 321 | | | | YSB1148 |
| 322 | | | | YSB1149 |

For parallel in vitro and in vivo phenotypic analysis, the present inventors deleted 53 TF genes, which were previously deleted in the CMO18 strain (a less virulent H99 strain, Non-Patent Document 12), and derived more than two independent mutants. Certain known transcription factors, including RIM101, ADA2, CUF1, SXL1, SP-1/CRZ1, NRG1, STE12, BWC2, SRE1, ZNF2 and HAP1/HAP2, were also independently deleted here to accurately compare phenotypes. When two independent transcription factor mutants showed inconsistent phenotypes, additional transcription factor mutants were generated to exclude outlier mutants. The present inventors found that about 8% of gene knockouts (13 transcription factors) exhibited inconsistent phenotypes, potentially attributable to unexpected alterations in the genome. This level (7%) was highly similar to that reported in a similar study on the ascomycete fungal pathogen Candida albicans (Non-Patent Document 13). For the remaining 23 transcription factors, transcription factor mutants were not generated. In summary, the present inventors constructed a Cryptococcus neoformans transcription factor mutant collection that covers 155 transcription factors and 322 transcription factor mutant strains in total.

Out of the 156 transcription factors whose mutants were constructed, 58 transcription factor genes possess names designated in published studies or reserved by other researchers through registration in FungiDB (www.fungidb.org). For the remaining 98 transcription factors, the present inventors provided gene names by following the systematic genetic nomenclature flowchart in Cryptococcus neoformans recently reported (Non-Patent Document 14).

1.2: Construction of Transcription Factor Mutants

Cryptococcus neoformans transcription factor knockout mutants (hereinafter referred to as TFKO) were constructed in the C. neoformans serotype A H99S strain background. Gene-disruption cassettes containing the nourseothricin-resistance marker (NAT) and signature-tagged sequences were generated by overlap polymerase chain reaction (hereinafter referred to as PCR) or double-joint PCR strategies using the primer sets shown in the Sequence List and Table 2 (Non-Patent Document 15 and Non-Patent Document 16). In the overlap PCR process, the 5′- and 3′-flanking regions of the transcription factor genes were amplified by using primers L1 and R1 and primers L2 and R2 (see Table 1), respectively, together with H99 genomic DNA in the first round of PCR. Primers M13Fe (M13 forward extended) and M13Re (M13 reverse extended) were used for amplifying the dominant selectable marker (NAT) containing unique signature-tagged sequences. In the second round of PCR, the TF gene-disruption cassettes were generated by means of overlap PCR performed using primers L1 and R2 and the first-round PCR products as templates. In the double-joint PCR method, the 5′- and 3′-flanking regions of the transcription factor genes were amplified using, respectively, the primer pairs L1/L2 and R1/R2 with H99 genomic DNA in the first round of PCR. The 5′- and 3′-regions of NAT-split markers were amplified using primers M13Fe and NSL and primers M13Re and NSR, respectively, together with pNATSTM (obtained from Joeseph Heitman' Laboratory at Duke University), which harbored unique signature-tagged sequences. The amplified gene-disruption cassettes were combined with 600 μg of gold microcarrier beads (0.6 μm, Bio-Rad) and treated with 10 μL of 2.5M calcium chloride and 2 μL of 1M spermidine. The gold beads combined with the gene-disruption cassettes were introduced into the H99S strains (obtained from Joeseph Heitman' Laboratory at Duke University) using the biolistic transformation apparatus (Non-Patent Document 17). After 4 hours of culture required for recovery, the cells were scraped, transferred onto an yeast extract-peptone dextrose (hereinafter referred to as YPD) medium containing 100 μg/ml nourseothricin, and then incubated at 30° C. for 3 to 5 days. Stable nourseothricin-resistant transformants were screened by diagnostic PCR using the primer sets listed in Table 3 and the Sequence List.

All transcription factor mutant strains were deposited in the Korean Culture Collection of Microorganisms (KCCM) and the Center of Microbial Pathogenesis at Duke University in USA.

TABLE 3
Construction of primer sets for genes

| Gene name | Primer name | Detailed description of primers |
|---|---|---|
| FZC5 | L1 | CNAG_03132 5′ flanking region primer 1 |
| | L2 | CNAG_03132 5′ flanking region primer 2 |
| | R1 | CNAG_03132 3′ flanking region primer 1 |
| | R2 | CNAG_03132 3′ flanking region primer 2 |
| | SO1 | CNAG_03132 diagnostic screening |
| | | primer, pairing with B79 |
| | PO2 | CNAG_03132 Southern blot probe primer |
| | STM | NAT#5 STM primer |
| | STM common | STM common primer |

### Example 2: Phenotypic Profiling

For the 322 transcription factor mutants constructed, the present inventors performed a series of in vivo and in vitro phenotypic analyses for the phenotypic classes as follows: growth, differentiation, morphology, stress responses, antifungal drug resistance, virulence-factor production and in vivo virulence. This overall phenome data set is illustrated together with a color scale, and data for transcript levels of each transcription factor measured by RNA sequencing analyses under six distinct growth conditions were measured. Red and blue in a thermal map showed decrease and increase, respectively. Phenotype strengths (strong, intermediate and weak) are distinguished in gradients of red or blue. The phenotypic analysis revealed that about 93% of the transcription factor mutants (145/155) exhibited at least one discernable phenotype, suggesting a high functional coverage of this transcription factor mutant collection. 85% of the transcription factors (132/155) have not been functionally characterized before in Cryptococcus neoformans strains.

All of these phenome data are publicly available in the Cryptococcus neoformans transcription factor database (http://tf.cryptococcus.org).

2.1: Genotypic Analysis

The accuracy of the genotypes of the positive transformants was validated by means of Southern blot analysis. Cryptococcus genomic DNA was extracted using the CTAB (cetyl trimethyl ammonium bromide) method (Non-Patent Document 33). Isolated genomic DNA from each TFKO mutant was digested with the indicated restriction enzyme (see Table 4). The digested genomic DNAs were separated by 1% agarose gel electrophoresis. The agarose gel was transferred into the denatured buffer containing 0.5M NaOH and 1.5M NaCl and allowed to stand for 45 min. Next, the agarose gel was transferred into the neutralization buffer containing 1.5M NaCl and 0.5M Tris buffer adjusted with pH 8 and allowed to stand for 45 min. The digested genomic DNAs were transferred to the nylon membrane using 10×SSC (saline sodium citrate) buffer and fixed by 1,200 J/m² ultraviolet exposure. The membrane was hybridized with a gene-specific and radioactively labeled probe using modified church hybridization buffer (1 mM EDTA, 0.25M Na₂HPO₄, 1% hydrolysated casein, 7% SDS, 6% H₃PO₄). The membrane was washed for 15 minutes with washing buffer 1 (containing 2×SSC and 0.1% SDS) and washing buffer 2 (containing 1×SSC and 0.1% SDS). Next, the membrane was exposed to autography film for 1 day.

TABLE 4
List of transcription factor knockout mutants and restriction
enzymes used

| | H99 1D | Designated name | Restriction enzyme cut |
|---|---|---|---|
| 1 | 02566 | FKH2 | BamHI |
| 2 | 00791 | HLH1 | BglII |
| 3 | 01069 | FZC11 | SphI |
| 4 | 07464 | MBS1 | SphI |
| 5 | 03401 | GAT203 | HindIII |
| 6 | 04588 | ERT1 | EcoRV |
| 7 | 00828 | SIP401 | EcoRI |
| 8 | 03561 | FZC33 | EcoRV |
| 9 | 06762 | GAT204 | KpnI |
| 10 | 06276 | CEP3 | SphI |
| 11 | 05785 | STB4 | BamHI |
| 12 | 03132 | FZC5 | PstI |
| 13 | 01438 | MBS2 | EcoRI |
| 14 | 07593 | YAP4 | SmaI |
| 15 | 04837 | LN1 | SmaI |
| 16 | 05093 | HOB6 | KpnI |
| 17 | 05642 | FZC37 | XmaI |
| 18 | 05431 | RIM101 | ClaI |
| 19 | 04398 | ARO80 | HincII |
| 20 | 04878 | FZC1 | EcoRI |
| 21 | 03183 | FZC24 | EcoRV |
| 22 | 03710 | ECM22 | HindIII |
| 23 | 03279 | CCD4 | HindIII |
| 24 | 04637 | MBF1 | HindIII |
| 25 | 06425 | PPR1 | EcoRV |
| 26 | 04345 | ARO8001 | BamHI |
| 27 | 04184 | FZC47 | BglII |
| 28 | 02774 | MAL13 | EcoRV |
| 29 | 00670 | FZC12 | StyI |
| 30 | 00068 | MET32 | AfeI |
| 31 | 05010 | ZFC7 | ScaI |
| 32 | 04090 | ATF1 | PstI |
| 33 | 06134 | RZP1(HXL1) | BamHI |
| 34 | 04630 | YAP2 | HindIII |
| 35 | 07901 | FZC29 | EcoRV |
| 36 | 01173 | PAN1 | SalI |
| 37 | 03115 | FZC46 | SacI |
| 38 | 07924 | MCM1 | XbaI |
| 39 | 07435 | HAP2 | SalI |
| 40 | 02555 | SIP402 | MfeI |
| 41 | 04594 | FZC27 | HindIII |
| 42 | 06188 | FZC15 | BamHI |
| 43 | 05170 | PIP2 | BamHI |
| 44 | 02241 | HOB5 | PstI |
| 45 | 06921 | HOB4 | KpnI |
| 46 | 05186 | GRF1 | EcoRI |
| 47 | 008913 | FZC34 | HindIII |
| 48 | 00039 | ZFC6 | KpnI |
| 49 | 07940 | BZP5 | BamHI |
| 50 | 06814 | SXI1alpha | SacI |
| 51 | 01454 | STE12 | BamHI |
| 52 | 03527 | HEL2 | BamHI |
| 53 | 05255 | FZC2 | HindIII |
| 54 | 05112 | FZC42 | BglII, EcoRV |
| 55 | 01883 | GAT8 | SacI |
| 56 | 04353 | CLR1 | SacI |
| 57 | 05375 | HLH2 | HindIII |
| 58 | 03998 | RLM1 | StyI |
| 59 | 00239 | YAP1 | BamHI |
| 60 | 06871 | FZC41 | BamHI, XbaI |
| 61 | 00156 | SP1(CRZ1) | EcoRV |
| 62 | 04268 | APN2 | EcoRV |
| 63 | 05420 | USV101 | SphI |
| 64 | 00018 | FZC6 | SphI |
| 65 | 03346 | BZP4 | PstI |
| 66 | 00514 | GAT6 | EcoRV |
| 67 | 05538 | JJJ1 | SphI |
| 68 | 03409 | SKN7 | BamHI |
| 69 | 06339 | FZC35 | HindIII |
| 70 | 07011 | FZC22 | PstI |
| 71 | 07506 | FAP1 | HindIII |
| 72 | 04807 | FZC8 | PstI |
| 73 | 02435 | BWC2 | HindIII |
| 74 | 02364 | FZC19 | BamHI |
| 75 | 03116 | HCM1 | SphI |
| 76 | 02877 | FZC51 | SphI |
| 77 | 00559 | BZP3 | SphI |
| 78 | 03914 | FZC14 | PstI |
| 79 | 00871 | CLR3 | XbaI |
| 80 | 06483 | FZC25 | PstI |
| 81 | 07797 | CRL6 | EcoRI |
| 82 | 05019 | FZC21 | PstI |
| 83 | 05380 | FZC44 | SacI |
| 84 | 04518 | ZFC5 | EcoRI |
| 85 | 05176 | HOB3 | XhoI |
| 86 | 05861 | FKH101 | EcoRV |
| 87 | 00460 | LIV1 | SphI |
| 88 | 02305 | FZC45 | EcoRV |
| 89 | 03849 | ASG1 | KpnI |
| 90 | 01014 | ZFC4 | SalI |
| 91 | 01858 | HOB2 | SphI |
| 92 | 06719 | FZC49 | PstI |
| 93 | 04093 | YRM103 | SphI |
| 94 | 04176 | HSF2 | KpnI |
| 95 | 01431 | HOB1 | HindIII |
| 96 | 07443 | HLH4 | SphI |
| 97 | 04916 | FZC16 | EcoRI |
| 98 | 05392 | ZAP104 | SalI |
| 99 | 02322 | FZC17 | XbaI |
| 100 | 04012 | FZC18 | HindIII |
| 101 | 00506 | FZC28 | SmaI |
| 102 | 06751 | HLH3 | SalI |
| 103 | 04841 | FZC43 | XbaI |
| 104 | 03212 | HCM101 | SacI, SalI |
| 105 | 01626 | ADA2 | BamHI |
| 106 | 03894 | PDR802 | SphI |
| 107 | 02066 | FZC13 | BamHI |
| 108 | 06818 | HAP1 | HindIII |
| 109 | 05940 | ZFC3 | EcoRI |
| 110 | 01948 | FZC36 | XhoI |
| 111 | 04804 | SRE1 | BamHI |
| 112 | 04352 | ZAP103 | SacI, SalI |
| 113 | 03768 | FZC32 | BamHI |
| 114 | 01977 | FZC39 | KpnI |
| 115 | 04895 | FZC3 | KpnI |
| 116 | 04583 | DDT1 | SphI |
| 117 | 01973 | ZFC2 | SphI |
| 118 | 02603 | ZFC1 | HindIII |
| 119 | 04036 | HSF3 | SphI |
| 120 | 06156 | FZC7 | EcoRI |
| 121 | 03431 | FZC48 | EcoRV |
| 122 | 07922 | FZC4 | EcoRV |
| 123 | 00031 | MLR1 | PstI, XmaI |
| 124 | 03018 | ASG101 | XhoI |
| 125 | 04263 | BZP2 | EcoRV |
| 126 | 01708 | GAT7 | SacI |
| 127 | 00332 | SIP4 | PstI |
| 128 | 07724 | CUF1 | KpnI |
| 129 | 03902 | RDS2 | EcoRV, EcoRI |
| 130 | 03366 | ZNF2 | EcoRV |
| 131 | 05222 | NRG1 | EcoRV |
| 132 | 05049 | PIP201 | BamHI |
| 133 | 02516 | HLH5 | BamHI |
| 134 | 04774 | FZC26 | Pst1 |
| 135 | 03059 | FZC9 | EcoRV |
| 136 | 00193 | GAT1 | SphI |
| 137 | 03336 | FZC50 | HindIII |
| 138 | 03086 | FZC20 | KpnI |
| 139 | 03229 | YOX101 | XbaI |
| 140 | 01841 | GLN3 | EcoRI |
| 141 | 02476 | YRM101 | XhoI |
| 142 | 05153 | GAT5 | SalI |
| 143 | 02700 | ZFC8 | EcoRV |
| 144 | 4586 | HOB7 | SphI |
| 145 | 2723 | FZC23 | EcoRV |
| 146 | 3741 | FZC31 | C1aI |
| 147 | 4457 | FZC30 | BamHI |
| 148 | 6283 | LIV4 | EcoRV |
| 149 | 7411 | RUM1 | EcoRV |
| 150 | 4836 | FZC10 | EcoRV |
| 151 | 841 | FZC40 | EcoRI |
| 152 | 6223 | MIZ1 | EcoRV |
| 153 | 830 | FZC38 | SphI |
| 154 | 1551 | GAT201 | XbaI |
| 155 | 4908 | CLR4 | HindIII |

2.2: Analysis of Gene Expression

Gene expression was analyzed by Northern blot analysis. Total RNA was extracted from each sample using Trizol reagent. 10 μg of the RNA was separated in 1% agarose gel made with DEPC (diethyl pyrocarbonate)-treated water and 1×MOPS (3-(N-morpholino)propane sulfonic acid) running buffer by electrophoresis. The gel was washed three times with distilled water, transferred to a nylon membrane (Millipore, INYC00010) using 20×SSC buffer, and fixed by 1200 J/m² ultraviolet exposure. The membrane was hybridized with a gene-specific and radioactively labeled probe using modified church hybridization buffer (1 mM EDTA; Biosesang Co., Ltd,. E1002), 0.25M Na₂HPO₄ (Sigma, S9763), 1% N-2-Amine (Sigma, C0626), 7% SDS (Bioshop, SDS001) and 0.17% H₃PO₄ (Sigma, #438081)). The membrane was washed with washing buffer 1 (2×SSC and 0.1% SDS) and washing buffer 2 (1× SSC and 0.1% SDS). Next, the membrane was exposed to autography film for 1 to 2 days.

### Example 3: Transcription Factors (TFs) Governing Growth

C. neoformans undergoes both saprobic and pathogenic life cycles in natural and animal host environments. Therefore, it must be capable of growing at temperatures ranging from ambient temperature (25° C.) to high temperature (37 to 39° C.). In order to analyze the growth phenotypes of the transcription factor mutants at various temperatures, the growth of each mutant on yeast extract-peptone dextrose (YPD) medium [yeast extract (Becton, Dickison and company #212750), peptone (Becton, Dickison and company #211677), glucose (Duchefa, #G0802)] at various temperatures (25° C., 30° C., 37° C. and 39° C.) was observed. Deletion of some transcription factors (BZP2, CUF1, LIV4, GAT5, FZC6 and NRG1) resulted in temperature-independent growth defects. The growth defect of the cuf1 mutant was due to its inability to uptake copper, because external addition of CuSO₄ restored its wild-type (WT) growth.

In the present invention, the growth-defect transcription factor mutants (24 mutants) were classified into two groups: (1) temperature-independent growth-defect transcription factor mutants; and (2) temperature-dependent growth-defect transcription factors. The first transcription factor group (temperature-independent) includes BZP2, CUF1, HOB1, GAT5, FZC6 and NRG1. Deletion of the transcription factors of the second group, including HXL1, CRX1, ATF1, ADA2, LIV4, AR080, USV101, FZC31, MLN1, FZC30, FZC1, MIZ1, FZC46, APN2, GAT6, MBS2, SRE1, and ERT1, caused growth defects only at high temperature (37 to 39° C.). Among these, only HXL1, which is a transcription factor downstream of the Irel kinase in the UPR signaling pathway, exhibited a severe growth defect at host physiological temperature. By contrast, deletion of MLN1, MCM1 and FZC46 promoted the growth of C. neoformans at 39° C. Collectively, these results suggest that multiple transcription factors (27 transcription factors) control—both positively and negatively—the growth and thermotolerance of C. neoformans.

### Example 4: Transcription Factors Governing Mating

In a natural environment, C. neoformans exists mainly in the yeast form but undergoes either bisexual differentiation with cells of the opposite mating type or unisexual differentiation with cells of the same mating type to produce filamentous forms and generate infectious basidiospores. These developmental processes contribute to the generation of the genetic diversity of the pathogen (Non-Patent Document 17).

To analyze mating phenotypes, the present inventors set up unilateral mating crosses by co-culturing each TF mutant (the serotype A MAT strain) with serotype A MATa wild-type KN99a strain (obtained from Joeseph Heitman's laboratory at Duke University). Each strain was cultured in YPD medium at 30° C. for 16 hours, and equal concentration of cells (10⁷ cells per ml) were mixed, spotted onto V8 mating media (pH 5; per 1 L: 50 ml V8 juice (Campbell), 0.5 g KH₂PO₄ (Bioshop, PPM302), 40 g Agar (Bioshop, AGRO01.500)) and incubated in a dark at room temperature for 1 to 2 weeks. Filamentous growth was monitored weekly and photographed using an Olympus BX51 microscope equipped with a SPOT Insight digital camera (Diagnostic Instrument Inc.).

To monitor the expression of pheromone gene, cell fusion assay and Northern blot analysis were performed. For the cell fusion assay, each MAT transcription factor mutant or control strain (YSB119; obtained from Joeseph Heitman's Laboratory at Duke University) containing NAT^{R} marker and MATa control strain (YSB121; obtained from Joeseph Heitman's Laboratory at Duke University) containing neomycin-resistant (Neon) marker were cultured at 30° C. in liquid YPD medium for 16 hours, and the concentration of cells was adjusted to 10⁷ cells per ml with distilled water. Each MAT strain and MATa strain were mixed in an equal volume, spotted onto V8 medium and incubated in a dark at room temperature for 24 hours. Then, the cells were scraped, resuspended in 1 ml distilled water and spread onto YPD medium containing both nourseothricin (100 μg/ml) and G418 (50 μg/ml, Geneticin, Life technologies). The plates were further incubated at 30° C. and the number of colonies on each plate was determined. In monitoring of pheromone gene expression, the MAT and KN99a strains were mixed with equal concentration of cells (10⁸ cells per ml), spread onto the V8 medium and incubated in the dark at room temperature for 18 to 24 hours. Then, cells were scraped from the V8 medium, pelleted at 4° C., frozen in liquid nitrogen, and lyophilized overnight. Total RNA was isolated using Trizol reagent according to the protocol described in the prior art document. The RNA was electrophoresed, and then transferred to a membrane. The membrane was hybridized with a mating pheromone gene (MF1)-specific probe amplified with primer B1894 (5′-TTTTACGCTTTTTGCAGATTCCGCCAAA-3′), B195 (5′-GACCACTGTTTCTTTCGTTCT-3′) and JEC21 genomic DNA (genomic DNA extracted from the JEC21 strain).

To analyze mating phenotypes, the present inventors set up unilateral mating crosses by coculturing each transcription factor mutant with serotype A MATa KN99a strain. The novel mating-regulating transcription factors in the present invention, deletion of BZP2, USV101, FZC1 and ZAP104 severely reduced mating, even in unilateral matings, whereas deletion of HLH1, HAP2 and GAT1 highly enhanced mating efficiency. To determine the mating steps in which these transcription factors are involved, the present inventors measured the efficiency of cell fusion and pheromone production, which precede the filamentation step. The bzp2, usv101, fzc1 and zap104 mutants lacked the ability to engage in cell fusion with the MATa control strain and also failed to induce pheromone gene (MF1) expression upon mating. Such results suggest that Bzp2, Usv101, Fzc1 and Zap104 transcription factors promote pheromone gene expression, which results in a subsequent increase in cell fusion. Conversely, in the hlh1, hap2 and gat1 mutants, cell-fusion efficiency was increased two- to three-fold, and pheromone gene expression was highly enhanced. SKN7, whose deletion promoted mating, was dispensable for both pheromone gene expression and cell fusion, indicating that it is likely involved in a later stage of mating. Analysis performed according to the present invention suggested that 34 transcription factors are involved in mating.

### Example 5: Transcription Factors Modulating Virulence-Factor Production

To support survival and proliferation within the host, C. neoformans is armed with several virulence factors, which include capsule and melanin. Capsule is a glucuronoxylomannan- or galactoxylomannan-based polysaccharide that protects cells from being phagocytosed by host phagocytic cells (Non-Patent Document 18). Melanin, a black-brown pigment made of polyphenol complexes, confers both antiphagocytic and antioxidant activity to cells (Non-Patent Document 19).

5.1: Capsule Production

Capsule production was measured in both qualitative and quantitative manners as described in the prior art documents (Non-Patent Document 20 and Non-Patent Document 21). Cells were grown at 30° C. in liquid YPD medium for 16 hours, spotted onto Dulbecco's Modified Eagle's (DME) solid medium and incubated at 37° C. for 2 days. Then, the cells were scraped from DME solid medium and washed with PBS (phosphate buffered saline). For qualitative measurement, capsules were stained by India ink (Bactidrop; Remel), and visualized using an Olympus BX51 microscope equipped with a Spot insight digital camera (Diagnostic Instrument Inc.). For quantitative measurement, the cells collected from DME solid medium were fixed with 10% formalin, and an equal number of cells (2.5×10⁷ cells per ml) were loaded into a haematocrit capillary tube, which was subsequently placed vertically to allow the cells to be packed by gravity for 10 days. The packed cell volume ratio was measured by calculating the ratio of the length of the packed cell volume phase to the length of the total volume phase (cells+medium). The relative packed cell volume of each mutant was measured by calculating the ratio of the mutant packed cell volume ratio to the wild-type packed cell volume ratio.

Triplicate technical experiments with two or more independent strains were performed. Statistical difference in relative packed cell volume was determined by one-way analysis of variance with Bonferroni's multiple-comparison test using Prism 6 (GraphPad software).

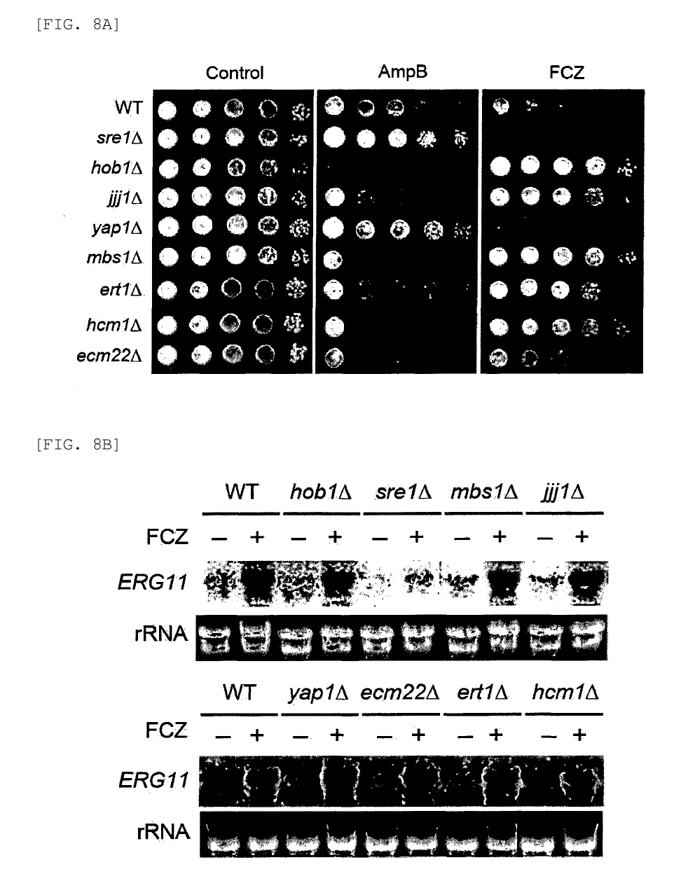

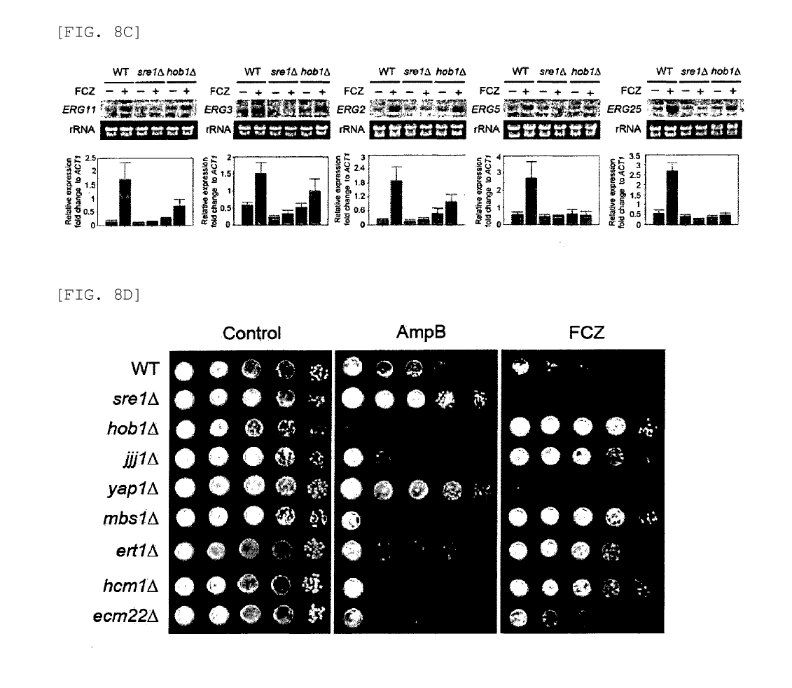

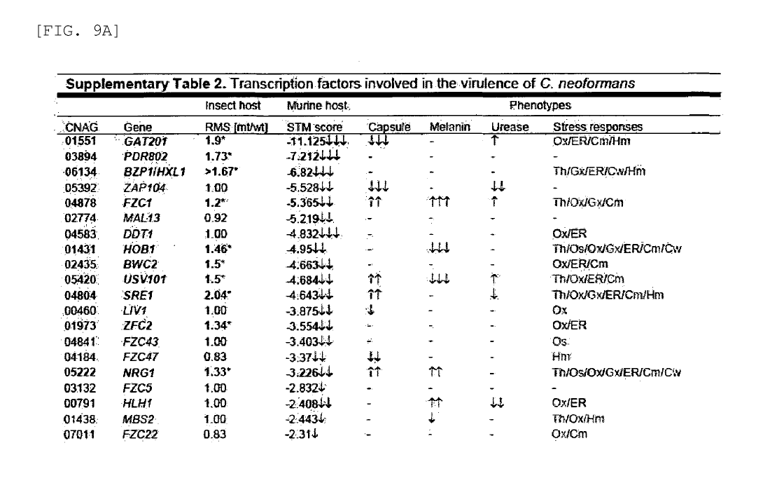

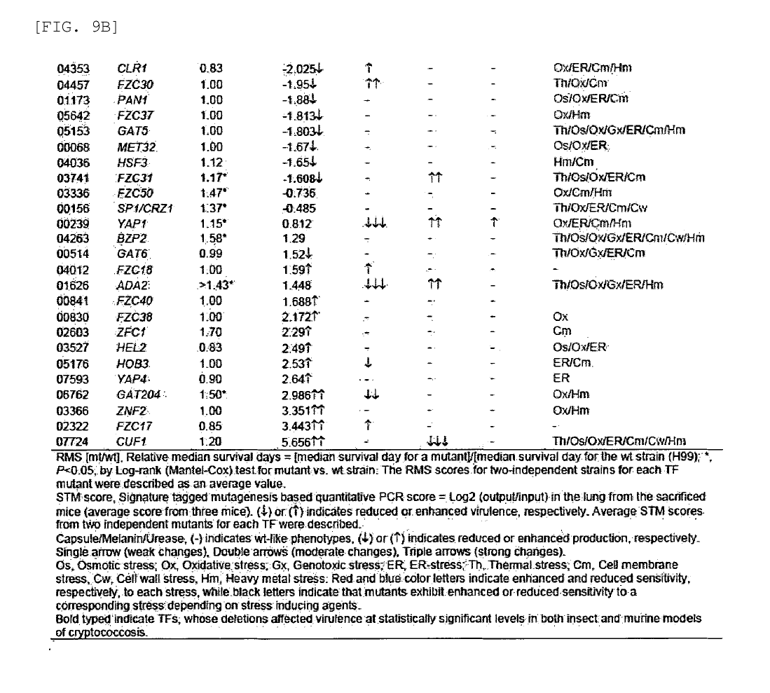

In the present invention, it was found that 49 transcription factors are involved in capsule production (FIG. 8A; 8B—29 negative regulators, 8C—20 positive regulators). Such transcription factors include previously reported capsule-regulating transcription factors, such as Atf1 (Non-Patent Document 10), Mbs1 (Non-Patent Document 11), Gat201 (Non-Patent Document 12) and Ada2 (Non-Patent Document 22). In addition to such capsule-regulating transcription factors, the present inventors identified several novel capsule-regulating transcription factors. The zap104Δ, bap1Δ and rds2Δ mutants also exhibited severely reduced capsule production compared to the results observed in the previously reported gat201Δ and ada2Δ mutants. Thus, Ada2, Gat201, Zap104, Bap1 and Rds2 together with 15 other positive regulators were predicted as major positive regulators in capsule production. By contrast, deletion of HOB7, CLR3 and FZC51 greatly enhanced capsule production, suggesting that these transcription factors together 26 other negative regulators are major negative regulators in capsule production.

5.2: Measurement of Melanin Production and Analysis of LAC1 Expression

Each transcription factor mutant strain and wild-type strain were cultured in liquid YPD medium at 30° C. overnight, spotted on Niger seed agar medium containing 0.1% or 0.3% glucose, and then cultured at 37° C., and the plates were photographed daily to determine melanin production. The present inventors uncovered 27 transcription factors (11 positive regulators and 16 negative regulators) involved in melanin production. A few of transcription factors, including Cuf1, Stel2, Mbs1, Skn7 and Atf1, are previously reported transcription factors (Non-Patent Document 10, Non-Patent Document 11, Non-Patent Document 23, Non-Patent Document 24 and Non-Patent Document 25). In addition to such results, the fzc8Δ, hob1Δ and bzp4Δ mutants exhibited greatly reduced melanin production; the reduction was similar to that of the cuf1Δ mutant.

In addition, in the present invention, the correlation between transcription factors and the expression of LAC1, which is the major laccase involved in melanin synthesis, was examined. First, Northern blot analysis was performed to monitor the induction of LAC1. Each wild-type and each transcription mutant were cultured in YPD medium at 30° C. for 16 hours. The cultured cells were adjusted to an OD₆₀₀ of 0.15 (optical density at 600 nm) and inoculated into 150 ml of fresh YPD liquid medium. The cell culture was further cultured at 30° C. until it reached at an OD₆₀₀ of about 0.6. To prepare the zero-time sample, 50 ml of 150 ml cell culture was sampled and 100 ml of the remaining culture was pelleted by centrifugation. After removal of the supernatant, the cells were re-suspended in glucose-free YNB liquid medium (Becton, Dickison and company, #291940). During culture, 50 ml of the cell culture was sampled at 1 hr and 2 hr. Total RNA was extracted from each sample, amplified by PCR using H99 genomic DNA, primer B3662 (5′-CTTTCAATCGTCCAAGCG-3′) and primer B3663 (5′-CCCCAGTTATCCAAAAAGTC-3′), and subjected to Northern blot analysis using an LAC1-specific probe. As a result, the present inventors found that Hob1 and Fzc8 promote the expression of LAC1, which is the major laccase involved in melanin synthesis (Non-Patent Document 26), under glucose-starvation conditions, whereas Bzp4 and Cuf1 are not directly involved in LAC1 expression. By contrast, deletion of HLH1, HLH2, BAP1 and FZC1 greatly enhanced melanin production, although only Hlh1 negatively regulated LAC1 expression. Therefore, the present inventors identified novel positive regulators (Hob1 and Fzc45) and negative regulator (Hlh1) of LAC1 in Cryptococcus neoformans.

5.3: Urease Production

In addition to capsule and melanin, crucial factors involved in the virulence of Cryptococcus neoformans include urease, a nickel-dependent protein complex (Ure1, Ure4, Ure6 and Ure7) that converts urea into ammonia, which serves as a nitrogen source.

Each wild-type strain and transcription factor mutant strain were cultured at 30° C. in liquid YPD medium for 16 hours (overnight) and washed with distilled water, and then the cell density was adjusted to 1×10⁷ cells per ml. Next, 5 μl of the cells (5×10⁴ cells) were spotted onto Christensen's agar medium. Then, the cells were incubated for 7 to 10 days at 30° C. and photographed during incubation.

The present inventors found that 19 transcription factors are involved in either positively (15 transcription factors) or negatively (4 transcription factors) regulating urease production.

### Example 6: Transcription Factors Affecting Infectivity and Virulence of C. neoformans

Identification of transcription factors required for the pathogenicity of C. neoformans is critical for future development of novel antifungal drugs and therapeutic methods. The present inventors employed two large-scale assays: (1) a virulence assay conducted in the invertebrate insect larval model system Galleria mellonella; and (2) a signature-tagged mutagenesis (STM)-based infectivity assay conducted in a murine inhalation model. These assays using one insect host and one mammalian host model have been widely adopted for large-scale virulence/infectivity assays in other fungi as well as C. neoformans (Non-Patent Document 12).

6.1: Insect-Based Virulence Assay

Insect-based virulence assay was performed using a modification of the previously known method (Non-Patent Document 21). For the insect-based virulence assay, 15 Galleria mellonella caterpillars (body weight: 250±50 mg) in the final instar larval stage, reached within 7 days from the day of shipment (Vanderhorst Inc., St Marys, Ohio, USA), were randomly sorted into each group. Each C. neoformans strain was grown overnight at 30° C. in YPD medium, washed three times, re-suspended with PBS, and used in hematocyte calculation performed using a hemocytometer. The present inventors inoculated 4,000 C. neoformans cells per larva through the second to last prolegs of larvae using a 100-μl Hamilton syringe equipped with a 10-μl-size needle and a repeating dispenser (PB600-1, Hamilton). As a non-infection control, PBS was injected. After injection, larvae were incubated in Petri dishes in humidified plastic containers and monitored daily. Infected larvae were incubated at 37° C. and monitored daily. Larvae were considered dead when they displayed no movement when touched. Larvae that transformed into pupae during experiments were censored for statistical analysis. Survival curves were prepared using Prism 6 (GraphPad) and statistically analyzed using the Log-rank (Mantel-Cox) test. The present inventors first monitored the survival curve for a single mutant strain for each transcription factor gene (total 155) and statistically compared it with that of the wild-type strain. In the case of transcription factor mutant that showed statistically significant reduction or enhancement of virulence (P<0.05; Log-rank test) by gene deletion, the present inventors examined a second independent strain.

Each panel indicates virulence assay results for two independent mutants of each transcription factor. The identified mutants include nine transcription factor mutants (hxl1, ada2, sre1, nrg1, bwc2, crz1, pdr802, gat201 and gat204) that were previously reported to show reduced virulence in a murine model of systemic cryptococcosis (Non-Patent Document 9, Non-Patent Document 12, Non-Patent Document 22, Non-Patent Document 27, Non-Patent Document 28, Non-Patent Document 29, Non-Patent Document 30, Non-Patent Document and Non-Patent Document 32). This further indicated a strong correlation between the insect and murine models in terms of the pathogenicity of C. neoformans. Besides the deletion of these known TFs, deletion of HOB1, BZP2, USV101, BAP1, ZFC2, FZC1, FZC50 and FZC31 significantly reduced the virulence of C. neoformans. The present inventors identified 17 transcription factor genes involved in the virulence of Cryptococcus neoformans using the insect host model.

6.2: Animal Study

Animal care and all experiments were conducted in accordance with the ethical guidelines of the Institutional Animal Care and Use Committee (IACUC) of Yonsei University. The Yonsei University IACUC approved all of the vertebrate studies.

In the signature-tagged mutagenesis (STM)-based mouse infectivity test, transcription factor strains tagged with 44 distinct signature tags (Table 1) were grown at 30° C. in YPD medium, washed three times with PBS and then pooled; the same number of cells of each strain were used after counting cells using a haemocytometer. The ste50 and ire1 mutants tagged with STM#282 and STM#169 sequences were used as virulent and non-virulent control strains, respectively (Non-Patent Document 9 and Non-Patent Document 33). Thus, the number of transcription factors (TFs) exhibiting the same signature tag is smaller than 4. In the present invention, four STM-based virulence tests were performed. To obtain the input TF genomic DNA library, the pooled TF mutants were 10-fold serially diluted, plated on YPD media, incubated at 30° C. for 3 days and collected by scraping for use in isolating genomic DNA. The output TF genomic DNA library was obtained as follows. Seven-week-old female A/Jcr mice (Jackson Laboratory) anaesthetized with intraperitoneal injection of Avertin (2,2,2-tribromoethanol) were infected through intranasal inhalation of 5×10⁵ cells (in 50 μl volume) of the pooled TF mutants and sacrificed with an overdose of Avertin at 15 days post infection. For each set of assays, the present inventors used five mice. Two independent mutants for each TF were tested in a separate STM set assay. Mouse lungs were dissected and homogenized in PBS. Each lung-tissue lysate was spread on YPD media containing 100 μg/ml of chloramphenicol, incubated at 30° C. for 3 days, and then collected by scraping to isolate output genomic DNA. Both input and output genomic DNAs were extracted using the CTAB method (Non-Patent Document 21). Quantitative PCR analysis was performed using various tag-specific primers listed in Table S1 and a MyiQ2 Real-Time PCR detection system (Bio-Rad). The STM score (Log₂[output/input] was calculated according to the method described in the prior art documents (Non-Patent Document 12 and Non-Patent Document 34).

6.3: Results of Animal Study

Using the STM-based murine host model, the present inventors identified 40 virulence genes. The STM score for each mutant was calculated based on the quantitative PCR score=Log₂ (output/input) in the lung from the sacrificed mice (average score from three mice). Among all the sets studied, the ire1Δ mutant, which is a non-virulent control strain, exhibited a highly reduced STM score (—7.03±1.99), whereas the ste50Δ mutant, a virulent control strain, showed an STM score of 0.11±1.13. For supporting the quality of the STM assay, 11 of the 40 transcription factor genes identified in the present invention were previously reported to be involved in virulence (Non-Patent Document 9, Non-Patent Document 12, Non-Patent Document 29 and Non-Patent Document 30). The gat201Δ and pdr802Δ mutants exhibited drastically reduced STM scores (−11.125 and −7.212, respectively) compared to those described in the prior art document (Non-Patent Document 12). Similarly, the STM scores of the zap104Δ and liv1Δ mutants were also decreased (−5.528 and −3.875, respectively). However, the zap103Δ mutant showed a very high virulence as described in the prior art document (Non-Patent Document 12) (STM score=2.51). Furthermore, the hxl1Δ, nrg1Δ and bwc2Δ mutants also showed highly reduced STM scores. 11 of the 40 transcription factors identified by the STM analysis (GAT201, PDR802, HXL1, BWC2, NRG1, FZC1, HOB1, USV101, ZFC2, SRE1 and FZC31) were also discovered using the insect model. The virulence assay data from the insect model were statistically significantly correlated with the STM-based infectivity data from the murine model based on the Pearson correlation coefficient (PCC) analysis. Among the 26 novel virulence-related transcription factors that were screened using only the STM-based murine model, the fzc31Δ and ddt1Δ mutants exhibited highly reduced STM scores (−4.328 and −4.832, respectively). The phenome database according to the present invention revealed that the fzc31Δ mutant exhibited increased susceptibility to osmotic, oxidative and cell membrane stresses, which might collectively affect virulence. By contrast, the only notable phenotype observed in the case of the ddt1Δ mutant was a weak dithiothreitol (DTT) sensitivity, which is not likely responsible for the marked decrease in the survival of the mutant in the lung because several other DTT-sensitive TF mutants were as virulent as the WT strain.

### Example 7: Examination of Antifungal Drug Resistance and Susceptibility

For the treatment of cryptococcosis, amphotericin B (AmpB) with or without flucytosine (5-FC) and fluconazole (FCZ) are widely used (Non-Patent Document 2). However, in addition to the toxic side effects of such drugs, the emergence of antifungal drug-resistant Cryptococcus strains has caused serious clinical problems (Non-Patent Document 35). To identify any transcription factors involved in antifungal drug resistance, the present inventors monitored the alteration of antifungal drug susceptibility among the C. neoformans TFKO mutant strains.

Cells were grown at 30° C. in liquid YPD medium for 16 hours, 10-fold serially diluted (1 to 10⁴ dilutions), and spotted on YPD medium containing the indicated concentrations of the following antifungal drugs: fludiooxonil, fluconazole, amphotericin B, and flucytosine. The cells were incubated at 30° C. and photographed for 2 to 5 days.

Numerous transcription factors were found to be involved in antifungal drug resistance, implying that Cryptococcus strains can potentially adapt to antifungal drugs in versatile manners. In response to FCZ, 35.5% of transcription factor mutants (55/155) exhibited either increased susceptibility (35 transcription factors) or resistance (20 transcription factors), suggesting that resistance to the azole-based antifungal drug could readily occur through the modulation of diverse transcription factors. However, in response to amphotericin B (AmpB), mutants of 56 genes exhibited differential susceptibility, with 47 transcription factor mutants showing increased susceptibility and only 8 transcription factor mutants exhibiting increased drug resistance. These data support the clinical observation that compared with azole resistance, polyene resistance is rarely observed. Furthermore, it is known that the 5-FC readily elicits the development of drug-resistant strains (Non-Patent Document 36). Supporting such results, the present inventors found that 27 transcription factors differentially regulate flucytosine resistance.

The present inventors noted that the deletion of some transcription factors negatively regulated azole and polyene susceptibility (Table 5 and FIG. 8A), possibly because these transcription factors might directly control ERG11 expression and sterol biosynthesis and affect polyene-binding capacity. Two of these transcription factors were previously reported to be Erg11 regulators. Sre1, a key sterol regulatory transcription factor, forms a complex with Scp1 as a part of the sterol regulatory element-binding protein pathway in C. neoformans (Non-Patent Document 27 and Non-Patent Document 28). Mbs1 negatively regulates basal ERG11 expression and therefore its deletion increases azole resistance but decreases polyene resistance in C. neoformans (Non-Patent Document 11). To further test whether other transcription factors are also involved in ERG11 regulation, the present inventors measured ERG11 expression levels in these TF mutants under both sterol-replete and sterol-depleted conditions (FIG. 8B). To analyze the expression of ERG2, ERG3, ERG5, ERG11 and ERG25, the wild-type, sre1 and hob1 mutants were grown in liquid YPD medium overnight. The overnight culture was then inoculated in 100 ml of fresh YPD medium at 30° C. and grown until the OD₆₀₀ of the culture reached about 1.0. To prepare the zero-time sample, 50 ml of cell culture was sampled, and the remaining culture was treated with fluconazole (final concentration: 10 μg/ml) for 90 min. Total RNA was isolated using TRIzol reagent, and cDNA was synthesized using M-MuLV reverse transcriptase (Thermo scientific). Northern blot analysis was performed with each ERG gene-specific probe that was amplified with ERG gene-specific primers with the total RNA in cells treated or not treated with fluconazole. Primers: B5789 (5′-CAAGAAATGGAGCGTGAG-3′) and B5790 (5′-CAGTGTTGTAAAGCGTGATG-3′) for ERG2; B1720 (5′-ATCCCTTTTCACCGTCGCTC-3′) and B1721 (5′-CGTCGTGGATGAGAATAGTCC-3′) for ERG3; B671 (5′-GTTTGTTGCCTGAGAACTGGG-3′) and B674 (5′-ATCACTCAACTCGGTCCTCTCGTG-3′) for ERG5; B678 (5′-TTCAGGGAACTTGGGAACAGC-3′) and B1598 (5′-CAGGAGCAGAAACAAAGC-3′) for ERG11; B1718 (5′-TGACCGCCTGTAGATTGTC-3′) and B1719 (5′-TAGTCCCACCACCTGAAAC-3′) for ERG25. Quantitative real-time PCR was performed with each gene-specific primer using a MyiQ2 Real-Time PCR detection system (Bio-Rad). Primers: B5789 (5′-CAAGAAATGGAGCGTGAG-3′) and B6838 (5′-GGGAGGGTCGAGGATGTAGA-3′) for ERG2; B1720 (5′-ATCCCTTTTCACCGTCGCTC-3′) and B6838 (5′-GGGAGGGTCGAGGATGTAGA-3′) for ERG3; B671 (5′-GTTTGTTGCCTGAGAACTGGG-3′) and B672 (5′-GTAGATACTGAGAGCCTGCTTGGTG-3′) for ERG5; B677 (5′-AATCTCCTTACCAGCCATTCGG-3′) and B678 (5′-TTCAGGGAACTTGGGAACAGC-3′) for ERG11; B2695 (5′-TCGTCTTTGGCAAGCAGTC-3′) and B6840 ((5′-GAAGTCGTGGTGGTCAGCA-3′) for ERG25; and B679 (5′-CGCCCTTGCTCCTTCTTCTATG-3′) and B680 (5′-TACTCGTCGTATTCGCTCTTCG-3′) for ACT1. As expected, basal and induced ERG11 levels were substantially lower in the sre1 mutant than in the wild-type strain. Notably, deleting HOB1 markedly increased the basal expression levels of ERG11. To determine whether Hob1 is involved in the regulation of other ERG genes, the present inventors monitored the expression of ERG2, ERG3, ERG5 and ERG25 in the wild-type strain, hob1Δ and sre1Δ strains under sterol-replete and -depleted conditions. The expression of all of these ERG genes was induced in response to sterol depletion through fluconazole treatment in the wild-strain strain, but not in the sre1Δ strain (FIG. 8C). Deletion of HOB1 markedly induced the basal expression of ERG2 (FIG. 8C). By contrast, under sterol depletion, the induction of ERG2, ERG3, ERG5, ERG11 and ERG25 was decreased in the hob1Δ mutant (FIG. 8C). The tight regulation of ERG expression appeared to be mostly absent in the hob1Δ mutant, indicating that Hob1 is a key regulator of ergosterol gene expression (FIG. 8D). Notably, the transcription factors involved in sterol biosynthesis also appeared to be involved in environmental stress responses and adaptation, which are critical for the survival and proliferation of C. neoformans within the host because the pathogen encounters drastic environmental changes during infection (Non-Patent Document 3).

TABLE 5
Transcription factors involved in antifungal agent resistance
in C. neoformans

| | TF mutants showing | TF mutants showing increased |
|---|---|---|
| Antifungal agents | increased resistance | susceptibility |
| Azole | HOB1, HAP2, SKN7, NRG1, | BZP3, HLH3, BZP1/HXL1, SRE1, |
| (Fluconazole) | MBS1, PPR1, JJJ1, HCM1, | RIM101, YAP2, HLH1, YAP4, |
| | ADA2, FZC9, GAT7, ERT1, | PIP2, MIZ1, MLN1, HOB6, MBF1, |
| | FKH2, ECM22, DDT1, GAT5, | MET32, FZC46, YAP1, FZC14, |
| | YRM103, CUF1, FZC51, | FZC2, HSF2, ZFC6, FZC45, |
| | LIV4, | FZC30, ASG1, STE12, LIV1, |
| | | FZC22, FZC31, PAN1, BZP2, |
| | | SP1/CRZ1, BZP5, SXI1alpha, |
| | | FZC34,, FZC17, HLH2 |
| Polyene | SRE1, YAP1, FZC51, SKN7, | HOB1, MBS1, JJJ1, ERT1, |
| (Amphotericin B) | CLR1, BZP4, ATF1, FZC4 | ECM22, GAT201, ZAP104, |
| | | SP1/CRZ1, FZC6, BZP5, HLH1, |
| | | PIP2, HCM1, BZP2, USV101, |
| | | HOB4, STE12, HOB5, GRF1, |
| | | HEL2, FZC45, ASG1, FZC22, |
| | | HOB6, PAN1, CUF1, FZC49, FZC1, |
| | | BWC2, FAP1, FZC44, FZC8, |
| | | FZC23, GAT204, NRG1, PIP201, |
| | | RIM101, HLH3, BZP3, MLN1, |
| | | MET32, ZFC2, FZC31, RUM1, |
| | | PDR802, FZC10, HLH2 |
| 5-flucyotosine | HLH3, RIM101, GAT204, | NRG1, ZFC2, YAP1, MBS1, FZC6, |
| | HOB3, FZC50, ZNF2, RDS2, | YAP2, BZP3, JJJ1, HLH1, PIP2, |
| | FZC31 | APN2, FZC46, HAP2, FZC51, |
| | | BZP5, HCM1, FZC19, BZP2, |
| | | FZC44 |
| Phenylpyrrole | NRG1, JJJ1, SP1/CRZ1, | USV101, ADA2, YAP1, FZC6, |
| Fungicide | SKN7, GAT7, FAP1, ZFC2, | HLH1, PIP2, FZC46, HAP2, |
| (Fludioxonil) | GAT204, ZNF2, HEL2, | BZP1/HXL1, FKH2, LIV1, YAP2, |
| | FZC50, SRE1 | BZP2, FZC21, HLH3, YRM101, |
| | | BZP5, GLN3, ZFC8, DDT1, |
| | | FZC22, HOB6, RLM1, MLN1, |
| | | PAN1, FZC35, YRM103, ZFC3, |
| | | ASG1, FZC41, FZC43, FZC51, |
| | | HAP1, MET32, FZC32 |

### Example 8: Examination of Responses to External Stress

To analyze external stress-related phenotypes, cells were grown at 30° C. in liquid YPD medium for 16 hours, 10-fold serially diluted (1 to 10⁴ dilutions), and spotted on YPD medium containing the indicated concentrations of the following stress inducers: A: osmotic stress (sorbitol); B: oxidative stress [hydrogen peroxide (H₂O₂), tert-butyl hydroperoxide (TH) (an organic peroxide), menadione, diamide]; C: endoplasmic reticulum (ER) stress [tunicamycin), DTT (dithiothreitol)]; D: genotoxic stress [methyl methanesulfonate (MMS), hydroxyurea (HU)]; E: cell membrane/wall-destabilizing stress [calcofluor white (CFW), Congo red (CR), and sodium dodecyl sulfate (SDS)]; F: heavy-metal stress (CdSO₄). Cells were incubated at 30° C. and photographed for 2 to 5 days. The osmotic stress applied was a stress induced by any one selected from the group consisting of 1M to 1.5M sodium chloride (NaCl), 1M to 1.5M potassium chloride (KCl) and 2M sorbitol. It was shown that the antifungal agent-targeting genes against the osmotic stress induced by the sodium chloride were ada2, aro8001, bap1, bzp2, fzc13, fzc19, fzc34, fzc42, fzc43, fzc51, gat5, gat7, hap2, hcm1, hob1, hob6, met32, pan1, rim101 and skn7 genes, and the antifungal agent-targeting genes against the osmotic stress induced by the potassium chloride were ada2, bzp2, bzp4, fzc32, fzc35, fzc44, fzc6, hap2, hob1, hob2, nrg1, rim101 and yrm10. It was shown that the antifungal agent-targeting genes against the osmotic stress induced by sorbitol were bzp2, fzc6 and hob1.

The oxidative stress was examined by applying each of 2.5 mM to 3.5 mM hydrogen peroxide H₂O₂), 0.7 mM to 0.8 mM tert-butyl hydroperoxide (TH), 0.02 mM to 0.03 mM menadione, and 2 mM to 3 mM diamide (DA).

It was shown that antifungal agent-targeting genes against the oxidative stress induced by the hydrogen peroxide were ada2, bap1, bzp2, bzp3, cuf1, fzc13, fzc21, fzc22, fzc27, fzc31, fzc4, fzc46, fzc50, fzc9, gat1, gat201, gat204, gat5, hlh1, hob1, hob4, hob5, hob6, liv1, met32, miz1, nrg1, pan1, rim101, sip402, sp1(crz1), sre1, ste12 and usv101, and antifungal agent-targeting genes for the oxidative stress induced by the tert-butyl hydroperoxide were ada2, asg1, atf1, bap1, bap2, bzp5, clr1, ecm22, fzc1, fzc15, fzc21, fzc31, fzc34, fzc44, fzc49, fzc51, fzc6, gat5, gat8, grf1, hcm1, hlh2, hob1, hob2, hob4, liv1, met32, mwc2, pan1, ppr1, rim101, rlm1, skn7, sre1, usv101, yrm103, zap103, zfc2 and zfc4. It was shown that antifungal agent-targeting genes for the oxidative stress induced by the menadione were bap1, bzp2, ecm22, fzc26, fzc3, fzc34, fzc35, fzc37, fzc4, fzc44, fzc6, gat6, hel2, jjj1, nrg1 and usv101, and antifungal agent-targeting genes for the oxidative stress induced by the diamide were bap1, bap2, bzp2, bzp5, fap1, fkh2, fzc19, fzc21, fzc27, fzc3, fzc30, fzc31, fzc34, fzc38, fzc4, fzc46, fzc49, fzc8, gat5, gat6, hap2, hlh1, hlh3, hob1, hob6, hsf2, met32, miz1, mln1, pan1, pip2, rum1, sip402, sre1 and zfc2.

The endoplasmic reticulum (ER) stress was induced by each of 0.2 μg/ml to 0.3 μg/ml tunicamycin (TM) and 15 mM to 20 mM dithiothreitol (DTT), and as a result, it was shown that antifungal agent-targeting genes for the endoplasmic reticulum (ER) stress induced by the tunicamycin were bzp1(hxl1), bzp3, fzc2, fzc21, fzc38, fzc40, fzc44, gat7, hlh1, liv4, met32, mln1, pip2, rim101, rlm1, sp1(crz1), sre1 and ste12. In addition, it was shown that antifungal agent-targeting genes for the endoplasmic reticulum (ER) stress induced by the dithiothreitol were ada2, apn2, bzp1(hxl1), bzp2, clr1, cuf1, ddt1, fzc25, fzc31, gat201, gat5, hap2, hlh2, hob1, nrg1, rlm1, sre1 and usv101.

The genotoxic stress was induced by each of 0.03% to 0.06% methyl methanesulfonate (MMS) and 50 mM to 100 mM hydroxyurea (HU). As a result, it was shown that an antifungal agent-targeting gene for the genotoxic stress induced by the methyl methanesulfonate was any one gene selected from the group consisting of apn2, bzp1(hxl1), bzp2, fzc1, fzc38, fzc4, fzc40, fzc6, gat5, gat6, hcm101, hob1, jjj1, miz1 and sre1, and the antifungal agent-targeting genes against the genotoxic stress induced by the hydroxyurea were ada2, bzp1(hxl1), bzp2, fzc6, gat5, gat6, hcm1, hlh2, hob1, jjj1, mbs1, nrg1, skn7 and sre1.

The cell wall or cell membrane stress was induced by each of 3 mg/ml to 5 mg/mg calcofluor white (CFW), 0.8% to 1% Congo red (CR) and 0.03% sodium dodecyl sulfate (SDS). As a result, it was shown that an antifungal agent-targeting gene for the cell wall or cell membrane stress induced by the calcofluor white (CFW) was any one gene selected from the group consisting of bap2, bzp1(hxl1), bzp2, hap2, hob1, nrg1, pip2, rim101 and sp1(crz1), and antifungal agent-targeting genes for the cell wall or cell membrane stress induced by the Congo red were bap2, bzp1(hxl1), bzp2, cuf1, hap2, hlh3, hob1, nrg1, rim101 and sp1(crz1), and antifungal agent-targeting genes for the cell wall or cell membrane stress induced by the sodium dodecyl sulfate (SDS) were alpha, asg1, asg101, bap1, bzp2, bzp3, bzp5, clr1, clr4, cuf1, ecm22, ert1, fap1, fzc21, fzc26, fzc30, fzc31, fzc7, gat1, gat201, gat5, gat6, gat7, hap2, hob1, hob3, hob5, jjj1, nrg1, pan1, pip2, rds2, rlm1, rum1, sip4, sp1(crz1), sre1, sxi1, usv101, zfc4 and zfc6.

The heavy-metal stress was induced by 20 μM to 30 μM cadmium sulfate (CdSO₄), and as a result, it was shown that antifungal agent-targeting genes for the stress induced by the heavy-metal stress were aro80, aro8001, bzp2, bzp4, ccd4, cuf1, fap1, fzc10, fzc19, fzc35, fzc37, fzc46, fzc47, fzc51, fzc6, fzc8, gat5, gln3, hap2, hcm1, hob5, hob6, hob7, mbs2, mln1, pip2, pip201, rum1, skn7, yox101, yrm101 and zfc8.

Reflecting diverse types of external stresses, 145 transcription factors were identified to be involved in sensing and responding to at least one type of stress. Among these transcription factors, the two sterol regulators, Sre1 and Hob1, appeared to be general stress-responsive transcription factors that govern multiple stress responses and adaptations. Strikingly, the deletion of HOB1 or SRE1 substantially reduced resistance to osmotic/salt, oxidizing/reducing, genotoxic, endoplasmic reticulum (ER) and cell wall/membrane stresses. The hob1 and sre1 mutants exhibited similar stress resistance and susceptibility patterns under most of the various environmental stresses, and this is in stark contrast to their opposite resistance patterns towards fluconazole (FCZ) and amphotericin B (AmpB). This result strongly suggested that sterol homeostasis is a very important factor in controlling stress response and adaptation in C. neoformans.

### Example 9: Analysis of Functional Correlation

To understand the correlation among phenotypic traits and virulence, the present inventors measured the degree of linear dependence by calculating all Pearson Correlation Coefficients (PCCs) between two possible in vitro and in vivo phenotypic combinations tested in the present invention. These correlations are illustrated in a combined network. The correlation network revealed that the virulence of C. neoformans is highly correlated to growth at distinct temperatures and osmotic and cell wall/membrane-stress responses and moderately related to oxidative, genotoxic and ER stress responses. Notably, these stress phenotypes were also highly inter-correlated, suggesting that several core stress signaling networks might exist, including the known Hog1, Pkc1/Mpk1 and UPR signaling pathways. By contrast, mating and resistance to antifungal drugs, except to resistance to AmpB, were not significantly related to the virulence of C. neoformans. The production of virulence factors did not appear to be correlated to in vivo virulence, and this is likely because increased virulence-factor production often did not result in increased virulence. Supporting this, reduced capsule production was found to be highly correlated to reduced virulence by PCC analysis (P<0.05).

### Example 10: Construction of C. neoformans Transcription Factor Database

The genome and transcriptome data collected for 155 transcription factors were processed using the protocol of the standardized genome data in Comparative Fungal Genomics Platform (CFGP 2.0; Non-Patent Document 37). For detailed information of the predicted genes, pre-computed results of eight bioinformatics programs (InterPro scan, Signalp 3.0, PSortII, TargetP, ChloroP, SecretomeP, predictsNLS and TMHMM2) were provided (Non-Patent Document 38, Non-Patent Document 39, Non-Patent Document 40, Non-Patent Document 41, Non-Patent Document 42, Non-Patent Document 43, Non-Patent Document 44 and Non-Patent Document 45).

To browse genomics contexts together with key biological features, Seoul National University Genome Browser (SNUGB; Non-Patent Document 46) was incorporated for use with the Cryptococcus Transcription Factor Database. In the pages of Browse Scaffolds, Browse Gene Models and 3 gene-family browsers, direct links to the SNUGB module were provided. MySQL 5.0.81 (source code distribution) and PHP 5.2.6 were used for administrating the database and developing web interfaces, respectively. Web pages were provided through Apache 2.2.9 web server.

### Example 11: Construction of Pearson's Correlation Networks

The present inventors calculated Pearson Correlation Coefficient (PCC) PCC scores by using Prism 5.0 (GraphPad Software Inc.) based on the results of phenotypic tests (strongly resistant phenotype: 3; moderately resistant phenotype: 2; weakly resistant phenotype: 1; wild type-like phenotype: 0; weakly sensitive phenotype: −1; moderately sensitive phenotype: −2; and strongly sensitive phenotype: −3). Networks were visualized using Cytoscape software 3.2.0 based on the PCC scores.

Accession Number

Name of Depositary Institution: Korean Culture Center of Microorganisms;

Accession Number: KCCM51291;

Date of Deposit: Mar. 23, 2015.

### INDUSTRIAL APPLICABILITY

The present invention relates to novel genes that regulate the virulence of Cryptococcus neoformans strains, and to the use thereof. According to the present invention, a novel antifungal agent and/or a novel agent for treating meningitis can be screened.

All the documents referred to in the present invention are incorporated herein by reference in its entirety.

### SEQUENCE LIST TEXT

General Deposition Number Notice for Microorganism
No. 2015-21
For the microorganism to which you applied for general
deposition under number 2015-21 on Mar. 27, 2015, the general
deposit was accepted, and the general deposit number of the
microorganism is notified as follows.
Follows

| | |
|---|---|
| 1. Name of microorganism | Cryptococcus neoformans var. |
| deposited | grubil H99S and transcription |
| | factor mutant library |
| 2. Name of microorganism at | |
| listing | |
| 3. Microorganism deposit number | KCCM 51291 |
| 4. Date of deposit | Mar. 23, 2015 |

⋄ The above microorganism can be distributed without restrictions to researchers both in Korea and abroad for academic and industrial purposes.

Korean Culture Center of Microorganisms

1. A method for screening an antifungal agent, comprising the steps of:
(a) bringing a sample to be analyzed into contact with a Cryptococcus neoformans cell containing an antifungal agent-targeting gene;
(b) measuring expression of the antifungal agent-targeting gene in the cell; and
(c) determining that the sample is an antifungal agent, when the expression of the antifungal agent-targeting gene is down-regulated, wherein the antifungal agent-targeting gene is any one gene selected from the group consisting of an antibacterial agent resistance regulatory gene, a growth regulatory gene, a mating regulatory gene, and a gene that regulates responses to external stress,
wherein the antibacterial agent resistance regulatory gene is a gene that regulates resistance to any one antifungal agent selected from the group consisting of azole-, polyene-, 5-flucytocin- and phenylpyrazole-based antifungal agents, wherein the gene that regulates resistance to the azole-based antifungal agent is any one gene selected from the group consisting of alpha, asg1, bap1, bap2, bzp/hxl1, bzp2, bzp3, bzp5, fzc14, fzcl7, fzc2, fzc22, fzc30, fzc31, fzc34, fzc38, fzc40, fzc45, fzc46, hlh1, hlh2, hlh3, hob6, hsf2, liv1, liv4, mbf1, met32, miz1, mln1, pan1, pip2, rim101, sp1/crz1, sre1, ste12, sxi1, yap4 and zfc6; the gene that regulates resistance to the polyene-based antifungal agent is any one gene selected from the group consisting of asg1, bwc2, bzp2, bzp3, bzp5, cuf1, ecm22, ert1, fap1, fzc1, fzc22, fzc23, fzc31, fzc38, fzc40, fzc44, fzc45, fzc49, fzc6, fzc8, gat201, gat204, grf1, hcm1, hel2, hlh1, hlh2, hlh3, hob1, hob4, hob5, hob6, jjj1, liv4, mbs1, met32, mln1, nrg1, pan1, pip2, pip201, rim101, rum1, sp1/crz1, ste12, usv101, zap104 and zfc2; the gene that regulates resistance to the 5-flucytocin-based antifungal agent is any one gene selected from the group consisting of apn2, bap1, bap2, bzp3, bzp5, fzc19, fzc46, fzc51, fzc6, hap2, hcm1, hlh1, jjj1, mbs1, nrg1, pip2 and zfc2; and the gene that regulates resistance to the phenylpyrazole-based antifungal agent is any one gene selected from the group consisting of ada2, asg1, bap1, bap2, bzp1/hxl1, bzp2, bzp5, ddt1, fkh2, fzc21, fzc22, fzc3, fzc32, fzc35, fzc38, fzc41, fzc43, fzc46, fzc51, fzc6, gln3, hap1, hap2, hlh1, hlh3, hob6, liv1, liv4, met32, mln1, pan1, pip2, rlm1, usv101, yrm101, yrm103 and zfc8,
the growth regulatory gene is temperature-independent or temperature-dependent, wherein the temperature-independent growth regulatory gene is any one gene selected from the group consisting of bzp2, cuf1, fzc6, gat5, hob1 and nrg1, and the temperature-dependent growth regulatory gene is a gene that regulates growth at a temperature of 37° C. to 39° C. and is any one gene selected from the group consisting of ada2, apn2, aro80, atf1, crz1, ert1, fzc1, fzc30, fzc31, gat6, hxl1, liv4, mbs2, miz1, mln1, sre1 and usv101,
the mating regulatory gene is any one gene selected from the group consisting of bzp2, fzc1, skn7, usv101 and zap104,
the gene that regulates responses to external stress is a gene that regulates responses to any one stress selected from the group consisting of osmotic stress, oxidative stress, endoplasmic reticulum stress, genotoxic stress, cell wall or cell membrane stress, and heavy-metal stress, wherein the osmotic stress is a stress induced by any one selected from the group consisting of 1M to 1.5M sodium chloride (NaCl), 1M to 1.5M potassium chloride (KCl) and 2M sorbitol, wherein the antifungal agent-targeting gene that regulates responses to the osmotic stress induced by the sodium chloride is any one gene selected from the group consisting of ada2, aro8001, bap1, bzp2, fzc13, fzc19, fzc34, fzc42, fzc43, fzc51, gat5, gat7, hap2, hcm1, hob1, hob6, met32, pan1, rim101 and skn7; the antifungal agent-targeting gene that regulates responses to the osmotic stress induced by the potassium chloride is any one gene selected from the group consisting of ada2, bzp2, bzp4, fzc32, fzc35, fzc44, fzc6, hap2, hob1, hob2, nrg1, rim101 and yrm103; and the antifungal agent-targeting gene that regulates responses to the osmotic stress induced by the sorbitol is any one gene selected from the group consisting of bzp2, fzc6 and hob1,
the oxidative stress is a stress induced by any one selected from the group consisting of 2.5 mM to 3.5 mM hydrogen peroxide (H₂O₂), 0.7 mM to 0.8 mM tert-butyl hydroperoxide (TH), 0.02 mM to 0.03 mM menadione, and 2 mM to 3 mM diamide (DA), the antifungal agent-targeting gene that regulates responses to the oxidative stress induced by the hydrogen peroxide is any one gene selected from the group consisting of ada2, bap1, bzp2, bzp3, cuf1, fzc13, fzc21, fzc22, fzc27, fzc31, fzc4, fzc46, fzc50, fzc9, gat1, gat201, gat204, gat5, hlh1, hob1, hob4, hob5, hob6, liv1, met32, miz1, nrg1, pan1, rim101, sip402, sp1(crz1), sre1, ste12 and usv101; the antifungal agent-targeting gene that regulates responses to the oxidative stress induced by the tert-butyl hydroperoxide is any one gene selected from the group consisting of ada2, asg1, atf1, bap1, bap2, bzp5, clr1, ecm22, fzc1, fzc15, fzc21, fzc31, fzc34, fzc44, fzc49, fzc51, fzc6, gat5, gat8, grf1, hcm1, hlh2, hob1, hob2, hob4, liv1, met32, mwc2, pan1, ppr1, rim101, rlm1, skn7, sre1, usv101, yrm103, zap103, zfc2 and zfc4; the antifungal agent-targeting gene that regulates responses to the oxidative stress induced by the menadione is any one gene selected from the group consisting of bap1, bzp2, ecm22, fzc26, fzc3, fzc34, fzc35, fzc37, fzc4, fzc44, fzc6, gat6, hel2, jjj1, nrg1 and usv101, and the antifungal agent-targeting gene that regulates responses to the oxidative stress induced by the diamide is any one gene selected from the group consisting of bap1, bap2, bzp2, bzp5, fap1, fkh2, fzc19, fzc21, fzc27, fzc3, fzc30, fzc31, fzc34, fzc38, fzc4, fzc46, fzc49, fzc8, gat5, gat6, bap2, hlh1, hlh3, hob1, hob6, hsf2, met32, miz1, mln1, pan1, pip2, rum1, sip402, sre1 and zfc2,
the endoplasmic reticulum stress is a stress induced by 0.2 μg/ml to 0.3 μg/ml tunicamycin and 15 mM to 20 mM dithiothreitol (DTT), wherein the antifungal agent-targeting gene that regulates responses to the endoplasmic reticulum stress induced by the tunicamycin is any one gene selected from the group consisting of bzp1(hxl1), bzp3, fzc2, fzc21, fzc38, fzc40, fzc44, gat7, hlh1, liv4, met32, mln1, pip2, rim101, rlm1, sp1(crz1), sre1 and ste12; and the antifungal agent-targeting gene that regulates responses to the endoplasmic reticulum stress induced by the dithiothreitol is any one gene selected from the group consisting of ada2, apn2, bzp1(hxl1), bzp2, clr1, cuf1, ddt1, fzc25, fzc31, gat201, gat5, hap2, hlh2, hob1, nrg1, rlm1, sre1 and usv101,
the genotoxic stress is a stress induced by any one selected from the group consisting of 0.03% to 0.06% methyl methanesulfonate (MMS) and 50 mM to 100 mM hydroxyurea (HU), wherein the antifungal agent-targeting gene that regulates responses to the genotoxic stress induced by the methyl methanesulfonate is any one gene selected from the group consisting of apn2, bzp1(hxl1), bzp2, fzc1, fzc38, fzc4, fzc40, fzc6, gat5, gat6, hcm101, hob1, jjj1, miz1 and sre1, and the antifungal agent-targeting gene that regulates responses to the genotoxic stress induced by the hydroxyurea is any one gene selected from the group consisting of ada2, bzp1(hxl1), bzp2, fzc6, gat5, gat6, hcm1, hlh2, hob1, jjj1, mbs1, nrg1, skn7 and sre1,
the cell wall or cell membrane stress is a stress induced by any one selected from the group consisting of 3 mg/ml to 5 mg/ml calcofluor white (CFW), 0.8% to 1% Congo red (CR) and 0.03% sodium dodecyl sulfate (SDS), wherein the antifungal agent-targeting gene that regulates responses to the cell wall or cell membrane stress induced by the calcofluor white (CFW) is any one gene selected from the group consisting of bap2, bzp1(hxl1), bzp2, hap2, hob1, nrg1, pip2, rim101 and sp1(crz1); the antifungal agent-targeting gene that regulates responses to the cell wall or cell membrane stress induced by the Congo red (CR) is any one gene selected from the group consisting of bap2, bzp1(hxl1), bzp2, cuf1, hap2, hlh3, hob1, nrg1, rim101 and sp1(crz1); and the antifungal agent-targeting gene that regulates responses to the cell wall or cell membrane stress induced by the sodium dodecyl sulfate (SDS) is any one gene selected from the group consisting of alpha, asg1, asg101, bap1, bzp2, bzp3, bzp5, clr1, clr4, cuf1, ecm22, ert1, fap1, fzc21, fzc26, fzc30, fzc31, fzc7, gat1, gat201, gat5, gat6, gat7, hap2, hob1, hob3, hob5, jjj1, nrg1, pan1, pip2, rds2, rlm1, rum1, sip4, sp1(crz1), sre1, sxi1, usv101, zfc4 and zfc6, and
the heavy-metal stress is induced by 20 μM to 30 μM cadmium sulfate (CdSO₄), wherein the antifungal agent-targeting gene that regulates responses to the heavy-metal stress induced by 20 μM to 30 μM cadmium sulfate (CdSO₄) is any one gene selected from the group consisting of aro80, aro8001, bzp2, bzp4, ccd4, cuf1, fap1, fzc10, fzc19, fzc35, fzc37, fzc46, fzc47, fzc51, fzc6, fzc8, gat5, gln3, hap2, hcm1, hob5, hob6, hob7, mbs2, mln1, pip2, pip201, rum1, skn7, yox101, yrm101 and zfc8.

2. The method of claim 1, wherein the resistance is down-regulated.

3. A method for screening an antifungal agent, comprising the steps of:
(a) bringing a sample to be analyzed into contact with a Cryptococcus neoformans cell containing an antifungal agent-targeting gene;
(b) measuring expression of the antifungal agent-targeting gene in the cell; and
(c) determining that the sample is an antifungal agent, when the expression of the antifungal agent-targeting gene is up-regulated, wherein the antifungal agent-targeting gene is any one gene selected from the group consisting of an antibacterial agent resistance regulatory gene, a growth regulatory gene, a mating regulatory gene, and a gene that regulates responses to external stress,
wherein the antibacterial agent resistance regulatory gene is a gene that regulates resistance to any one antifungal agent selected from the group consisting of azole-, polyene-, 5-flucytocin- and phenylpyrazole-based antifungal agents, wherein the gene that regulates resistance to the azole-based antifungal agent is any one gene selected from the group consisting of ada2, cuf1, ddt1, ecm22, ert1, fkh2, fzc51, fzc9, gat1, gat7, hap2, hcm1, hob1, jjj1, mbs1, nrg1, ppr1, skn7 and yrm103; the gene that regulates resistance to the polyene-based antifungal agent is any one gene selected from the group consisting of atf1, bap1, bzp5, clr1, fzc4, fzc51, snk7 and sre1; the gene that regulates resistance to the 5-flucytocin-based antifungal agent is any one gene selected from the group consisting of fzc50, gat204, hlh3, hob3, rds2, rim101 and znf2; and the gene that regulates resistance to the phenylpyrazole-based antifungal agent is any one gene selected from the group consisting of fap1, fzc50, gat204, gat7, hel2, jjj1, nrg1, skn7, sp1/crz1, sre1, zfc2 and znf2,
the growth regulatory gene is any one of fzc46 and mini and regulates growth at 39° C.,
the mating regulatory gene is any one gene selected from the group consisting of gat1, hap2, hlh1 and skn7,
the antifungal agent-targeting gene that regulates responses to external stress is a gene that regulates responses to any one stress selected from the group consisting of osmotic stress, oxidative stress, endoplasmic reticulum stress, genotoxic stress, cell wall or cell membrane stress, and heavy-metal stress, wherein the osmotic stress is a stress induced by any one selected from the group consisting of 1M to 1.5M sodium chloride (NaCl), 1M to 1.5M potassium chloride (KCl) and 2M sorbitol, wherein the antifungal agent-targeting gene that regulates responses to the osmotic stress induced by the sodium chloride is any one gene selected from the group consisting of hlh3, hel2 and cuf1; the antifungal agent-targeting gene that regulates responses to the osmotic stress induced by the potassium chloride is any one gene of fzc36 and yrm103; and the antifungal agent-targeting gene that regulates responses to the osmotic stress induced by the sorbitol is fzc36,
the oxidative stress is a stress induced by any one selected from the group consisting of 2.5 mM to 3.5 mM hydrogen peroxide (H₂O₂), 0.7 mM to 0.8 mM tert-butyl hydroperoxide (TH), 0.02 mM to 0.03 mM menadione, and 2 mM to 3 mM diamide (DA), wherein the antifungal agent-targeting gene that regulates responses to the oxidative stress induced by the hydrogen peroxide is any one gene selected from the group consisting of asg101, bwc2, fzc35, fzc45, fzc7 and znf2; the antifungal agent-targeting gene that regulates responses to the oxidative stress induced by the tert-butyl hydroperoxide is any one gene selected from the group consisting of clr3, ddt1, fap1 and fzc33; the antifungal agent-targeting gene that regulates responses to the oxidative stress induced by the menadione is any one gene selected from the group consisting of cuf1, fzc50, hap2, sip4 and zfc2; and the antifungal agent-targeting gene that regulates responses to the oxidative stress induced by the diamide is any one gene selected from the group consisting of asg101, fzc20, fzc26, fzc50, gat1, gat7, hlh5, jjj1, nrg1, pip201, sip4, skn7 and znf2,
the endoplasmic reticulum stress is a stress induced by 0.2 μg/ml to 0.3 μg/ml tunicamycin and 15 mM to 20 mM dithiothreitol (DTT), wherein the antifungal agent-targeting gene that regulates responses to the endoplasmic reticulum stress induced by the tunicamycin is any one gene selected from the group consisting of bap1, bwc2, bzp2, clr1, clr4, cuf1, fzc6, gat5, gat6, hap2, hcm1, hel2, hob1, hob3, jjj1, mbs1, nrg1, ppr1, skn7, zfc2, zfc3 and zfc4, and the antifungal agent-targeting gene that regulates responses to the endoplasmic reticulum stress induced by the dithiothreitol is any one gene selected from the group consisting of bzp3, fkh2, fzc11, fzc20, gat1, gat203, hlh1, mbs1, met32, pan1, pip2, sip401, stb4 and yap4,
the genotoxic stress is a stress induced by any one of 0.03% to 0.06% methyl methanesulfonate (MMS) and 50 mM to 100 mM hydroxyurea (HU), wherein the antifungal agent-targeting gene that regulates responses to the genotoxic stress induced by the methyl methanesulfonate is yox1, and the antifungal agent-targeting gene that regulates responses to the genotoxic stress induced by the hydroxyurea is fzc20,
the cell wall or cell membrane stress is a stress induced by any one of 3 mg/ml to 5 mg/ml calcofluor white (CFW) and 0.03% sodium dodecyl sulfate (SDS), wherein the antifungal agent-targeting gene that regulates responses to the cell wall or cell membrane stress induced by the calcofluor white (CFW) is any one of fzc9 and grf1, and the antifungal agent-targeting gene that regulates responses to the cell wall or cell membrane stress induced by the sodium dodecyl sulfate is any one gene selected from the group consisting of bwc2, fzc1, fzc22, fzc50, fzc51, fzc6, fzc8, hsf3, skn7 and zfc1, and
the heavy-metal stress is induced by 20 to 30 cadmium sulfate (CdSO₄), wherein the antifungal agent-targeting gene that regulates responses to the heavy-metal stress induced by 20 μM to 30 μM cadmium sulfate (CdSO₄) is any one gene selected from the group consisting of ada2, asg101, atf1, bzp1(hxl1), clr1, fzc39, fzc7, gat201, gat 204, gat7, hlh2, hsf3, rds2, rlm1, sip4, sre1, zfc3 and znf2.

4. The method of claim 3, wherein the resistance is down-regulated.

5. The method of claim 1 or 3, wherein the cell in step (a) is any one cell selected from a cell line library deposited under accession number KCCM51291.

6. A method for screening an antifungal agent, comprising the steps of:
(a) bringing a sample to be analyzed into contact with a Cryptococcus neoformans cell containing a virulence regulatory gene;
(b) measuring expression of the virulence regulatory gene in the cell; and
(c) determining that the sample is an antifungal agent, when the expression of the virulence regulatory gene is down-regulated,
wherein the virulence regulatory gene comprises one or more genes selected from the group consisting of a gene that regulates cellular pathogenicity and a gene that regulates virulence-factor production,
wherein the gene that regulates virulence-factor production comprises one or more genes selected from the group consisting of a gene that regulates capsule production, a gene that regulates melanin production, and a gene that regulates urease production, and the gene that regulates cellular pathogenicity comprises one or more genes selected from the group consisting of aro80, bap1, bzp2, cef3, clr1, ddt1, fzc1, fzc12, fzc2, fzc22, fzc31, fzc33, fzc37, fzc43, fzc49, fzc5, fzc50, fzc9, gat5, hlh1, hob1, mal13, mbs2, pip2, rum1, usv101, zfc2 and zfc5.

7. The method of claim 6, wherein the gene that regulates capsule production comprises one or more genes selected from the group consisting of bap1, bzp4, fzc16, fzc33, fzc45, fzc47, gat204, hob3, hob4, hob5, hsf2, liv1, liv4, mcm1, rds2, zap104 and zfc4; the gene that regulates melanin production comprises one or more genes selected from the group consisting of bzp4, ert1, fzc25, fzc5, fzc8, hob1, liv1, mbs2 and usv101; and the gene that regulates urease production comprises one or more genes selected from the group consisting of zap104, sre1, gat201, fzc46, hlh1 and fzc21.

8. A method for screening an antifungal agent, comprising the steps of:
(a) bringing a sample to be analyzed into contact with a Cryptococcus neoformans cell containing a virulence regulatory gene;
(b) measuring expression of the virulence regulatory gene in the cell; and
(c) determining that the sample is an antifungal agent, when the expression of the virulence regulatory gene is up-regulated,
wherein the virulence regulatory gene comprises one or more genes selected from the group consisting of a gene that regulates cellular pathogenicity and a gene that regulates virulence-factor production,
wherein the gene that regulates virulence-factor production comprises one or more genes selected from the group consisting of a gene that regulates capsule production, a gene that regulates melanin production, and a gene that regulates urease production, and
the gene that regulates cellular pathogenicity comprises one or more genes selected from the group consisting of aro8001, ert1, fzcl7, fzc24, fzc38 and fzc40.

9. The method of claim 8, wherein the gene that regulates capsule production comprises one or more genes selected from the group consisting of bzp3, clr1, clr3, crl6, fkh2, fzc1, fzc14, fzcl7, fzc18, fzc24, fzc29, fzc30, fzc36, fzc46, fzc49, fzc51, hcm1, hlh3, hlh4, hob7, hpa1, jjj1, mln1, nrg1, sre1, usv101 and zfc3; the gene that regulates melanin production comprises one or more genes selected from the group consisting of ada2, bap1, bzp2, bzp3, fkh2, fzc1, fzc31, gat1, hlh1, hlh2, nrg1, rds2, sip4 and sip401; and the gene that regulates urease production comprises one or more genes selected from the group consisting of atf1, bap1, fkh2, fzc1, fzc14 fzc26, hob4, hob7, mln1, rim1, skn7, sxilalpha, usv101 and zfc7.

10. The method of claim 6 or 8, wherein the cell in step (a) is any one cell selected from a cell line library deposited under accession number KCCM51291.

11. A method for screening a meningitis-treating agent, comprising the steps of:
(a) bringing a sample to be analyzed into contact with a Cryptococcus neoformans cell containing a virulence regulatory gene;
(b) measuring expression of the virulence regulatory gene in the cell; and
(c) determining that the sample is a meningitis-treating agent, when the expression of the virulence regulatory gene is down-regulated,
wherein the virulence regulatory gene comprises one or more genes selected from the group consisting of a gene that regulates cellular pathogenicity and a gene that regulates virulence-factor production,
wherein the gene that regulates virulence-factor production comprises one or more genes selected from the group consisting of a gene that regulates capsule production, a gene that regulates melanin production, and a gene that regulates urease production, and
the gene that regulates cellular pathogenicity comprises one or more genes selected from the group consisting of aro80, bap1, bzp2, cef3, clr1, ddt1, fzc1, fzc12, fzc2, fzc22, fzc31, fzc33, fzc37, fzc43, fzc49, fzc5, fzc50, fzc9, gat5, hlh1, hob1, mal13, mbs2, pip2, rum1, usv101, zfc2 and zfc5.

12. The method of claim 11, wherein the gene that regulates capsule production comprises one or more genes selected from the group consisting of bap1, bzp4, fzc16, fzc33, fzc45, fzc47, gat204, hob3, hob4, hob5, hsf2, liv1, liv4, mcm1, rds2, zap104 and zfc4; the gene that regulates melanin production comprises one or more genes selected from the group consisting of bzp4, ert1, fzc25, fzc5, fzc8, hob1, liv1, mbs2 and usv101; and the gene that regulates urease production comprises one or more genes selected from the group consisting of fzc21, fzc46, gat201, hlh1, sre1 and zap104.

13. A method for screening a meningitis-treating agent, comprising the steps of:
(a) bringing a sample to be analyzed into contact with a Cryptococcus neoformans cell containing a virulence regulatory gene;
(b) measuring expression of the virulence regulatory gene in the cell; and
(c) determining that the sample is a meningitis-treating agent, when the expression of the virulence regulatory gene is up-regulated,
wherein the virulence regulatory gene comprises one or more genes selected from the group consisting of a gene that regulates cellular pathogenicity and a gene that regulates virulence-factor production,
wherein the gene that regulates virulence-factor production comprises one or more genes selected from the group consisting of a gene that regulates capsule production, a gene that regulates melanin production, and a gene that regulates urease production, and
the gene that regulates cellular pathogenicity comprises one or more genes selected from the group consisting of aro8001, ert1, fzcl7, fzc24, fzc38 and fzc40.

14. The method of claim 13, wherein the gene that regulates capsule production comprises one or more gene selected from the group consisting of bzp3, clr1, clr3, crl6, fkh2, fzc1, fzc14, fzcl7, fzc18, fzc24, fzc29, fzc30, fzc36, fzc46, fzc49, fzc51, hcm1, hlh3, hlh4, hob7, hpa1, jjj1, mln1, nrg1, sre1, usv101 and zfc3; the gene that regulates melanin production comprises one or more gene selected from the group consisting of ada2, bap1, bzp2, bzp3, fkh2, fzc1, fzc31, gat1, hlh1, hlh2, nrg1, rds2, sip4 and sip401; and the gene that regulates urease production comprises one or more gene selected from the group consisting of atf1, bap1, fkh2, fzc1, fzc14, fzc26, hob4, hob7, mln1, rim1, skn7, sxilalpha, usv101 and zfc7.

15. The method of claim 11 or 13, wherein the cell in step (a) is any one cell selected from a cell line library deposited under accession number KCCM51291.

16. A method for screening an antifungal agent for co-administration, comprising the step:
(a) bringing an antifungal agent into contact with a Cryptococcus neoformans cell including a virulence regulatory gene, and measuring expression level of the gene to obtain a first measurement value;
(b) bringing a sample to be analyzed and the antifungal agent into contact with a Cryptococcus neoformans cell including a virulence regulatory gene, and measuring expression level of the gene to obtain a second measurement value; and
(c) comparing the first measurement value with the second measurement value, and determining that the sample is an antifungal agent for co-administration, when the second measurement value is down-regulated compared to the second measurement value,
wherein the virulence regulatory gene comprises one or more genes selected from the group consisting of a gene that regulates cellular pathogenicity and a gene that regulates virulence-factor production,
wherein the gene that regulates virulence-factor production comprises one or more genes selected from the group consisting of a gene that regulates capsule production, a gene that regulates melanin production, and a gene that regulates urease production, and
the gene that regulates cellular pathogenicity comprises one or more genes selected from the group consisting of aro80, bap1, bzp2, cef3, clr1, ddt1, fzc1, fzc12, fzc2, fzc22, fzc31, fzc33, fzc37, fzc43, fzc49, fzc5, fzc50, fzc9, gat5, hlh1, hob1, mal13, mbs2, pip2, rum1, usv101, zfc2 and zfc5.

17. The method of claim 16, wherein the gene that regulates capsule production comprises one or more genes selected from the group consisting of bap1, bzp4, fzc16, fzc33, fzc45, fzc47, gat204, hob3, hob4, hob5, hsf2, liv1, liv4, mcm1, rds2, zap104 and zfc4; the gene that regulates melanin production comprises one or more genes selected from the group consisting of bzp4, ert1, fzc25, fzc5, fzc8, hob1, liv1, mbs2 and usv101; and the gene that regulates urease production comprises one or more genes selected from the group consisting of fzc21, fzc46, gat201, hlh1, sre1 and zap/04.

18. A method for screening an antifungal agent for co-administration, comprising the step:
(a) bringing an antifungal agent into contact with a Cryptococcus neoformans cell including a virulence regulatory gene, and measuring expression level of the gene to obtain a first measurement value;
(b) bringing a sample to be analyzed and the antifungal agent into contact with a Cryptococcus neoformans cell including a virulence regulatory gene, and measuring expression level of the gene to obtain a second measurement value; and
(c) comparing the first measurement value with the second measurement value, and determining that the sample is an antifungal agent for co-administration, when the second measurement value is up-regulated compared to the second measurement value,
wherein the virulence regulatory gene comprises one or more genes selected from the group consisting of a gene that regulates cellular pathogenicity and a gene that regulates virulence-factor production,
wherein the gene that regulates virulence-factor production comprises one or more genes selected from the group consisting of a gene that regulates capsule production, a gene that regulates melanin production, and a gene that regulates urease production, and
the gene that regulates cellular pathogenicity comprises one or more genes selected from the group consisting of aro8001, ert1, fzcl7, fzc24, fzc38 and fzc40.

19. The method of claim 18, wherein the gene that regulates capsule production comprise one or more genes selected from the group consisting of bzp3, clr1, clr3, crl6, fkh2, fzc1, fzc14, fzcl7, fzc18, fzc24, fzc29, fzc30, fzc36, fzc46, fzc49, fzc51, hcm1, hlh3, hlh4, hob7, hpa1, jjj1, mln1, nrg1, sre1, usv101 and zfc3; the gene that regulates melanin production comprise one or more genes selected from the group consisting of ada2, bap1, bzp2, bzp3, fkh2, fzc1, fzc31, gat1, hlh1, hlh2, nrg1, rds2, sip4 and sip401; and the gene that regulates urease production comprise one or more genes selected from the group consisting of atf1, bap1, fkh2, fzc1, fzc14, fzc26, hob4, hob7, mln1, rim1, skn7, sxilalpha, usv101 and zfc7.

20. The method of claim 16 or 18, wherein the cell in steps (a) and (b) is any one cell selected from a cell line library deposited under accession number KCCM51291.
